SEQUENCE LISTING (789 sequences)

SEQ ID NO: 1 - protein, 29 aa, synthetic construct
  REGION 1..29: note = Modified bacterial sequence
EVLEAWDEIDRLPNLTIEQWLAFINKLDD

SEQ ID NO: 2 - protein, 29 aa, synthetic construct
  REGION 1..29: note = Modified bacterial sequence
EVLEAWNEIDRLPNLTIEQWLAFINKLDD

SEQ ID NO: 3 - protein, 29 aa, synthetic construct
  REGION 1..29: note = Modified bacterial sequence
EVIEAWNEIDRLPNLTIEQWLAFINKLDD

SEQ ID NO: 4 - protein, 29 aa, synthetic construct
  REGION 1..29: note = Modified bacterial sequence
EVLEAWDEIDRLPNLTLDQWLAFINKLDD

SEQ ID NO: 5 - protein, 29 aa, synthetic construct
  REGION 1..29: note = Modified bacterial sequence
EVLDAWDEIDALPNLTLEQWLAFINKLDD

SEQ ID NO: 6 - protein, 29 aa, synthetic construct
  REGION 1..29: note = Modified bacterial sequence
EVIDAWDEIDRLPNLTLDQWLAFINKLDD

SEQ ID NO: 7 - protein, 29 aa, synthetic construct
  REGION 1..29: note = Modified bacterial sequence
ETLEAWDEIDRLPNLTIEQWLAFINKLDD

SEQ ID NO: 8 - protein, 29 aa, synthetic construct
  REGION 1..29: note = Modified bacterial sequence
EVIDAWNEIDALPNLTLDQWLAFINKLDD

SEQ ID NO: 9 - protein, 29 aa, synthetic construct
  REGION 1..29: note = Modified bacterial sequence
EVLDAWDEIDKLPNLTIDQWLAFINKLDD

SEQ ID NO: 10 - protein, 29 aa, synthetic construct
  REGION 1..29: note = Modified bacterial sequence
EVLEAWDEIDHLPNLTLDQWLAFINKLDD

SEQ ID NO: 11 - protein, 29 aa, synthetic construct
  REGION 1..29: note = Modified bacterial sequence
EVLEAWDEIDALPNLTIEQWLAFINKLDD

SEQ ID NO: 12 - protein, 29 aa, synthetic construct
  REGION 1..29: note = Modified bacterial sequence
EVLDAWDEIDKLPNLTLEQWLAFINKLDD

SEQ ID NO: 13 - protein, 29 aa, synthetic construct
  REGION 1..29: note = Modified bacterial sequence
ETITAWDEIDKLPNLTIEQWLAFIGKLED

SEQ ID NO: 14 - protein, 29 aa, synthetic construct
  REGION 1..29: note = Modified bacterial sequence
ESMKAWDEIDRLPNLNINQWVAFIDSLYD

SEQ ID NO: 15 - protein, 29 aa, synthetic construct
  REGION 1..29: note = Modified bacterial sequence
ESIEAWTEIDHLPNLTIEQWLAFINKLTD

SEQ ID NO: 16 - protein, 29 aa, synthetic construct
  REGION 1..29: note = Modified bacterial sequence
EVLDAWHEIDTLPNLTVRQWLAFISKLED

SEQ ID NO: 17 - protein, 29 aa, synthetic construct
  REGION 1..29: note = Modified bacterial sequence
EHIQANEEIDRLPNLTIKQWLAFINKLHD

SEQ ID NO: 18 - protein, 29 aa, synthetic construct
  REGION 1..29: note = Modified bacterial sequence
EVLHAWAEIDALPNLTIEQWLAFINKLDD

SEQ ID NO: 19 - protein, 29 aa, synthetic construct
  REGION 1..29: note = Modified bacterial sequence
EVLAAWDEIDSLPNLTLQQWLAFINKLDD

SEQ ID NO: 20 - protein, 29 aa, synthetic construct
  REGION 1..29: note = Modified bacterial sequence
EVIDAWNEIDALPNLTLEQWLAFINKLDD

SEQ ID NO: 21 - protein, 29 aa, synthetic construct
  REGION 1..29: note = Modified bacterial sequence
EVLDAWNEIDALPNLTIDQWLAFINKLSD

SEQ ID NO: 22 - protein, 29 aa, synthetic construct
  REGION 1..29: note = Modified bacterial sequence
EVIEAWDEIDGLPNLTIEQWLAFINKLDD

SEQ ID NO: 23 - protein, 29 aa, synthetic construct
  REGION 1..29: note = Modified bacterial sequence
EVLEAWDEIDHLPNLTLQQWLAFINKLDD

SEQ ID NO: 24 - protein, 29 aa, synthetic construct
  REGION 1..29: note = Modified bacterial sequence
EVIEAWNEIDALPNLTIEQWLAFINKLDD

SEQ ID NO: 25 - protein, 29 aa, synthetic construct
  REGION 1..29: note = Modified bacterial sequence
EVIAAWNEIDRLPNLTLTQWLAFINKLDD

SEQ ID NO: 26 - protein, 29 aa, synthetic construct
  REGION 1..29: note = Modified bacterial sequence
EVIEAWDEIDALPNLTLQQWLAFINKLDD

SEQ ID NO: 27 - protein, 29 aa, synthetic construct
  REGION 1..29: note = Modified bacterial sequence
EVIAAWDEIDKLPNLTIEQWLAFINKLDD

SEQ ID NO: 28 - protein, 29 aa, synthetic construct
  REGION 1..29: note = Modified bacterial sequence
EVIAAWDEIDKLPNLTLQQWLAFINKLDD

SEQ ID NO: 29 - protein, 29 aa, synthetic construct
  REGION 1..29: note = Modified bacterial sequence
ETIAAWDEIDKLPNLTIEQWLAFINKLDD

SEQ ID NO: 30 - protein, 29 aa, synthetic construct
  REGION 1..29: note = Modified bacterial sequence
ETIEAWNEIDRLPNLTIEQWLAFINKLDD

SEQ ID NO: 31 - protein, 29 aa, synthetic construct
  REGION 1..29: note = Modified bacterial sequence
EVLEAWREIDALPNLTIQQWLAFINKLDD

SEQ ID NO: 32 - protein, 29 aa, synthetic construct
  REGION 1..29: note = Modified bacterial sequence
EVIEAWDEIDQLPNLTIEQWLAFINKLDD

SEQ ID NO: 33 - protein, 29 aa, synthetic construct
  REGION 1..29: note = Modified bacterial sequence
EVLRAWDEIDHLPNLTLEQWLAFINKLDD

SEQ ID NO: 34 - protein, 29 aa, synthetic construct
  REGION 1..29: note = Modified bacterial sequence
EVLEAWDEIDRLPNLTLNQWLAFINKLDD

SEQ ID NO: 35 - protein, 29 aa, synthetic construct
  REGION 1..29: note = Modified bacterial sequence
EVLDAWNEIDHLPNLTIEQWLAFINKLDD

SEQ ID NO: 36 - protein, 29 aa, synthetic construct
  REGION 1..29: note = Modified bacterial sequence
EVIDAWNEIDKLPNLTIEQWLAFINKLDD

SEQ ID NO: 37 - protein, 29 aa, synthetic construct
  REGION 1..29: note = Modified bacterial sequence
ETLEAWDEIDQLPNLTLQQWLAFINKLDD

SEQ ID NO: 38 - protein, 29 aa, synthetic construct
  REGION 1..29: note = Modified bacterial sequence
EVIEAWNEIDALPNLTLDQWLAFINKLDD

SEQ ID NO: 39 - protein, 29 aa, synthetic construct
  REGION 1..29: note = Modified bacterial sequence
EVIDAWNEIDRLPNLTLQQWLAFINKLDD

SEQ ID NO: 40 - protein, 29 aa, synthetic construct
  REGION 1..29: note = Modified bacterial sequence
EVIDAWNEIDQLPNLTLEQWLAFINKLDD

SEQ ID NO: 41 - protein, 29 aa, synthetic construct
  REGION 1..29: note = Modified bacterial sequence
ETIAAWDEIDHLPNLTLEQWLAFINKLDD

SEQ ID NO: 42 - protein, 29 aa, synthetic construct
  REGION 1..29: note = Modified bacterial sequence
EVLQAWDEIDHLPNLTIQQWLAFINKLSD

SEQ ID NO: 43 - protein, 29 aa, synthetic construct
  REGION 1..29: note = Modified bacterial sequence
ETLHAWAEIDRLPNLTIEQWLAFINKLDD

SEQ ID NO: 44 - protein, 29 aa, synthetic construct
  REGION 1..29: note = Modified bacterial sequence
EVLEAWNEIDHLPNLTLAQWLAFINKLDD

SEQ ID NO: 45 - protein, 29 aa, synthetic construct
  REGION 1..29: note = Modified bacterial sequence
EVIEAWDEIDKLPNLTIAQWLAFINKLDD

SEQ ID NO: 46 - protein, 29 aa, synthetic construct
  REGION 1..29: note = Modified bacterial sequence
EVLDAWDEIDHLPNLTLQQWLAFINKLDD

SEQ ID NO: 47 - protein, 29 aa, synthetic construct
  REGION 1..29: note = Modified bacterial sequence
ETIEAWNEIDKLPNLTLTQWLAFINKLDD

SEQ ID NO: 48 - protein, 29 aa, synthetic construct
  REGION 1..29: note = Modified bacterial sequence
EVLEAWNEIDLLPNLTIEQWLAFINKLDD

SEQ ID NO: 49 - protein, 29 aa, synthetic construct
  REGION 1..29: note = Modified bacterial sequence
EVIEAWDEIDHLPNLTIDQWLAFINKLDD

SEQ ID NO: 50 - protein, 29 aa, synthetic construct
  REGION 1..29: note = Modified bacterial sequence
EVISAWNEIDALPNLTLQQWLAFINKLDD

SEQ ID NO: 51 - protein, 29 aa, synthetic construct
  REGION 1..29: note = Modified bacterial sequence
EVIAAWNEIDKLPNLTLEQWLAFINKLDD

SEQ ID NO: 52 - protein, 29 aa, synthetic construct
  REGION 1..29: note = Modified bacterial sequence
ETIEAWNEIDSLPNLTLDQWLAFINKLDD

SEQ ID NO: 53 - protein, 29 aa, synthetic construct
  REGION 1..29: note = Modified bacterial sequence
EVLDAWNEIDQLPNLTLQQWLAFINKLDD

SEQ ID NO: 54 - protein, 29 aa, synthetic construct
  REGION 1..29: note = Modified bacterial sequence
EVLAAWNEIDHLPNLTIEQWLAFINKLDD

SEQ ID NO: 55 - protein, 29 aa, synthetic construct
  REGION 1..29: note = Modified bacterial sequence
EVLEAWDEIDHLPNLTITQWLAFINKLDD

SEQ ID NO: 56 - protein, 29 aa, synthetic construct
  REGION 1..29: note = Modified bacterial sequence
ETIDAWNEIDHLPNLTIEQWLAFINKLDD

SEQ ID NO: 57 - protein, 29 aa, synthetic construct
  REGION 1..29: note = Modified bacterial sequence
EVIEAWNEIDHLPNLTIQQWLAFINKLDD

SEQ ID NO: 58 - protein, 29 aa, synthetic construct
  REGION 1..29: note = Modified bacterial sequence
EVIQAWNEIDALPNLTISQWLAFINKLDD

SEQ ID NO: 59 - protein, 29 aa, synthetic construct
  REGION 1..29: note = Modified bacterial sequence
EVIAAWDEIDSLPNLTIEQWLAFINKLDD

SEQ ID NO: 60 - protein, 29 aa, synthetic construct
  REGION 1..29: note = Modified bacterial sequence
EHIEAWNEIDALPNLTIEQWLAFINKLQD

SEQ ID NO: 61 - protein, 29 aa, synthetic construct
  REGION 1..29: note = Modified bacterial sequence
EVLEAWNEIDKLPNLTLDQWLAFINKLDD

SEQ ID NO: 62 - protein, 29 aa, synthetic construct
  REGION 1..29: note = Modified bacterial sequence
EVIDAWNEIDHLPNLTIEQWLAFINKLAD

SEQ ID NO: 63 - protein, 29 aa, synthetic construct
  REGION 1..29: note = Modified bacterial sequence
ETIDAWDEIDKLPNLTIEQWLAFINKLDD

SEQ ID NO: 64 - protein, 29 aa, synthetic construct
  REGION 1..29: note = Modified bacterial sequence
EVIAAWDEIDLLPNLTLQQWLAFINKLAD

SEQ ID NO: 65 - protein, 29 aa, synthetic construct
  REGION 1..29: note = Modified bacterial sequence
EVIHAWDEIDKLPNLTIEQWLAFINKLDD

SEQ ID NO: 66 - protein, 29 aa, synthetic construct
  REGION 1..29: note = Modified bacterial sequence
EVIAAWNEIDHLPNLTLEQWLAFINKLDD

SEQ ID NO: 67 - protein, 29 aa, synthetic construct
  REGION 1..29: note = Modified bacterial sequence
ETLDAWNEIDKLPNLTLSQWLAFINKLDD

SEQ ID NO: 68 - protein, 29 aa, synthetic construct
  REGION 1..29: note = Modified bacterial sequence
EVLEAWNEIDALPNLTLEQWLAFINKLDD

SEQ ID NO: 69 - protein, 29 aa, synthetic construct
  REGION 1..29: note = Modified bacterial sequence
EVIQAWDEIDHLPNLTISQWLAFINKLDD

SEQ ID NO: 70 - protein, 29 aa, synthetic construct
  REGION 1..29: note = Modified bacterial sequence
EVLQAWDEIDSLPNLTIEQWLAFINKLDD

SEQ ID NO: 71 - protein, 29 aa, synthetic construct
  REGION 1..29: note = Modified bacterial sequence
ETLEAWDEIDHLPNLTIAQWLAFINKLDD

SEQ ID NO: 72 - protein, 29 aa, synthetic construct
  REGION 1..29: note = Modified bacterial sequence
ETIDAWNEIDRLPNLTISQWLAFINKLDD

SEQ ID NO: 73 - protein, 29 aa, synthetic construct
  REGION 1..29: note = Modified bacterial sequence
EVLDAWHEIDHLPNLTIQQWLAFINKLDD

SEQ ID NO: 74 - protein, 29 aa, synthetic construct
  REGION 1..29: note = Modified bacterial sequence
EQIRAWDEIDKLPNLTIEQWLAFINKLAD

SEQ ID NO: 75 - protein, 29 aa, synthetic construct
  REGION 1..29: note = Modified bacterial sequence
ETLYAWNEIDKLPNLTIEQWLAFIEKLQD

SEQ ID NO: 76 - protein, 29 aa, synthetic construct
  REGION 1..29: note = Modified bacterial sequence
EVIEAWNEIDALPNLTIDQWLAFINKLDD

SEQ ID NO: 77 - protein, 29 aa, synthetic construct
  REGION 1..29: note = Modified bacterial sequence
EVLEAWNEIDHLPNLTIQQWLAFINKLDD

SEQ ID NO: 78 - protein, 29 aa, synthetic construct
  REGION 1..29: note = Modified bacterial sequence
ETIEAWDEIDALPNLTIEQWLAFINKLDD

SEQ ID NO: 79 - protein, 29 aa, synthetic construct
  REGION 1..29: note = Modified bacterial sequence
EVIEAWNEIDHLPNLTLQQWLAFINKLDD

SEQ ID NO: 80 - protein, 29 aa, synthetic construct
  REGION 1..29: note = Modified bacterial sequence
EVIEAWNEIDKLPNLTIQQWLAFINKLDD

SEQ ID NO: 81 - protein, 29 aa, synthetic construct
  REGION 1..29: note = Modified bacterial sequence
ETLDAWAEIDHLPNLTLDQWLAFINKLDD

SEQ ID NO: 82 - protein, 29 aa, synthetic construct
  REGION 1..29: note = Modified bacterial sequence
EHIDAWNEIDALPNLTLSQWLAFINKLDD

SEQ ID NO: 83 - protein, 29 aa, synthetic construct
  REGION 1..29: note = Modified bacterial sequence
EVLDAWNEIDKLPNLTIAQWLAFINKLDD

SEQ ID NO: 84 - protein, 29 aa, synthetic construct
  REGION 1..29: note = Modified bacterial sequence
EVIEAWTEIDYLPNLTLQQWLAFINKLDD

SEQ ID NO: 85 - protein, 29 aa, synthetic construct
  REGION 1..29: note = Modified bacterial sequence
ETIEAWNEIDHLPNLTIAQWLAFINKLDD

SEQ ID NO: 86 - protein, 29 aa, synthetic construct
  REGION 1..29: note = Modified bacterial sequence
EVIQAWNEIDKLPNLTLEQWLAFINKLDD

SEQ ID NO: 87 - protein, 29 aa, synthetic construct
  REGION 1..29: note = Modified bacterial sequence
EVIEAWDEIDHLPNLTIEQWLAFINKLDD

SEQ ID NO: 88 - protein, 29 aa, synthetic construct
  REGION 1..29: note = Modified bacterial sequence
ETIDAWNEIDLLPNLTIEQWLAFINKLDD

SEQ ID NO: 89 - protein, 29 aa, synthetic construct
  REGION 1..29: note = Modified bacterial sequence
EHIDAWNEIDKLPNLTLDQWLAFINKLDD

SEQ ID NO: 90 - protein, 29 aa, synthetic construct
  REGION 1..29: note = Modified bacterial sequence
EVVAAWNEIDALPNLTIEQWLAFINKLND

SEQ ID NO: 91 - protein, 29 aa, synthetic construct
  REGION 1..29: note = Modified bacterial sequence
EVIEAWNEIDALPNLTLAQWLAFINKLDD

SEQ ID NO: 92 - protein, 29 aa, synthetic construct
  REGION 1..29: note = Modified bacterial sequence
EVLQAWDEIDRLPNLTLDQWLAFINKLDD

SEQ ID NO: 93 - protein, 29 aa, synthetic construct
  REGION 1..29: note = Modified bacterial sequence
EVIDAWDEIDHLPNLTIEQWLAFINKLSD

SEQ ID NO: 94 - protein, 29 aa, synthetic construct
  REGION 1..29: note = Modified bacterial sequence
EVVEAWNEIDQLPNLTIEQWLAFINKLDD

SEQ ID NO: 95 - protein, 29 aa, synthetic construct
  REGION 1..29: note = Modified bacterial sequence
EVIQAWNEIDALPNLTIEQWLAFINKLDD

SEQ ID NO: 96 - protein, 29 aa, synthetic construct
  REGION 1..29: note = Modified bacterial sequence
EVIQAWDEIDKLPNLTIDQWLAFINKLAD

SEQ ID NO: 97 - protein, 29 aa, synthetic construct
  REGION 1..29: note = Modified bacterial sequence
EVVAAWDEIDALPNLTLTQWLAFINKLDD

SEQ ID NO: 98 - protein, 29 aa, synthetic construct
  REGION 1..29: note = Modified bacterial sequence
EVIQAWNEIDGLPNLTLSQWLAFINKLDD

SEQ ID NO: 99 - protein, 29 aa, synthetic construct
  REGION 1..29: note = Modified bacterial sequence
ETIEAWDEIDALPNLTITQWLAFINKLDD

SEQ ID NO: 100 - protein, 29 aa, synthetic construct
  REGION 1..29: note = Modified bacterial sequence
EVIDAWNEIDHLPNLTIQQWLAFINKLAD

SEQ ID NO: 101 - protein, 29 aa, synthetic construct
  REGION 1..29: note = Modified bacterial sequence
ETIEAWNEIDALPNLTLDQWLAFINKLED

SEQ ID NO: 102 - protein, 29 aa, synthetic construct
  REGION 1..29: note = Modified bacterial sequence
EHIHAWNEIDELPNLTIEQWLAFINKLAD

SEQ ID NO: 103 - protein, 29 aa, synthetic construct
  REGION 1..29: note = Modified bacterial sequence
EVIDAWDEIDHLPNLTIDQWLAFINKLSD

SEQ ID NO: 104 - protein, 29 aa, synthetic construct
  REGION 1..29: note = Modified bacterial sequence
EVIDANDEIDALPNLTIAQWLAFINKLHD

SEQ ID NO: 105 - protein, 29 aa, synthetic construct
  REGION 1..29: note = Modified bacterial sequence
ETIEAWDEIDKLPNLTIEQWLAFINKLDD

SEQ ID NO: 106 - protein, 29 aa, synthetic construct
  REGION 1..29: note = Modified bacterial sequence
EVLLAWDEIDHLPNLTLEQWLAFINKLDD

SEQ ID NO: 107 - protein, 29 aa, synthetic construct
  REGION 1..29: note = Modified bacterial sequence
EHIDAWNEIDGLPNLTLEQWLAFINKLDD

SEQ ID NO: 108 - protein, 29 aa, synthetic construct
  REGION 1..29: note = Modified bacterial sequence
EVIEAWSEIDALPNLTIDQWLAFINKLAD

SEQ ID NO: 109 - protein, 29 aa, synthetic construct
  REGION 1..29: note = Modified bacterial sequence
EQLNAWAEIDALPNLTIEQWLAFINKLDD

SEQ ID NO: 110 - protein, 29 aa, synthetic construct
  REGION 1..29: note = Modified bacterial sequence
EVIDAWNEIDALPNLTIAQWLAFINKLDD

SEQ ID NO: 111 - protein, 29 aa, synthetic construct
  REGION 1..29: note = Modified bacterial sequence
ETIDAWNEIDQLPNLTIEQWLAFINKLDD

SEQ ID NO: 112 - protein, 29 aa, synthetic construct
  REGION 1..29: note = Modified bacterial sequence
EVIEAWDEIDKLPNLTLAQWLAFINKLDD

SEQ ID NO: 113 - protein, 29 aa, synthetic construct
  REGION 1..29: note = Modified bacterial sequence
EVLYAWAEIDHLPNLTIEQWLAFINKLDD

SEQ ID NO: 114 - protein, 29 aa, synthetic construct
  REGION 1..29: note = Modified bacterial sequence
EQIDAWNEIDRLPNLTIQQWLAFINKLDD

SEQ ID NO: 115 - protein, 29 aa, synthetic construct
  REGION 1..29: note = Modified bacterial sequence
EVLAAWDEIDRLPNLTIEQWLAFINKLDD

SEQ ID NO: 116 - protein, 29 aa, synthetic construct
  REGION 1..29: note = Modified bacterial sequence
EVIEAWDEIDHLPNLTLHQWLAFINKLDD

SEQ ID NO: 117 - protein, 29 aa, synthetic construct
  REGION 1..29: note = Modified bacterial sequence
EVIEAWNEIDKLPNLTLQQWLAFINKLDD

SEQ ID NO: 118 - protein, 29 aa, synthetic construct
  REGION 1..29: note = Modified bacterial sequence
EVIDANDEIDALPNLTIEQWLAFINKLHD

SEQ ID NO: 119 - protein, 29 aa, synthetic construct
  REGION 1..29: note = Modified bacterial sequence
EVIAAWDEIDALPNLTIEQWLAFINKLDD

SEQ ID NO: 120 - protein, 29 aa, synthetic construct
  REGION 1..29: note = Modified bacterial sequence
EVIEAWTEIDQLPNLTLDQWLAFINKLDD

SEQ ID NO: 121 - protein, 29 aa, synthetic construct
  REGION 1..29: note = Modified bacterial sequence
EVINAWNEIDALPNLTLQQWLAFINKLDD

SEQ ID NO: 122 - protein, 29 aa, synthetic construct
  REGION 1..29: note = Modified bacterial sequence
EHIEAWDEIDHLPNLTIDQWLAFINKLAD

SEQ ID NO: 123 - protein, 29 aa, synthetic construct
  REGION 1..29: note = Modified bacterial sequence
EHLEAWREIDALPNLTIEQWLAFINKLDD

SEQ ID NO: 124 - protein, 29 aa, synthetic construct
  REGION 1..29: note = Modified bacterial sequence
EVLDAWNEIDKLPNLTLQQWLAFINKLDD

SEQ ID NO: 125 - protein, 29 aa, synthetic construct
  REGION 1..29: note = Modified bacterial sequence
EVIAAWDEIDHLPNLTIQQWLAFINKLDD

SEQ ID NO: 126 - protein, 29 aa, synthetic construct
  REGION 1..29: note = Modified bacterial sequence
EVIQAWNEIDALPNLTLEQWLAFINKLDD

SEQ ID NO: 127 - protein, 29 aa, synthetic construct
  REGION 1..29: note = Modified bacterial sequence
EVIDAWNEIDHLPNLTIAQWLAFINKLDD

SEQ ID NO: 128 - protein, 29 aa, synthetic construct
  REGION 1..29: note = Modified bacterial sequence
EQLDAWDEIDHLPNLTIDQWLAFINKLSD

SEQ ID NO: 129 - protein, 29 aa, synthetic construct
  REGION 1..29: note = Modified bacterial sequence
EVLNAWDEIDKLPNLTIEQWLAFINKLDD

SEQ ID NO: 130 - protein, 29 aa, synthetic construct
  REGION 1..29: note = Modified bacterial sequence
EVLEAWNEIDHLPNLTIDQWLAFINKLDD

SEQ ID NO: 131 - protein, 29 aa, synthetic construct
  REGION 1..29: note = Modified bacterial sequence
EVLLAWDEIDRLPNLTIDQWLAFINKLAD

SEQ ID NO: 132 - protein, 29 aa, synthetic construct
  REGION 1..29: note = Modified bacterial sequence
EVIAAWNEIDQLPNLTLDQWLAFINKLDD

SEQ ID NO: 133 - protein, 29 aa, synthetic construct
  REGION 1..29: note = Modified bacterial sequence
ETLLAWDEIDALPNLTIEQWLAFINKLDD

SEQ ID NO: 134 - protein, 29 aa, synthetic construct
  REGION 1..29: note = Modified bacterial sequence
EVIDAWNEIDTLPNLTLEQWLAFINKLDD

SEQ ID NO: 135 - protein, 29 aa, synthetic construct
  REGION 1..29: note = Modified bacterial sequence
EVLHAWNEIDHLPNLTLNQWLAFINKLQD

SEQ ID NO: 136 - protein, 29 aa, synthetic construct
  REGION 1..29: note = Modified bacterial sequence
EVIQAWNEIDALPNLTIAQWLAFINKLDD

SEQ ID NO: 137 - protein, 29 aa, synthetic construct
  REGION 1..29: note = Modified bacterial sequence
ETVDAWNEIDALPNLTIEQWLAFINKLDD

SEQ ID NO: 138 - protein, 29 aa, synthetic construct
  REGION 1..29: note = Modified bacterial sequence
EVIQAWDEIDHLPNLTIDQWLAFINKLDD

SEQ ID NO: 139 - protein, 29 aa, synthetic construct
  REGION 1..29: note = Modified bacterial sequence
EVLDAWNEIDQLPNLTIQQWLAFINKLDD

SEQ ID NO: 140 - protein, 29 aa, synthetic construct
  REGION 1..29: note = Modified bacterial sequence
ETIEAWNEIDALPNLTLDQWLAFINKLDD

SEQ ID NO: 141 - protein, 29 aa, synthetic construct
  REGION 1..29: note = Modified bacterial sequence
EVIEAWDEIDALPNLTIDQWLAFINKLDD

SEQ ID NO: 142 - protein, 29 aa, synthetic construct
  REGION 1..29: note = Modified bacterial sequence
EVIEAWNEIDQLPNLTIQQWLAFINKLDD

SEQ ID NO: 143 - protein, 29 aa, synthetic construct
  REGION 1..29: note = Modified bacterial sequence
EVIEAWTEIDHLPNLTIEQWLAFINKLDD

SEQ ID NO: 144 - protein, 29 aa, synthetic construct
  REGION 1..29: note = Modified bacterial sequence
EVIQAWNEIDHLPNLTLQQWLAFINKLED

SEQ ID NO: 145 - protein, 29 aa, synthetic construct
  REGION 1..29: note = Modified bacterial sequence
EVIQANNEIDQLPNLTIEQWLAFINKLHD

SEQ ID NO: 146 - protein, 29 aa, synthetic construct
  REGION 1..29: note = Modified bacterial sequence
EVLHAWSEIDKLPNLTIEQWLAFINKLDD

SEQ ID NO: 147 - protein, 29 aa, synthetic construct
  REGION 1..29: note = Modified bacterial sequence
ETIQAWDEIDKLPNLTLDQWLAFINKLSD

SEQ ID NO: 148 - protein, 29 aa, synthetic construct
  REGION 1..29: note = Modified bacterial sequence
ETLRAWDEIDKLPNLTIQQWLAFINKLAD

SEQ ID NO: 149 - protein, 29 aa, synthetic construct
  REGION 1..29: note = Modified bacterial sequence
EVIDAWNEIDHLPNLTIEQWLAFINKLED

SEQ ID NO: 150 - protein, 29 aa, synthetic construct
  REGION 1..29: note = Modified bacterial sequence
EVIDAWNEIDHLPNLTLQQWLAFINKLAD

SEQ ID NO: 151 - protein, 29 aa, synthetic construct
  REGION 1..29: note = Modified bacterial sequence
ETIDAWNEIDALPNLTLDQWLAFINKLDD

SEQ ID NO: 152 - protein, 29 aa, synthetic construct
  REGION 1..29: note = Modified bacterial sequence
EVIEAWNEIDQLPNLTIEQWLAFINKLDD

SEQ ID NO: 153 - protein, 29 aa, synthetic construct
  REGION 1..29: note = Modified bacterial sequence
EVIRAWDEIDQLPNLTLSQWLAFINKLDD

SEQ ID NO: 154 - protein, 29 aa, synthetic construct
  REGION 1..29: note = Modified bacterial sequence
EVIEAWNEIDRLPNLTIHQWLAFINKLDD

SEQ ID NO: 155 - protein, 29 aa, synthetic construct
  REGION 1..29: note = Modified bacterial sequence
ETIEAWNEIDQLPNLTIEQWLAFINKLDD

SEQ ID NO: 156 - protein, 29 aa, synthetic construct
  REGION 1..29: note = Modified bacterial sequence
EVLTAWAEIDALPNLTLSQWLAFINKLDD

SEQ ID NO: 157 - protein, 29 aa, synthetic construct
  REGION 1..29: note = Modified bacterial sequence
EVIEAWDEIDKLPNLTVDQWLAFINKLDD

SEQ ID NO: 158 - protein, 29 aa, synthetic construct
  REGION 1..29: note = Modified bacterial sequence
EVIDAWNEIDHLPNLTLTQWLAFINKLDD

SEQ ID NO: 159 - protein, 29 aa, synthetic construct
  REGION 1..29: note = Modified bacterial sequence
EVIEAWNEIDQLPNLTLDQWLAFINKLDD

SEQ ID NO: 160 - protein, 29 aa, synthetic construct
  REGION 1..29: note = Modified bacterial sequence
ETLQAWDEIDHLPNLTLNQWLAFINKLDD

SEQ ID NO: 161 - protein, 29 aa, synthetic construct
  REGION 1..29: note = Modified bacterial sequence
EVIDAWNEIDHLPNLTIEQWLAFINKLDD

SEQ ID NO: 162 - protein, 29 aa, synthetic construct
  REGION 1..29: note = Modified bacterial sequence
EVIEAWNEIDLLPNLTLSQWLAFINKLDD

SEQ ID NO: 163 - protein, 29 aa, synthetic construct
  REGION 1..29: note = Modified bacterial sequence
EVIDAWDEIDRLPNLTLKQWLAFINKLDD

SEQ ID NO: 164 - protein, 29 aa, synthetic construct
  REGION 1..29: note = Modified bacterial sequence
ETLHAWDEIDKLPNLTIEQWLAFINKLDD

SEQ ID NO: 165 - protein, 29 aa, synthetic construct
  REGION 1..29: note = Modified bacterial sequence
EVIKAWDEIDHLPNLTLNQWLAFINKLDD

SEQ ID NO: 166 - protein, 29 aa, synthetic construct
  REGION 1..29: note = Modified bacterial sequence
EVIEAWNEIDHLPNLTLAQWLAFINKLDD

SEQ ID NO: 167 - protein, 29 aa, synthetic construct
  REGION 1..29: note = Modified bacterial sequence
EVIQAWNEIDHLPNLTIDQWLAFITKLED

SEQ ID NO: 168 - protein, 29 aa, synthetic construct
  REGION 1..29: note = Modified bacterial sequence
EVIEAWNEIDRLPNLTIKQWLAFINKLDD

SEQ ID NO: 169 - protein, 29 aa, synthetic construct
  REGION 1..29: note = Modified bacterial sequence
EVIEAWNEIDSLPNLTLQQWLAFINKLDD

SEQ ID NO: 170 - protein, 29 aa, synthetic construct
  REGION 1..29: note = Modified bacterial sequence
ETIDAWNEIDKLPNLTIEQWLAFINKLDD

SEQ ID NO: 171 - protein, 29 aa, synthetic construct
  REGION 1..29: note = Modified bacterial sequence
EVLEAWAEIDALPNLTIAQWLAFINKLDD

SEQ ID NO: 172 - protein, 29 aa, synthetic construct
  REGION 1..29: note = Modified bacterial sequence
ETIDAWNEIDRLPNLTIEQWLAFINKLDD

SEQ ID NO: 173 - protein, 29 aa, synthetic construct
  REGION 1..29: note = Modified bacterial sequence
ETLKAWDEIDRLPNLTLEQWLAFINKLDD

SEQ ID NO: 174 - protein, 29 aa, synthetic construct
  REGION 1..29: note = Modified bacterial sequence
ETIAAWNEIDALPNLTLQQWLAFINKLDD

SEQ ID NO: 175 - protein, 29 aa, synthetic construct
  REGION 1..29: note = Modified bacterial sequence
EVLQAWNEIDHLPNLTIQQWLAFINKLDD

SEQ ID NO: 176 - protein, 29 aa, synthetic construct
  REGION 1..29: note = Modified bacterial sequence
EVIEAWSEIDHLPNLTLQQWLAFINKLDD

SEQ ID NO: 177 - protein, 29 aa, synthetic construct
  REGION 1..29: note = Modified bacterial sequence
EVIDAWNEIDGLPNLTIEQWLAFINKLDD

SEQ ID NO: 178 - protein, 29 aa, synthetic construct
  REGION 1..29: note = Modified bacterial sequence
EVIHAWNEIDHLPNLTLNQWLAFINKLED

SEQ ID NO: 179 - protein, 29 aa, synthetic construct
  REGION 1..29: note = Modified bacterial sequence
EVLDAWNEIDSLPNLTLDQWLAFINKLDD

SEQ ID NO: 180 - protein, 29 aa, synthetic construct
  REGION 1..29: note = Modified bacterial sequence
EQIEAWNEIDRLPNLTLEQWLAFINKLDD

SEQ ID NO: 181 - protein, 29 aa, synthetic construct
  REGION 1..29: note = Modified bacterial sequence
EVVDAWNEIDALPNLTLQQWLAFINKLDD

SEQ ID NO: 182 - protein, 29 aa, synthetic construct
  REGION 1..29: note = Modified bacterial sequence
EVIEAWNEIDKLPNLTIEQWLAFINKLDD

SEQ ID NO: 183 - protein, 29 aa, synthetic construct
  REGION 1..29: note = Modified bacterial sequence
EVIEANDEIDRLPNLTIEQWLAFINKLHD

SEQ ID NO: 184 - protein, 29 aa, synthetic construct
  REGION 1..29: note = Modified bacterial sequence
ETLQAWDEIDKLPNLTIEQWLAFINKLDD

SEQ ID NO: 185 - protein, 29 aa, synthetic construct
  REGION 1..29: note = Modified bacterial sequence
EVIEAWDEIDHLPNLTIDQWLAFINKLAD

SEQ ID NO: 186 - protein, 29 aa, synthetic construct
  REGION 1..29: note = Modified bacterial sequence
ETIDAWNEIDHLPNLTLQQWLAFINKLAD

SEQ ID NO: 187 - protein, 29 aa, synthetic construct
  REGION 1..29: note = Modified bacterial sequence
EVIDAWDEIDKLPNLTIEQWLAFINKLDD

SEQ ID NO: 188 - protein, 29 aa, synthetic construct
  REGION 1..29: note = Modified bacterial sequence
EVIEAWNEIDKLPNLTLAQWLAFINKLDD

SEQ ID NO: 189 - protein, 29 aa, synthetic construct
  REGION 1..29: note = Modified bacterial sequence
EVLQAWDEIDKLPNLTIQQWLAFINKLDD

SEQ ID NO: 190 - protein, 29 aa, synthetic construct
  REGION 1..29: note = Modified bacterial sequence
EVIAAWNEIDGLPNLTLQQWLAFINKLDD

SEQ ID NO: 191 - protein, 29 aa, synthetic construct
  REGION 1..29: note = Modified bacterial sequence
ETLNAWNEIDALPNLTLQQWLAFINKLDD

SEQ ID NO: 192 - protein, 29 aa, synthetic construct
  REGION 1..29: note = Modified bacterial sequence
EVLSAWNEIDQLPNLTLEQWLAFINKLDD

SEQ ID NO: 193 - protein, 29 aa, synthetic construct
  REGION 1..29: note = Modified bacterial sequence
ETLEAWDEIDHLPNLTLHQWLAFINKLDD

SEQ ID NO: 194 - protein, 29 aa, synthetic construct
  REGION 1..29: note = Modified bacterial sequence
EQIEAWNEIDHLPNLTLQQWLAFINKLAD

SEQ ID NO: 195 - protein, 29 aa, synthetic construct
  REGION 1..29: note = Modified bacterial sequence
EVVEAWDEIDKLPNLTIEQWLAFINKLDD

SEQ ID NO: 196 - protein, 29 aa, synthetic construct
  REGION 1..29: note = Modified bacterial sequence
EVLEAWNEIDELPNLTIEQWLAFINKLDD

SEQ ID NO: 197 - protein, 29 aa, synthetic construct
  REGION 1..29: note = Modified bacterial sequence
EVIDAWNEIDQLPNLTLQQWLAFINKLDD

SEQ ID NO: 198 - protein, 29 aa, synthetic construct
  REGION 1..29: note = Modified bacterial sequence
ETIDAWDEIDKLPNLTLSQWLAFINKLDD

SEQ ID NO: 199 - protein, 29 aa, synthetic construct
  REGION 1..29: note = Modified bacterial sequence
ETIDAWNEIDQLPNLTLQQWLAFINKLDD

SEQ ID NO: 200 - protein, 29 aa, synthetic construct
  REGION 1..29: note = Modified bacterial sequence
EVIQAWDEIDALPNLTLNQWLAFINKLDD

SEQ ID NO: 201 - protein, 29 aa, synthetic construct
  REGION 1..29: note = Modified bacterial sequence
EVLDAWAEIDQLPNLTLQQWLAFINKLDD

SEQ ID NO: 202 - protein, 29 aa, synthetic construct
  REGION 1..29: note = Modified bacterial sequence
EHIAAWDEIDALPNLTIEQWLAFINKLDD

SEQ ID NO: 203 - protein, 29 aa, synthetic construct
  REGION 1..29: note = Modified bacterial sequence
EVIRAWDEIDALPNLTIEQWLAFINKLDD

SEQ ID NO: 204 - protein, 29 aa, synthetic construct
  REGION 1..29: note = Modified bacterial sequence
EVIDAWDEIDALPNLTIDQWLAFINKLAD

SEQ ID NO: 205 - protein, 29 aa, synthetic construct
  REGION 1..29: note = Modified bacterial sequence
EVIDAWNEIDRLPNLTIQQWLAFINKLDD

SEQ ID NO: 206 - protein, 29 aa, synthetic construct
  REGION 1..29: note = Modified bacterial sequence
EVITAWNEIDHLPNLTLSQWLAFINKLDD

SEQ ID NO: 207 - protein, 29 aa, synthetic construct
  REGION 1..29: note = Modified bacterial sequence
EVIDAWNEIDALPNLTIHQWLAFINKLDD

SEQ ID NO: 208 - protein, 29 aa, synthetic construct
  REGION 1..29: note = Modified bacterial sequence
EQLKAWDEIDKLPNLTIEQWLAFIEKLQD

SEQ ID NO: 209 - protein, 29 aa, synthetic construct
  REGION 1..29: note = Modified bacterial sequence
EHIDAWTEIDHLPNLTIEQWLAFINKLDD

SEQ ID NO: 210 - protein, 29 aa, synthetic construct
  REGION 1..29: note = Modified bacterial sequence
EQLRAWDEIDKLPNLTIEQWLAFINKLQD

SEQ ID NO: 211 - protein, 29 aa, synthetic construct
  REGION 1..29: note = Modified bacterial sequence
EVLEAWREIDSLPNLTIAQWLAFINKLDD

SEQ ID NO: 212 - protein, 29 aa, synthetic construct
  REGION 1..29: note = Modified bacterial sequence
EVIQAWNEIDKLPNLTIEQWLAFINKLDD

SEQ ID NO: 213 - protein, 29 aa, synthetic construct
  REGION 1..29: note = Modified bacterial sequence
EHVEAWNEIDQLPNLTIEQWLAFINKLAD

SEQ ID NO: 214 - protein, 29 aa, synthetic construct
  REGION 1..29: note = Modified bacterial sequence
EVIDAWDEIDALPNLTIDQWLAFINKLSD

SEQ ID NO: 215 - protein, 29 aa, synthetic construct
  REGION 1..29: note = Modified bacterial sequence
EVIEAWNEIDHLPNLTIEQWLAFINKLDD

SEQ ID NO: 216 - protein, 29 aa, synthetic construct
  REGION 1..29: note = Modified bacterial sequence
EVLQAWDEIDKLPNLTIEQWLAFINKLSD

SEQ ID NO: 217 - protein, 29 aa, synthetic construct
  REGION 1..29: note = Modified bacterial sequence
EVIKAWNEIDSLPNLTIEQWLAFINKLDD

SEQ ID NO: 218 - protein, 29 aa, synthetic construct
  REGION 1..29: note = Modified bacterial sequence
EVLEAWHEIDLLPNLTIQQWLAFINKLDD

SEQ ID NO: 219 - protein, 29 aa, synthetic construct
  REGION 1..29: note = Modified bacterial sequence
EVLEAWTEIDRLPNLTLDQWLAFINKLDD

SEQ ID NO: 220 - protein, 29 aa, synthetic construct
  REGION 1..29: note = Modified bacterial sequence
EQLYAWNEIDHLPNLTIEQWLAFIEKLQD

SEQ ID NO: 221 - protein, 29 aa, synthetic construct
  REGION 1..29: note = Modified bacterial sequence
EVLNAWDEIDKLPNLTIKQWLAFINKLDD

SEQ ID NO: 222 - protein, 29 aa, synthetic construct
  REGION 1..29: note = Modified bacterial sequence
EVIRAWDEIDKLPNLTVEQWLAFINKLDD

SEQ ID NO: 223 - protein, 29 aa, synthetic construct
  REGION 1..29: note = Modified bacterial sequence
EVVQAWDEIDQLPNLTLEQWLAFINKLDD

SEQ ID NO: 224 - protein, 29 aa, synthetic construct
  REGION 1..29: note = Modified bacterial sequence
EVIRAWDEIDQLPNLTLEQWLAFINKLDD

SEQ ID NO: 225 - protein, 29 aa, synthetic construct
  REGION 1..29: note = Modified bacterial sequence
ETIDAWNEIDHLPNLTLDQWLAFINKLDD

SEQ ID NO: 226 - protein, 29 aa, synthetic construct
  REGION 1..29: note = Modified bacterial sequence
EVVAAWTEIDLLPNLTLDQWLAFINKLED

SEQ ID NO: 227 - protein, 29 aa, synthetic construct
  REGION 1..29: note = Modified bacterial sequence
EVVAAWDEIDALPNLTIEQWLAFINKLSD

SEQ ID NO: 228 - protein, 29 aa, synthetic construct
  REGION 1..29: note = Modified bacterial sequence
ETLEAWREIDSLPNLTLEQWLAFINKLDD

SEQ ID NO: 229 - protein, 29 aa, synthetic construct
  REGION 1..29: note = Modified bacterial sequence
EVIKAWNEIDHLPNLTLDQWLAFINKLDD

SEQ ID NO: 230 - protein, 29 aa, synthetic construct
  REGION 1..29: note = Modified bacterial sequence
EVLEAWTEIDKLPNLTIDQWLAFINKLDD

SEQ ID NO: 231 - protein, 29 aa, synthetic construct
  REGION 1..29: note = Modified bacterial sequence
ETLEAWDEIDKLPNLTIDQWLAFINKLDD

SEQ ID NO: 232 - protein, 29 aa, synthetic construct
  REGION 1..29: note = Modified bacterial sequence
EVIEAWNEIDKLPNLTIDQWLAFINKLDD

SEQ ID NO: 233 - protein, 29 aa, synthetic construct
  REGION 1..29: note = Modified bacterial sequence
ETIDAWNEIDKLPNLTLEQWLAFINKLDD

SEQ ID NO: 234 - protein, 29 aa, synthetic construct
  REGION 1..29: note = Modified bacterial sequence
ETLDAWDEIDALPNLTIDQWLAFINKLED

SEQ ID NO: 235 - protein, 29 aa, synthetic construct
  REGION 1..29: note = Modified bacterial sequence
EVLSAWNEIDHLPNLTIQQWLAFINKLDD

SEQ ID NO: 236 - protein, 29 aa, synthetic construct
  REGION 1..29: note = Modified bacterial sequence
EVIQANDEIDKLPNLTIEQWLAFIHKLHD

SEQ ID NO: 237 - protein, 29 aa, synthetic construct
  REGION 1..29: note = Modified bacterial sequence
EHLDAWDEIDHLPNLTIQQWLAFINKLAD

SEQ ID NO: 238 - protein, 29 aa, synthetic construct
  REGION 1..29: note = Modified bacterial sequence
EVIQAWNEIDQLPNLTIEQWLAFINKLDD

SEQ ID NO: 239 - protein, 29 aa, synthetic construct
  REGION 1..29: note = Modified bacterial sequence
EVIEAWNEIDYLPNLTIAQWIAFINKLDD

SEQ ID NO: 240 - protein, 29 aa, synthetic construct
  REGION 1..29: note = Modified bacterial sequence
ETIQAWDEIDRLPNLTLQQWLAFINKLDD

SEQ ID NO: 241 - protein, 29 aa, synthetic construct
  REGION 1..29: note = Modified bacterial sequence
ETIQAWDEIDKLPNLTIEQWLAFINKLDD

SEQ ID NO: 242 - protein, 29 aa, synthetic construct
  REGION 1..29: note = Modified bacterial sequence
ETLDAWAEIDHLPNLTIEQWLAFINKLDD

SEQ ID NO: 243 - protein, 29 aa, synthetic construct
  REGION 1..29: note = Modified bacterial sequence
EVIEAWDEIDKLPNLTLNQWLAFINKLDD

SEQ ID NO: 244 - protein, 29 aa, synthetic construct
  REGION 1..29: note = Modified bacterial sequence
EVLDAWNEIDQLPNLTIEQWLAFINKLDD

SEQ ID NO: 245 - protein, 29 aa, synthetic construct
  REGION 1..29: note = Modified bacterial sequence
EVLHAWNEIDHLPNLTIEQWLAFIEKLED

SEQ ID NO: 246 - protein, 29 aa, synthetic construct
  REGION 1..29: note = Modified bacterial sequence
EVIEAWQEIDKLPNLTIDQWLAFINKLDD

SEQ ID NO: 247 - protein, 29 aa, synthetic construct
  REGION 1..29: note = Modified bacterial sequence
EVVDAWNEIDQLPNLTIEQWLAFINKLDD

SEQ ID NO: 248 - protein, 29 aa, synthetic construct
  REGION 1..29: note = Modified bacterial sequence
EQIEAWNEIDALPNLTIEQWLAFINKLAD

SEQ ID NO: 249 - protein, 49 aa, synthetic construct
  REGION 1..49: note = Modified bacterial sequence
KEVLEAWDEIDRLPNLTIEQWLAFINKLDDDPSQSSELLSEAKKLNDSQ

SEQ ID NO: 250 - protein, 49 aa, synthetic construct
  REGION 1..49: note = Modified bacterial sequence
KEVLEAWNEIDRLPNLTIEQWLAFINKLDDDPSQSSELLSEAKKLNDSQ

SEQ ID NO: 251 - protein, 49 aa, synthetic construct
  REGION 1..49: note = Modified bacterial sequence
KEVIEAWNEIDRLPNLTIEQWLAFINKLDDDPSQSSELLSEAKKLNDSQ

SEQ ID NO: 252 - protein, 49 aa, synthetic construct
  REGION 1..49: note = Modified bacterial sequence
KEVLEAWDEIDRLPNLTLDQWLAFINKLDDDPSQSSELLSEAKKLNDSQ

SEQ ID NO: 253 - protein, 49 aa, synthetic construct
  REGION 1..49: note = Modified bacterial sequence
KEVLDAWDEIDALPNLTLEQWLAFINKLDDDPSQSSELLSEAKKLNDSQ

SEQ ID NO: 254 - protein, 49 aa, synthetic construct
  REGION 1..49: note = Modified bacterial sequence
KEVIDAWDEIDRLPNLTLDQWLAFINKLDDDPSQSSELLSEAKKLNDSQ

SEQ ID NO: 255 - protein, 49 aa, synthetic construct
  REGION 1..49: note = Modified bacterial sequence
KETLEAWDEIDRLPNLTIEQWLAFINKLDDDPSQSSELLSEAKKLNDSQ

SEQ ID NO: 256 - protein, 49 aa, synthetic construct
  REGION 1..49: note = Modified bacterial sequence
KEVIDAWNEIDALPNLTLDQWLAFINKLDDDPSQSSELLSEAKKLNDSQ

SEQ ID NO: 257 - protein, 49 aa, synthetic construct
  REGION 1..49: note = Modified bacterial sequence
KEVLDAWDEIDKLPNLTIDQWLAFINKLDDDPSQSSELLSEAKKLNDSQ

SEQ ID NO: 258 - protein, 49 aa, synthetic construct
  REGION 1..49: note = Modified bacterial sequence
KEVLEAWDEIDHLPNLTLDQWLAFINKLDDDPSQSSELLSEAKKLNDSQ

SEQ ID NO: 259 - protein, 49 aa, synthetic construct
  REGION 1..49: note = Modified bacterial sequence
KEVLEAWDEIDALPNLTIEQWLAFINKLDDDPSQSSELLSEAKKLNDSQ

SEQ ID NO: 260 - protein, 49 aa, synthetic construct
  REGION 1..49: note = Modified bacterial sequence
KEVLDAWDEIDKLPNLTLEQWLAFINKLDDDPSQSSELLSEAKKLNDSQ

SEQ ID NO: 261 - protein, 49 aa, synthetic construct
  REGION 1..49: note = Modified bacterial sequence
KETITAWDEIDKLPNLTIEQWLAFIGKLEDDPSQSSELLAEAKKLNDAQ

SEQ ID NO: 262 - protein, 49 aa, synthetic construct
  REGION 1..49: note = Modified bacterial sequence
KESMKAWDEIDRLPNLNINQWVAFIDSLYDDPSQSANLLAEAKKLNDAQ

SEQ ID NO: 263 - protein, 49 aa, synthetic construct
  REGION 1..49: note = Modified bacterial sequence
KESIEAWTEIDHLPNLTIEQWLAFINKLTDDPSQSSELLAEAKKLNDAQ

SEQ ID NO: 264 - protein, 49 aa, synthetic construct
  REGION 1..49: note = Modified bacterial sequence
KEVLDAWHEIDTLPNLTVRQWLAFISKLEDDPSQSSELLAEAKKLNDAQ

SEQ ID NO: 265 - protein, 49 aa, synthetic construct
  REGION 1..49: note = Modified bacterial sequence
KEHIQANEEIDRLPNLTIKQWLAFINKLHDDPSQSSELLAEAKKLNDAQ

SEQ ID NO: 266 - protein, 49 aa, synthetic construct
  REGION 1..49: note = Modified bacterial sequence
KEVLHAWAEIDALPNLTIEQWLAFINKLDDDPSQSSELLSEAKKLNDSQ

SEQ ID NO: 267 - protein, 49 aa, synthetic construct
  REGION 1..49: note = Modified bacterial sequence
KEVLAAWDEIDSLPNLTLQQWLAFINKLDDDPSQSSELLSEAKKLNDSQ

SEQ ID NO: 268 - protein, 49 aa, synthetic construct
  REGION 1..49: note = Modified bacterial sequence
KEVIDAWNEIDALPNLTLEQWLAFINKLDDDPSQSSELLSEAKKLNDSQ

SEQ ID NO: 269 - protein, 49 aa, synthetic construct
  REGION 1..49: note = Modified bacterial sequence
KEVLDAWNEIDALPNLTIDQWLAFINKLSDDPSQSSELLSEAKKLNDSQ

SEQ ID NO: 270 - protein, 49 aa, synthetic construct
  REGION 1..49: note = Modified bacterial sequence
KEVIEAWDEIDGLPNLTIEQWLAFINKLDDDPSQSSELLSEAKKLNDSQ

SEQ ID NO: 271 - protein, 49 aa, synthetic construct
  REGION 1..49: note = Modified bacterial sequence
KEVLEAWDEIDHLPNLTLQQWLAFINKLDDDPSQSSELLSEAKKLNDSQ

SEQ ID NO: 272 - protein, 49 aa, synthetic construct
  REGION 1..49: note = Modified bacterial sequence
KEVIEAWNEIDALPNLTIEQWLAFINKLDDDPSQSSELLSEAKKLNDSQ

SEQ ID NO: 273 - protein, 49 aa, synthetic construct
  REGION 1..49: note = Modified bacterial sequence
KEVIAAWNEIDRLPNLTLTQWLAFINKLDDDPSQSSELLSEAKKLNDSQ

SEQ ID NO: 274 - protein, 49 aa, synthetic construct
  REGION 1..49: note = Modified bacterial sequence
KEVIEAWDEIDALPNLTLQQWLAFINKLDDDPSQSSELLSEAKKLNDSQ

SEQ ID NO: 275 - protein, 49 aa, synthetic construct
  REGION 1..49: note = Modified bacterial sequence
KEVIAAWDEIDKLPNLTIEQWLAFINKLDDDPSQSSELLSEAKKLNDSQ

SEQ ID NO: 276 - protein, 49 aa, synthetic construct
  REGION 1..49: note = Modified bacterial sequence
KEVIAAWDEIDKLPNLTLQQWLAFINKLDDDPSQSSELLSEAKKLNDSQ

SEQ ID NO: 277 - protein, 49 aa, synthetic construct
  REGION 1..49: note = Modified bacterial sequence
KETIAAWDEIDKLPNLTIEQWLAFINKLDDDPSQSSELLSEAKKLNDSQ

SEQ ID NO: 278 - protein, 49 aa, synthetic construct
  REGION 1..49: note = Modified bacterial sequence
KETIEAWNEIDRLPNLTIEQWLAFINKLDDDPSQSSELLSEAKKLNDSQ

SEQ ID NO: 279 - protein, 49 aa, synthetic construct
  REGION 1..49: note = Modified bacterial sequence
KEVLEAWREIDALPNLTIQQWLAFINKLDDDPSQSSELLSEAKKLNDSQ

SEQ ID NO: 280 - protein, 49 aa, synthetic construct
  REGION 1..49: note = Modified bacterial sequence
KEVIEAWDEIDQLPNLTIEQWLAFINKLDDDPSQSSELLSEAKKLNDSQ

SEQ ID NO: 281 - protein, 49 aa, synthetic construct
  REGION 1..49: note = Modified bacterial sequence
KEVLRAWDEIDHLPNLTLEQWLAFINKLDDDPSQSSELLSEAKKLNDSQ

SEQ ID NO: 282 - protein, 49 aa, synthetic construct
  REGION 1..49: note = Modified bacterial sequence
KEVLEAWDEIDRLPNLTLNQWLAFINKLDDDPSQSSELLSEAKKLNDSQ

SEQ ID NO: 283 - protein, 49 aa, synthetic construct
  REGION 1..49: note = Modified bacterial sequence
KEVLDAWNEIDHLPNLTIEQWLAFINKLDDDPSQSSELLSEAKKLNDSQ

SEQ ID NO: 284 - protein, 49 aa, synthetic construct
  REGION 1..49: note = Modified bacterial sequence
KEVIDAWNEIDKLPNLTIEQWLAFINKLDDDPSQSSELLSEAKKLNDSQ

SEQ ID NO: 285 - protein, 49 aa, synthetic construct
  REGION 1..49: note = Modified bacterial sequence
KETLEAWDEIDQLPNLTLQQWLAFINKLDDDPSQSSELLSEAKKLNDSQ

SEQ ID NO: 286 - protein, 49 aa, synthetic construct
  REGION 1..49: note = Modified bacterial sequence
KEVIEAWNEIDALPNLTLDQWLAFINKLDDDPSQSSELLSEAKKLNDSQ

SEQ ID NO: 287 - protein, 49 aa, synthetic construct
  REGION 1..49: note = Modified bacterial sequence
KEVIDAWNEIDRLPNLTLQQWLAFINKLDDDPSQSSELLSEAKKLNDSQ

SEQ ID NO: 288 - protein, 49 aa, synthetic construct
  REGION 1..49: note = Modified bacterial sequence
KEVIDAWNEIDQLPNLTLEQWLAFINKLDDDPSQSSELLSEAKKLNDSQ

SEQ ID NO: 289 - protein, 49 aa, synthetic construct
  REGION 1..49: note = Modified bacterial sequence
KETIAAWDEIDHLPNLTLEQWLAFINKLDDDPSQSSELLSEAKKLNDSQ

SEQ ID NO: 290 - protein, 49 aa, synthetic construct
  REGION 1..49: note = Modified bacterial sequence
KEVLQAWDEIDHLPNLTIQQWLAFINKLSDDPSQSSELLSEAKKLNDSQ

SEQ ID NO: 291 - protein, 49 aa, synthetic construct
  REGION 1..49: note = Modified bacterial sequence
KETLHAWAEIDRLPNLTIEQWLAFINKLDDDPSQSSELLSEAKKLNDSQ

SEQ ID NO: 292 - protein, 49 aa, synthetic construct
  REGION 1..49: note = Modified bacterial sequence
KEVLEAWNEIDHLPNLTLAQWLAFINKLDDDPSQSSELLSEAKKLNDSQ

SEQ ID NO: 293 - protein, 49 aa, synthetic construct
  REGION 1..49: note = Modified bacterial sequence
KEVIEAWDEIDKLPNLTIAQWLAFINKLDDDPSQSSELLSEAKKLNDSQ

SEQ ID NO: 294 - protein, 49 aa, synthetic construct
  REGION 1..49: note = Modified bacterial sequence
KEVLDAWDEIDHLPNLTLQQWLAFINKLDDDPSQSSELLSEAKKLNDSQ

SEQ ID NO: 295 - protein, 49 aa, synthetic construct
  REGION 1..49: note = Modified bacterial sequence
KETIEAWNEIDKLPNLTLTQWLAFINKLDDDPSQSSELLSEAKKLNDSQ

SEQ ID NO: 296 - protein, 49 aa, synthetic construct
  REGION 1..49: note = Modified bacterial sequence
KEVLEAWNEIDLLPNLTIEQWLAFINKLDDDPSQSSELLSEAKKLNDSQ

SEQ ID NO: 297 - protein, 49 aa, synthetic construct
  REGION 1..49: note = Modified bacterial sequence
KEVIEAWDEIDHLPNLTIDQWLAFINKLDDDPSQSSELLSEAKKLNDSQ

SEQ ID NO: 298 - protein, 49 aa, synthetic construct
  REGION 1..49: note = Modified bacterial sequence
KEVISAWNEIDALPNLTLQQWLAFINKLDDDPSQSSELLSEAKKLNDSQ

SEQ ID NO: 299 - protein, 49 aa, synthetic construct
  REGION 1..49: note = Modified bacterial sequence
KEVIAAWNEIDKLPNLTLEQWLAFINKLDDDPSQSSELLSEAKKLNDSQ

SEQ ID NO: 300 - protein, 49 aa, synthetic construct
  REGION 1..49: note = Modified bacterial sequence
KETIEAWNEIDSLPNLTLDQWLAFINKLDDDPSQSSELLSEAKKLNDSQ

SEQ ID NO: 301 - protein, 49 aa, synthetic construct
  REGION 1..49: note = Modified bacterial sequence
KEVLDAWNEIDQLPNLTLQQWLAFINKLDDDPSQSSELLSEAKKLNDSQ

SEQ ID NO: 302 - protein, 49 aa, synthetic construct
  REGION 1..49: note = Modified bacterial sequence
KEVLAAWNEIDHLPNLTIEQWLAFINKLDDDPSQSSELLSEAKKLNDSQ

SEQ ID NO: 303 - protein, 49 aa, synthetic construct
  REGION 1..49: note = Modified bacterial sequence
KEVLEAWDEIDHLPNLTITQWLAFINKLDDDPSQSSELLSEAKKLNDSQ

SEQ ID NO: 304 - protein, 49 aa, synthetic construct
  REGION 1..49: note = Modified bacterial sequence
KETIDAWNEIDHLPNLTIEQWLAFINKLDDDPSQSSELLSEAKKLNDSQ

SEQ ID NO: 305 - protein, 49 aa, synthetic construct
  REGION 1..49: note = Modified bacterial sequence
KEVIEAWNEIDHLPNLTIQQWLAFINKLDDDPSQSSELLSEAKKLNDSQ

SEQ ID NO: 306 - protein, 49 aa, synthetic construct
  REGION 1..49: note = Modified bacterial sequence
KEVIQAWNEIDALPNLTISQWLAFINKLDDDPSQSSELLSEAKKLNDSQ

SEQ ID NO: 307 - protein, 49 aa, synthetic construct
  REGION 1..49: note = Modified bacterial sequence
KEVIAAWDEIDSLPNLTIEQWLAFINKLDDDPSQSSELLSEAKKLNDSQ

SEQ ID NO: 308 - protein, 49 aa, synthetic construct
  REGION 1..49: note = Modified bacterial sequence
KEHIEAWNEIDALPNLTIEQWLAFINKLQDDPSQSSELLSEAKKLNDSQ

SEQ ID NO: 309 - protein, 49 aa, synthetic construct
  REGION 1..49: note = Modified bacterial sequence
KEVLEAWNEIDKLPNLTLDQWLAFINKLDDDPSQSSELLSEAKKLNDSQ

SEQ ID NO: 310 - protein, 49 aa, synthetic construct
  REGION 1..49: note = Modified bacterial sequence
KEVIDAWNEIDHLPNLTIEQWLAFINKLADDPSQSSELLSEAKKLNDSQ

SEQ ID NO: 311 - protein, 49 aa, synthetic construct
  REGION 1..49: note = Modified bacterial sequence
KETIDAWDEIDKLPNLTIEQWLAFINKLDDDPSQSSELLSEAKKLNDSQ

SEQ ID NO: 312 - protein, 49 aa, synthetic construct
  REGION 1..49: note = Modified bacterial sequence
KEVIAAWDEIDLLPNLTLQQWLAFINKLADDPSQSSELLSEAKKLNDSQ

SEQ ID NO: 313 - protein, 49 aa, synthetic construct
  REGION 1..49: note = Modified bacterial sequence
KEVIHAWDEIDKLPNLTIEQWLAFINKLDDDPSQSSELLSEAKKLNDSQ

SEQ ID NO: 314 - protein, 49 aa, synthetic construct
  REGION 1..49: note = Modified bacterial sequence
KEVIAAWNEIDHLPNLTLEQWLAFINKLDDDPSQSSELLSEAKKLNDSQ

SEQ ID NO: 315 - protein, 49 aa, synthetic construct
  REGION 1..49: note = Modified bacterial sequence
KETLDAWNEIDKLPNLTLSQWLAFINKLDDDPSQSSELLSEAKKLNDSQ

SEQ ID NO: 316 - protein, 49 aa, synthetic construct
  REGION 1..49: note = Modified bacterial sequence
KEVLEAWNEIDALPNLTLEQWLAFINKLDDDPSQSSELLSEAKKLNDSQ

SEQ ID NO: 317 - protein, 49 aa, synthetic construct
  REGION 1..49: note = Modified bacterial sequence
KEVIQAWDEIDHLPNLTISQWLAFINKLDDDPSQSSELLSEAKKLNDSQ

SEQ ID NO: 318 - protein, 49 aa, synthetic construct
  REGION 1..49: note = Modified bacterial sequence
KEVLQAWDEIDSLPNLTIEQWLAFINKLDDDPSQSSELLSEAKKLNDSQ

SEQ ID NO: 319 - protein, 49 aa, synthetic construct
  REGION 1..49: note = Modified bacterial sequence
KETLEAWDEIDHLPNLTIAQWLAFINKLDDDPSQSSELLSEAKKLNDSQ

SEQ ID NO: 320 - protein, 49 aa, synthetic construct
  REGION 1..49: note = Modified bacterial sequence
KETIDAWNEIDRLPNLTISQWLAFINKLDDDPSQSSELLSEAKKLNDSQ

SEQ ID NO: 321 - protein, 49 aa, synthetic construct
  REGION 1..49: note = Modified bacterial sequence
KEVLDAWHEIDHLPNLTIQQWLAFINKLDDDPSQSSELLSEAKKLNDSQ

SEQ ID NO: 322 - protein, 49 aa, synthetic construct
  REGION 1..49: note = Modified bacterial sequence
KEQIRAWDEIDKLPNLTIEQWLAFINKLADDPSQSSELLSEAKKLNDSQ

SEQ ID NO: 323 - protein, 49 aa, synthetic construct
  REGION 1..49: note = Modified bacterial sequence
KETLYAWNEIDKLPNLTIEQWLAFIEKLQDDPSQSSELLSEAKKLNDSQ

SEQ ID NO: 324 - protein, 49 aa, synthetic construct
  REGION 1..49: note = Modified bacterial sequence
KEVIEAWNEIDALPNLTIDQWLAFINKLDDDPSQSSELLSEAKKLNDSQ

SEQ ID NO: 325 - protein, 49 aa, synthetic construct
  REGION 1..49: note = Modified bacterial sequence
KEVLEAWNEIDHLPNLTIQQWLAFINKLDDDPSQSSELLSEAKKLNDSQ

SEQ ID NO: 326 - protein, 49 aa, synthetic construct
  REGION 1..49: note = Modified bacterial sequence
KETIEAWDEIDALPNLTIEQWLAFINKLDDDPSQSSELLSEAKKLNDSQ

SEQ ID NO: 327 - protein, 49 aa, synthetic construct
  REGION 1..49: note = Modified bacterial sequence
KEVIEAWNEIDHLPNLTLQQWLAFINKLDDDPSQSSELLSEAKKLNDSQ

SEQ ID NO: 328 - protein, 49 aa, synthetic construct
  REGION 1..49: note = Modified bacterial sequence
KEVIEAWNEIDKLPNLTIQQWLAFINKLDDDPSQSSELLSEAKKLNDSQ

SEQ ID NO: 329 - protein, 49 aa, synthetic construct
  REGION 1..49: note = Modified bacterial sequence
KETLDAWAEIDHLPNLTLDQWLAFINKLDDDPSQSSELLSEAKKLNDSQ

SEQ ID NO: 330 - protein, 49 aa, synthetic construct
  REGION 1..49: note = Modified bacterial sequence
KEHIDAWNEIDALPNLTLSQWLAFINKLDDDPSQSSELLSEAKKLNDSQ

SEQ ID NO: 331 - protein, 49 aa, synthetic construct
  REGION 1..49: note = Modified bacterial sequence
KEVLDAWNEIDKLPNLTIAQWLAFINKLDDDPSQSSELLSEAKKLNDSQ

SEQ ID NO: 332 - protein, 49 aa, synthetic construct
  REGION 1..49: note = Modified bacterial sequence
KEVIEAWTEIDYLPNLTLQQWLAFINKLDDDPSQSSELLSEAKKLNDSQ

SEQ ID NO: 333 - protein, 49 aa, synthetic construct
  REGION 1..49: note = Modified bacterial sequence
KETIEAWNEIDHLPNLTIAQWLAFINKLDDDPSQSSELLSEAKKLNDSQ

SEQ ID NO: 334 - protein, 49 aa, synthetic construct
  REGION 1..49: note = Modified bacterial sequence
KEVIQAWNEIDKLPNLTLEQWLAFINKLDDDPSQSSELLSEAKKLNDSQ

SEQ ID NO: 335 - protein, 49 aa, synthetic construct
  REGION 1..49: note = Modified bacterial sequence
KEVIEAWDEIDHLPNLTIEQWLAFINKLDDDPSQSSELLSEAKKLNDSQ

SEQ ID NO: 336 - protein, 49 aa, synthetic construct
  REGION 1..49: note = Modified bacterial sequence
KETIDAWNEIDLLPNLTIEQWLAFINKLDDDPSQSSELLSEAKKLNDSQ

SEQ ID NO: 337 - protein, 49 aa, synthetic construct
  REGION 1..49: note = Modified bacterial sequence
KEHIDAWNEIDKLPNLTLDQWLAFINKLDDDPSQSSELLSEAKKLNDSQ

SEQ ID NO: 338 - protein, 49 aa, synthetic construct
  REGION 1..49: note = Modified bacterial sequence
KEVVAAWNEIDALPNLTIEQWLAFINKLNDDPSQSSELLSEAKKLNDSQ

SEQ ID NO: 339 - protein, 49 aa, synthetic construct
  REGION 1..49: note = Modified bacterial sequence
KEVIEAWNEIDALPNLTLAQWLAFINKLDDDPSQSSELLSEAKKLNDSQ

SEQ ID NO: 340 - protein, 49 aa, synthetic construct
  REGION 1..49: note = Modified bacterial sequence
KEVLQAWDEIDRLPNLTLDQWLAFINKLDDDPSQSSELLSEAKKLNDSQ

SEQ ID NO: 341 - protein, 49 aa, synthetic construct
  REGION 1..49: note = Modified bacterial sequence
KEVIDAWDEIDHLPNLTIEQWLAFINKLSDDPSQSSELLSEAKKLNDSQ

SEQ ID NO: 342 - protein, 49 aa, synthetic construct
  REGION 1..49: note = Modified bacterial sequence
KEVVEAWNEIDQLPNLTIEQWLAFINKLDDDPSQSSELLSEAKKLNDSQ

SEQ ID NO: 343 - protein, 49 aa, synthetic construct
  REGION 1..49: note = Modified bacterial sequence
KEVIQAWNEIDALPNLTIEQWLAFINKLDDDPSQSSELLSEAKKLNDSQ

SEQ ID NO: 344 - protein, 49 aa, synthetic construct
  REGION 1..49: note = Modified bacterial sequence
KEVIQAWDEIDKLPNLTIDQWLAFINKLADDPSQSSELLSEAKKLNDSQ

SEQ ID NO: 345 - protein, 49 aa, synthetic construct
  REGION 1..49: note = Modified bacterial sequence
KEVVAAWDEIDALPNLTLTQWLAFINKLDDDPSQSSELLSEAKKLNDSQ

SEQ ID NO: 346 - protein, 49 aa, synthetic construct
  REGION 1..49: note = Modified bacterial sequence
KEVIQAWNEIDGLPNLTLSQWLAFINKLDDDPSQSSELLSEAKKLNDSQ

SEQ ID NO: 347 - protein, 49 aa, synthetic construct
  REGION 1..49: note = Modified bacterial sequence
KETIEAWDEIDALPNLTITQWLAFINKLDDDPSQSSELLSEAKKLNDSQ

SEQ ID NO: 348 - protein, 49 aa, synthetic construct
  REGION 1..49: note = Modified bacterial sequence
KEVIDAWNEIDHLPNLTIQQWLAFINKLADDPSQSSELLSEAKKLNDSQ

SEQ ID NO: 349 - protein, 49 aa, synthetic construct
  REGION 1..49: note = Modified bacterial sequence
KETIEAWNEIDALPNLTLDQWLAFINKLEDDPSQSSELLSEAKKLNDSQ

SEQ ID NO: 350 - protein, 49 aa, synthetic construct
  REGION 1..49: note = Modified bacterial sequence
KEHIHAWNEIDELPNLTIEQWLAFINKLADDPSQSSELLSEAKKLNDSQ

SEQ ID NO: 351 - protein, 49 aa, synthetic construct
  REGION 1..49: note = Modified bacterial sequence
KEVIDAWDEIDHLPNLTIDQWLAFINKLSDDPSQSSELLSEAKKLNDSQ

SEQ ID NO: 352 - protein, 49 aa, synthetic construct
  REGION 1..49: note = Modified bacterial sequence
KEVIDANDEIDALPNLTIAQWLAFINKLHDDPSQSSELLSEAKKLNDSQ

SEQ ID NO: 353 - protein, 49 aa, synthetic construct
  REGION 1..49: note = Modified bacterial sequence
KETIEAWDEIDKLPNLTIEQWLAFINKLDDDPSQSSELLSEAKKLNDSQ

SEQ ID NO: 354 - protein, 49 aa, synthetic construct
  REGION 1..49: note = Modified bacterial sequence
KEVLLAWDEIDHLPNLTLEQWLAFINKLDDDPSQSSELLSEAKKLNDSQ

SEQ ID NO: 355 - protein, 49 aa, synthetic construct
  REGION 1..49: note = Modified bacterial sequence
KEHIDAWNEIDGLPNLTLEQWLAFINKLDDDPSQSSELLSEAKKLNDSQ

SEQ ID NO: 356 - protein, 49 aa, synthetic construct
  REGION 1..49: note = Modified bacterial sequence
KEVIEAWSEIDALPNLTIDQWLAFINKLADDPSQSSELLSEAKKLNDSQ

SEQ ID NO: 357 - protein, 49 aa, synthetic construct
  REGION 1..49: note = Modified bacterial sequence
KEQLNAWAEIDALPNLTIEQWLAFINKLDDDPSQSSELLSEAKKLNDSQ

SEQ ID NO: 358 - protein, 49 aa, synthetic construct
  REGION 1..49: note = Modified bacterial sequence
KEVIDAWNEIDALPNLTIAQWLAFINKLDDDPSQSSELLSEAKKLNDSQ

SEQ ID NO: 359 - protein, 49 aa, synthetic construct
  REGION 1..49: note = Modified bacterial sequence
KETIDAWNEIDQLPNLTIEQWLAFINKLDDDPSQSSELLSEAKKLNDSQ

SEQ ID NO: 360 - protein, 49 aa, synthetic construct
  REGION 1..49: note = Modified bacterial sequence
KEVIEAWDEIDKLPNLTLAQWLAFINKLDDDPSQSSELLSEAKKLNDSQ

SEQ ID NO: 361 - protein, 49 aa, synthetic construct
  REGION 1..49: note = Modified bacterial sequence
KEVLYAWAEIDHLPNLTIEQWLAFINKLDDDPSQSSELLSEAKKLNDSQ

SEQ ID NO: 362 - protein, 49 aa, synthetic construct
  REGION 1..49: note = Modified bacterial sequence
KEQIDAWNEIDRLPNLTIQQWLAFINKLDDDPSQSSELLSEAKKLNDSQ

SEQ ID NO: 363 - protein, 49 aa, synthetic construct
  REGION 1..49: note = Modified bacterial sequence
KEVLAAWDEIDRLPNLTIEQWLAFINKLDDDPSQSSELLSEAKKLNDSQ

SEQ ID NO: 364 - protein, 49 aa, synthetic construct
  REGION 1..49: note = Modified bacterial sequence
KEVIEAWDEIDHLPNLTLHQWLAFINKLDDDPSQSSELLSEAKKLNDSQ

SEQ ID NO: 365 - protein, 49 aa, synthetic construct
  REGION 1..49: note = Modified bacterial sequence
KEVIEAWNEIDKLPNLTLQQWLAFINKLDDDPSQSSELLSEAKKLNDSQ

SEQ ID NO: 366 - protein, 49 aa, synthetic construct
  REGION 1..49: note = Modified bacterial sequence
KEVIDANDEIDALPNLTIEQWLAFINKLHDDPSQSSELLSEAKKLNDSQ

SEQ ID NO: 367 - protein, 49 aa, synthetic construct
  REGION 1..49: note = Modified bacterial sequence
KEVIAAWDEIDALPNLTIEQWLAFINKLDDDPSQSSELLSEAKKLNDSQ

SEQ ID NO: 368 - protein, 49 aa, synthetic construct
  REGION 1..49: note = Modified bacterial sequence
KEVIEAWTEIDQLPNLTLDQWLAFINKLDDDPSQSSELLSEAKKLNDSQ

SEQ ID NO: 369 - protein, 49 aa, synthetic construct
  REGION 1..49: note = Modified bacterial sequence
KEVINAWNEIDALPNLTLQQWLAFINKLDDDPSQSSELLSEAKKLNDSQ

SEQ ID NO: 370 - protein, 49 aa, synthetic construct
  REGION 1..49: note = Modified bacterial sequence
KEHIEAWDEIDHLPNLTIDQWLAFINKLADDPSQSSELLSEAKKLNDSQ

SEQ ID NO: 371 - protein, 49 aa, synthetic construct
  REGION 1..49: note = Modified bacterial sequence
KEHLEAWREIDALPNLTIEQWLAFINKLDDDPSQSSELLSEAKKLNDSQ

SEQ ID NO: 372 - protein, 49 aa, synthetic construct
  REGION 1..49: note = Modified bacterial sequence
KEVLDAWNEIDKLPNLTLQQWLAFINKLDDDPSQSSELLSEAKKLNDSQ

SEQ ID NO: 373 - protein, 49 aa, synthetic construct
  REGION 1..49: note = Modified bacterial sequence
KEVIAAWDEIDHLPNLTIQQWLAFINKLDDDPSQSSELLSEAKKLNDSQ

SEQ ID NO: 374 - protein, 49 aa, synthetic construct
  REGION 1..49: note = Modified bacterial sequence
KEVIQAWNEIDALPNLTLEQWLAFINKLDDDPSQSSELLSEAKKLNDSQ

SEQ ID NO: 375 - protein, 49 aa, synthetic construct
  REGION 1..49: note = Modified bacterial sequence
KEVIDAWNEIDHLPNLTIAQWLAFINKLDDDPSQSSELLSEAKKLNDSQ

SEQ ID NO: 376 - protein, 49 aa, synthetic construct
  REGION 1..49: note = Modified bacterial sequence
KEQLDAWDEIDHLPNLTIDQWLAFINKLSDDPSQSSELLSEAKKLNDSQ

SEQ ID NO: 377 - protein, 49 aa, synthetic construct
  REGION 1..49: note = Modified bacterial sequence
KEVLNAWDEIDKLPNLTIEQWLAFINKLDDDPSQSSELLSEAKKLNDSQ

SEQ ID NO: 378 - protein, 49 aa, synthetic construct
  REGION 1..49: note = Modified bacterial sequence
KEVLEAWNEIDHLPNLTIDQWLAFINKLDDDPSQSSELLSEAKKLNDSQ

SEQ ID NO: 379 - protein, 49 aa, synthetic construct
  REGION 1..49: note = Modified bacterial sequence
KEVLLAWDEIDRLPNLTIDQWLAFINKLADDPSQSSELLSEAKKLNDSQ

SEQ ID NO: 380 - protein, 49 aa, synthetic construct
  REGION 1..49: note = Modified bacterial sequence
KEVIAAWNEIDQLPNLTLDQWLAFINKLDDDPSQSSELLSEAKKLNDSQ

SEQ ID NO: 381 - protein, 49 aa, synthetic construct
  REGION 1..49: note = Modified bacterial sequence
KETLLAWDEIDALPNLTIEQWLAFINKLDDDPSQSSELLSEAKKLNDSQ

SEQ ID NO: 382 - protein, 49 aa, synthetic construct
  REGION 1..49: note = Modified bacterial sequence
KEVIDAWNEIDTLPNLTLEQWLAFINKLDDDPSQSSELLSEAKKLNDSQ

SEQ ID NO: 383 - protein, 49 aa, synthetic construct
  REGION 1..49: note = Modified bacterial sequence
KEVLHAWNEIDHLPNLTLNQWLAFINKLQDDPSQSSELLSEAKKLNDSQ

SEQ ID NO: 384 - protein, 49 aa, synthetic construct
  REGION 1..49: note = Modified bacterial sequence
KEVIQAWNEIDALPNLTIAQWLAFINKLDDDPSQSSELLSEAKKLNDSQ

SEQ ID NO: 385 - protein, 49 aa, synthetic construct
  REGION 1..49: note = Modified bacterial sequence
KETVDAWNEIDALPNLTIEQWLAFINKLDDDPSQSSELLSEAKKLNDSQ

SEQ ID NO: 386 - protein, 49 aa, synthetic construct
  REGION 1..49: note = Modified bacterial sequence
KEVIQAWDEIDHLPNLTIDQWLAFINKLDDDPSQSSELLSEAKKLNDSQ

SEQ ID NO: 387 - protein, 49 aa, synthetic construct
  REGION 1..49: note = Modified bacterial sequence
KEVLDAWNEIDQLPNLTIQQWLAFINKLDDDPSQSSELLSEAKKLNDSQ

SEQ ID NO: 388 - protein, 49 aa, synthetic construct
  REGION 1..49: note = Modified bacterial sequence
KETIEAWNEIDALPNLTLDQWLAFINKLDDDPSQSSELLSEAKKLNDSQ

SEQ ID NO: 389 - protein, 49 aa, synthetic construct
  REGION 1..49: note = Modified bacterial sequence
KEVIEAWDEIDALPNLTIDQWLAFINKLDDDPSQSSELLSEAKKLNDSQ

SEQ ID NO: 390 - protein, 49 aa, synthetic construct
  REGION 1..49: note = Modified bacterial sequence
KEVIEAWNEIDQLPNLTIQQWLAFINKLDDDPSQSSELLSEAKKLNDSQ

SEQ ID NO: 391 - protein, 49 aa, synthetic construct
  REGION 1..49: note = Modified bacterial sequence
KEVIEAWTEIDHLPNLTIEQWLAFINKLDDDPSQSSELLSEAKKLNDSQ

SEQ ID NO: 392 - protein, 49 aa, synthetic construct
  REGION 1..49: note = Modified bacterial sequence
KEVIQAWNEIDHLPNLTLQQWLAFINKLEDDPSQSSELLSEAKKLNDSQ

SEQ ID NO: 393 - protein, 49 aa, synthetic construct
  REGION 1..49: note = Modified bacterial sequence
KEVIQANNEIDQLPNLTIEQWLAFINKLHDDPSQSSELLSEAKKLNDSQ

SEQ ID NO: 394 - protein, 49 aa, synthetic construct
  REGION 1..49: note = Modified bacterial sequence
KEVLHAWSEIDKLPNLTIEQWLAFINKLDDDPSQSSELLSEAKKLNDSQ

SEQ ID NO: 395 - protein, 49 aa, synthetic construct
  REGION 1..49: note = Modified bacterial sequence
KETIQAWDEIDKLPNLTLDQWLAFINKLSDDPSQSSELLSEAKKLNDSQ

SEQ ID NO: 396 - protein, 49 aa, synthetic construct
  REGION 1..49: note = Modified bacterial sequence
KETLRAWDEIDKLPNLTIQQWLAFINKLADDPSQSSELLSEAKKLNDSQ

SEQ ID NO: 397 - protein, 49 aa, synthetic construct
  REGION 1..49: note = Modified bacterial sequence
KEVIDAWNEIDHLPNLTIEQWLAFINKLEDDPSQSSELLSEAKKLNDSQ

SEQ ID NO: 398 - protein, 49 aa, synthetic construct
  REGION 1..49: note = Modified bacterial sequence
KEVIDAWNEIDHLPNLTLQQWLAFINKLADDPSQSSELLSEAKKLNDSQ

SEQ ID NO: 399 - protein, 49 aa, synthetic construct
  REGION 1..49: note = Modified bacterial sequence
KETIDAWNEIDALPNLTLDQWLAFINKLDDDPSQSSELLSEAKKLNDSQ

SEQ ID NO: 400 - protein, 49 aa, synthetic construct
  REGION 1..49: note = Modified bacterial sequence
KEVIEAWNEIDQLPNLTIEQWLAFINKLDDDPSQSSELLSEAKKLNDSQ

SEQ ID NO: 401 - protein, 49 aa, synthetic construct
  REGION 1..49: note = Modified bacterial sequence
KEVIRAWDEIDQLPNLTLSQWLAFINKLDDDPSQSSELLSEAKKLNDSQ

SEQ ID NO: 402 - protein, 49 aa, synthetic construct
  REGION 1..49: note = Modified bacterial sequence
KEVIEAWNEIDRLPNLTIHQWLAFINKLDDDPSQSSELLSEAKKLNDSQ

SEQ ID NO: 403 - protein, 49 aa, synthetic construct
  REGION 1..49: note = Modified bacterial sequence
KETIEAWNEIDQLPNLTIEQWLAFINKLDDDPSQSSELLSEAKKLNDSQ

SEQ ID NO: 404 - protein, 49 aa, synthetic construct
  REGION 1..49: note = Modified bacterial sequence
KEVLTAWAEIDALPNLTLSQWLAFINKLDDDPSQSSELLSEAKKLNDSQ

SEQ ID NO: 405 - protein, 49 aa, synthetic construct
  REGION 1..49: note = Modified bacterial sequence
KEVIEAWDEIDKLPNLTVDQWLAFINKLDDDPSQSSELLSEAKKLNDSQ

SEQ ID NO: 406 - protein, 49 aa, synthetic construct
  REGION 1..49: note = Modified bacterial sequence
KEVIDAWNEIDHLPNLTLTQWLAFINKLDDDPSQSSELLSEAKKLNDSQ

SEQ ID NO: 407 - protein, 49 aa, synthetic construct
  REGION 1..49: note = Modified bacterial sequence
KEVIEAWNEIDQLPNLTLDQWLAFINKLDDDPSQSSELLSEAKKLNDSQ

SEQ ID NO: 408 - protein, 49 aa, synthetic construct
  REGION 1..49: note = Modified bacterial sequence
KETLQAWDEIDHLPNLTLNQWLAFINKLDDDPSQSSELLSEAKKLNDSQ

SEQ ID NO: 409 - protein, 49 aa, synthetic construct
  REGION 1..49: note = Modified bacterial sequence
KEVIDAWNEIDHLPNLTIEQWLAFINKLDDDPSQSSELLSEAKKLNDSQ

SEQ ID NO: 410 - protein, 49 aa, synthetic construct
  REGION 1..49: note = Modified bacterial sequence
KEVIEAWNEIDLLPNLTLSQWLAFINKLDDDPSQSSELLSEAKKLNDSQ

SEQ ID NO: 411 - protein, 49 aa, synthetic construct
  REGION 1..49: note = Modified bacterial sequence
KEVIDAWDEIDRLPNLTLKQWLAFINKLDDDPSQSSELLSEAKKLNDSQ

SEQ ID NO: 412 - protein, 49 aa, synthetic construct
  REGION 1..49: note = Modified bacterial sequence
KETLHAWDEIDKLPNLTIEQWLAFINKLDDDPSQSSELLSEAKKLNDSQ

SEQ ID NO: 413 - protein, 49 aa, synthetic construct
  REGION 1..49: note = Modified bacterial sequence
KEVIKAWDEIDHLPNLTLNQWLAFINKLDDDPSQSSELLSEAKKLNDSQ

SEQ ID NO: 414 - protein, 49 aa, synthetic construct
  REGION 1..49: note = Modified bacterial sequence
KEVIEAWNEIDHLPNLTLAQWLAFINKLDDDPSQSSELLSEAKKLNDSQ

SEQ ID NO: 415 - protein, 49 aa, synthetic construct
  REGION 1..49: note = Modified bacterial sequence
KEVIQAWNEIDHLPNLTIDQWLAFITKLEDDPSQSSELLSEAKKLNDSQ

SEQ ID NO: 416 - protein, 49 aa, synthetic construct
  REGION 1..49: note = Modified bacterial sequence
KEVIEAWNEIDRLPNLTIKQWLAFINKLDDDPSQSSELLSEAKKLNDSQ

SEQ ID NO: 417 - protein, 49 aa, synthetic construct
  REGION 1..49: note = Modified bacterial sequence
KEVIEAWNEIDSLPNLTLQQWLAFINKLDDDPSQSSELLSEAKKLNDSQ

SEQ ID NO: 418 - protein, 49 aa, synthetic construct
  REGION 1..49: note = Modified bacterial sequence
KETIDAWNEIDKLPNLTIEQWLAFINKLDDDPSQSSELLSEAKKLNDSQ

SEQ ID NO: 419 - protein, 49 aa, synthetic construct
  REGION 1..49: note = Modified bacterial sequence
KEVLEAWAEIDALPNLTIAQWLAFINKLDDDPSQSSELLSEAKKLNDSQ

SEQ ID NO: 420 - protein, 49 aa, synthetic construct
  REGION 1..49: note = Modified bacterial sequence
KETIDAWNEIDRLPNLTIEQWLAFINKLDDDPSQSSELLSEAKKLNDSQ

SEQ ID NO: 421 - protein, 49 aa, synthetic construct
  REGION 1..49: note = Modified bacterial sequence
KETLKAWDEIDRLPNLTLEQWLAFINKLDDDPSQSSELLSEAKKLNDSQ

SEQ ID NO: 422 - protein, 49 aa, synthetic construct
  REGION 1..49: note = Modified bacterial sequence
KETIAAWNEIDALPNLTLQQWLAFINKLDDDPSQSSELLSEAKKLNDSQ

SEQ ID NO: 423 - protein, 49 aa, synthetic construct
  REGION 1..49: note = Modified bacterial sequence
KEVLQAWNEIDHLPNLTIQQWLAFINKLDDDPSQSSELLSEAKKLNDSQ

SEQ ID NO: 424 - protein, 49 aa, synthetic construct
  REGION 1..49: note = Modified bacterial sequence
KEVIEAWSEIDHLPNLTLQQWLAFINKLDDDPSQSSELLSEAKKLNDSQ

SEQ ID NO: 425 - protein, 49 aa, synthetic construct
  REGION 1..49: note = Modified bacterial sequence
KEVIDAWNEIDGLPNLTIEQWLAFINKLDDDPSQSSELLSEAKKLNDSQ

SEQ ID NO: 426 - protein, 49 aa, synthetic construct
  REGION 1..49: note = Modified bacterial sequence
KEVIHAWNEIDHLPNLTLNQWLAFINKLEDDPSQSSELLSEAKKLNDSQ

SEQ ID NO: 427 - protein, 49 aa, synthetic construct
  REGION 1..49: note = Modified bacterial sequence
KEVLDAWNEIDSLPNLTLDQWLAFINKLDDDPSQSSELLSEAKKLNDSQ

SEQ ID NO: 428 - protein, 49 aa, synthetic construct
  REGION 1..49: note = Modified bacterial sequence
KEQIEAWNEIDRLPNLTLEQWLAFINKLDDDPSQSSELLSEAKKLNDSQ

SEQ ID NO: 429 - protein, 49 aa, synthetic construct
  REGION 1..49: note = Modified bacterial sequence
KEVVDAWNEIDALPNLTLQQWLAFINKLDDDPSQSSELLSEAKKLNDSQ

SEQ ID NO: 430 - protein, 49 aa, synthetic construct
  REGION 1..49: note = Modified bacterial sequence
KEVIEAWNEIDKLPNLTIEQWLAFINKLDDDPSQSSELLSEAKKLNDSQ

SEQ ID NO: 431 - protein, 49 aa, synthetic construct
  REGION 1..49: note = Modified bacterial sequence
KEVIEANDEIDRLPNLTIEQWLAFINKLHDDPSQSSELLSEAKKLNDSQ

SEQ ID NO: 432 - protein, 49 aa, synthetic construct
  REGION 1..49: note = Modified bacterial sequence
KETLQAWDEIDKLPNLTIEQWLAFINKLDDDPSQSSELLSEAKKLNDSQ

SEQ ID NO: 433 - protein, 49 aa, synthetic construct
  REGION 1..49: note = Modified bacterial sequence
KEVIEAWDEIDHLPNLTIDQWLAFINKLADDPSQSSELLSEAKKLNDSQ

SEQ ID NO: 434 - protein, 49 aa, synthetic construct
  REGION 1..49: note = Modified bacterial sequence
KETIDAWNEIDHLPNLTLQQWLAFINKLADDPSQSSELLSEAKKLNDSQ

SEQ ID NO: 435 - protein, 49 aa, synthetic construct
  REGION 1..49: note = Modified bacterial sequence
KEVIDAWDEIDKLPNLTIEQWLAFINKLDDDPSQSSELLSEAKKLNDSQ

SEQ ID NO: 436 - protein, 49 aa, synthetic construct
  REGION 1..49: note = Modified bacterial sequence
KEVIEAWNEIDKLPNLTLAQWLAFINKLDDDPSQSSELLSEAKKLNDSQ

SEQ ID NO: 437 - protein, 49 aa, synthetic construct
  REGION 1..49: note = Modified bacterial sequence
KEVLQAWDEIDKLPNLTIQQWLAFINKLDDDPSQSSELLSEAKKLNDSQ

SEQ ID NO: 438 - protein, 49 aa, synthetic construct
  REGION 1..49: note = Modified bacterial sequence
KEVIAAWNEIDGLPNLTLQQWLAFINKLDDDPSQSSELLSEAKKLNDSQ

SEQ ID NO: 439 - protein, 49 aa, synthetic construct
  REGION 1..49: note = Modified bacterial sequence
KETLNAWNEIDALPNLTLQQWLAFINKLDDDPSQSSELLSEAKKLNDSQ

SEQ ID NO: 440 - protein, 49 aa, synthetic construct
  REGION 1..49: note = Modified bacterial sequence
KEVLSAWNEIDQLPNLTLEQWLAFINKLDDDPSQSSELLSEAKKLNDSQ

SEQ ID NO: 441 - protein, 49 aa, synthetic construct
  REGION 1..49: note = Modified bacterial sequence
KETLEAWDEIDHLPNLTLHQWLAFINKLDDDPSQSSELLSEAKKLNDSQ

SEQ ID NO: 442 - protein, 49 aa, synthetic construct
  REGION 1..49: note = Modified bacterial sequence
KEQIEAWNEIDHLPNLTLQQWLAFINKLADDPSQSSELLSEAKKLNDSQ

SEQ ID NO: 443 - protein, 49 aa, synthetic construct
  REGION 1..49: note = Modified bacterial sequence
KEVVEAWDEIDKLPNLTIEQWLAFINKLDDDPSQSSELLSEAKKLNDSQ

SEQ ID NO: 444 - protein, 49 aa, synthetic construct
  REGION 1..49: note = Modified bacterial sequence
KEVLEAWNEIDELPNLTIEQWLAFINKLDDDPSQSSELLSEAKKLNDSQ

SEQ ID NO: 445 - protein, 49 aa, synthetic construct
  REGION 1..49: note = Modified bacterial sequence
KEVIDAWNEIDQLPNLTLQQWLAFINKLDDDPSQSSELLSEAKKLNDSQ

SEQ ID NO: 446 - protein, 49 aa, synthetic construct
  REGION 1..49: note = Modified bacterial sequence
KETIDAWDEIDKLPNLTLSQWLAFINKLDDDPSQSSELLSEAKKLNDSQ

SEQ ID NO: 447 - protein, 49 aa, synthetic construct
  REGION 1..49: note = Modified bacterial sequence
KETIDAWNEIDQLPNLTLQQWLAFINKLDDDPSQSSELLSEAKKLNDSQ

SEQ ID NO: 448 - protein, 49 aa, synthetic construct
  REGION 1..49: note = Modified bacterial sequence
KEVIQAWDEIDALPNLTLNQWLAFINKLDDDPSQSSELLSEAKKLNDSQ

SEQ ID NO: 449 - protein, 49 aa, synthetic construct
  REGION 1..49: note = Modified bacterial sequence
KEVLDAWAEIDQLPNLTLQQWLAFINKLDDDPSQSSELLSEAKKLNDSQ

SEQ ID NO: 450 - protein, 49 aa, synthetic construct
  REGION 1..49: note = Modified bacterial sequence
KEHIAAWDEIDALPNLTIEQWLAFINKLDDDPSQSSELLSEAKKLNDSQ

SEQ ID NO: 451 - protein, 49 aa, synthetic construct
  REGION 1..49: note = Modified bacterial sequence
KEVIRAWDEIDALPNLTIEQWLAFINKLDDDPSQSSELLSEAKKLNDSQ

SEQ ID NO: 452 - protein, 49 aa, synthetic construct
  REGION 1..49: note = Modified bacterial sequence
KEVIDAWDEIDALPNLTIDQWLAFINKLADDPSQSSELLSEAKKLNDSQ

SEQ ID NO: 453 - protein, 49 aa, synthetic construct
  REGION 1..49: note = Modified bacterial sequence
KEVIDAWNEIDRLPNLTIQQWLAFINKLDDDPSQSSELLSEAKKLNDSQ

SEQ ID NO: 454 - protein, 49 aa, synthetic construct
  REGION 1..49: note = Modified bacterial sequence
KEVITAWNEIDHLPNLTLSQWLAFINKLDDDPSQSSELLSEAKKLNDSQ

SEQ ID NO: 455 - protein, 49 aa, synthetic construct
  REGION 1..49: note = Modified bacterial sequence
KEVIDAWNEIDALPNLTIHQWLAFINKLDDDPSQSSELLSEAKKLNDSQ

SEQ ID NO: 456 - protein, 49 aa, synthetic construct
  REGION 1..49: note = Modified bacterial sequence
KEQLKAWDEIDKLPNLTIEQWLAFIEKLQDDPSQSSELLSEAKKLNDSQ

SEQ ID NO: 457 - protein, 49 aa, synthetic construct
  REGION 1..49: note = Modified bacterial sequence
KEHIDAWTEIDHLPNLTIEQWLAFINKLDDDPSQSSELLSEAKKLNDSQ

SEQ ID NO: 458 - protein, 49 aa, synthetic construct
  REGION 1..49: note = Modified bacterial sequence
KEQLRAWDEIDKLPNLTIEQWLAFINKLQDDPSQSSELLSEAKKLNDSQ

SEQ ID NO: 459 - protein, 49 aa, synthetic construct
  REGION 1..49: note = Modified bacterial sequence
KEVLEAWREIDSLPNLTIAQWLAFINKLDDDPSQSSELLSEAKKLNDSQ

SEQ ID NO: 460 - protein, 49 aa, synthetic construct
  REGION 1..49: note = Modified bacterial sequence
KEVIQAWNEIDKLPNLTIEQWLAFINKLDDDPSQSSELLSEAKKLNDSQ

SEQ ID NO: 461 - protein, 49 aa, synthetic construct
  REGION 1..49: note = Modified bacterial sequence
KEHVEAWNEIDQLPNLTIEQWLAFINKLADDPSQSSELLSEAKKLNDSQ

SEQ ID NO: 462 - protein, 49 aa, synthetic construct
  REGION 1..49: note = Modified bacterial sequence
KEVIDAWDEIDALPNLTIDQWLAFINKLSDDPSQSSELLSEAKKLNDSQ

SEQ ID NO: 463 - protein, 49 aa, synthetic construct
  REGION 1..49: note = Modified bacterial sequence
KEVIEAWNEIDHLPNLTIEQWLAFINKLDDDPSQSSELLSEAKKLNDSQ

SEQ ID NO: 464 - protein, 49 aa, synthetic construct
  REGION 1..49: note = Modified bacterial sequence
KEVLQAWDEIDKLPNLTIEQWLAFINKLSDDPSQSSELLSEAKKLNDSQ

SEQ ID NO: 465 - protein, 49 aa, synthetic construct
  REGION 1..49: note = Modified bacterial sequence
KEVIKAWNEIDSLPNLTIEQWLAFINKLDDDPSQSSELLSEAKKLNDSQ

SEQ ID NO: 466 - protein, 49 aa, synthetic construct
  REGION 1..49: note = Modified bacterial sequence
KEVLEAWHEIDLLPNLTIQQWLAFINKLDDDPSQSSELLSEAKKLNDSQ

SEQ ID NO: 467 - protein, 49 aa, synthetic construct
  REGION 1..49: note = Modified bacterial sequence
KEVLEAWTEIDRLPNLTLDQWLAFINKLDDDPSQSSELLSEAKKLNDSQ

SEQ ID NO: 468 - protein, 49 aa, synthetic construct
  REGION 1..49: note = Modified bacterial sequence
KEQLYAWNEIDHLPNLTIEQWLAFIEKLQDDPSQSSELLSEAKKLNDSQ

SEQ ID NO: 469 - protein, 49 aa, synthetic construct
  REGION 1..49: note = Modified bacterial sequence
KEVLNAWDEIDKLPNLTIKQWLAFINKLDDDPSQSSELLSEAKKLNDSQ

SEQ ID NO: 470 - protein, 49 aa, synthetic construct
  REGION 1..49: note = Modified bacterial sequence
KEVIRAWDEIDKLPNLTVEQWLAFINKLDDDPSQSSELLSEAKKLNDSQ

SEQ ID NO: 471 - protein, 49 aa, synthetic construct
  REGION 1..49: note = Modified bacterial sequence
KEVVQAWDEIDQLPNLTLEQWLAFINKLDDDPSQSSELLSEAKKLNDSQ

SEQ ID NO: 472 - protein, 49 aa, synthetic construct
  REGION 1..49: note = Modified bacterial sequence
KEVIRAWDEIDQLPNLTLEQWLAFINKLDDDPSQSSELLSEAKKLNDSQ

SEQ ID NO: 473 - protein, 49 aa, synthetic construct
  REGION 1..49: note = Modified bacterial sequence
KETIDAWNEIDHLPNLTLDQWLAFINKLDDDPSQSSELLSEAKKLNDSQ

SEQ ID NO: 474 - protein, 49 aa, synthetic construct
  REGION 1..49: note = Modified bacterial sequence
KEVVAAWTEIDLLPNLTLDQWLAFINKLEDDPSQSSELLSEAKKLNDSQ

SEQ ID NO: 475 - protein, 49 aa, synthetic construct
  REGION 1..49: note = Modified bacterial sequence
KEVVAAWDEIDALPNLTIEQWLAFINKLSDDPSQSSELLSEAKKLNDSQ

SEQ ID NO: 476 - protein, 49 aa, synthetic construct
  REGION 1..49: note = Modified bacterial sequence
KETLEAWREIDSLPNLTLEQWLAFINKLDDDPSQSSELLSEAKKLNDSQ

SEQ ID NO: 477 - protein, 49 aa, synthetic construct
  REGION 1..49: note = Modified bacterial sequence
KEVIKAWNEIDHLPNLTLDQWLAFINKLDDDPSQSSELLSEAKKLNDSQ

SEQ ID NO: 478 - protein, 49 aa, synthetic construct
  REGION 1..49: note = Modified bacterial sequence
KEVLEAWTEIDKLPNLTIDQWLAFINKLDDDPSQSSELLSEAKKLNDSQ

SEQ ID NO: 479 - protein, 49 aa, synthetic construct
  REGION 1..49: note = Modified bacterial sequence
KETLEAWDEIDKLPNLTIDQWLAFINKLDDDPSQSSELLSEAKKLNDSQ

SEQ ID NO: 480 - protein, 49 aa, synthetic construct
  REGION 1..49: note = Modified bacterial sequence
KEVIEAWNEIDKLPNLTIDQWLAFINKLDDDPSQSSELLSEAKKLNDSQ

SEQ ID NO: 481 - protein, 49 aa, synthetic construct
  REGION 1..49: note = Modified bacterial sequence
KETIDAWNEIDKLPNLTLEQWLAFINKLDDDPSQSSELLSEAKKLNDSQ

SEQ ID NO: 482 - protein, 49 aa, synthetic construct
  REGION 1..49: note = Modified bacterial sequence
KETLDAWDEIDALPNLTIDQWLAFINKLEDDPSQSSELLSEAKKLNDSQ

SEQ ID NO: 483 - protein, 49 aa, synthetic construct
  REGION 1..49: note = Modified bacterial sequence
KEVLSAWNEIDHLPNLTIQQWLAFINKLDDDPSQSSELLSEAKKLNDSQ

SEQ ID NO: 484 - protein, 49 aa, synthetic construct
  REGION 1..49: note = Modified bacterial sequence
KEVIQANDEIDKLPNLTIEQWLAFIHKLHDDPSQSSELLSEAKKLNDSQ

SEQ ID NO: 485 - protein, 49 aa, synthetic construct
  REGION 1..49: note = Modified bacterial sequence
KEHLDAWDEIDHLPNLTIQQWLAFINKLADDPSQSSELLSEAKKLNDSQ

SEQ ID NO: 486 - protein, 49 aa, synthetic construct
  REGION 1..49: note = Modified bacterial sequence
KEVIQAWNEIDQLPNLTIEQWLAFINKLDDDPSQSSELLSEAKKLNDSQ

SEQ ID NO: 487 - protein, 49 aa, synthetic construct
  REGION 1..49: note = Modified bacterial sequence
KEVIEAWNEIDYLPNLTIAQWIAFINKLDDDPSQSSELLSEAKKLNDSQ

SEQ ID NO: 488 - protein, 49 aa, synthetic construct
  REGION 1..49: note = Modified bacterial sequence
KETIQAWDEIDRLPNLTLQQWLAFINKLDDDPSQSSELLSEAKKLNDSQ

SEQ ID NO: 489 - protein, 49 aa, synthetic construct
  REGION 1..49: note = Modified bacterial sequence
KETIQAWDEIDKLPNLTIEQWLAFINKLDDDPSQSSELLSEAKKLNDSQ

SEQ ID NO: 490 - protein, 49 aa, synthetic construct
  REGION 1..49: note = Modified bacterial sequence
KETLDAWAEIDHLPNLTIEQWLAFINKLDDDPSQSSELLSEAKKLNDSQ

SEQ ID NO: 491 - protein, 49 aa, synthetic construct
  REGION 1..49: note = Modified bacterial sequence
KEVIEAWDEIDKLPNLTLNQWLAFINKLDDDPSQSSELLSEAKKLNDSQ

SEQ ID NO: 492 - protein, 49 aa, synthetic construct
  REGION 1..49: note = Modified bacterial sequence
KEVLDAWNEIDQLPNLTIEQWLAFINKLDDDPSQSSELLSEAKKLNDSQ

SEQ ID NO: 493 - protein, 49 aa, synthetic construct
  REGION 1..49: note = Modified bacterial sequence
KEVLHAWNEIDHLPNLTIEQWLAFIEKLEDDPSQSSELLSEAKKLNDSQ

SEQ ID NO: 494 - protein, 49 aa, synthetic construct
  REGION 1..49: note = Modified bacterial sequence
KEVIEAWQEIDKLPNLTIDQWLAFINKLDDDPSQSSELLSEAKKLNDSQ

SEQ ID NO: 495 - protein, 49 aa, synthetic construct
  REGION 1..49: note = Modified bacterial sequence
KEVVDAWNEIDQLPNLTIEQWLAFINKLDDDPSQSSELLSEAKKLNDSQ

SEQ ID NO: 496 - protein, 49 aa, synthetic construct
  REGION 1..49: note = Modified bacterial sequence
KEQIEAWNEIDALPNLTIEQWLAFINKLADDPSQSSELLSEAKKLNDSQ

SEQ ID NO: 497 - protein, 58 aa, synthetic construct
  REGION 1..58: note = Modified bacterial sequence
VDAKYAKEVLEAWDEIDRLPNLTIEQWLAFINKLDDDPSQSSELLSEAKKLNDSQAPK

SEQ ID NO: 498 - protein, 58 aa, synthetic construct
  REGION 1..58: note = Modified bacterial sequence
VDAKYAKEVLEAWNEIDRLPNLTIEQWLAFINKLDDDPSQSSELLSEAKKLNDSQAPK

SEQ ID NO: 499 - protein, 58 aa, synthetic construct
  REGION 1..58: note = Modified bacterial sequence
VDAKYAKEVIEAWNEIDRLPNLTIEQWLAFINKLDDDPSQSSELLSEAKKLNDSQAPK

SEQ ID NO: 500 - protein, 58 aa, synthetic construct
  REGION 1..58: note = Modified bacterial sequence
VDAKYAKEVLEAWDEIDRLPNLTLDQWLAFINKLDDDPSQSSELLSEAKKLNDSQAPK

SEQ ID NO: 501 - protein, 58 aa, synthetic construct
  REGION 1..58: note = Modified bacterial sequence
VDAKYAKEVLDAWDEIDALPNLTLEQWLAFINKLDDDPSQSSELLSEAKKLNDSQAPK

SEQ ID NO: 502 - protein, 58 aa, synthetic construct
  REGION 1..58: note = Modified bacterial sequence
VDAKYAKEVIDAWDEIDRLPNLTLDQWLAFINKLDDDPSQSSELLSEAKKLNDSQAPK

SEQ ID NO: 503 - protein, 58 aa, synthetic construct
  REGION 1..58: note = Modified bacterial sequence
VDAKYAKETLEAWDEIDRLPNLTIEQWLAFINKLDDDPSQSSELLSEAKKLNDSQAPK

SEQ ID NO: 504 - protein, 58 aa, synthetic construct
  REGION 1..58: note = Modified bacterial sequence
VDAKYAKEVIDAWNEIDALPNLTLDQWLAFINKLDDDPSQSSELLSEAKKLNDSQAPK

SEQ ID NO: 505 - protein, 58 aa, synthetic construct
  REGION 1..58: note = Modified bacterial sequence
VDAKYAKEVLDAWDEIDKLPNLTIDQWLAFINKLDDDPSQSSELLSEAKKLNDSQAPK

SEQ ID NO: 506 - protein, 58 aa, synthetic construct
  REGION 1..58: note = Modified bacterial sequence
VDAKYAKEVLEAWDEIDHLPNLTLDQWLAFINKLDDDPSQSSELLSEAKKLNDSQAPK

SEQ ID NO: 507 - protein, 58 aa, synthetic construct
  REGION 1..58: note = Modified bacterial sequence
VDAKYAKEVLEAWDEIDALPNLTIEQWLAFINKLDDDPSQSSELLSEAKKLNDSQAPK

SEQ ID NO: 508 - protein, 58 aa, synthetic construct
  REGION 1..58: note = Modified bacterial sequence
VDAKYAKEVLDAWDEIDKLPNLTLEQWLAFINKLDDDPSQSSELLSEAKKLNDSQAPK

SEQ ID NO: 509 - protein, 58 aa, synthetic construct
  REGION 1..58: note = Modified bacterial sequence
VDAKYAKETITAWDEIDKLPNLTIEQWLAFIGKLEDDPSQSSELLAEAKKLNDAQAPK

SEQ ID NO: 510 - protein, 58 aa, synthetic construct
  REGION 1..58: note = Modified bacterial sequence
VDNKFNKESMKAWDEIDRLPNLNINQWVAFIDSLYDDPSQSANLLAEAKKLNDAQAPK

SEQ ID NO: 511 - protein, 58 aa, synthetic construct
  REGION 1..58: note = Modified bacterial sequence
VDAKYAKESIEAWTEIDHLPNLTIEQWLAFINKLTDDPSQSSELLAEAKKLNDAQAPK

SEQ ID NO: 512 - protein, 58 aa, synthetic construct
  REGION 1..58: note = Modified bacterial sequence
VDAKYAKEVLDAWHEIDTLPNLTVRQWLAFISKLEDDPSQSSELLAEAKKLNDAQAPK

SEQ ID NO: 513 - protein, 58 aa, synthetic construct
  REGION 1..58: note = Modified bacterial sequence
VDAKYAKEHIQANEEIDRLPNLTIKQWLAFINKLHDDPSQSSELLAEAKKLNDAQAPK

SEQ ID NO: 514 - protein, 58 aa, synthetic construct
  REGION 1..58: note = Modified bacterial sequence
VDAKYAKEVLHAWAEIDALPNLTIEQWLAFINKLDDDPSQSSELLSEAKKLNDSQAPK

SEQ ID NO: 515 - protein, 58 aa, synthetic construct
  REGION 1..58: note = Modified bacterial sequence
VDAKYAKEVLAAWDEIDSLPNLTLQQWLAFINKLDDDPSQSSELLSEAKKLNDSQAPK

SEQ ID NO: 516 - protein, 58 aa, synthetic construct
  REGION 1..58: note = Modified bacterial sequence
VDAKYAKEVIDAWNEIDALPNLTLEQWLAFINKLDDDPSQSSELLSEAKKLNDSQAPK

SEQ ID NO: 517 - protein, 58 aa, synthetic construct
  REGION 1..58: note = Modified bacterial sequence
VDAKYAKEVLDAWNEIDALPNLTIDQWLAFINKLSDDPSQSSELLSEAKKLNDSQAPK

SEQ ID NO: 518 - protein, 58 aa, synthetic construct
  REGION 1..58: note = Modified bacterial sequence
VDAKYAKEVIEAWDEIDGLPNLTIEQWLAFINKLDDDPSQSSELLSEAKKLNDSQAPK

SEQ ID NO: 519 - protein, 58 aa, synthetic construct
  REGION 1..58: note = Modified bacterial sequence
VDAKYAKEVLEAWDEIDHLPNLTLQQWLAFINKLDDDPSQSSELLSEAKKLNDSQAPK

SEQ ID NO: 520 - protein, 58 aa, synthetic construct
  REGION 1..58: note = Modified bacterial sequence
VDAKYAKEVIEAWNEIDALPNLTIEQWLAFINKLDDDPSQSSELLSEAKKLNDSQAPK

SEQ ID NO: 521 - protein, 58 aa, synthetic construct
  REGION 1..58: note = Modified bacterial sequence
VDAKYAKEVIAAWNEIDRLPNLTLTQWLAFINKLDDDPSQSSELLSEAKKLNDSQAPK

SEQ ID NO: 522 - protein, 58 aa, synthetic construct
  REGION 1..58: note = Modified bacterial sequence
VDAKYAKEVIEAWDEIDALPNLTLQQWLAFINKLDDDPSQSSELLSEAKKLNDSQAPK

SEQ ID NO: 523 - protein, 58 aa, synthetic construct
  REGION 1..58: note = Modified bacterial sequence
VDAKYAKEVIAAWDEIDKLPNLTIEQWLAFINKLDDDPSQSSELLSEAKKLNDSQAPK

SEQ ID NO: 524 - protein, 58 aa, synthetic construct
  REGION 1..58: note = Modified bacterial sequence
VDAKYAKEVIAAWDEIDKLPNLTLQQWLAFINKLDDDPSQSSELLSEAKKLNDSQAPK

SEQ ID NO: 525 - protein, 58 aa, synthetic construct
  REGION 1..58: note = Modified bacterial sequence
VDAKYAKETIAAWDEIDKLPNLTIEQWLAFINKLDDDPSQSSELLSEAKKLNDSQAPK

SEQ ID NO: 526 - protein, 58 aa, synthetic construct
  REGION 1..58: note = Modified bacterial sequence
VDAKYAKETIEAWNEIDRLPNLTIEQWLAFINKLDDDPSQSSELLSEAKKLNDSQAPK

SEQ ID NO: 527 - protein, 58 aa, synthetic construct
  REGION 1..58: note = Modified bacterial sequence
VDAKYAKEVLEAWREIDALPNLTIQQWLAFINKLDDDPSQSSELLSEAKKLNDSQAPK

SEQ ID NO: 528 - protein, 58 aa, synthetic construct
  REGION 1..58: note = Modified bacterial sequence
VDAKYAKEVIEAWDEIDQLPNLTIEQWLAFINKLDDDPSQSSELLSEAKKLNDSQAPK

SEQ ID NO: 529 - protein, 58 aa, synthetic construct
  REGION 1..58: note = Modified bacterial sequence
VDAKYAKEVLRAWDEIDHLPNLTLEQWLAFINKLDDDPSQSSELLSEAKKLNDSQAPK

SEQ ID NO: 530 - protein, 58 aa, synthetic construct
  REGION 1..58: note = Modified bacterial sequence
VDAKYAKEVLEAWDEIDRLPNLTLNQWLAFINKLDDDPSQSSELLSEAKKLNDSQAPK

SEQ ID NO: 531 - protein, 58 aa, synthetic construct
  REGION 1..58: note = Modified bacterial sequence
VDAKYAKEVLDAWNEIDHLPNLTIEQWLAFINKLDDDPSQSSELLSEAKKLNDSQAPK

SEQ ID NO: 532 - protein, 58 aa, synthetic construct
  REGION 1..58: note = Modified bacterial sequence
VDAKYAKEVIDAWNEIDKLPNLTIEQWLAFINKLDDDPSQSSELLSEAKKLNDSQAPK

SEQ ID NO: 533 - protein, 58 aa, synthetic construct
  REGION 1..58: note = Modified bacterial sequence
VDAKYAKETLEAWDEIDQLPNLTLQQWLAFINKLDDDPSQSSELLSEAKKLNDSQAPK

SEQ ID NO: 534 - protein, 58 aa, synthetic construct
  REGION 1..58: note = Modified bacterial sequence
VDAKYAKEVIEAWNEIDALPNLTLDQWLAFINKLDDDPSQSSELLSEAKKLNDSQAPK

SEQ ID NO: 535 - protein, 58 aa, synthetic construct
  REGION 1..58: note = Modified bacterial sequence
VDAKYAKEVIDAWNEIDRLPNLTLQQWLAFINKLDDDPSQSSELLSEAKKLNDSQAPK

SEQ ID NO: 536 - protein, 58 aa, synthetic construct
  REGION 1..58: note = Modified bacterial sequence
VDAKYAKEVIDAWNEIDQLPNLTLEQWLAFINKLDDDPSQSSELLSEAKKLNDSQAPK

SEQ ID NO: 537 - protein, 58 aa, synthetic construct
  REGION 1..58: note = Modified bacterial sequence
VDAKYAKETIAAWDEIDHLPNLTLEQWLAFINKLDDDPSQSSELLSEAKKLNDSQAPK

SEQ ID NO: 538 - protein, 58 aa, synthetic construct
  REGION 1..58: note = Modified bacterial sequence
VDAKYAKEVLQAWDEIDHLPNLTIQQWLAFINKLSDDPSQSSELLSEAKKLNDSQAPK

SEQ ID NO: 539 - protein, 58 aa, synthetic construct
  REGION 1..58: note = Modified bacterial sequence
VDAKYAKETLHAWAEIDRLPNLTIEQWLAFINKLDDDPSQSSELLSEAKKLNDSQAPK

SEQ ID NO: 540 - protein, 58 aa, synthetic construct
  REGION 1..58: note = Modified bacterial sequence
VDAKYAKEVLEAWNEIDHLPNLTLAQWLAFINKLDDDPSQSSELLSEAKKLNDSQAPK

SEQ ID NO: 541 - protein, 58 aa, synthetic construct
  REGION 1..58: note = Modified bacterial sequence
VDAKYAKEVIEAWDEIDKLPNLTIAQWLAFINKLDDDPSQSSELLSEAKKLNDSQAPK

SEQ ID NO: 542 - protein, 58 aa, synthetic construct
  REGION 1..58: note = Modified bacterial sequence
VDAKYAKEVLDAWDEIDHLPNLTLQQWLAFINKLDDDPSQSSELLSEAKKLNDSQAPK

SEQ ID NO: 543 - protein, 58 aa, synthetic construct
  REGION 1..58: note = Modified bacterial sequence
VDAKYAKETIEAWNEIDKLPNLTLTQWLAFINKLDDDPSQSSELLSEAKKLNDSQAPK

SEQ ID NO: 544 - protein, 58 aa, synthetic construct
  REGION 1..58: note = Modified bacterial sequence
VDAKYAKEVLEAWNEIDLLPNLTIEQWLAFINKLDDDPSQSSELLSEAKKLNDSQAPK

SEQ ID NO: 545 - protein, 58 aa, synthetic construct
  REGION 1..58: note = Modified bacterial sequence
VDAKYAKEVIEAWDEIDHLPNLTIDQWLAFINKLDDDPSQSSELLSEAKKLNDSQAPK

SEQ ID NO: 546 - protein, 58 aa, synthetic construct
  REGION 1..58: note = Modified bacterial sequence
VDAKYAKEVISAWNEIDALPNLTLQQWLAFINKLDDDPSQSSELLSEAKKLNDSQAPK

SEQ ID NO: 547 - protein, 58 aa, synthetic construct
  REGION 1..58: note = Modified bacterial sequence
VDAKYAKEVIAAWNEIDKLPNLTLEQWLAFINKLDDDPSQSSELLSEAKKLNDSQAPK

SEQ ID NO: 548 - protein, 58 aa, synthetic construct
  REGION 1..58: note = Modified bacterial sequence
VDAKYAKETIEAWNEIDSLPNLTLDQWLAFINKLDDDPSQSSELLSEAKKLNDSQAPK

SEQ ID NO: 549 - protein, 58 aa, synthetic construct
  REGION 1..58: note = Modified bacterial sequence
VDAKYAKEVLDAWNEIDQLPNLTLQQWLAFINKLDDDPSQSSELLSEAKKLNDSQAPK

SEQ ID NO: 550 - protein, 58 aa, synthetic construct
  REGION 1..58: note = Modified bacterial sequence
VDAKYAKEVLAAWNEIDHLPNLTIEQWLAFINKLDDDPSQSSELLSEAKKLNDSQAPK

SEQ ID NO: 551 - protein, 58 aa, synthetic construct
  REGION 1..58: note = Modified bacterial sequence
VDAKYAKEVLEAWDEIDHLPNLTITQWLAFINKLDDDPSQSSELLSEAKKLNDSQAPK

SEQ ID NO: 552 - protein, 58 aa, synthetic construct
  REGION 1..58: note = Modified bacterial sequence
VDAKYAKETIDAWNEIDHLPNLTIEQWLAFINKLDDDPSQSSELLSEAKKLNDSQAPK

SEQ ID NO: 553 - protein, 58 aa, synthetic construct
  REGION 1..58: note = Modified bacterial sequence
VDAKYAKEVIEAWNEIDHLPNLTIQQWLAFINKLDDDPSQSSELLSEAKKLNDSQAPK

SEQ ID NO: 554 - protein, 58 aa, synthetic construct
  REGION 1..58: note = Modified bacterial sequence
VDAKYAKEVIQAWNEIDALPNLTISQWLAFINKLDDDPSQSSELLSEAKKLNDSQAPK

SEQ ID NO: 555 - protein, 58 aa, synthetic construct
  REGION 1..58: note = Modified bacterial sequence
VDAKYAKEVIAAWDEIDSLPNLTIEQWLAFINKLDDDPSQSSELLSEAKKLNDSQAPK

SEQ ID NO: 556 - protein, 58 aa, synthetic construct
  REGION 1..58: note = Modified bacterial sequence
VDAKYAKEHIEAWNEIDALPNLTIEQWLAFINKLQDDPSQSSELLSEAKKLNDSQAPK

SEQ ID NO: 557 - protein, 58 aa, synthetic construct
  REGION 1..58: note = Modified bacterial sequence
VDAKYAKEVLEAWNEIDKLPNLTLDQWLAFINKLDDDPSQSSELLSEAKKLNDSQAPK

SEQ ID NO: 558 - protein, 58 aa, synthetic construct
  REGION 1..58: note = Modified bacterial sequence
VDAKYAKEVIDAWNEIDHLPNLTIEQWLAFINKLADDPSQSSELLSEAKKLNDSQAPK

SEQ ID NO: 559 - protein, 58 aa, synthetic construct
  REGION 1..58: note = Modified bacterial sequence
VDAKYAKETIDAWDEIDKLPNLTIEQWLAFINKLDDDPSQSSELLSEAKKLNDSQAPK

SEQ ID NO: 560 - protein, 58 aa, synthetic construct
  REGION 1..58: note = Modified bacterial sequence
VDAKYAKEVIAAWDEIDLLPNLTLQQWLAFINKLADDPSQSSELLSEAKKLNDSQAPK

SEQ ID NO: 561 - protein, 58 aa, synthetic construct
  REGION 1..58: note = Modified bacterial sequence
VDAKYAKEVIHAWDEIDKLPNLTIEQWLAFINKLDDDPSQSSELLSEAKKLNDSQAPK

SEQ ID NO: 562 - protein, 58 aa, synthetic construct
  REGION 1..58: note = Modified bacterial sequence
VDAKYAKEVIAAWNEIDHLPNLTLEQWLAFINKLDDDPSQSSELLSEAKKLNDSQAPK

SEQ ID NO: 563 - protein, 58 aa, synthetic construct
  REGION 1..58: note = Modified bacterial sequence
VDAKYAKETLDAWNEIDKLPNLTLSQWLAFINKLDDDPSQSSELLSEAKKLNDSQAPK

SEQ ID NO: 564 - protein, 58 aa, synthetic construct
  REGION 1..58: note = Modified bacterial sequence
VDAKYAKEVLEAWNEIDALPNLTLEQWLAFINKLDDDPSQSSELLSEAKKLNDSQAPK

SEQ ID NO: 565 - protein, 58 aa, synthetic construct
  REGION 1..58: note = Modified bacterial sequence
VDAKYAKEVIQAWDEIDHLPNLTISQWLAFINKLDDDPSQSSELLSEAKKLNDSQAPK

SEQ ID NO: 566 - protein, 58 aa, synthetic construct
  REGION 1..58: note = Modified bacterial sequence
VDAKYAKEVLQAWDEIDSLPNLTIEQWLAFINKLDDDPSQSSELLSEAKKLNDSQAPK

SEQ ID NO: 567 - protein, 58 aa, synthetic construct
  REGION 1..58: note = Modified bacterial sequence
VDAKYAKETLEAWDEIDHLPNLTIAQWLAFINKLDDDPSQSSELLSEAKKLNDSQAPK

SEQ ID NO: 568 - protein, 58 aa, synthetic construct
  REGION 1..58: note = Modified bacterial sequence
VDAKYAKETIDAWNEIDRLPNLTISQWLAFINKLDDDPSQSSELLSEAKKLNDSQAPK

SEQ ID NO: 569 - protein, 58 aa, synthetic construct
  REGION 1..58: note = Modified bacterial sequence
VDAKYAKEVLDAWHEIDHLPNLTIQQWLAFINKLDDDPSQSSELLSEAKKLNDSQAPK

SEQ ID NO: 570 - protein, 58 aa, synthetic construct
  REGION 1..58: note = Modified bacterial sequence
VDAKYAKEQIRAWDEIDKLPNLTIEQWLAFINKLADDPSQSSELLSEAKKLNDSQAPK

SEQ ID NO: 571 - protein, 58 aa, synthetic construct
  REGION 1..58: note = Modified bacterial sequence
VDAKYAKETLYAWNEIDKLPNLTIEQWLAFIEKLQDDPSQSSELLSEAKKLNDSQAPK

SEQ ID NO: 572 - protein, 58 aa, synthetic construct
  REGION 1..58: note = Modified bacterial sequence
VDAKYAKEVIEAWNEIDALPNLTIDQWLAFINKLDDDPSQSSELLSEAKKLNDSQAPK

SEQ ID NO: 573 - protein, 58 aa, synthetic construct
  REGION 1..58: note = Modified bacterial sequence
VDAKYAKEVLEAWNEIDHLPNLTIQQWLAFINKLDDDPSQSSELLSEAKKLNDSQAPK

SEQ ID NO: 574 - protein, 58 aa, synthetic construct
  REGION 1..58: note = Modified bacterial sequence
VDAKYAKETIEAWDEIDALPNLTIEQWLAFINKLDDDPSQSSELLSEAKKLNDSQAPK

SEQ ID NO: 575 - protein, 58 aa, synthetic construct
  REGION 1..58: note = Modified bacterial sequence
VDAKYAKEVIEAWNEIDHLPNLTLQQWLAFINKLDDDPSQSSELLSEAKKLNDSQAPK

SEQ ID NO: 576 - protein, 58 aa, synthetic construct
  REGION 1..58: note = Modified bacterial sequence
VDAKYAKEVIEAWNEIDKLPNLTIQQWLAFINKLDDDPSQSSELLSEAKKLNDSQAPK

SEQ ID NO: 577 - protein, 58 aa, synthetic construct
  REGION 1..58: note = Modified bacterial sequence
VDAKYAKETLDAWAEIDHLPNLTLDQWLAFINKLDDDPSQSSELLSEAKKLNDSQAPK

SEQ ID NO: 578 - protein, 58 aa, synthetic construct
  REGION 1..58: note = Modified bacterial sequence
VDAKYAKEHIDAWNEIDALPNLTLSQWLAFINKLDDDPSQSSELLSEAKKLNDSQAPK

SEQ ID NO: 579 - protein, 58 aa, synthetic construct
  REGION 1..58: note = Modified bacterial sequence
VDAKYAKEVLDAWNEIDKLPNLTIAQWLAFINKLDDDPSQSSELLSEAKKLNDSQAPK

SEQ ID NO: 580 - protein, 58 aa, synthetic construct
  REGION 1..58: note = Modified bacterial sequence
VDAKYAKEVIEAWTEIDYLPNLTLQQWLAFINKLDDDPSQSSELLSEAKKLNDSQAPK

SEQ ID NO: 581 - protein, 58 aa, synthetic construct
  REGION 1..58: note = Modified bacterial sequence
VDAKYAKETIEAWNEIDHLPNLTIAQWLAFINKLDDDPSQSSELLSEAKKLNDSQAPK

SEQ ID NO: 582 - protein, 58 aa, synthetic construct
  REGION 1..58: note = Modified bacterial sequence
VDAKYAKEVIQAWNEIDKLPNLTLEQWLAFINKLDDDPSQSSELLSEAKKLNDSQAPK

SEQ ID NO: 583 - protein, 58 aa, synthetic construct
  REGION 1..58: note = Modified bacterial sequence
VDAKYAKEVIEAWDEIDHLPNLTIEQWLAFINKLDDDPSQSSELLSEAKKLNDSQAPK

SEQ ID NO: 584 - protein, 58 aa, synthetic construct
  REGION 1..58: note = Modified bacterial sequence
VDAKYAKETIDAWNEIDLLPNLTIEQWLAFINKLDDDPSQSSELLSEAKKLNDSQAPK

SEQ ID NO: 585 - protein, 58 aa, synthetic construct
  REGION 1..58: note = Modified bacterial sequence
VDAKYAKEHIDAWNEIDKLPNLTLDQWLAFINKLDDDPSQSSELLSEAKKLNDSQAPK

SEQ ID NO: 586 - protein, 58 aa, synthetic construct
  REGION 1..58: note = Modified bacterial sequence
VDAKYAKEVVAAWNEIDALPNLTIEQWLAFINKLNDDPSQSSELLSEAKKLNDSQAPK

SEQ ID NO: 587 - protein, 58 aa, synthetic construct
  REGION 1..58: note = Modified bacterial sequence
VDAKYAKEVIEAWNEIDALPNLTLAQWLAFINKLDDDPSQSSELLSEAKKLNDSQAPK

SEQ ID NO: 588 - protein, 58 aa, synthetic construct
  REGION 1..58: note = Modified bacterial sequence
VDAKYAKEVLQAWDEIDRLPNLTLDQWLAFINKLDDDPSQSSELLSEAKKLNDSQAPK

SEQ ID NO: 589 - protein, 58 aa, synthetic construct
  REGION 1..58: note = Modified bacterial sequence
VDAKYAKEVIDAWDEIDHLPNLTIEQWLAFINKLSDDPSQSSELLSEAKKLNDSQAPK

SEQ ID NO: 590 - protein, 58 aa, synthetic construct
  REGION 1..58: note = Modified bacterial sequence
VDAKYAKEVVEAWNEIDQLPNLTIEQWLAFINKLDDDPSQSSELLSEAKKLNDSQAPK

SEQ ID NO: 591 - protein, 58 aa, synthetic construct
  REGION 1..58: note = Modified bacterial sequence
VDAKYAKEVIQAWNEIDALPNLTIEQWLAFINKLDDDPSQSSELLSEAKKLNDSQAPK

SEQ ID NO: 592 - protein, 58 aa, synthetic construct
  REGION 1..58: note = Modified bacterial sequence
VDAKYAKEVIQAWDEIDKLPNLTIDQWLAFINKLADDPSQSSELLSEAKKLNDSQAPK

SEQ ID NO: 593 - protein, 58 aa, synthetic construct
  REGION 1..58: note = Modified bacterial sequence
VDAKYAKEVVAAWDEIDALPNLTLTQWLAFINKLDDDPSQSSELLSEAKKLNDSQAPK

SEQ ID NO: 594 - protein, 58 aa, synthetic construct
  REGION 1..58: note = Modified bacterial sequence
VDAKYAKEVIQAWNEIDGLPNLTLSQWLAFINKLDDDPSQSSELLSEAKKLNDSQAPK

SEQ ID NO: 595 - protein, 58 aa, synthetic construct
  REGION 1..58: note = Modified bacterial sequence
VDAKYAKETIEAWDEIDALPNLTITQWLAFINKLDDDPSQSSELLSEAKKLNDSQAPK

SEQ ID NO: 596 - protein, 58 aa, synthetic construct
  REGION 1..58: note = Modified bacterial sequence
VDAKYAKEVIDAWNEIDHLPNLTIQQWLAFINKLADDPSQSSELLSEAKKLNDSQAPK

SEQ ID NO: 597 - protein, 58 aa, synthetic construct
  REGION 1..58: note = Modified bacterial sequence
VDAKYAKETIEAWNEIDALPNLTLDQWLAFINKLEDDPSQSSELLSEAKKLNDSQAPK

SEQ ID NO: 598 - protein, 58 aa, synthetic construct
  REGION 1..58: note = Modified bacterial sequence
VDAKYAKEHIHAWNEIDELPNLTIEQWLAFINKLADDPSQSSELLSEAKKLNDSQAPK

SEQ ID NO: 599 - protein, 58 aa, synthetic construct
  REGION 1..58: note = Modified bacterial sequence
VDAKYAKEVIDAWDEIDHLPNLTIDQWLAFINKLSDDPSQSSELLSEAKKLNDSQAPK

SEQ ID NO: 600 - protein, 58 aa, synthetic construct
  REGION 1..58: note = Modified bacterial sequence
VDAKYAKEVIDANDEIDALPNLTIAQWLAFINKLHDDPSQSSELLSEAKKLNDSQAPK

SEQ ID NO: 601 - protein, 58 aa, synthetic construct
  REGION 1..58: note = Modified bacterial sequence
VDAKYAKETIEAWDEIDKLPNLTIEQWLAFINKLDDDPSQSSELLSEAKKLNDSQAPK

SEQ ID NO: 602 - protein, 58 aa, synthetic construct
  REGION 1..58: note = Modified bacterial sequence
VDAKYAKEVLLAWDEIDHLPNLTLEQWLAFINKLDDDPSQSSELLSEAKKLNDSQAPK

SEQ ID NO: 603 - protein, 58 aa, synthetic construct
  REGION 1..58: note = Modified bacterial sequence
VDAKYAKEHIDAWNEIDGLPNLTLEQWLAFINKLDDDPSQSSELLSEAKKLNDSQAPK

SEQ ID NO: 604 - protein, 58 aa, synthetic construct
  REGION 1..58: note = Modified bacterial sequence
VDAKYAKEVIEAWSEIDALPNLTIDQWLAFINKLADDPSQSSELLSEAKKLNDSQAPK

SEQ ID NO: 605 - protein, 58 aa, synthetic construct
  REGION 1..58: note = Modified bacterial sequence
VDAKYAKEQLNAWAEIDALPNLTIEQWLAFINKLDDDPSQSSELLSEAKKLNDSQAPK

SEQ ID NO: 606 - protein, 58 aa, synthetic construct
  REGION 1..58: note = Modified bacterial sequence
VDAKYAKEVIDAWNEIDALPNLTIAQWLAFINKLDDDPSQSSELLSEAKKLNDSQAPK

SEQ ID NO: 607 - protein, 58 aa, synthetic construct
  REGION 1..58: note = Modified bacterial sequence
VDAKYAKETIDAWNEIDQLPNLTIEQWLAFINKLDDDPSQSSELLSEAKKLNDSQAPK

SEQ ID NO: 608 - protein, 58 aa, synthetic construct
  REGION 1..58: note = Modified bacterial sequence
VDAKYAKEVIEAWDEIDKLPNLTLAQWLAFINKLDDDPSQSSELLSEAKKLNDSQAPK

SEQ ID NO: 609 - protein, 58 aa, synthetic construct
  REGION 1..58: note = Modified bacterial sequence
VDAKYAKEVLYAWAEIDHLPNLTIEQWLAFINKLDDDPSQSSELLSEAKKLNDSQAPK

SEQ ID NO: 610 - protein, 58 aa, synthetic construct
  REGION 1..58: note = Modified bacterial sequence
VDAKYAKEQIDAWNEIDRLPNLTIQQWLAFINKLDDDPSQSSELLSEAKKLNDSQAPK

SEQ ID NO: 611 - protein, 58 aa, synthetic construct
  REGION 1..58: note = Modified bacterial sequence
VDAKYAKEVLAAWDEIDRLPNLTIEQWLAFINKLDDDPSQSSELLSEAKKLNDSQAPK

SEQ ID NO: 612 - protein, 58 aa, synthetic construct
  REGION 1..58: note = Modified bacterial sequence
VDAKYAKEVIEAWDEIDHLPNLTLHQWLAFINKLDDDPSQSSELLSEAKKLNDSQAPK

SEQ ID NO: 613 - protein, 58 aa, synthetic construct
  REGION 1..58: note = Modified bacterial sequence
VDAKYAKEVIEAWNEIDKLPNLTLQQWLAFINKLDDDPSQSSELLSEAKKLNDSQAPK

SEQ ID NO: 614 - protein, 58 aa, synthetic construct
  REGION 1..58: note = Modified bacterial sequence
VDAKYAKEVIDANDEIDALPNLTIEQWLAFINKLHDDPSQSSELLSEAKKLNDSQAPK

SEQ ID NO: 615 - protein, 58 aa, synthetic construct
  REGION 1..58: note = Modified bacterial sequence
VDAKYAKEVIAAWDEIDALPNLTIEQWLAFINKLDDDPSQSSELLSEAKKLNDSQAPK

SEQ ID NO: 616 - protein, 58 aa, synthetic construct
  REGION 1..58: note = Modified bacterial sequence
VDAKYAKEVIEAWTEIDQLPNLTLDQWLAFINKLDDDPSQSSELLSEAKKLNDSQAPK

SEQ ID NO: 617 - protein, 58 aa, synthetic construct
  REGION 1..58: note = Modified bacterial sequence
VDAKYAKEVINAWNEIDALPNLTLQQWLAFINKLDDDPSQSSELLSEAKKLNDSQAPK

SEQ ID NO: 618 - protein, 58 aa, synthetic construct
  REGION 1..58: note = Modified bacterial sequence
VDAKYAKEHIEAWDEIDHLPNLTIDQWLAFINKLADDPSQSSELLSEAKKLNDSQAPK

SEQ ID NO: 619 - protein, 58 aa, synthetic construct
  REGION 1..58: note = Modified bacterial sequence
VDAKYAKEHLEAWREIDALPNLTIEQWLAFINKLDDDPSQSSELLSEAKKLNDSQAPK

SEQ ID NO: 620 - protein, 58 aa, synthetic construct
  REGION 1..58: note = Modified bacterial sequence
VDAKYAKEVLDAWNEIDKLPNLTLQQWLAFINKLDDDPSQSSELLSEAKKLNDSQAPK

SEQ ID NO: 621 - protein, 58 aa, synthetic construct
  REGION 1..58: note = Modified bacterial sequence
VDAKYAKEVIAAWDEIDHLPNLTIQQWLAFINKLDDDPSQSSELLSEAKKLNDSQAPK

SEQ ID NO: 622 - protein, 58 aa, synthetic construct
  REGION 1..58: note = Modified bacterial sequence
VDAKYAKEVIQAWNEIDALPNLTLEQWLAFINKLDDDPSQSSELLSEAKKLNDSQAPK

SEQ ID NO: 623 - protein, 58 aa, synthetic construct
  REGION 1..58: note = Modified bacterial sequence
VDAKYAKEVIDAWNEIDHLPNLTIAQWLAFINKLDDDPSQSSELLSEAKKLNDSQAPK

SEQ ID NO: 624 - protein, 58 aa, synthetic construct
  REGION 1..58: note = Modified bacterial sequence
VDAKYAKEQLDAWDEIDHLPNLTIDQWLAFINKLSDDPSQSSELLSEAKKLNDSQAPK

SEQ ID NO: 625 - protein, 58 aa, synthetic construct
  REGION 1..58: note = Modified bacterial sequence
VDAKYAKEVLNAWDEIDKLPNLTIEQWLAFINKLDDDPSQSSELLSEAKKLNDSQAPK

SEQ ID NO: 626 - protein, 58 aa, synthetic construct
  REGION 1..58: note = Modified bacterial sequence
VDAKYAKEVLEAWNEIDHLPNLTIDQWLAFINKLDDDPSQSSELLSEAKKLNDSQAPK

SEQ ID NO: 627 - protein, 58 aa, synthetic construct
  REGION 1..58: note = Modified bacterial sequence
VDAKYAKEVLLAWDEIDRLPNLTIDQWLAFINKLADDPSQSSELLSEAKKLNDSQAPK

SEQ ID NO: 628 - protein, 58 aa, synthetic construct
  REGION 1..58: note = Modified bacterial sequence
VDAKYAKEVIAAWNEIDQLPNLTLDQWLAFINKLDDDPSQSSELLSEAKKLNDSQAPK

SEQ ID NO: 629 - protein, 58 aa, synthetic construct
  REGION 1..58: note = Modified bacterial sequence
VDAKYAKETLLAWDEIDALPNLTIEQWLAFINKLDDDPSQSSELLSEAKKLNDSQAPK

SEQ ID NO: 630 - protein, 58 aa, synthetic construct
  REGION 1..58: note = Modified bacterial sequence
VDAKYAKEVIDAWNEIDTLPNLTLEQWLAFINKLDDDPSQSSELLSEAKKLNDSQAPK

SEQ ID NO: 631 - protein, 58 aa, synthetic construct
  REGION 1..58: note = Modified bacterial sequence
VDAKYAKEVLHAWNEIDHLPNLTLNQWLAFINKLQDDPSQSSELLSEAKKLNDSQAPK

SEQ ID NO: 632 - protein, 58 aa, synthetic construct
  REGION 1..58: note = Modified bacterial sequence
VDAKYAKEVIQAWNEIDALPNLTIAQWLAFINKLDDDPSQSSELLSEAKKLNDSQAPK

SEQ ID NO: 633 - protein, 58 aa, synthetic construct
  REGION 1..58: note = Modified bacterial sequence
VDAKYAKETVDAWNEIDALPNLTIEQWLAFINKLDDDPSQSSELLSEAKKLNDSQAPK

SEQ ID NO: 634 - protein, 58 aa, synthetic construct
  REGION 1..58: note = Modified bacterial sequence
VDAKYAKEVIQAWDEIDHLPNLTIDQWLAFINKLDDDPSQSSELLSEAKKLNDSQAPK

SEQ ID NO: 635 - protein, 58 aa, synthetic construct
  REGION 1..58: note = Modified bacterial sequence
VDAKYAKEVLDAWNEIDQLPNLTIQQWLAFINKLDDDPSQSSELLSEAKKLNDSQAPK

SEQ ID NO: 636 - protein, 58 aa, synthetic construct
  REGION 1..58: note = Modified bacterial sequence
VDAKYAKETIEAWNEIDALPNLTLDQWLAFINKLDDDPSQSSELLSEAKKLNDSQAPK

SEQ ID NO: 637 - protein, 58 aa, synthetic construct
  REGION 1..58: note = Modified bacterial sequence
VDAKYAKEVIEAWDEIDALPNLTIDQWLAFINKLDDDPSQSSELLSEAKKLNDSQAPK

SEQ ID NO: 638 - protein, 58 aa, synthetic construct
  REGION 1..58: note = Modified bacterial sequence
VDAKYAKEVIEAWNEIDQLPNLTIQQWLAFINKLDDDPSQSSELLSEAKKLNDSQAPK

SEQ ID NO: 639 - protein, 58 aa, synthetic construct
  REGION 1..58: note = Modified bacterial sequence
VDAKYAKEVIEAWTEIDHLPNLTIEQWLAFINKLDDDPSQSSELLSEAKKLNDSQAPK

SEQ ID NO: 640 - protein, 58 aa, synthetic construct
  REGION 1..58: note = Modified bacterial sequence
VDAKYAKEVIQAWNEIDHLPNLTLQQWLAFINKLEDDPSQSSELLSEAKKLNDSQAPK

SEQ ID NO: 641 - protein, 58 aa, synthetic construct
  REGION 1..58: note = Modified bacterial sequence
VDAKYAKEVIQANNEIDQLPNLTIEQWLAFINKLHDDPSQSSELLSEAKKLNDSQAPK

SEQ ID NO: 642 - protein, 58 aa, synthetic construct
  REGION 1..58: note = Modified bacterial sequence
VDAKYAKEVLHAWSEIDKLPNLTIEQWLAFINKLDDDPSQSSELLSEAKKLNDSQAPK

SEQ ID NO: 643 - protein, 58 aa, synthetic construct
  REGION 1..58: note = Modified bacterial sequence
VDAKYAKETIQAWDEIDKLPNLTLDQWLAFINKLSDDPSQSSELLSEAKKLNDSQAPK

SEQ ID NO: 644 - protein, 58 aa, synthetic construct
  REGION 1..58: note = Modified bacterial sequence
VDAKYAKETLRAWDEIDKLPNLTIQQWLAFINKLADDPSQSSELLSEAKKLNDSQAPK

SEQ ID NO: 645 - protein, 58 aa, synthetic construct
  REGION 1..58: note = Modified bacterial sequence
VDAKYAKEVIDAWNEIDHLPNLTIEQWLAFINKLEDDPSQSSELLSEAKKLNDSQAPK

SEQ ID NO: 646 - protein, 58 aa, synthetic construct
  REGION 1..58: note = Modified bacterial sequence
VDAKYAKEVIDAWNEIDHLPNLTLQQWLAFINKLADDPSQSSELLSEAKKLNDSQAPK

SEQ ID NO: 647 - protein, 58 aa, synthetic construct
  REGION 1..58: note = Modified bacterial sequence
VDAKYAKETIDAWNEIDALPNLTLDQWLAFINKLDDDPSQSSELLSEAKKLNDSQAPK

SEQ ID NO: 648 - protein, 58 aa, synthetic construct
  REGION 1..58: note = Modified bacterial sequence
VDAKYAKEVIEAWNEIDQLPNLTIEQWLAFINKLDDDPSQSSELLSEAKKLNDSQAPK

SEQ ID NO: 649 - protein, 58 aa, synthetic construct
  REGION 1..58: note = Modified bacterial sequence
VDAKYAKEVIRAWDEIDQLPNLTLSQWLAFINKLDDDPSQSSELLSEAKKLNDSQAPK

SEQ ID NO: 650 - protein, 58 aa, synthetic construct
  REGION 1..58: note = Modified bacterial sequence
VDAKYAKEVIEAWNEIDRLPNLTIHQWLAFINKLDDDPSQSSELLSEAKKLNDSQAPK

SEQ ID NO: 651 - protein, 58 aa, synthetic construct
  REGION 1..58: note = Modified bacterial sequence
VDAKYAKETIEAWNEIDQLPNLTIEQWLAFINKLDDDPSQSSELLSEAKKLNDSQAPK

SEQ ID NO: 652 - protein, 58 aa, synthetic construct
  REGION 1..58: note = Modified bacterial sequence
VDAKYAKEVLTAWAEIDALPNLTLSQWLAFINKLDDDPSQSSELLSEAKKLNDSQAPK

SEQ ID NO: 653 - protein, 58 aa, synthetic construct
  REGION 1..58: note = Modified bacterial sequence
VDAKYAKEVIEAWDEIDKLPNLTVDQWLAFINKLDDDPSQSSELLSEAKKLNDSQAPK

SEQ ID NO: 654 - protein, 58 aa, synthetic construct
  REGION 1..58: note = Modified bacterial sequence
VDAKYAKEVIDAWNEIDHLPNLTLTQWLAFINKLDDDPSQSSELLSEAKKLNDSQAPK

SEQ ID NO: 655 - protein, 58 aa, synthetic construct
  REGION 1..58: note = Modified bacterial sequence
VDAKYAKEVIEAWNEIDQLPNLTLDQWLAFINKLDDDPSQSSELLSEAKKLNDSQAPK

SEQ ID NO: 656 - protein, 58 aa, synthetic construct
  REGION 1..58: note = Modified bacterial sequence
VDAKYAKETLQAWDEIDHLPNLTLNQWLAFINKLDDDPSQSSELLSEAKKLNDSQAPK

SEQ ID NO: 657 - protein, 58 aa, synthetic construct
  REGION 1..58: note = Modified bacterial sequence
VDAKYAKEVIDAWNEIDHLPNLTIEQWLAFINKLDDDPSQSSELLSEAKKLNDSQAPK

SEQ ID NO: 658 - protein, 58 aa, synthetic construct
  REGION 1..58: note = Modified bacterial sequence
VDAKYAKEVIEAWNEIDLLPNLTLSQWLAFINKLDDDPSQSSELLSEAKKLNDSQAPK

SEQ ID NO: 659 - protein, 58 aa, synthetic construct
  REGION 1..58: note = Modified bacterial sequence
VDAKYAKEVIDAWDEIDRLPNLTLKQWLAFINKLDDDPSQSSELLSEAKKLNDSQAPK

SEQ ID NO: 660 - protein, 58 aa, synthetic construct
  REGION 1..58: note = Modified bacterial sequence
VDAKYAKETLHAWDEIDKLPNLTIEQWLAFINKLDDDPSQSSELLSEAKKLNDSQAPK

SEQ ID NO: 661 - protein, 58 aa, synthetic construct
  REGION 1..58: note = Modified bacterial sequence
VDAKYAKEVIKAWDEIDHLPNLTLNQWLAFINKLDDDPSQSSELLSEAKKLNDSQAPK

SEQ ID NO: 662 - protein, 58 aa, synthetic construct
  REGION 1..58: note = Modified bacterial sequence
VDAKYAKEVIEAWNEIDHLPNLTLAQWLAFINKLDDDPSQSSELLSEAKKLNDSQAPK

SEQ ID NO: 663 - protein, 58 aa, synthetic construct
  REGION 1..58: note = Modified bacterial sequence
VDAKYAKEVIQAWNEIDHLPNLTIDQWLAFITKLEDDPSQSSELLSEAKKLNDSQAPK

SEQ ID NO: 664 - protein, 58 aa, synthetic construct
  REGION 1..58: note = Modified bacterial sequence
VDAKYAKEVIEAWNEIDRLPNLTIKQWLAFINKLDDDPSQSSELLSEAKKLNDSQAPK

SEQ ID NO: 665 - protein, 58 aa, synthetic construct
  REGION 1..58: note = Modified bacterial sequence
VDAKYAKEVIEAWNEIDSLPNLTLQQWLAFINKLDDDPSQSSELLSEAKKLNDSQAPK

SEQ ID NO: 666 - protein, 58 aa, synthetic construct
  REGION 1..58: note = Modified bacterial sequence
VDAKYAKETIDAWNEIDKLPNLTIEQWLAFINKLDDDPSQSSELLSEAKKLNDSQAPK

SEQ ID NO: 667 - protein, 58 aa, synthetic construct
  REGION 1..58: note = Modified bacterial sequence
VDAKYAKEVLEAWAEIDALPNLTIAQWLAFINKLDDDPSQSSELLSEAKKLNDSQAPK

SEQ ID NO: 668 - protein, 58 aa, synthetic construct
  REGION 1..58: note = Modified bacterial sequence
VDAKYAKETIDAWNEIDRLPNLTIEQWLAFINKLDDDPSQSSELLSEAKKLNDSQAPK

SEQ ID NO: 669 - protein, 58 aa, synthetic construct
  REGION 1..58: note = Modified bacterial sequence
VDAKYAKETLKAWDEIDRLPNLTLEQWLAFINKLDDDPSQSSELLSEAKKLNDSQAPK

SEQ ID NO: 670 - protein, 58 aa, synthetic construct
  REGION 1..58: note = Modified bacterial sequence
VDAKYAKETIAAWNEIDALPNLTLQQWLAFINKLDDDPSQSSELLSEAKKLNDSQAPK

SEQ ID NO: 671 - protein, 58 aa, synthetic construct
  REGION 1..58: note = Modified bacterial sequence
VDAKYAKEVLQAWNEIDHLPNLTIQQWLAFINKLDDDPSQSSELLSEAKKLNDSQAPK

SEQ ID NO: 672 - protein, 58 aa, synthetic construct
  REGION 1..58: note = Modified bacterial sequence
VDAKYAKEVIEAWSEIDHLPNLTLQQWLAFINKLDDDPSQSSELLSEAKKLNDSQAPK

SEQ ID NO: 673 - protein, 58 aa, synthetic construct
  REGION 1..58: note = Modified bacterial sequence
VDAKYAKEVIDAWNEIDGLPNLTIEQWLAFINKLDDDPSQSSELLSEAKKLNDSQAPK

SEQ ID NO: 674 - protein, 58 aa, synthetic construct
  REGION 1..58: note = Modified bacterial sequence
VDAKYAKEVIHAWNEIDHLPNLTLNQWLAFINKLEDDPSQSSELLSEAKKLNDSQAPK

SEQ ID NO: 675 - protein, 58 aa, synthetic construct
  REGION 1..58: note = Modified bacterial sequence
VDAKYAKEVLDAWNEIDSLPNLTLDQWLAFINKLDDDPSQSSELLSEAKKLNDSQAPK

SEQ ID NO: 676 - protein, 58 aa, synthetic construct
  REGION 1..58: note = Modified bacterial sequence
VDAKYAKEQIEAWNEIDRLPNLTLEQWLAFINKLDDDPSQSSELLSEAKKLNDSQAPK

SEQ ID NO: 677 - protein, 58 aa, synthetic construct
  REGION 1..58: note = Modified bacterial sequence
VDAKYAKEVVDAWNEIDALPNLTLQQWLAFINKLDDDPSQSSELLSEAKKLNDSQAPK

SEQ ID NO: 678 - protein, 58 aa, synthetic construct
  REGION 1..58: note = Modified bacterial sequence
VDAKYAKEVIEAWNEIDKLPNLTIEQWLAFINKLDDDPSQSSELLSEAKKLNDSQAPK

SEQ ID NO: 679 - protein, 58 aa, synthetic construct
  REGION 1..58: note = Modified bacterial sequence
VDAKYAKEVIEANDEIDRLPNLTIEQWLAFINKLHDDPSQSSELLSEAKKLNDSQAPK

SEQ ID NO: 680 - protein, 58 aa, synthetic construct
  REGION 1..58: note = Modified bacterial sequence
VDAKYAKETLQAWDEIDKLPNLTIEQWLAFINKLDDDPSQSSELLSEAKKLNDSQAPK

SEQ ID NO: 681 - protein, 58 aa, synthetic construct
  REGION 1..58: note = Modified bacterial sequence
VDAKYAKEVIEAWDEIDHLPNLTIDQWLAFINKLADDPSQSSELLSEAKKLNDSQAPK

SEQ ID NO: 682 - protein, 58 aa, synthetic construct
  REGION 1..58: note = Modified bacterial sequence
VDAKYAKETIDAWNEIDHLPNLTLQQWLAFINKLADDPSQSSELLSEAKKLNDSQAPK

SEQ ID NO: 683 - protein, 58 aa, synthetic construct
  REGION 1..58: note = Modified bacterial sequence
VDAKYAKEVIDAWDEIDKLPNLTIEQWLAFINKLDDDPSQSSELLSEAKKLNDSQAPK

SEQ ID NO: 684 - protein, 58 aa, synthetic construct
  REGION 1..58: note = Modified bacterial sequence
VDAKYAKEVIEAWNEIDKLPNLTLAQWLAFINKLDDDPSQSSELLSEAKKLNDSQAPK

SEQ ID NO: 685 - protein, 58 aa, synthetic construct
  REGION 1..58: note = Modified bacterial sequence
VDAKYAKEVLQAWDEIDKLPNLTIQQWLAFINKLDDDPSQSSELLSEAKKLNDSQAPK

SEQ ID NO: 686 - protein, 58 aa, synthetic construct
  REGION 1..58: note = Modified bacterial sequence
VDAKYAKEVIAAWNEIDGLPNLTLQQWLAFINKLDDDPSQSSELLSEAKKLNDSQAPK

SEQ ID NO: 687 - protein, 58 aa, synthetic construct
  REGION 1..58: note = Modified bacterial sequence
VDAKYAKETLNAWNEIDALPNLTLQQWLAFINKLDDDPSQSSELLSEAKKLNDSQAPK

SEQ ID NO: 688 - protein, 58 aa, synthetic construct
  REGION 1..58: note = Modified bacterial sequence
VDAKYAKEVLSAWNEIDQLPNLTLEQWLAFINKLDDDPSQSSELLSEAKKLNDSQAPK

SEQ ID NO: 689 - protein, 58 aa, synthetic construct
  REGION 1..58: note = Modified bacterial sequence
VDAKYAKETLEAWDEIDHLPNLTLHQWLAFINKLDDDPSQSSELLSEAKKLNDSQAPK

SEQ ID NO: 690 - protein, 58 aa, synthetic construct
  REGION 1..58: note = Modified bacterial sequence
VDAKYAKEQIEAWNEIDHLPNLTLQQWLAFINKLADDPSQSSELLSEAKKLNDSQAPK

SEQ ID NO: 691 - protein, 58 aa, synthetic construct
  REGION 1..58: note = Modified bacterial sequence
VDAKYAKEVVEAWDEIDKLPNLTIEQWLAFINKLDDDPSQSSELLSEAKKLNDSQAPK

SEQ ID NO: 692 - protein, 58 aa, synthetic construct
  REGION 1..58: note = Modified bacterial sequence
VDAKYAKEVLEAWNEIDELPNLTIEQWLAFINKLDDDPSQSSELLSEAKKLNDSQAPK

SEQ ID NO: 693 - protein, 58 aa, synthetic construct
  REGION 1..58: note = Modified bacterial sequence
VDAKYAKEVIDAWNEIDQLPNLTLQQWLAFINKLDDDPSQSSELLSEAKKLNDSQAPK

SEQ ID NO: 694 - protein, 58 aa, synthetic construct
  REGION 1..58: note = Modified bacterial sequence
VDAKYAKETIDAWDEIDKLPNLTLSQWLAFINKLDDDPSQSSELLSEAKKLNDSQAPK

SEQ ID NO: 695 - protein, 58 aa, synthetic construct
  REGION 1..58: note = Modified bacterial sequence
VDAKYAKETIDAWNEIDQLPNLTLQQWLAFINKLDDDPSQSSELLSEAKKLNDSQAPK

SEQ ID NO: 696 - protein, 58 aa, synthetic construct
  REGION 1..58: note = Modified bacterial sequence
VDAKYAKEVIQAWDEIDALPNLTLNQWLAFINKLDDDPSQSSELLSEAKKLNDSQAPK

SEQ ID NO: 697 - protein, 58 aa, synthetic construct
  REGION 1..58: note = Modified bacterial sequence
VDAKYAKEVLDAWAEIDQLPNLTLQQWLAFINKLDDDPSQSSELLSEAKKLNDSQAPK

SEQ ID NO: 698 - protein, 58 aa, synthetic construct
  REGION 1..58: note = Modified bacterial sequence
VDAKYAKEHIAAWDEIDALPNLTIEQWLAFINKLDDDPSQSSELLSEAKKLNDSQAPK

SEQ ID NO: 699 - protein, 58 aa, synthetic construct
  REGION 1..58: note = Modified bacterial sequence
VDAKYAKEVIRAWDEIDALPNLTIEQWLAFINKLDDDPSQSSELLSEAKKLNDSQAPK

SEQ ID NO: 700 - protein, 58 aa, synthetic construct
  REGION 1..58: note = Modified bacterial sequence
VDAKYAKEVIDAWDEIDALPNLTIDQWLAFINKLADDPSQSSELLSEAKKLNDSQAPK

SEQ ID NO: 701 - protein, 58 aa, synthetic construct
  REGION 1..58: note = Modified bacterial sequence
VDAKYAKEVIDAWNEIDRLPNLTIQQWLAFINKLDDDPSQSSELLSEAKKLNDSQAPK

SEQ ID NO: 702 - protein, 58 aa, synthetic construct
  REGION 1..58: note = Modified bacterial sequence
VDAKYAKEVITAWNEIDHLPNLTLSQWLAFINKLDDDPSQSSELLSEAKKLNDSQAPK

SEQ ID NO: 703 - protein, 58 aa, synthetic construct
  REGION 1..58: note = Modified bacterial sequence
VDAKYAKEVIDAWNEIDALPNLTIHQWLAFINKLDDDPSQSSELLSEAKKLNDSQAPK

SEQ ID NO: 704 - protein, 58 aa, synthetic construct
  REGION 1..58: note = Modified bacterial sequence
VDAKYAKEQLKAWDEIDKLPNLTIEQWLAFIEKLQDDPSQSSELLSEAKKLNDSQAPK

SEQ ID NO: 705 - protein, 58 aa, synthetic construct
  REGION 1..58: note = Modified bacterial sequence
VDAKYAKEHIDAWTEIDHLPNLTIEQWLAFINKLDDDPSQSSELLSEAKKLNDSQAPK

SEQ ID NO: 706 - protein, 58 aa, synthetic construct
  REGION 1..58: note = Modified bacterial sequence
VDAKYAKEQLRAWDEIDKLPNLTIEQWLAFINKLQDDPSQSSELLSEAKKLNDSQAPK

SEQ ID NO: 707 - protein, 58 aa, synthetic construct
  REGION 1..58: note = Modified bacterial sequence
VDAKYAKEVLEAWREIDSLPNLTIAQWLAFINKLDDDPSQSSELLSEAKKLNDSQAPK

SEQ ID NO: 708 - protein, 58 aa, synthetic construct
  REGION 1..58: note = Modified bacterial sequence
VDAKYAKEVIQAWNEIDKLPNLTIEQWLAFINKLDDDPSQSSELLSEAKKLNDSQAPK

SEQ ID NO: 709 - protein, 58 aa, synthetic construct
  REGION 1..58: note = Modified bacterial sequence
VDAKYAKEHVEAWNEIDQLPNLTIEQWLAFINKLADDPSQSSELLSEAKKLNDSQAPK

SEQ ID NO: 710 - protein, 58 aa, synthetic construct
  REGION 1..58: note = Modified bacterial sequence
VDAKYAKEVIDAWDEIDALPNLTIDQWLAFINKLSDDPSQSSELLSEAKKLNDSQAPK

SEQ ID NO: 711 - protein, 58 aa, synthetic construct
  REGION 1..58: note = Modified bacterial sequence
VDAKYAKEVIEAWNEIDHLPNLTIEQWLAFINKLDDDPSQSSELLSEAKKLNDSQAPK

SEQ ID NO: 712 - protein, 58 aa, synthetic construct
  REGION 1..58: note = Modified bacterial sequence
VDAKYAKEVLQAWDEIDKLPNLTIEQWLAFINKLSDDPSQSSELLSEAKKLNDSQAPK

SEQ ID NO: 713 - protein, 58 aa, synthetic construct
  REGION 1..58: note = Modified bacterial sequence
VDAKYAKEVIKAWNEIDSLPNLTIEQWLAFINKLDDDPSQSSELLSEAKKLNDSQAPK

SEQ ID NO: 714 - protein, 58 aa, synthetic construct
  REGION 1..58: note = Modified bacterial sequence
VDAKYAKEVLEAWHEIDLLPNLTIQQWLAFINKLDDDPSQSSELLSEAKKLNDSQAPK

SEQ ID NO: 715 - protein, 58 aa, synthetic construct
  REGION 1..58: note = Modified bacterial sequence
VDAKYAKEVLEAWTEIDRLPNLTLDQWLAFINKLDDDPSQSSELLSEAKKLNDSQAPK

SEQ ID NO: 716 - protein, 58 aa, synthetic construct
  REGION 1..58: note = Modified bacterial sequence
VDAKYAKEQLYAWNEIDHLPNLTIEQWLAFIEKLQDDPSQSSELLSEAKKLNDSQAPK

SEQ ID NO: 717 - protein, 58 aa, synthetic construct
  REGION 1..58: note = Modified bacterial sequence
VDAKYAKEVLNAWDEIDKLPNLTIKQWLAFINKLDDDPSQSSELLSEAKKLNDSQAPK

SEQ ID NO: 718 - protein, 58 aa, synthetic construct
  REGION 1..58: note = Modified bacterial sequence
VDAKYAKEVIRAWDEIDKLPNLTVEQWLAFINKLDDDPSQSSELLSEAKKLNDSQAPK

SEQ ID NO: 719 - protein, 58 aa, synthetic construct
  REGION 1..58: note = Modified bacterial sequence
VDAKYAKEVVQAWDEIDQLPNLTLEQWLAFINKLDDDPSQSSELLSEAKKLNDSQAPK

SEQ ID NO: 720 - protein, 58 aa, synthetic construct
  REGION 1..58: note = Modified bacterial sequence
VDAKYAKEVIRAWDEIDQLPNLTLEQWLAFINKLDDDPSQSSELLSEAKKLNDSQAPK

SEQ ID NO: 721 - protein, 58 aa, synthetic construct
  REGION 1..58: note = Modified bacterial sequence
VDAKYAKETIDAWNEIDHLPNLTLDQWLAFINKLDDDPSQSSELLSEAKKLNDSQAPK

SEQ ID NO: 722 - protein, 58 aa, synthetic construct
  REGION 1..58: note = Modified bacterial sequence
VDAKYAKEVVAAWTEIDLLPNLTLDQWLAFINKLEDDPSQSSELLSEAKKLNDSQAPK

SEQ ID NO: 723 - protein, 58 aa, synthetic construct
  REGION 1..58: note = Modified bacterial sequence
VDAKYAKEVVAAWDEIDALPNLTIEQWLAFINKLSDDPSQSSELLSEAKKLNDSQAPK

SEQ ID NO: 724 - protein, 58 aa, synthetic construct
  REGION 1..58: note = Modified bacterial sequence
VDAKYAKETLEAWREIDSLPNLTLEQWLAFINKLDDDPSQSSELLSEAKKLNDSQAPK

SEQ ID NO: 725 - protein, 58 aa, synthetic construct
  REGION 1..58: note = Modified bacterial sequence
VDAKYAKEVIKAWNEIDHLPNLTLDQWLAFINKLDDDPSQSSELLSEAKKLNDSQAPK

SEQ ID NO: 726 - protein, 58 aa, synthetic construct
  REGION 1..58: note = Modified bacterial sequence
VDAKYAKEVLEAWTEIDKLPNLTIDQWLAFINKLDDDPSQSSELLSEAKKLNDSQAPK

SEQ ID NO: 727 - protein, 58 aa, synthetic construct
  REGION 1..58: note = Modified bacterial sequence
VDAKYAKETLEAWDEIDKLPNLTIDQWLAFINKLDDDPSQSSELLSEAKKLNDSQAPK

SEQ ID NO: 728 - protein, 58 aa, synthetic construct
  REGION 1..58: note = Modified bacterial sequence
VDAKYAKEVIEAWNEIDKLPNLTIDQWLAFINKLDDDPSQSSELLSEAKKLNDSQAPK

SEQ ID NO: 729 - protein, 58 aa, synthetic construct
  REGION 1..58: note = Modified bacterial sequence
VDAKYAKETIDAWNEIDKLPNLTLEQWLAFINKLDDDPSQSSELLSEAKKLNDSQAPK

SEQ ID NO: 730 - protein, 58 aa, synthetic construct
  REGION 1..58: note = Modified bacterial sequence
VDAKYAKETLDAWDEIDALPNLTIDQWLAFINKLEDDPSQSSELLSEAKKLNDSQAPK

SEQ ID NO: 731 - protein, 58 aa, synthetic construct
  REGION 1..58: note = Modified bacterial sequence
VDAKYAKEVLSAWNEIDHLPNLTIQQWLAFINKLDDDPSQSSELLSEAKKLNDSQAPK

SEQ ID NO: 732 - protein, 58 aa, synthetic construct
  REGION 1..58: note = Modified bacterial sequence
VDAKYAKEVIQANDEIDKLPNLTIEQWLAFIHKLHDDPSQSSELLSEAKKLNDSQAPK

SEQ ID NO: 733 - protein, 58 aa, synthetic construct
  REGION 1..58: note = Modified bacterial sequence
VDAKYAKEHLDAWDEIDHLPNLTIQQWLAFINKLADDPSQSSELLSEAKKLNDSQAPK

SEQ ID NO: 734 - protein, 58 aa, synthetic construct
  REGION 1..58: note = Modified bacterial sequence
VDAKYAKEVIQAWNEIDQLPNLTIEQWLAFINKLDDDPSQSSELLSEAKKLNDSQAPK

SEQ ID NO: 735 - protein, 58 aa, synthetic construct
  REGION 1..58: note = Modified bacterial sequence
VDAKYAKEVIEAWNEIDYLPNLTIAQWIAFINKLDDDPSQSSELLSEAKKLNDSQAPK

SEQ ID NO: 736 - protein, 58 aa, synthetic construct
  REGION 1..58: note = Modified bacterial sequence
VDAKYAKETIQAWDEIDRLPNLTLQQWLAFINKLDDDPSQSSELLSEAKKLNDSQAPK

SEQ ID NO: 737 - protein, 58 aa, synthetic construct
  REGION 1..58: note = Modified bacterial sequence
VDAKYAKETIQAWDEIDKLPNLTIEQWLAFINKLDDDPSQSSELLSEAKKLNDSQAPK

SEQ ID NO: 738 - protein, 58 aa, synthetic construct
  REGION 1..58: note = Modified bacterial sequence
VDAKYAKETLDAWAEIDHLPNLTIEQWLAFINKLDDDPSQSSELLSEAKKLNDSQAPK

SEQ ID NO: 739 - protein, 58 aa, synthetic construct
  REGION 1..58: note = Modified bacterial sequence
VDAKYAKEVIEAWDEIDKLPNLTLNQWLAFINKLDDDPSQSSELLSEAKKLNDSQAPK

SEQ ID NO: 740 - protein, 58 aa, synthetic construct
  REGION 1..58: note = Modified bacterial sequence
VDAKYAKEVLDAWNEIDQLPNLTIEQWLAFINKLDDDPSQSSELLSEAKKLNDSQAPK

SEQ ID NO: 741 - protein, 58 aa, synthetic construct
  REGION 1..58: note = Modified bacterial sequence
VDAKYAKEVLHAWNEIDHLPNLTIEQWLAFIEKLEDDPSQSSELLSEAKKLNDSQAPK

SEQ ID NO: 742 - protein, 58 aa, synthetic construct
  REGION 1..58: note = Modified bacterial sequence
VDAKYAKEVIEAWQEIDKLPNLTIDQWLAFINKLDDDPSQSSELLSEAKKLNDSQAPK

SEQ ID NO: 743 - protein, 58 aa, synthetic construct
  REGION 1..58: note = Modified bacterial sequence
VDAKYAKEVVDAWNEIDQLPNLTIEQWLAFINKLDDDPSQSSELLSEAKKLNDSQAPK

SEQ ID NO: 744 - protein, 58 aa, synthetic construct
  REGION 1..58: note = Modified bacterial sequence
VDAKYAKEQIEAWNEIDALPNLTIEQWLAFINKLADDPSQSSELLSEAKKLNDSQAPK

SEQ ID NO: 745 - protein, 60 aa, synthetic construct
  REGION 1..60: note = Modified bacterial sequence
VDAKYAKETITAWDEIDKLPNLTIEQWLAFIGKLEDDPSQSSELLAEAKKLNDAQAPKVD

SEQ ID NO: 746 - protein, 60 aa, synthetic construct
  REGION 1..60: note = Modified bacterial sequence
AEAKYAKEVIEAWNEIDRLPNLTIEQWLAFINKLDDDPSQSSELLSEAKKLNDSQAPKVD

SEQ ID NO: 747 - protein, 60 aa, synthetic construct
  REGION 1..60: note = Modified bacterial sequence
AEAKYAKEVLEAWNEIDRLPNLTIEQWLAFINKLDDDPSQSSELLSEAKKLNDSQAPKVD

SEQ ID NO: 748 - protein, 60 aa, synthetic construct
  REGION 1..60: note = Modified bacterial sequence
AEAKYAKEVLEAWDEIDRLPNLTLDQWLAFINKLDDDPSQSSELLSEAKKLNDSQAPKVD

SEQ ID NO: 749 - protein, 60 aa, synthetic construct
  REGION 1..60: note = Modified bacterial sequence
AEAKYAKEVLDAWDEIDKLPNLTLEQWLAFINKLDDDPSQSSELLSEAKKLNDSQAPKVD

SEQ ID NO: 750 - protein, 60 aa, synthetic construct
  REGION 1..60: note = Modified bacterial sequence
AEAKYAKEVLDAWDEIDALPNLTLEQWLAFINKLDDDPSQSSELLSEAKKLNDSQAPKVD

SEQ ID NO: 751 - protein, 60 aa, synthetic construct
  REGION 1..60: note = Modified bacterial sequence
AEAKYAKEVIDAWDEIDRLPNLTLDQWLAFINKLDDDPSQSSELLSEAKKLNDSQAPKVD

SEQ ID NO: 752 - protein, 60 aa, synthetic construct
  REGION 1..60: note = Modified bacterial sequence
AEAKYAKETLEAWDEIDRLPNLTIEQWLAFINKLDDDPSQSSELLSEAKKLNDSQAPKVD

SEQ ID NO: 753 - protein, 60 aa, synthetic construct
  REGION 1..60: note = Modified bacterial sequence
AEAKYAKEVLEAWDEIDRLPNLTIEQWLAFINKLDDDPSQSSELLSEAKKLNDSQAPKVD

SEQ ID NO: 754 - protein, 60 aa, synthetic construct
  REGION 1..60: note = Modified bacterial sequence
AEAKYAKEVIDAWNEIDALPNLTLDQWLAFINKLDDDPSQSSELLSEAKKLNDSQAPKVD

SEQ ID NO: 755 - protein, 60 aa, synthetic construct
  REGION 1..60: note = Modified bacterial sequence
AEAKYAKEVLDAWDEIDKLPNLTIDQWLAFINKLDDDPSQSSELLSEAKKLNDSQAPKVD

SEQ ID NO: 756 - protein, 60 aa, synthetic construct
  REGION 1..60: note = Modified bacterial sequence
AEAKYAKEVLEAWDEIDHLPNLTLDQWLAFINKLDDDPSQSSELLSEAKKLNDSQAPKVD

SEQ ID NO: 757 - protein, 60 aa, synthetic construct
  REGION 1..60: note = Modified bacterial sequence
AEAKYAKEVLEAWDEIDALPNLTIEQWLAFINKLDDDPSQSSELLSEAKKLNDSQAPKVD

SEQ ID NO: 758 - protein, 58 aa, synthetic construct [sequence omitted]

SEQ ID NO: 759 - protein, 46 aa, synthetic construct
  REGION 1..46: note = Modified bacterial sequence
LAEAKEAANAELDSYGVSDFYKRLIDKAKTVEGVEALKDAILAALP

SEQ ID NO: 760 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 761 - protein, 170 aa, Ornithodoros moubata
MDSESDCTGSEPVDAFQAFSEGKEAYVLVRSTDPKARDCLKGEPAGEKQDNTLPVMMTFKNGTDWASTDWTFTLDGAKVTATLGNLTQNREVVYDSQSHHCHVDKVEKEVPDYEMWMLDAGGLEVEVECCRQKLEELASGRNQMYPHLKDCGGGGSENLYFQGSHHHHHH

SEQ ID NO: 762 - protein, 1676 aa, Macaca fascicularis
  VARIANT 63: note = misc_feature - Xaa can be any naturally occurring amino acid
  VARIANT 1346: note = misc_feature - Xaa can be any naturally occurring amino acid
MGLLGILCFLIFLGKTWGQEQTYVISAPKIFRVGASENIVIQVYGYTEAFDATISIKSYPDKXFSYSSGHVHLSSENKFQNSAVLTIQPKQLPGGQNQVSYVYLEVVSKHFSKSKKIPITYDNGFLFIHTDKPVYTPDQSVKVRVYSLNDDLKPAKRETVLTFIDPEGSEIDMVEEIDHIGIISFPDFKIPSNPRYGMWTIQAKYKEDFSTTGTAFFEVKEYVLPHFSVSVEPESNFIGYKNFKNFEITIKARIFYNKVVTEADVYITFGIREDLKDDQKEMMQTAMQNTMLINGIAEVTFDSETAVKELSYYSLEDLNNKYLYIAVTVIESTGGFSEEAEIPGIKYVLSPYKLNLVATPLFLKPGIPYSIKVQVKDALDQLVGGVPVTLNAQTIDVNQETSDLEPRKSVTRVDDGVASFVVNLPSGVTVLEFNVKTDAPDLPDENQAREGYRAIAYSSLSQSYLYIDWTDNHKALLVGEYLNIIVTPKSPYIDKITHYNYLILSKGKIIHFGTREKLSDASYQSINIPVTQNMVPSSRLLVYYIVTGEQTAELVSDSVWLNIEEKCGNQLQVHLSPDADTYSPGQTVSLNMVTGMDSWVALTAVDSAVYGVQRRAKKPLERVFQFLEKSDLGCGAGGGLNNANVFHLAGLTFLTNANADDSQENDEPCKEIIRPRRMLQEKIEEIAAKYKHLVVKKCCYDGVRINHDETCEQRAARISVGPRCVKAFTECCVVASQLRANNSHKDLQLGRLHMKTLLPVSKPEIRSYFPESWLWEVHLVPRRKQLQFALPDSVTTWEIQGVGISNSGICVADTIKAKVFKDVFLEMNIPYSVVRGEQVQLKGTVYNYRTSGMQFCVKMSAVEGICTSESPVIDHQGTKSSKCVRQKVEGSSNHLVTFTVLPLEIGLQNINFSLETSFGKEILVKSLRVVPEGVKRESYSGITLDPRGIYGTISRRKEFPYRIPLDLVPKTEIKRILSVKGLLVGEILSAVLSREGINILTHLPKGSAEAELMSVVPVFYVFHYLETGNHWNIFHSDPLIEKRNLEKKLKEGMVSIMSYRNADYSYSVWKGGSASTWLTAFALRVLGQVHKYVEQNQNSICNSLLWLVENYQLDNGSFKENSQYQPIKLQGTLPVEARENSLYLTAFTVIGIRKAFDICPLVKINTALIKADTFLLENTLPAQSTFTLAISAYALSLGDKTHPQFRSIVSALKREALVKGNPPIYRFWKDSLQHKDSSVPNTGTARMVETTAYALLTSLNLKDINYVNPIIKWLSEEQRYGGGFYSTQDTINAIEGLTEYSLLVKQLRLNMDIDVAYKHKGPLHNYKMTDKNFLGRPVEVLLNDDXVVSTGFGSGLATVHVTTVVHKTSTSEEVCSFYLKIDTQDVEASHYRGYGNSDYKRIVACASYKPSKEESSSGSSHAVMDISLPTGINANEEDLKALVEGVDQLFTDYQIKDGHVILQLNSIPSSDFLCVRFRIFELFEVGFLSPATFTVYEYHRPDKQCTMFYSTSNIKIQKVCEGATCKCIEADCGQMQKELDLTISAETRKQTACNPEIAYAYKVIITSITTENVFVKYKATLLDIYKTGEAVAEKDSEITFIKKVTCTNAELVKGRQYLIMGKEALQIKYNFTFRYIYPLDSLTWIEYWPRDTTCSSCQAFLANLDEFAEDIFLNGC

SEQ ID NO: 763 - protein, 29 aa, synthetic construct
  REGION 1..29: note = Engineered Polypeptide
  VARIANT 2: note = MISC_FEATURE - Amino acid selected from H, Q, S, T and V
  VARIANT 3: note = MISC_FEATURE - Amino acid selected from I, L, M, and V
  VARIANT 4: note = MISC_FEATURE - Amino acid selected from A, D, E, H, K, L, N, Q, R, S, T and Y
  VARIANT 6: note = MISC_FEATURE - Amino acid selected from N and W
  VARIANT 7: note = MISC_FEATURE - Amino acid selected from A, D, E, H, N, Q, R, S and T
  VARIANT 11: note = MISC_FEATURE - Amino acid selected from A, E, G, H, K, L, Q, R, S, T and Y
  VARIANT 16: note = MISC_FEATURE - Amino acid selected from N and T
  VARIANT 17: note = MISC_FEATURE - Amino acid selected from I, L and V
  VARIANT 18: note = MISC_FEATURE - Amino acid selected from A, D, E, H, K, N, Q, R, S and T
  VARIANT 21: note = MISC_FEATURE - Amino acid selected from I, L and V
  VARIANT 25: note = MISC_FEATURE - Amino acid selected from D, E, G, H, N, S and T
  VARIANT 26: note = MISC_FEATURE - Amino acid selected from K and S
  VARIANT 28: note = MISC_FEATURE - Amino acid selected from A, D, E, H, N, Q, S, T and Y
EXXXAXXEIDXLPNLXXXQWXAFIXXLXD

SEQ ID NO: 764 - protein, 20 aa, synthetic construct
  REGION 1..20: note = Engineered Polypeptide
  REGION 1..2: note = MISC_FEATURE - BM is a C5 binding motif
  VARIANT 7: note = MISC_FEATURE - Xaa selected from A and S
  VARIANT 8: note = MISC_FEATURE - Xaa selected from N and E
  VARIANT 11: note = MISC_FEATURE - Xaa selected from A, S and C
  VARIANT 19: note = MISC_FEATURE - Xaa selected from A and S
KDPSQSXXLLXEAKKLNDXQ

SEQ ID NO: 765 - protein, 24 aa, synthetic construct
  REGION 1..24: note = Engineered Polypeptide
  REGION 3..4: note = MISC_FEATURE - BM is a C5 binding motif
  VARIANT 13: note = MISC_FEATURE - Xaa selected from S and C
YAKDPSQSSELLXEAKKLNDSQAP

SEQ ID NO: 766 - protein, 24 aa, synthetic construct
  REGION 1..24: note = Engineered Polypeptide
  REGION 3..4: note = MISC_FEATURE - BM is a C5 binding motif
  VARIANT 13: note = MISC_FEATURE - Xaa selected from A and C
FNKDPSQSANLLXEAKKLNDAQAP

SEQ ID NO: 767 - protein, 16 aa, synthetic construct
  REGION 1..16: note = Engineered Polypeptide
  REGION 8..9: note = MISC_FEATURE - Individual Z variant
  REGION 12..13: note = MISC_FEATURE - Albumin binding domain
AQHDEALEVDYVYVPG

SEQ ID NO: 768 - protein, 16 aa, synthetic construct
  REGION 1..16: note = Engineered Polypeptide
  REGION 8..9: note = MISC_FEATURE - Individual Z variant
  REGION 12..13: note = MISC_FEATURE - Z variant Z03639
AQHDEALEVDYVYVPG

SEQ ID NO: 769 - protein, 16 aa, synthetic construct
  REGION 1..16: note = Engineered Polypeptide
  REGION 8..9: note = MISC_FEATURE - Z variant Z03938
  REGION 12..13: note = MISC_FEATURE - Z variant Z03639
AQHDEALEVDYVYVPG

SEQ ID NO: 770 - DNA, 24 bp, synthetic construct
  misc_feature 1..24: note = Primer Sequence
  source 1..24: mol_type = other DNA
tgcttccggctcgtatgttgtgtg

SEQ ID NO: 771 - DNA, 21 bp, synthetic construct
  misc_feature 1..21: note = Primer sequence
  source 1..21: mol_type = other DNA
cggaaccagagccaccaccgg

SEQ ID NO: 772 - DNA, 21 bp, synthetic construct
  misc_feature 1..21: note = Primer sequence
  misc_feature 1: note = 5' biotin cap
  source 1..21: mol_type = other DNA
cggaaccagagccaccaccgg

SEQ ID NO: 773 - protein, 14 aa, synthetic construct
  REGION 1..14: note = Engineered Polypeptide
  REGION 12..13: note = MISC_FEATURE - Two individual Z variants
MGSSHHHHHHLQVD

SEQ ID NO: 774 - DNA, 183 bp, synthetic construct
  misc_feature 1..183: note = Library coding sequence
  misc_feature 42..44: note = Randomized codon
  misc_feature 45..47: note = Randomized codon
  misc_feature 48..50: note = Randomized codon
  misc_feature 55..59: note = Randomized codon
  misc_feature 60..62: note = Randomized codon
  misc_feature 75..77: note = Randomized codon
  misc_feature 78..80: note = Randomized codon
  misc_feature 102..104: note = Randomized codon
  misc_feature 105..107: note = Randomized codon
  misc_feature 117..119: note = Randomized codon
  misc_feature 135..137: note = Randomized codon
  misc_feature 150..152: note = Randomized codon
  source 1..183: mol_type = other DNA
aaataaatctcgaggtagatgccaaatacgccaaagaagagnnnnnnnnngcagccnnnnnngaggaaatcattnnnnnnttactgcctaacttaaccactnnnnnncaacagtggnnngccgcgttcatcattnnnaaaaagttactgnnngatgacgacccaagccagagctcattattta

SEQ ID NO: 775 - protein, 14 aa, synthetic construct
  REGION 1..14: note = Engineered Polypeptide
  REGION 12..13: note = MISC_FEATURE - Individual Z variant
MGSSHHHHHHLQVD

SEQ ID NO: 776 - protein, 12 aa, synthetic construct
  REGION 1..12: note = Engineered polypeptide
  REGION 1: note = MISC_FEATURE - Individual Z variant Z06175a
  REGION 12: note = MISC_FEATURE - Albumin Binding Domain 094
GSGGGGSGGGGS

SEQ ID NO: 777 - protein, 14 aa, synthetic construct
  REGION 1..14: note = Engineered Polypeptide
  REGION 1: note = MISC_FEATURE - Z is an individual Z variant Z06175a
  REGION 2..3: note = MISC_FEATURE - Albumin binding domain 094
  REGION 14: note = MISC_FEATURE - Z is an individual Z variant Z06175a
GSGSGGGGSGGGGS

SEQ ID NO: 778 - protein, 12 aa, synthetic construct
  REGION 1..12: note = Engineered Polypeptide
  REGION 1: note = MISC_FEATURE - Z is an individual Z variant Z06175a
  REGION 12: note = MISC_FEATURE - Albumin binding domain 094
GSGGGGSGGGGS

SEQ ID NO: 779 - protein, 12 aa, synthetic construct
  REGION 1..12: note = Engineered Polypeptide
  REGION 1: note = MISC_FEATURE - Z is individual Z variant Z06175a
  REGION 12: note = MISC_FEATURE - Albumin binding domain 094
GSGGGGSGGGGS

SEQ ID NO: 780 - protein, 15 aa, synthetic construct
  REGION 1..15: note = Engineered Polypeptide
  REGION 1: note = MISC_FEATURE - Individual Z variant is Z06175a
  REGION 2..3: note = MISC_FEATURE - Albumin binding domain 094
  REGION 14: note = MISC_FEATURE - Individual Z variant is Z06175a
AGSGSGGGGSGGGGS

SEQ ID NO: 781 - protein, 15 aa, synthetic construct
  REGION 1..15: note = Artificial Sequence
  REGION 1: note = MISC_FEATURE - Individual Z variant is Z06175a
  REGION 2..3: note = MISC_FEATURE - Albumin binding domain 094
  REGION 14: note = MISC_FEATURE - Individual Z variant is Z06175a
AGSGSGGGGSGGGGS

SEQ ID NO: 782 - protein, 14 aa, synthetic construct
  REGION 1..14: note = Artificial Sequence
  REGION 2: note = MISC_FEATURE - Sequence contains carbon 13 and nitrogen 15
INKLDDDPSQSSEL

SEQ ID NO: 783 - protein, 4 aa, synthetic construct
  REGION 1..4: note = linking moiety
  VARIANT 1..4: note = MISC_FEATURE - Repeated 0-7 times
GGGS

SEQ ID NO: 784 - protein, 5 aa, synthetic construct
  REGION 1..5: note = linking moiety
  VARIANT 1..5: note = MISC_FEATURE - Repeated 0-7 times
GGGGS

SEQ ID NO: 785 - protein, 7 aa, synthetic construct
  REGION 1..7: note = linking moiety
  VARIANT 3..7: note = MISC_FEATURE - Repeated 0-7 times
GSGGGGS

SEQ ID NO: 786 - protein, 4 aa, synthetic construct
  REGION 1..4: note = linking moiety
  VARIANT 1..4: note = MISC_FEATURE - Repeated 0-7 times
SSSG

SEQ ID NO: 787 - protein, 5 aa, synthetic construct
  REGION 1..5: note = linking moiety
  VARIANT 1..5: note = MISC_FEATURE - Repeated 0-7 times
SSSSG

SEQ ID NO: 788 - protein, 4 aa, synthetic construct
  REGION 1..4: note = linking moiety
VDGS

SEQ ID NO: 789 - protein, 13 aa, synthetic construct
  REGION 1..13: note = C5 binding moiety
  REGION 1: note = MISC_FEATURE - Individual Z variant Z06175a
  REGION 2..3: note = MISC_FEATURE - Albumin Binding Domain 094
  REGION 13: note = MISC_FEATURE - Individual Z variant Z06175a
GSGSGGGGSGGGS

(19) United States
(12) Patent Application Publication
(10) Pub. No.: US 2025/0332214 A1
(43) Pub. Date: Oct. 30, 2025

(54) POLYPEPTIDES BINDING TO HUMAN COMPLEMENT C5

(71) Applicant: IPC Research, LLC, Wilmington, DE (US)

(72) Inventors: Charlotta Berghard, Stockholm (SE); Magnus Berglund, Vendelsö (SE); Patrik Strömberg, Sollentuna (SE); Malin Lindborg, Saltsjö-Boo (SE); Elin Gunneriusson, Saltsjöbaden (SE); Joachim Feldwisch, Tyresö (SE)

(21) Appl. No.: 18/828,700

(22) Filed: Sep. 9, 2024

Related U.S. Application Data
(63) Continuation of application No. 17/479,101, filed on Sep. 20, 2021, now Pat. No. 12,083,160.
Application No. 17/479,101 is a continuation of application No. 16/228,181, filed on Dec. 20, 2018, now Pat. No. 11,123,401.
Application No. 16/228,181 is a continuation of application No. 15/721,507, filed on Sep. 29, 2017, now Pat. No. 10,206,975.
Application No. 15/721,507 is a continuation of application No. 14/378,522, filed on Aug. 13, 2014, now Pat. No. 9,808,502.

(30) Foreign Application Priority Data
Feb. 20, 2012 (SE) 1250145-8

(51) Int. Cl. A61K 38/16; A61K 38/00; A61K 47/64; A61K 47/68; C07K 14/00; C07K 14/195; C07K 14/315; C07K 14/47

(52) U.S. Cl. CPC A61K 38/164; A61K 38/16; A61K 47/64; A61K 47/68; A61K 47/6801; C07K 14/00; C07K 14/195; C07K 14/315; C07K 14/47; A61K 38/00; C07K 2319/00; C07K 2319/31

(57) ABSTRACT

The present invention relates to C5 binding polypeptides, comprising a C5 binding motif, BM, which motif consists of an amino acid sequence selected from

EX₂X₃X₄A X₆X₇EID X₁₁LPNL X₁₆X₁₇X₁₈QW X₂₁AFIX₂₅ X₂₆LX₂₈D,

and
ii) an amino acid sequence which has at least 86% identity to the sequence defined in i), wherein the polypeptide binds to C5. The present invention moreover relates to C5 binding polypeptides for use in therapy, such as for use in treatment of a C5 related condition, and to methods of treatments.

### CROSS-REFERENCE TO RELATED APPLICATIONS

This Application is a continuation of, and claims the benefit under 35 U.S.C. § 120 to, U.S. application Ser. No. 17/479,101, filed Sep. 20, 2021, now U.S. Pat. No. 12,083,160; which is a continuation of, and claims the benefit under 35 U.S.C. § 120 to, U.S. application Ser. No. 16/228,181, filed Dec. 20, 2018, now U.S. Pat. No. 11,123,401;
which is a continuation of, and claims the benefit under 35 U.S.C. § 120 to U.S. application Ser. No. 15/721,507, filed Sep. 29, 2017, now U.S. Pat. No. 10,206,975; which is a continuation of and claims the benefit under 35 U.S.C. § 120 to U.S. application Ser. No. 14/378,522, filed Aug. 13, 2014, now U.S. Pat. No. 9,808,502; which is a U.S. National Stage Application of PCT/SE2013/050139 filed Feb. 19, 2013, which claims foreign priority benefits under 35 U.S.C. § 119(a)-(d) or 35 U.S.C. § 365(b) of Swedish Application Number 1250145-8, filed Feb. 20, 2012.

### SEQUENCE LISTING

The instant application contains a Sequence Listing which has been submitted electronically in XML format and is hereby incorporated by reference in its entirety. Said XML copy, created on May 21, 2025, is named IPC_001_US5_SL.xml and is 1,048,700 bytes in size.

### TECHNICAL FIELD

The present disclosure relates to polypeptides that bind to human complement component 5 (C5) and to use of such polypeptides in therapy.

### BACKGROUND

The complement protein C5 is a central component of the complement system; a key part of the innate immune system. The complement system is an intricate immune survival system with numerous tasks in tightly controlled, diverse processes. One of its functions is as first line host defense against infection by other organisms by discriminating healthy host tissues from cellular debris and apoptotic and necrotic cells. Furthermore, it is involved in clearance of immune complexes, regulation of the adaptive immune response, promotion of tissue regeneration, angiogenesis, mobilization of stem cells and development of the central nervous system (Woodruff et al. Mol Immunol 2011, 48 (14):1631-1642); Ricklin et al. Nat Immunol 2010, 11(9):785-795). Any trigger, for example erroneous or unrestricted activation or insufficient regulation, that disturbs the fine balance of complement activation and regulation may lead to pathologic conditions including self-attack of the host's cells leading to extensive tissue damage.

The complement system consists of about 30 proteins. There are three pathways to initiate complement immunity; the classical pathway that employs Clq to recognize immune complexes on the surface of cells, the lectin pathway that is initiated when mannose-binding lectin (MBL) recognizes certain sugars and the alternative pathway that is initiated spontaneously by hydrolysis of complement factor 3 (C3), a process suppressed by certain mammalian cell surface molecules not present on invading pathogens. The alternative pathway also acts as an amplification loop for the complement system. All three pathways converge at the level of C3. Cleavage of C3 into C3a and C3b leads to the formation of a convertase that in turn cleaves complement factor 5 (C5) into C5a and C5b. C5a is a very potent attractant of various immune cells while C5b oligomerizes with C6-9 to form a pore known as the membrane attack complex (MAC) or sometimes the terminal complement complex (TCC). Activation of the complement system leads to a number of mechanisms with the purpose of neutralizing the pathogen; formation of MAC on the surface of a cell such as an invading bacteria lead to lysis, deposition of C3 and C4 cleavage products C3b and C4b aids opsonization leading to phagocytosis of the pathogen by macrophages and anaphylatoxins such as C3a and C5a attracts monocytes and neutrophils to the site of activation, upregulates surface markers leading to increased immunologic susceptibility and to the release of cytokines.

C5 is a 190-kDa glycoprotein comprised of 2 disulfide-linked polypeptide chains, alpha and beta, with a molecular mass of 115 and 75 kDa, respectively (Tack et al. Biochem 1979, 18:1490-1497). Haviland et al. (J Immun 1991, 146: 362-368) constructed the complete cDNA sequence of human complement pro-C5, which is predicted to encode a 1,676-amino acid pro-molecule that contains an 18-amino acid leader peptide and a 4-amino acid linker separating the beta and alpha chains. Blockade of C5 cleavage into C5a and C5b prevents MAC formation and formation of the pro-inflammatory C5a but leaves the upstream complement effector system intact allowing the C3/C4 mediated opsonization.

The complement system's key role in the defense against pathogens in general makes it an interesting target for pharmaceutical intervention. This is emphasized by the fact that many mutations or impaired regulation of complement is involved in various diseases and conditions. These include increased susceptibility to auto-immune diseases such as systemic lupus erythematosis (SLE) where deposition of immune complexes triggers the classical pathway (Manderson et al. Annu Rev Immunol 2004, 22:431-456). In addition, mutations of the complement proteins C1-C5 often result in SLE or SLE like symptoms. Other autoimmune diseases with a strong involvement of the complement system are rheumatoid arthritis (RA) where immune complexes may activate complement in the RA joint, Sjögren's syndrome, dermatomyositis and other autoantibody driven diseases such as Guillain-Barré syndrome (GBS), Fisher syndrome (Kaida et al. J. Neuroimmun 2010, 223:5-12) different types of vasculitis, systemic sclerosis, anti-glomerular basement membrane (anti-GBM) and anti-phospholipid syndrome (APS) (Chen et al. J Autoimmun 2010, 34:J276-J286).

The complement system is furthermore involved in neurodegenerative disorders such as Alzheimer's disease (AD) where Aβ plaques directly activate the complement system leading to C5a mediated recruitment of microglia. This was further confirmed when a C5aR antagonist was shown to be neuroprotective in a mouse model of AD (Fonseca et al. J Immunol 2009, 183:1375-1383). Auto-antibodies against the acetylcholine receptor and subsequent complement activation is the most common cause to myasthenia gravis, a disease that affects the neuromuscular junction (Toyka and Gold, Schweizer Archive Neurol Psych 2007, 158:309-321). MAC formation is involved in the pathophysiology of multiple sclerosis (MS) (Oh et al. Immunol Res 2008, 40:224-234). Also in Parkinson's disease, Huntington's disease and prion diseases such as Creutzfeld-Jacob disease, complement activation is a part of the pathology (Bonifati and Kishore, Mol Immunol 2007, 44:999-1010). In wound healing, inflammatory responses are a key component to restore tissue homeostasis and the complement system is involved in the early recognition of damaged tissue. However, in models of chronic wounds and severe burns, for example, inhibition of complement by e.g. C1 inhibitor resulted in improved healing and decreased tissue damage suggesting that complement. Furthermore, various complement deficiencies, such as exemplified by the C4 knockout mouse, have been found to be protective against long-term tissue damage resulting from wounds (reviewed in Cazender et al. Clinical and Developmental Immunology 2012, on-line publication). Lately it has been shown that tumor growth and proliferation is facilitated by complement activation, in particular by C5a, and that blockade of the C5a receptor slows down this process. In addition, mice lacking C3 display significantly slower tumor growth than wild-type littermates (Markiewski et al. Nat Immunol 2008, 9:1225-1235).

Dysfunctional complement regulation is the cause of several rare to ultra-rare conditions, such as paroxysmal nocturnal hemoglobinuria (PNH) and atypical hemolytic uremic syndrome (aHUS), where hemolysis is a key feature in the pathology. In PNH, a clone of hematopoetic stem cells with mutated PIG-A gene encoding phosphatidylinositol N-acetylglucosaminyltransferase subunit A take over the pool of blood cells. This mutation leads to loss of GPI anchored proteins such as the complement regulators CD55 and CD59. Red blood cells lacking CD55 and CD59 on the surface are exposed for complement mediated lysis by MAC. Clinically, PNH is manifested by hemolysis leading to anemia, thrombosis and bone marrow failure. Atypical HUS is caused by mutations in regulatory proteins of mainly the alternative pathway, such as by mutations in factor H.

The eye is strongly indicated as a site for complement driven pathology. The most common cause of visual loss is age-related macular degeneration (AMD) where, in its more severe form (exudative or wet AMD), pathologic choridal neurovascular membranes develop under the retina. In the US, about 10% of the population aged 65-74 shows sign of macular degeneration and as many as 5% have visual impairment as a result to AMD. These numbers increase dramatically with age, but there are also genetic factors. Among the genes strongest associated with AMD are complement factor H, factor B and C3 and the C1 inhibitor (Bradley et al. Eye 2011, 25:683-693). Furthermore, several studies and clinical trials using various complement blocking molecules have proven beneficial, suggesting that a C5 blocking molecule could help these patient groups. However, the current treatments of advanced AMD aims at inhibition of vascular endothelial growth factor (VEGF) induced vascularization by intravitreal injections of e.g. Ranibizumab (a monoclonal antibody fragment) and Bevacizumab (monoclonal antibody). In animal models of uveitis, inflammation of the eye due to immune responses to ocular antigens, blocking antibodies against alternative pathway factor B (Manickam et al. J Biol Chem 2011, 286:8472-8480) as well as against C5 (Copland et al. Clin Exp Immunol 2009, 159:303-314), improved the disease state.

In transplantation of solid organs, there are two major mechanistic pathways leading to rejection or delayed/impaired function of the graft: 1) the immunologic barriers between donor and recipient with respect to blood group (ABO) and MCH classes as well as extent of pre-sensitization of the recipient against the donor, i.e. occurrence of donor specific antibodies (DSA) leading to acute antibody mediated rejection (AMR); and 2) the condition of the transplanted organ as well as the period of time it has been kept without constant blood perfusion, i.e. the degree of ischemic damage or ischemia reperfusion injury (IRI) of the graft. In both AMR and IRI, the complement system is attacking the organ recognized as foreign and, therefore, an entity that should be rejected. In AMR, the pre-existing anti-donor antibodies rapidly form immune complexes on the surface of the foreign organ leading to recognition by Clq and subsequent activation of the complement system via the classical pathway. This process, known as hyper-acute rejection happens within minutes and, therefore modem transplantation of mismatched organs includes elimination of DSA prior to transplantation by plasmapheresis or plasma exchange and intravenous IgG combined with different immunosuppressants. Novel treatments also include B-cell depletion via usage of the anti-CD20 antibody Rituximab (Genberg et al. Transplant 2008, 85:1745-1754). These protocols have vastly eliminated the occurrence of hyper-acute rejection but still, in highly sensitized patients, the incidence of acute AMR (weeks-months) is as high as 40% (Burns et al. Am J Transplant 2008, 6:2684-2694; Stegall et al. Am J Transplant 2011, early on-line publication). With respect to IRI, most evidence points at the terminal pathway with subsequent MAC formation and lysis as the main cause of tissue damage. Thus, a C5 blocking polypeptide would be protective against rejection regardless of the cause being AMR, IRI or, as often happens, a combination of both AMR and IRI. As expected, highly perfused organs, such as the liver (Qin et al. Cell Mol Immunol 2006, 3:333-340), the heart and the kidneys are particularly susceptible to complement mediated damage.

The central placement of the C5 protein; connecting the proximal and the terminal parts of the complement cascade, makes it an attractive target for pharmaceutical intervention. Since C5 is common to all pathways of complement activation, blocking of C5 will stop the progression of the cascade regardless of the stimuli and thereby prevent the deleterious properties of terminal complement activation while leaving the immunoprotective and immunoregulatory functions of the proximal complement cascade intact.

Antibodies targeted to human complement C5 are known from, e.g., WO 95/29697; WO 02/30985; and WO 2004/007553. Eculizumab (Soliris™) is a humanized monoclonal antibody directed against protein C5 and prevents cleavage of C5 into C5a and C5b. Eculizumab has been shown to be effective in treating PNH, a rare and sometimes life threatening disease of the blood characterized by intravascular hemolytic anemia, thrombophilia and bone marrow failure, and is approved for this indication. Eculizumab was also recently approved by the FDA for treatment of atypical hemolytic syndrome (aHUS), a rare but life threatening disease caused by loss of control of the alternative complement pathway leading to over-activation manifested as thrombotic microangiopathy (TMA) leading to constant risk of damage to vital organs such as kidney, heart and the brain. In aHUS, transplantation of the damaged organ only temporarily helps the patient as the liver continues to produce the mutated form of controlling protein (most often complement factor H or other proteins of the alternative pathway). A related disease with a transient acute pathophysiology is HUS caused by infection of Shiga toxin positive E. coli (STEC-HUS) and there are promising clinical data suggesting efficacy also for this condition (Lapeyraque et al, N Engl J Med 2011, 364:2561-2563). Finally, the C5 blocking antibody Eculizumab has proven efficacious in preventing AMR in recipients of highly mismatched kidneys (Stegall, M. D. et al. Am J Transplant 2011, 11:2405-2413).

Apart from full length antibodies, single-chain variable fragments (scFV), minibodies and aptamers targeting C5 are described in literature. These C5 inhibitors may bind to different sites (epitopes) on the C5 molecule and may have different modes of action. For example, whereas Eculizumab interacts with C5 at some distance of the convertase cleavage site, the minibody Mubodina® interacts with the cleavage site of C5. The C5 inhibitory protein Ornithodoros moubata Complement Inhibitor (OmCI, Nunn, M. A. et al. J Immunol 2005, 174:2084-2091) from soft tic Ornithodoros moubata has been hypothesized to bind to the distal end of the CUB-C5d-MG8 superdomain, which is close to the convertase cleavage site (Fredslund et al. Nat Immunol 2008, 9 (7):753-760). In contrast to the three proteins mentioned above inhibiting cleavage of C5, the monoclonal antibody TNX-558 binds to a C5a epitope present both on intact C5 and released C5a without inhibiting the cleavage of C5. (Fung et al. Clin Exp Immunol 2003, 133 (2):160-169).

Antibodies with their large, multidomain structure, 12 intra-chain and 4 inter-chain disulfide bridges and complex glycosylation patterns, have a number of intrinsic disadvantages related to their molecular structure. For example, the size of Eculizumab is about 148 kDa. The concentration of C5 in human blood is about 400 nM and in order to block C5 activity entirely, the concentration of the inhibitor must be at least equal or higher than that. Therefore, the standard life-long treatment regimen of PNH using Soliris™ is intravenous infusions of 900 mg protein every second week, a treatment that mainly take place in the clinic leading to great inconvenience to the patient and cost to the society. Soliris™ has also been reported to cause chest pain, fever, chills, itching, hives, flushing of the face, rash, dizziness, troubled breathing, or swelling of the face, tongue, and throat, although the reasons for these side effects are not clear. Furthermore, Eculizumab is not active in any tested animal model, including primates, making animal studies with the active drug impossible. As mentioned above, the current treatments of AMD are also antibody dependent and, thus, treatments based on injections or other routes of administration with molecules of lower molecular weight, are highly required.

In addition, antibody production is more difficult and more expensive than production of small proteins (Kenanova et al. Expert Opin Drug Deliv 2006, 3 (1):53-70). Other drawbacks generally related to antibodies are listed by Reilly et al. (Clin Pharmacokinet 1995, 28:126-142), such as cross-reactivity and non-specific binding to normal tissues, increased metabolism of injected antibodies and formation of human anti-human antibodies (HAMA) causing decreased or loss of the therapeutic effect.

Thus, continued provision of agents with comparable C5 blocking activity remains a matter of substantial interest within the field. In particular, there is a continued need for molecules that prevent the terminal complement cascade as well as the formation of the pro-inflammatory molecule C5a. Of great interest is also a provision of uses of such molecules in the treatment of disease.

### DESCRIPTION

It is an object of the invention to provide new C5 binding agents. It is moreover an object of the invention to provide new C5 binding agents for use in therapeutic applications.

In one aspect, there is provided a C5 binding polypeptide, comprising a C5 binding motif, BM, which motif consists of the amino acid sequence selected from

i)
(SEQ ID NO. 763)
EX₂X₃X₄A X₆X₇EID X_{₁₁}LPNL X_{₁₆}X_{₁₇}X_{₁₈}QW X_{₂₁}AFIX_{₂₅}
X_{₂₆}LX₂₈D,

wherein, independently of each other,
X₂ is selected from H, Q, S, T and V;
X₃ is selected from I, L, M and V;
X₄ is selected from A, D, E, H, K, L, N, Q, R, S, T and Y;
X₆ is selected from N and W;
X₇ is selected from A, D, E, H, N, Q, R, S and T;
X₁₁ is selected from A, E, G, H, K, L, Q, R, S, T and Y;
X₁₆ is selected from N and T;
X₁₇ is selected from I, L and V;
X₁₈ is selected from A, D, E, H, K, N, Q, R, S and T;
X₂₁ is selected from I, L and V;
X₂₅ is selected from D, E, G, H, N, S and T;
X₂₆ is selected from K and S;
X₂₈ is selected from A, D, E, H, N, Q, S, T and Y; and
ii) an amino acid sequence which has at least 86% identity to the sequence defined in i), wherein the polypeptide binds to C5.

The above defined class of sequence related polypeptides having a binding affinity for C5 is derived from a common parent polypeptide sequence. More specifically, the definition of the class is based on an analysis of a large number of random polypeptide variants of the parent polypeptide that were selected for their interaction with C5 in selection experiments. The identified C5 binding motif, or “BM”, corresponds to the target binding region of the parent scaffold, which region constitutes two alpha helices within a three-helical bundle protein domain. In the parent scaffold, the varied amino acid residues of the two BM helices constitute a binding surface for interaction with the constant Fc part of antibodies. By random variation of binding surface residues and subsequent selection of variants, the Fc interaction capacity of the binding surface has been replaced with a capacity for interaction with C5.

As accounted for in the following Examples, selection of C5 binding polypeptide variants may for example be achieved by phage display for selection of näive variants of a protein scaffold optionally followed by affinity maturation and cell display for selection of affinity maturated C5 binding variants. It is however understood that any selection system, whether phage-based, bacterial-based, cell-based or other, may be used for selection of C5 binding polypeptides.

The terms “C5 binding” and “binding affinity for C5” as used in this specification refers to a property of a polypeptide which may be tested for example by the use of surface plasmon resonance technology, such as in a Biacore instrument (GE Healthcare). C5 binding affinity may e.g. be tested in an experiment in which C5 is immobilized on a sensor chip of a Biacore instrument, and the sample containing the polypeptide to be tested is passed over the chip. Alternatively, the polypeptide to be tested is immobilized on a sensor chip of the instrument, and a sample containing C5, or fragment thereof, is passed over the chip. The skilled person may then interpret the results obtained by such experiments to establish at least a qualitative measure of the binding of the polypeptide to C5. If a quantitative measure is desired, for example to determine the apparent equilibrium dissociation constant K_{D} for the interaction, surface plasmon resonance methods may also be used. Binding values may for example be defined in a Biacore 2000 instrument (GE Healthcare). C5 is immobilized on a sensor chip of the measurement, and samples of the polypeptide whose affinity is to be determined are prepared by serial dilution and injected over the chip. K_{D} values may then be calculated from the results using for example the 1:1 Langmuir binding model of the BIAevaluation software provided by the instrument manufacturer. The C5 or fragment thereof used in the K_{D} determination may for example comprise the amino acid sequence represented by SEQ ID NO:760.

In one embodiment of the C5 binding polypeptide according to the present invention, the C5 binding polypeptide binds to C5 such that the K_{D} value of the interaction is at most 1×10⁻⁶ M, such as at most 1×10⁻⁷ M, 1×10⁻⁸ M, or 1×10⁻⁹ M.

A C5 binding polypeptide according to the present invention may be used as an alternative to conventional antibodies or low molecular weight substances in various medical, veterinary and diagnostic applications. In particular, the C5 binding polypeptide may be useful in any method requiring affinity for C5 of a reagent. Accordingly, the C5 binding polypeptide may be used as a detection reagent, a capture reagent, a separation reagent, a diagnostic agent or a therapeutic agent in such methods.

As the skilled person will realize, the function of any polypeptide, such as the C5 binding capacity of the polypeptides as defined herein, is dependent on the tertiary structure of the polypeptide. It is therefore possible to make minor changes to the amino acid sequence of a polypeptide without largely affecting the tertiary structure and the function thereof. Thus, in one embodiment, the polypeptide comprises modified variants of the BM of i), which are such that the resulting sequence is at least 89% identical to a sequence belonging to the class defined by i), such as at least 93% identical, such as at least 96% identical to a sequence belonging to the class defined by i). For example, it is possible that an amino acid residue belonging to a certain functional grouping of amino acid residues (e.g. hydrophobic, hydrophilic, polar etc) could be exchanged for another amino acid residue from the same functional group.

In another embodiment of the C5 binding polypeptide as defined above, the amino acid sequence is selected from i) as defined above, and iii) an amino acid sequence which in the 13 variable positions as denoted by Xₙ, wherein n is 2-4, 6-7, 11, 16-18, 21, 25-26 and 28, has at least 84% identity to the sequence defined in i), and which in positions 1, 5, 8-10, 12-15, 19-20, 22-24, 27 and 29 has at least 87% identity to the sequence defined in i).

In one embodiment of the polypeptide according to the present invention, X₂ is selected from H, T and V. In another embodiment, X₂ is selected from T and V. In yet another embodiment, X₂ is V.

In one embodiment of the polypeptide according to the present invention, X₃ is selected from I, L and V. In another embodiment, X₃ is selected from I and L. In yet another embodiment, X₃ is I. In an alternative embodiment, X₃ is L.

In one embodiment of the polypeptide according to the present invention, X₄ is selected from A, D, E, K, L, Q and R. In another embodiment, X₄ is selected from A, D, E, K and R. In yet another related embodiment, X₄ is selected from D and E.

In one embodiment of the polypeptide according to the present invention, X₆ is W.

In one embodiment of the polypeptide according to the present invention, X₇ is selected from A, D, N and T. In another embodiment, X₇ is selected from D and N. In yet another related embodiment, X₇ is D. In an alternative embodiment, X₇ is N.

In one embodiment of the polypeptide according to the present invention, X₁₁ is selected from A, H, K, Q, R and S. In another embodiment, X₁₁ is selected from A, H, K and R. In yet another related embodiment, X₁₁ is selected from A, K and R. In yet another related embodiment, X₁₁ is selected from K and R.

In one embodiment of the polypeptide according to the present invention, X₁₆ is T.

In one embodiment of the polypeptide according to the present invention, X₁₇ is selected from I and L. In another embodiment, X₁₇ is I. In an alternative embodiment, X₁₇ is L.

In one embodiment of the polypeptide according to the present invention, X₁₈ is selected from A, D, E, N, Q, S and T. In another embodiment, X₁₈ is selected from A, D, E, Q and S. In yet another related embodiment, X₁₈ is selected from D, E and Q. In yet another related embodiment, X₁₈ is selected from D and E. In yet another related embodiment, X₁₈ is D. In an alternative embodiment, X₁₈ is E.

In one embodiment of the polypeptide according to the present invention, X₂₁ is selected from I and L. In another embodiment, X₂₁ is I. In an alternative embodiment, X₂₁ is L.

In one embodiment of the polypeptide according to the present invention, X₂₅ is selected from E, H, N and T. In another embodiment, X₂₅ is selected from E and N. In yet another related embodiment, X₂₅ is N.

In one embodiment of the polypeptide according to the present invention, X₂₆ is K.

In one embodiment of the polypeptide according to the present invention, X₂₈ is selected from A, D, E, H, N, Q and S. In another embodiment of the above disclosed polypeptide, X₂₈ is selected from A, D, E and S. In yet another related embodiment, X₂₈ is selected from A, D and E. In yet another related embodiment, X₂₈ is selected from D and E. In yet another related embodiment, X₂₈ is D.

In one embodiment of the polypeptide according to the present invention, X₃X₄ is selected from LE and LD.

In one embodiment of the polypeptide according to the present invention, X₁₇X₁₈ is selected from IE and LD.

In the above embodiments of the first aspect, examples of C5 binding polypeptides falling within the class of polypeptides are identified. It is contemplated that the above individual embodiments may be combined in all conceivable ways and still fall within the scope of the present invention. Such combinations of individual embodiments define a restricted, in one or more of the positions X₂-X₂₈, amino acid sequence as compared to the amino acid definition in i).

The above embodiments of a C5 binding polypeptide may for example be combined such that the amino acid i) fulfils at least four of the following eight conditions I-VIII:
I. X₂ is V;
II. X₃ is selected from I and L;
III. X₆ is W;
IV. X₇ is selected from D and N;
V. X₁₇ is selected from I and L;
VI. X₂₁ is L;
VII. X₂₅ is N;
VIII. X₂₈ is D.

In some examples of a C5 binding polypeptide according to the first aspect, the amino acid sequence i) fulfils at least five of the eight conditions I-VIII. More specifically, the amino acid sequence i) may fulfill at least six of the eight conditions I-VIII, such at least seven of the eight conditions I-VIII, such as all of the eight conditions I-VIII.

As described in the following Examples, the selection of C5 binding variants has led to the identification of individual C5 binding motif (BM) sequences. These sequences constitute individual embodiments of C5 binding polypeptides according to this aspect. The sequences of individual C5 binding motifs are presented in FIG. 1 and as SEQ ID NO:1-248. In some embodiments of this aspect, the BM sequence i) is selected from any one of SEQ ID NO:1-12, SEQ ID NO:20, SEQ ID NO:23-24, SEQ ID NO:26-28, SEQ ID NO:32-35, SEQ ID NO:38-39, SEQ ID NO:41, SEQ ID NO:46, SEQ ID NO:49, SEQ ID NO:56-57, SEQ ID NO:59, SEQ ID NO:66, SEQ ID NO:78-79, SEQ ID NO:87, SEQ ID NO:92, SEQ ID NO:106, SEQ ID NO:110, SEQ ID NO:119, SEQ ID NO:125, SEQ ID NO:141, SEQ ID NO:151, SEQ ID NO:161, SEQ ID NO: 166, SEQ ID NO: 187, SEQ ID NO: 197, SEQ ID NO:203, SEQ ID NO:205, SEQ ID NO:215 and SEQ ID NO:243. More specifically, the BM sequence i) is selected from any one of SEQ ID NO:1-12, such as from SEQ ID NO:1, SEQ ID NO:2, SEQ ID NO:3, SEQ ID NO:4 and SEQ ID NO:5. In particular, the BM sequence i) may be selected from SEQ ID NO:1 and SEQ ID NO:4.

In particular embodiments, the C5 binding motif (BM) forms part of a three-helix bundle protein domain. For example, the BM may essentially constitute two alpha helices with an interconnecting loop, within said three-helix bundle protein domain.

The three-helix bundle protein domain is, in another embodiment, selected from domains of bacterial receptor proteins. Non-limiting examples of such domains are the five different three-helical domains of Protein A from Staphylococcus aureus, such as domain B, and derivatives thereof. In some embodiments, the three-helical bundle protein domain is a variant of protein Z, which is derived from said domain B of staphylococcal Protein A.

In embodiments where the C5 binding polypeptide of the invention forms part of a three-helix bundle protein domain, the C5 binding polypeptide may comprise an amino acid sequence selected from:

i)
(SEQ ID NO. 764)
K-[BM]-DPSQS XₐX_{b}LLX_{c} EAKKL NDX_{d}Q;

wherein
[BM] is a C5 binding motif as defined above;
Xₐ is selected from A and S;
X_{b} is selected from N and E;
X_{c} is selected from A, S and C;
X_{d} is selected from A and S;
and
ii) an amino acid sequence which has at least 79% identity to any one of the sequences defined above. Said amino acid sequence may have at least 81%, such as at least 83%, such as at least 85%, such as at least 87%, such as at least 89%, such as at least 91%, such as at least 93%, such as at least 95%, such as at least 97% identity to any one of the sequences defined above.

In one embodiment of the C5 binding polypeptide as defined above, Xₐ is A. In an alternative embodiment of the C5 binding polypeptide as defined above, Xₐ is S.

In one embodiment of the C5 binding polypeptide as defined above, X_{b} is N. In an alternative embodiment, X_{b} is E.

In one embodiment of the C5 binding polypeptide as defined above, X_{c} is A. In an alternative embodiment, X_{c} is S. In yet another alternative embodiment, X_{c} is C.

In one embodiment of the C5 binding polypeptide as defined above, X_{d} is A. In an alternative embodiment, X_{d} is S.

In one embodiment of the C5 binding polypeptide as defined above, Xₐ is A; X_{b} is N; X_{c} is A and X_{d} is A.

In a further embodiment of the C5 binding polypeptide as defined above, Xₐ is A; X_{b} is N; X_{c} is C and X_{d} is A.

In a further embodiment of the C5 binding polypeptide as defined above, Xₐ is S; X_{b} is E; X_{c} is S and X_{d} is S.

In a further embodiment of the C5 binding polypeptide as defined above, Xₐ is S; X_{b} is E; X_{c} is C and X_{d} is S.

In yet a further embodiment, the amino acid sequence of the C5 binding polypeptide as defined above is selected from SEQ ID NO:249-496, in particular from SEQ ID NO:249-260, SEQ ID NO:268, SEQ ID NO:271-272, SEQ ID NO:274-276, SEQ ID NO:280-283, SEQ ID NO:286-287, SEQ ID NO:289, SEQ ID NO:294, SEQ ID NO:297, SEQ ID NO:304-305, SEQ ID NO:307, SEQ ID NO:314, SEQ ID NO:326-327, SEQ ID NO:335, SEQ ID NO:340, SEQ ID NO:354, SEQ ID NO:358, SEQ ID NO:367, SEQ ID NO:373, SEQ ID NO:389, SEQ ID NO:399, SEQ ID NO:409, SEQ ID NO:414, SEQ ID NO:435, SEQ ID NO:445, SEQ ID NO:451, SEQ ID NO:453, SEQ ID NO:463 and SEQ ID NO:491, such as from SEQ ID NO:249-260. In a further embodiment, the amino acid sequence is selected from SEQ ID NO:249, SEQ ID NO:250, SEQ ID NO:251, SEQ ID NO:252 and SEQ ID NO:253, such as from SEQ ID NO:249 and SEQ ID NO:252.

Thus, in a further embodiment, there is provided a C5 binding polypeptide which comprises an amino acid sequence selected from:

i)
(SEQ ID NO. 765)
YAK-[BM]-DPSQS SELLX_{c} EAKKL NDSQA P;

wherein [BM] is a C5 binding motif as defined above and
X_{c} is selected from S and C; and
ii) an amino acid sequence which has at least 81% identity to any one of the sequences defined in i) above.

Alternatively, there is provided a C5 binding polypeptide which comprises an amino acid sequence selected from:

i)
(SEQ ID NO. 766)
FNK-[BM]-DPSQS ANLLX_{c} EAKKL NDAQA P;

wherein [BM] is a C5 binding motif as defined above and
X_{c} is selected from A and C; and
ii) an amino acid sequence which has at least 81% identity to any one of the sequences defined in i) above.

As discussed above, polypeptides comprising minor changes as compared to the above amino acid sequences without largely affecting the tertiary structure and the function thereof are also within the scope of the present application. Thus, in some embodiments, the C5 binding polypeptides as defined above may for example have a sequence which is at least 83%, at least 84%, at least 86%, at least 88%, at least 90%, at least 92%, at least 94%, at least 96% or at least 98% identical to the sequence defined in i).

In some embodiments and as disclosed in the Examples below, the C5 binding motif may form part of a 58 or 60 amino acid polypeptide. Such a polypeptide may e.g. comprise a sequence selected from any one of SEQ ID NO:497-757, in particular a sequence selected from any one of SEQ ID NO:497-508, SEQ ID NO:516, SEQ ID NO:519-520, SEQ ID NO:522-524, SEQ ID NO:528-531, SEQ ID NO:534-535, SEQ ID NO:537, SEQ ID NO:542, SEQ ID NO:545, SEQ ID NO:552-553, SEQ ID NO:555, SEQ ID NO:562, SEQ ID NO:574-575, SEQ ID NO:583, SEQ ID NO:588, SEQ ID NO:602, SEQ ID NO:606, SEQ ID NO:615, SEQ ID NO:621, SEQ ID NO:637, SEQ ID NO:647, SEQ ID NO:657, SEQ ID NO:662, SEQ ID NO:683, SEQ ID NO:693, SEQ ID NO:699, SEQ ID NO:701, SEQ ID NO:711, SEQ ID NO:739 and SEQ ID NO:745-757, such as a sequence selected from SEQ ID NO:497-508 and SEQ ID NO:745-757. In another embodiment, the amino acid sequence is selected from SEQ ID NO:497, SEQ ID NO:498, SEQ ID NO:499, SEQ ID NO:500, SEQ ID NO:501, SEQ ID NO:746, SEQ ID NO:747, SEQ ID NO:748, SEQ ID NO:750 and SEQ ID NO:753, such as from any one of SEQ ID NO:497, SEQ ID NO:500, SEQ ID NO:748 and SEQ ID NO:753.

Binding of a molecule to C5 does not necessarily inhibit cleavage of C5. Inhibition is dependent on binding site, and since it is not entirely clear what effects interaction with specific regions of C5 have, some C5 binding molecules may interact with C5 without inhibiting its cleavage into C5a and C5b. In one embodiment of the present invention, the C5 binding polypeptide, when e.g. administered to a mammalian subject, inhibits cleavage of C5. The C5 binding polypeptide according to the invention may more specifically inhibit cleavage of human C5 when administered to a human subject.

The structure of C5 differs somewhat between species, and thus, a C5 binder found to bind C5 of one species may be inactive in another species. The humanized antibody Eculizumab, for example, binds to a domain of C5 often referred to as MG7. This region is highly variable between species and, therefore, Eculizumab binding is restricted to human C5. The C5 binding polypeptide of the present invention, however, is not restricted to human C5 but exhibits activity in animal models as well, as demonstrated in the appended Examples.

The skilled person will understand that various modifications and/or additions can be made to a C5 binding polypeptide according to any aspect disclosed herein in order to tailor the polypeptide to a specific application without departing from the scope of the present invention. For example, a C5 binding polypeptide according to any aspect may comprise further C terminal and/or N terminal amino acids. Such a polypeptide should be comprehended as a polypeptide having additional amino acids residues at the very first and/or the very last position in the polypeptide chain, i.e. at the N- and/or C-terminus. Thus, a C5 binding polypeptide may comprise any suitable number of additional amino acid residues, for example at least one additional amino acid residue. Each additional amino acid residue may individually or collectively be added in order to, for example, improve production, purification, stabilization in vivo or in vitro, coupling, or detection of the polypeptide. Such additional amino acid residues may comprise one or more amino acid residues added for the purpose of chemical coupling. One example of this is the addition of a cysteine residue. Such additional amino acid residues may also provide a “tag” for purification or detection of the polypeptide, such as a His₆ tag or a “myc” (c-myc) tag or a “FLAG” tag for interaction with antibodies specific to the tag or immobilized metal affinity chromatography (IMAC) in the case of the His₆-tag.

The further amino acids as discussed above may be coupled to the C5 binding polypeptide by means of chemical conjugation (using known organic chemistry methods) or by any other means, such as expression of the C5 binding polypeptide as a fusion protein.

The further amino acids as discussed above may for example comprise one or more polypeptide domain(s). A further polypeptide domain may provide the C5 binding polypeptide with another function, such as for example another binding function, or an enzymatic function, or a toxic function (e.g. an immunotoxin), or a fluorescent signaling function, or combinations thereof.

A further polypeptide domain may moreover provide the C5 binding polypeptide with the same binding function. Thus, in a further embodiment, there is provided a C5 binding polypeptide comprising at least two C5 binding polypeptide monomer units, the amino acid sequences of which may be the same or different. Multimeric forms of the polypeptides may comprise a suitable number of domains, each having a C5 binding motif, and each forming a “monomer” within the multimer. These domains may all have the same amino acid sequence, but alternatively, they may have different amino acid sequences. In particular, the C5 binding polypeptide of the invention may form homo- or heterodimers.

The further polypeptide domain(s) as described above may be joined to the C5 binding polypeptide by covalent coupling using known organic chemistry methods. Alternatively, the C5 binding polypeptide comprising the further polypeptide domain(s) may be expressed as one or more fusion polypeptides, for example in a system for recombinant expression of polypeptides, or joined in any other fashion, either directly or via a linker, for example an amino acid linker.

In some embodiments, the further polypeptide domain(s) may comprise a half-life extending moiety which increases the half life of the C5 binding polypeptide in vivo. As understood by the skilled person, increased, or extended, half life means slower clearance of a particular molecule from blood. There are a number of known strategies for prolonging the half life of a particular polypeptide in vivo, such as coupling to the Fc domain of an antibody (Fc conjugation) or coupling to albumin. Another example is coupling to a half life extending moiety, e.g. a peptide or protein, that will associate to serum albumin in vivo. In particular, the half life extending moiety may be an albumin binding moiety. An albumin binding moiety may e.g. consist of a naturally occurring polypeptide, or an albumin binding fragment thereof, or an engineered polypeptide. An engineered polypeptide may be derived from a naturally occurring starting polypeptide through subjecting it to protein engineering techniques, such as mutations and alterations in a site-directed or randomized approach, with a view to create novel or enhanced properties, such as binding affinity for a molecule such as albumin. Such an engineered albumin binding polypeptide may for example be a variant of a protein scaffold, which variant has been selected for its specific binding affinity for albumin. In a specific embodiment, the protein scaffold may be selected from domains of streptococcal Protein G or derivatives thereof, such as for example domain GA1, domain GA2 and domain GA3 of Protein G from Streptococcus strain G148, in particular domain GA3.

Accordingly, in one embodiment of the C5 binding polypeptide, the further amino acids improves stabilization in vivo or in vitro and comprise an albumin binding domain (ABD) of streptococcal protein G, or a derivative thereof. One example of an albumin binding domain which may be comprised as a further polypeptide domain in the C5 binding polypeptide of the invention is set out in SEQ ID NO:759. Other examples of suitable albumin binding domains are disclosed in WO 2009/016043 and WO 2012/004384. Such an ABD-extended polypeptide binds to serum albumin in vivo, and benefits from its longer half life, which increases the net half life of the polypeptide itself (see e.g. WO 91/01743). The pharmacokinetic profile of a C5 binding polypeptide comprising an albumin binding moiety as defined above thus resembles that of serum albumin when administered for example to a mammalian subject. ABD and derivatives thereof bind very strongly to human serum albumin (HSA) as well as to serum albumin from other species, such as mouse and rat.

ABD of streptococcal protein G is 46 amino acid long, and thus when a C5 binding polypeptide according to the invention comprises an ABD moiety or a derivative thereof, the overall size of the C5 binding polypeptide is relatively small. When administered for example to a mammalian subject, such as a human subject, the albumin binding part of the C5 binding polypeptide will associate non-covalently with serum albumin and the polypeptide may thereby benefit from decreased renal clearance and increased recirculation in epithelial cells. Tissue penetration may however still be fast due to extravasating properties of serum albumin. Furthermore, a C5 binding polypeptide comprising a half life extending moiety may not only display an extended half life in vivo, but also a reduced immunologic response in vivo, as compared to a polypeptide lacking a corresponding half life extending moiety (see e.g. WO 2005/097202).

In a related aspect, there is provided a C5 binding compound, comprising at least one C5 binding polypeptide according to any preceding claim; at least one albumin binding domain of streptococcal protein G, or a derivative thereof, and at least one linking moiety for linking said at least one domain or derivative thereof to the C or N terminus of said at least one C5 binding polypeptide. Such a C5 binding compound has high affinity for C5 as well as for serum albumin in vivo, when administered e.g. to a mammalian subject, and binding to serum albumin does not interfere with the interaction with C5, as demonstrated in the following Examples.

In one embodiment, the C5 binding compound has a structure selected from
[CBP1]-[L1]-[ALBD];
[CBP1]-[CBP2]-[L1]-[ALBD];
[CBP1]-[L1]-[ALBD]-[L2]-[CBP2];
[ALBD]-[L1]-[CBP1];
[ALBD]-[L1]-[CBP1]-[CBP2];
[CBP1]-[L1]-[CBP2]-[L2]-[ALBD]; and
[ALBD]-[L1]-[CBP1]-[L2]-[CBP2]
wherein, independently of each other,
[CBP1] and [CBP2] are C5 binding polypeptides which may be the same or different;
[L1] and [L2] are linking moieties which may be the same or different; and
[ALBD] is an albumin binding domain of streptococcal protein G, or derivative thereof.

Preferred C5 binding compounds have a structure selected from
[CBP1]-[CBP2]-[L1]-[ALBD];
[CBP1]-[L1]-[ALBD]-[L2]-[CBP2]; and most preferably,
[CBP1]-[L1]-[ALBD].

Examples of linking moieties that may be used in such C5 binding compounds are selected from G, GS; [G₂S]ₙ; [G₃S]ₙ (SEQ ID NO: 783); [G₄S]ₙ (SEQ ID NO: 784); GS[G₄S]ₙ (SEQ ID NO: 785), wherein n is 0-7 (preferably, n is 0-2); [S₂G]ₘ (SEQ ID NO: 786); [S₃G]ₘ (SEQ ID NO: 787);[S₄G]ₘ; wherein m is 0-7, and VDGS (SEQ ID NO: 788). Preferred linkers are GS and GS[G₄S]₂ (SEQ ID NO: 785).

Examples of albumin binding domains or derivatives thereof that may be comprised in a C5 binding compound are as described above. In particular, one example of an albumin binding domain is set out in SEQ ID NO:759.

Particularly preferred C5 binding compounds have the structure [CBP1]-[L1]-[ALBD], wherein [CBP1] is a polypeptide selected from SEQ ID NO:748 and SEQ ID NO:753, [L1] is GS, and [ALBD] is a polypeptide shown as SEQ ID NO:759.

The C5 binding polypeptide(s) comprised in a C5 binding polypeptide are, in one embodiment, independently selected from 58-mer or 60-mer C5 binding polypeptides as previously described. In particular, the C5 binding compound may comprise one or more C5 binding polypeptides independently selected from any one of SEQ ID NO:497-508, SEQ ID NO:516, SEQ ID NO:519-520, SEQ ID NO:522-524, SEQ ID NO:528-531, SEQ ID NO:534-535, SEQ ID NO:537, SEQ ID NO:542, SEQ ID NO:545, SEQ ID NO:552-553, SEQ ID NO:555, SEQ ID NO:562, SEQ ID NO:574-575, SEQ ID NO:583, SEQ ID NO:588, SEQ ID NO:602, SEQ ID NO:606, SEQ ID NO:615, SEQ ID NO:621, SEQ ID NO:637, SEQ ID NO:647, SEQ ID NO:657, SEQ ID NO:662, SEQ ID NO:683, SEQ ID NO:693, SEQ ID NO:699, SEQ ID NO:701, SEQ ID NO:711, SEQ ID NO:739 and SEQ ID NO:746-757, such as a sequence selected from SEQ ID NO:497-508 and SEQ ID NO:746-757. In another embodiment, the amino acid sequence is selected from SEQ ID NO:497, SEQ ID NO:498, SEQ ID NO: 499, SEQ ID NO:500, SEQ ID NO:501, SEQ ID NO:746, SEQ ID NO:747, SEQ ID NO: 748, SEQ ID NO:750 and SEQ ID NO:753, such as from any one of SEQ ID NO:497, SEQ ID NO:500, SEQ ID NO:748 and SEQ ID NO:753.

In a further aspect, there is provided a polynucleotide encoding a C5 binding polypeptide or a compound as described above. An expression vector comprising such a polynucleotide may enable production of a C5 binding polypeptide or a C5 binding compound, for example by expression in a host cell.

It should be understood that the C5 binding polypeptide according to the present invention may be useful as a therapeutic or diagnostic agent in its own right or as a means for targeting other therapeutic or diagnostic agents, with e.g. direct or indirect effects on the complement protein C5. A direct therapeutic effect may for example be accomplished by inhibiting C5 cleavage. In one embodiment, there is thus provided a combination of a C5 binding polypeptide or a C5 binding compound according to the invention with a therapeutic agent. Non-limiting examples of therapeutic agents that may prove useful in such a combination are immunostimulatory agents and radionuclides.

Thus, the C5 binding polypeptide as such, or as comprised in a C5 binding compound or a combination according to the invention, is in one embodiment provided for use in therapy, for example for the treatment of a C5 related condition, such as a C5 related condition in a mammal, such as a human subject. In one embodiment, said C5 related condition is selected from inflammatory disease, such as antigen-induced arthritis, sepsis, synovial inflammation, vasculitis and asthma; autoimmune disease, such as systemic lupus erythematosus (SLE), cold agglutinin disease, rheumatoid arthritis, multiple sclerosis (MS), Sjögren's syndrome, dermatomyositis, myasthenia gravis and other autoantibody driven diseases such as Guillain-Barré syndrome (GBS), Fisher syndrome, systemic sclerosis, anti-glomerular basement membrane (anti-GBM) and anti-phospholipid syndrome (APS); infectious disease, such as hemolytic-uremic syndrome (HUS), viral infections, bacterial infections and fungal infections; cardiovascular disease, such as (acute) myocardial infarction (undergoing revascularization either by fibrinolysis or percutaneous coronary intervention (PCI)); neurodegenerative disorders such as Alzheimer's disease (AD), Huntington's disease, Creutzfeld-Jacob disease and Parkinson's disease; cancers; wounds; graft injury, such as ischemia reperfusion injury (IRI) and acute antibody mediated rejection (AMR); eye disease, such as age-related macular degeneration (AMD), uveitis, diabetic ocular diseases and disorders, and retinopathy of prematurity; kidney disease, such as membranous glomerulonephritis, membranous nephritis, immunoglobulin A nephropathy, Lupus nephritis, Goodpasture syndrome and post-streptococcal glomerulonephritis; pulmonary diseases, such as adult respiratory distress syndrome, chronic obstructive pulmonary disease and cystic fibrosis; hematological diseases; such as hemolytic anaemia, paroxysmal cold hemoglobinuria, atypical hemolytic uremic syndrome (aHUS) and paroxysmal nocturnal hemoglobinuria (PNH); allergic diseases, such as anaphylactic shock, allergy and asthma; and dermatological diseases, such as pemphigus, bullous pemphigoid, phototoxic reactions and psoriasis. In a more particular embodiment, the C5 binding polypeptide, compound or combination according to the invention is used for treatment of paroxysmal nocturnal hemoglobinuria (PNH).

As mentioned when discussing organ transplantation in the background section above, differences between donor and recipient (e.g. ABO and MCH classes) as well as the condition of the transplanted organ may lead to delayed functioning or even rejection of the transplanted organ. Treatment may thus be necessary to eliminate anti-donor antibodies despite a positive donor-recipient crossmatch or to eliminate ABO antibodies when transplantation occurs against the ABO barrier. Such treatment typically includes immunoadsorption, e.g. by use of affinity chromatography techniques, prior to as well as after transplantation or plasmapheresis. Such procedures however runs the risk of eliminating nearly all antibodies present in the circulation, thus including therapeutic antibodies. The C5 binding polypeptides or compounds of the invention are however not affected by any antibody removing procedures, and may thus be exploited in these treatments.

In some C5 related conditions where a more local acute pathology in readily accessible tissues, such as lung and the blood stream, dominates rather than systemic pathologies, a drug with a very short half-life could be advantageous over one with a slow elimination. Thus, in such C5 related conditions, a C5 binding polypeptide without a half-life extending moiety may be beneficial. As previously accounted for, a C5 binding polypeptide according to the invention will, due to its relatively small size, exhibit a relatively rapid pharmacokinetic profile when administered to a mammal such as a human. The C5 binding polypeptide according to the invention may potentially be active in treatment of C5 related conditions such as asthma (Zhang et al. Expert Rev Clin Immunol 2010, 6:269-277), sepsis (Ward et al. The Sci World J 2010, 10:2395-2402), and hypersensitivity syndrome including the C activation-related pseudoallergy (CARPA, a reaction to certain therapeutic liposomes and lipid excipient-based drugs that in rare cases can lead to life threatening cardiopulmonary distress (Szebeni et al. Adv Drug Delivery Rev 2011, 63:1020-1030). In addition, a C5 binding polypeptide according to the invention may be used for complement inhibition when a recipient of blood transfusion has received blood of an incompatible type (a situation occurring in about 1:14000 transfusion units in the US which is associated with high mortality, Goodnough et al. Lancet 2003, 361:161-169).

In a related aspect, there is provided a method of treatment of a C5 related condition, comprising administering of a C5 binding polypeptide, or combination as described above to a mammalian subject in need thereof. Consequently, in the method of treatment, the subject is treated with a C5 binding polypeptide, a C5 binding compound or a combination according to the invention. In a more specific embodiment of said method, the binding of the C5 binding polypeptide or the combination, to a C5 expressed on a cell surface in the subject inhibits C5 cleavage. In one embodiment of the method of treatment, the C5 related condition is selected from inflammatory disease; autoimmune disease; infectious disease; cardiovascular disease; neurodegenerative disorders; cancers; wounds; graft injury; eye disease; kidney disease; pulmonary diseases; hematological diseases; allergic diseases and dermatological diseases. In particular the C5 related condition may be as defined above in relation to therapeutic use of a C5 binding polypeptide, compound or combination according to the invention. The C5 related condition may for example be paroxysmal nocturnal hemoglobinuria (PNH). In one embodiment of the method of treatment, the said C5 binding polypeptide is administered intravenously, subcutaneously, by inhalation, nasally, orally, intravitreally, or topically.

The invention will now be further illustrated by the following non-limiting Examples.

### BRIEF DESCRIPTION OF THE FIGURES

FIG. 1A-AH is a listing of the amino acid sequences of examples of C5 binding motifs comprised in C5 binding polypeptides of the invention (SEQ ID NO:1-248), examples of 49-mer C5 binding polypeptides according to the invention (SEQ ID NO:249-496), examples of 58-mer C5 binding polypeptides according to the invention (SEQ ID NO:497-744) and examples of 60-mer C5 binding polypeptides according to the invention (SEQ ID NO:745-757), as well as the sequences of protein Z (SEQ ID NO:758), an albumin binding domain (ABD094, SEQ ID NO:759), the Swiss-Prot entry P01031 of human C5 (amino acid residues 1-1676, SEQ ID NO:760: of which the α-chain corresponds to amino acid residues 678-1676 and the β-chain corresponds to amino acid residues 19-673), the sequence of the His₆-tagged tic protein OmCI used herein (SEQ ID NO:761) and cynomolgus C5 (SEQ ID NO:762) derived from genomic sequence (published on-line at www.ebi.ac.uk/ena; Ebeling et al. (2001) Genome Res. 21(10):1746-1756) using human C5 as template. The sequence contains two unknown amino acids “X” in positions 63 and 1346.

FIG. 2 shows the result of atypical binding analysis performed in a Biacore instrument as described in Example 2. Sensorgrams were obtained by injection of human C5 (hC5; black solid curve), cynomolgus C5 (cC5; black short-dashed curve), rat C5 (rC5; black long-dashed curve), human MG7 domain (hMG7; gray dotted curve), and human immunoglobulin G (hIgG; gray solid curve), respectively, over an immobilized dimeric Z variant (Z05477, SEQ ID NO:509).

FIG. 3 is a column chart showing the response in ELISA against hC5 and rC5, respectively, for selected maturated Z variants. The black columns corresponds to the absorbance at 450 nm obtained using 0.05 μg/ml hC5 (left column in each group) and to the absorbance at 450 nm obtained using 4 μg/ml rC5 for each Z variant (right column in each group), as described in Example 4. The responses for the Z variant Z05363 (SEQ ID NO:510) are plotted as a positive control.

FIG. 4 schematically shows different constructs encompassing one or several C5 binding Z variants selected from SEQ ID NO:745-757, optionally linked to ABD094 (SEQ ID NO:759).

FIG. 5 A-C shows SDS-PAGE analyses of purified C5 binding Z variants (reduced condition) visualized by Instant Blue, as described in Example 6. FIG. 5A) represents one example of dimeric Z-Z-ABD (lane 1 where Z is equal to SEQ ID NO:745 and ABD is equal to SEQ ID NO:759) compared with different Z-ABD fusion proteins (where Z is equal to SEQ ID NO:745 (lane 2), SEQ ID NO:748-757 (lanes 4-13) fused to ABD094 (SEQ ID NO:759) by a GS linker); FIG. 5B) represents one C5 binding Z variant (SEQ ID NO:753) in different constructs, and FIG. 5C) represents two different C5 binding Z variants (SEQ ID NO:748, lanes 2-3 and 6 and SEQ ID NO:753, lanes 4-5 and 7), in monomeric form (lanes 6-7) and in fusion with ABD094 (SEQ ID NO:759) via a GS(G₄S)₂ linker (lanes 2-5).

FIG. 6A-B are diagrams showing exemplary data of dose-response characterization of the potency of different C5 binding Z variants to inhibit complement activation as seen in a hemolytic assay, described in Example 6. C5 deficient serum was diluted 63-fold and supplemented with 0.1 nM hC5. FIG. 6A) shows effect of different Z-ABD fusion proteins (Z variants corresponding to SEQ ID NO:745, SEQ ID NO:748-753 and SEQ ID NO:756 fused to ABD094 (SEQ ID NO:759) by a GS linker) to hC5, whereas FIG. 6B) shows effect of different C5 binding constructs comprising the same C5 binding Z variant (Z06175a, SEQ ID NO:753) as monomer or dimer, in fusion with ABD094 (SEQ ID NO:759), or as provided with a His₆-tag (six histidine residues), compared to the C5 binding tick protein OmCI (SEQ ID NO:761).

FIG. 7A-B are diagrams showing exemplary data of equilibrium binding based on the displacement ECL technique described in Example 6. FIG. 7A shows C5 binding of different Z variants (SEQ ID NO:745, SEQ ID NO:748-757) in fusion with ABD094 (SEQ ID NO:759) compared to C5 binding of the tick protein OmCI (SEQ ID NO:761). FIG. 7B shows binding of different C5 binding constructs comprising the same C5 binding Z variant (SEQ ID NO:753) as monomer or dimer, in fusion with ABD094 (SEQ ID NO:759) or as provided with a His₆-tag.

FIG. 8A-B show interactions between Z-ABD variants and human serum albumin (HSA) studied as described in Example 7. FIG. 8A) Size exclusion chromatography (SEC) where Z-ABD (Z06175a (SEQ ID NO:753) fused to ABD094 (SEQ ID NO:759) by a GS linker) has been preincubated with equimolar amounts of HSA (1). As a comparison, the chromatograms for HSA alone (2) and Z-ABD alone (3) are also shown in the graph. FIG. 8B) Biacore sensorgrams of Z-ABD and Z-ABD-Z (Z06175a (SEQ ID NO:753) fused to ABD094 (SEQ ID NO:759) by linkers specified in FIG. 4, in construct 2 and construct 5, respectively) injected over an HSA coated surface. Each of the two constructs was injected at a concentration of 25, 100 and 400 nM.

FIG. 9 is a diagram showing the pharmacokinetic profiles for the C5 binding compounds Z-ABD and Z-ABD-Z (Z06175a, SEQ ID NO:753) fused to ABD094 (SEQ ID NO:759) by linkers specified in FIG. 4, in construct 2 and construct 5, respectively) in Male Spmgue Dawley rats overtime after intravenous (i.v., 0.25 μmol/kg) and subcutaneous (s.c., 0.5 μmol/kg) administration, as described in Example 8. Each data point represents an average from three individual animals at a specific time point ranging from five minutes to two weeks after dosing for animals dosed i.v and from 15 minutes to two weeks for animals dosed s.c.

FIG. 10 shows ex vivo hemolysis in sheep erythrocytes after exposure to serum diluted 1:5 from animal samples taken from Spmgue Dawley rats after intravenous (i.v.; 0.25 μmol/kg) administration of Z-ABD (Z06175a (SEQ ID NO:753) fused to ABD094 (SEQ ID NO:759) by a GS linker), as described in Example 8. Each dot represents one individual animal at a specific time point ranging from five minutes to two weeks after dosing.

FIG. 111 shows ex vivo hemolysis in sheep erythrocytes after exposure to serum diluted 1:5 from animal samples taken from Spmgue Dawley rats after subcutaneous (s.c.; 0.5 μmol/kg) administration of Z-ABD (Z06175a (SEQ ID NO:753) fused to ABD094 (SEQ ID NO:759) by a GS linker), as described in Example 8. Each dot represents one individual animal at a specific time point ranging from 15 minutes to two weeks.

FIG. 12 shows the hemolysis versus Z-ABD (Z06175a (SEQ ID NO:753) fused to ABD094 (SEQ ID NO:759) by a GS linker) serum concentration following i.v. and s.c. administration to male Sprague Dawley rats, as described in Example 8.

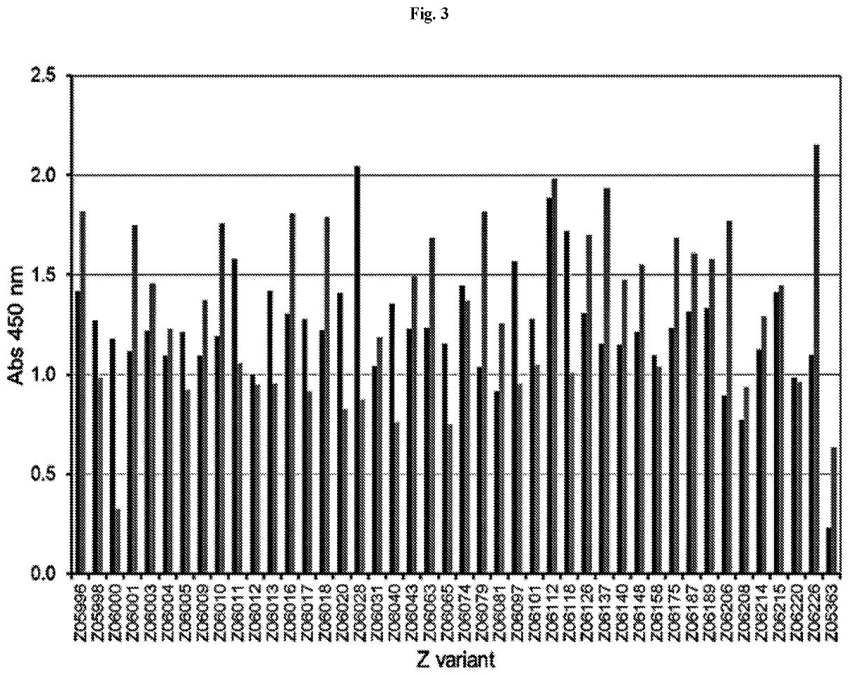

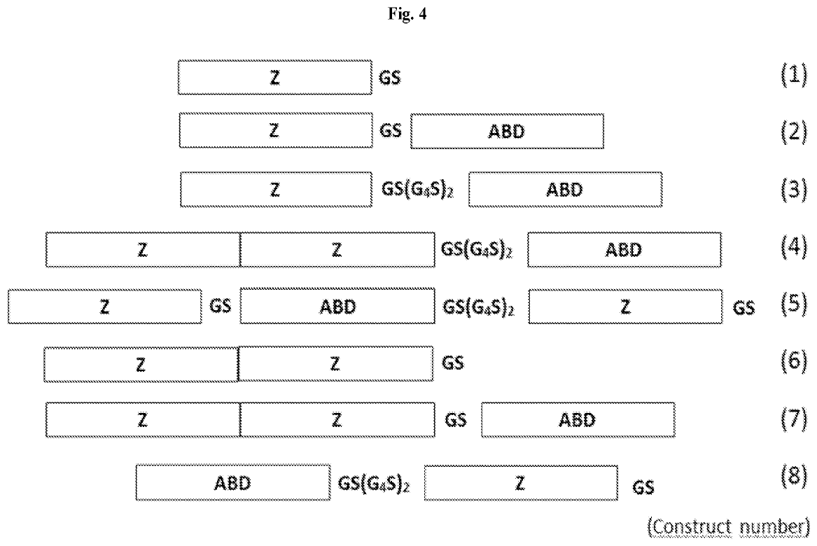

FIG. 13 shows hemolysis in sheep erythrocytes versus Z-ABD-Z (Z06175a (SEQ ID NO:753) fused to ABD094 (SEQ ID NO:759) by linkers specified in FIG. 4, construct 5) serum concentration following i.v. and s.c. administration to male Sprague Dawley rats, as described in Example 8.

FIG. 14 shows the serum exposure of Z-ABD (Z06175a (SEQ ID NO:753) fused to ABD094 (SEQ ID NO:759) by a GS linker) following i.v. (415 nmol/kg) and s.c. (1250 nmol/kg) administration in male Cynomolgus monkey, as described in Example 9. Each data point represents the mean of three individual animals.

FIG. 15 is a diagram showing the effect (C5a concentration in lavage) of the pro-inflammatory molecule zymosan (40 mg/kg i.p.) alone and in combination with a C5 binding Z-ABD fusion molecule (Z06175a (SEQ ID NO:753) fused to ABD094 (SEQ ID NO:759) by a GS linker)) or OmCI (SEQ ID NO:761) analyzed as described in Example 10. Z-ABD was administered at 20 nmol/kg (LD), 100 nmol/kg (MD) and 500 nmol/kg (HD) s.c. 18 h before induction with zymosan. OmCI (30 nmol/kg) was administered i.p. 1 h before zymosan treatment and samples were taken 1 h after zymosan induction.

FIG. 16A-B show the pharmacokinetic profile of Z-ABD (Z06175a (SEQ ID NO:753) fused to ABD094 (SEQ ID NO:759) by a GS linker) following intratracheal administration of 500 nmol/kg into female C57b1 mice, as described in Example 11. FIG. 16A) serum concentration in each animal (n=3 for each time point, 27 animals totally) and FIG. 16B) hemolysis in sheep erythrocytes exposed to these serum samples diluted 1:5.

### EXAMPLES

The following materials were used throughout this work except where otherwise noted.
Escherichia coli strain RR1ΔM15 (Ruther, Nucleic Acids Res 10:5765-5772, 1982).
Escherichia coli Strain XL1-Blue (Agilent Technologies, Cat. No. 200268).
Human complement protein C5 (hC5). The full 1676 amino acid pro-protein has GenBank accession number: NP_001726 (SEQ ID NO:760) wherein amino acids 19-673 is the beta chain and amino acids 678-1676 is the alpha chain. Human C5 used herein was purchased from Quidel (cat. no. A403)
Cynomolgus complement protein C5 (cC5; SEQ ID NO:762). The cC5 sequence was derived from the Cynomolgus (Macaca fascicularis) genomic sequence (www.ebi.ac.uk/ena; Ebeling et al. (2001) Genome Res. 21(10):1746-1756). The coding region of human C5 was retrieved from www.ensembl.org, and the C5 gene was localized to chromosome 15. The region containing the gene is approximately 110 000 bases long and is contained in contigs CAEC01154150 to CAEC01154178. The contigs were manually joined to a single file and used as a genomic context for the sim4 software to align the coding region of human C5 to the raw Cynomolgus genomic material. Cynomolgus C5 used herein was purified in-house from serum using a three-step procedure; PEG6000 precipitation, ion exchange and OmCI affinity chromatography.
Rat Complement protein C5 (rC5; GenBank accession number: XP_001079130) Rat C5 used herein was purified in-house from serum using a three-step procedure; PEG6000 precipitation, ion exchange and OmCI affinity chromatography.
Human MG7 (hMG7) domain of complement protein C5, corresponding to amino acid residues 822-931 of human C5 (SEQ ID NO:760; Fredslund et al. (2008) Nature Immunology 9: 753-760) produced in-house in Freestyle HEK293 cells.
hMG7 binding protein.
OmCI (AF2999, Nunn, M. A. et al. supra) from soft tick Ornithodoros moubata OmCI with a His₆ tag in the C-terminus (SEQ ID NO:761) was produced in-house in E. coli strain Origami(DE3) and purified on a HisTrap1 column.

### Example 1: Selection and Screening of Complement Protein C5 Binding Polypeptides

### Materials and Methods

Biotinylation of target protein hC5: hC5 was biotinylated according to the manufacturer's recommendations at room temperature (RT) for 40 min using No-Weigh EZ-Link Sulfo-NHS-LC-Biotin (Pierce, cat. no. 21327) at a ten times (10×) molar excess. Subsequent buffer exchange to PBS (10 mM phosphate, 137 mM NaCl, 2.68 mM KCl, pH 7.4) was performed using Protein Desalting Spin Columns (Pierce, cat. no. 89849) according to the manufacturer's instructions.

Phage display selection of C5-binding polypeptides: Libraries of random variants of protein Z displayed on bacteriophage, constructed in phagemid pAffi1/pAY00065/pAY02947/pAY02592 essentially as described in Grönwall et al. J Biotechnol 2007, 128:162-183), were used to select C5 binding polypeptides. Three different library vectors were used. Two of these utilize an albumin binding domain (ABD, GA3 of protein G from Streptococcus strain G148) as fusion partner to the Z variants generating the libraries Zlib003Naive.I and Zlib006Naive.II. The third library, Zlib004Naive.I utilizes the Taq DNA polymerase binding molecule Z03639 (denoted Z_{TaqS1-1} in Gunneriusson et al. Protein Eng 1999, 12:873-878) as fusion partner. The libraries had the following actual sizes: 3×10⁹ (Zlib003Naive.I); 1.5×10¹⁰ (Zlib006Naive.I); and 1.4×10¹⁰ (Zlib004Naive.I), the number referring to the amount of variants.

Phage stocks were prepared either in shake flasks (Zlib003Naive.I) as described in Grönwall et al. supra or in a 20 1 fermenter (Zlib006Naive.II and Zlib004Naive.I). Cells from a glycerol stock containing the phagemid library Zlib004Naive.I were inoculated in 20 1 of TSB-YE (Tryptic Soy Broth-Yeast Extract; 30 g/l TSB, 5 g/l yeast extract) supplemented with 2% glucose and 100 μg/ml ampicillin. Cells from a glycerol stock containing the phagemid library Zlib006Naive.II were inoculated in 20 1 of a defined proline free medium [dipotassium hydrogenphosphate 7 g/l, trisodium citrate dihydrate 1 g/l, uracil 0.02 g/l, YNB (Difco™ Yeast Nitrogen Base w/o amino acids, Becton Dickinson) 6.7 g/l, glucose monohydrate 5.5 g/l, L-alanine 0.3 g/l, L-arginine monohydrochloride 0.24 g/l, L-asparagine monohydrate 0.11 g/l, L-cysteine 0.1 g/l, L-glutamic acid 0.3 g/l, L-glutamine 0.1 g/l, glycine 0.2 g/l, L-histidine 0.05 g/l, L-isoleucine 0.1 g/l, L-leucine 0.1 g/l, L-lysine monohydrochloride 0.25 g/l, L-methionine 0.1 g/l, L-phenylalanine 0.2 g/l, L-serine 0.3 g/l, L-threonine 0.2 g/l, L-tryptophane 0.1 g/l, L-tyrosine 0.05 g/l, L-valine 0.1 g/l] supplemented with 100 μg/ml ampicillin. The cultivations were grown at 37° C. in a fermenter (Belach Bioteknik, BR20). When the cells reached an optical density (OD) of 0.7-0.8, approximately 2.6 1 of the cultivation was infected using a 10×molar excess of M13K07 helper phage (New England Biolabs #N0315S). The cells were incubated for 30 minutes, whereupon the fermenter were filled up to 20 1 with TSB-YE supplemented with 100 μM IPTG (isopropyl-β-D-1-thiogalactopyranoside, for induction of expression), 25 μg/ml kanamycin and 12.5 μg/ml carbenicillin and grown at 30° C. for 22 h. The cells in the cultivation were pelleted by centrifugation at 15,900 g and the phage particles remaining in the medium were thereafter precipitated twice in PEG/NaCl (polyethylene glycol/sodium chloride), filtered and dissolved in PBS and glycerol as described in Grönwall et al. supra. Phage stocks were stored at −80° C. before use.

Selections were performed in four cycles against biotinylated hC5. Phage stock preparation, selection procedure and amplification of phage between selection cycles were performed essentially as described in WO 2009/077175. PBS supplemented with 3% bovine serum albumin (BSA) and 0.1% Tween20 was used as selection buffer and the target-phage complexes were directly captured by Dynabeads® M-280 Streptavidin (Dynal, cat. no. 112.06). 1 mg beads per 10 μg complement protein C5 was used. E. coli strain RR1ΔM15 was used for phage amplification. In cycle 1 of the selections, 100 nM hC5 was used and two washes with PBST 0.1% (PBS supplemented with 0.1% Tween-20) were performed. An increased stringency, using a lowered target concentration and an increased number of washes, was applied in the subsequent three cycles. In cycle 2, 3 and 4; 50 or 33 nM hC5, 25 or 11 nM hC5 and 12.5 or 3.7 nM hC5 were used. In cycle 2, 3 and 4; 4, 6 and 8 washes were performed, using PBST 0.1% in all cycles or PBST 0.2%, 0.3% and 0.4% in cycle 2, 3 and 4.

ELISA screening of Z variants: To test if the selected Z variant molecules could indeed interact with human complement protein C5, ELISA assays were performed. The Z variants were produced by inoculating single colonies from the selections into 1 ml TSB-YE medium supplemented with 100 μg/ml ampicillin and 0.1 mM IPTG in deep-well plates (Nunc, cat. no. 278752). The plates were incubated for 18-24 h at 37° C. Cells were pelleted by centrifugation, re-suspended in 300 μl PBST 0.05% and frozen at −80° C. to release the periplasmic fraction of the cells. Frozen samples were thawed in a water bath and cells were pelleted by centrifugation. The periplasmic supernatant contained the Z variants as fusions to an albumin binding domain (GA3 of protein G from Streptococcus strain G148), expressed as AQHDEALE-[Z #####]-VDYV-[ABD]-YVPG (SEQ ID NO: 767) (Grönwall et al, supra), or to the Taq DNA polymerase binding molecule Z03639, expressed as AQHDEALE-[Z #####]-VDYV-[Z03639]-YVPG (SEQ ID NO: 768). Z #####refers to individual 58 amino acid residues Z variants.

Half-area 96-well ELISA plates (Costar, cat. no. 3690) were coated with 50 μl/well of coating buffer (50 mM sodium carbonate, pH 9.6) containing 4 μg/ml of an antibody specific for Z variants (Affibody, cat. no. 20.1000.01.0005) and incubated over-night at 4° C. The antibody solution was poured off and the wells were blocked with 100 μl of PBSC (PBS supplemented with 0.5% casein (Sigma, cat. no. C8654) for 1-2 h at RT. The blocking solution was discarded and 50 μl periplasmic solution was added to the wells and incubated for 1 h at RT under slow shaking. The supernatants were poured off and the wells were washed 4 times with PBST 0.05%. Then 50 μl of biotinylated complement protein hC5, at a concentration of 5 μg/ml in PBSC, was added to each well. The plates were incubated for 1.5 h at RT followed by washes as described above. Streptavidin-HRP (Horseradish peroxidase; Dako, cat. no. P0397) was diluted 1:10,000 in PBSC, added to the wells which were then incubated for 45 min. After washing as described above, 50 μl ImmunoPure TMB substrate (Thermo Scientific, cat. no. 34021) was added to the wells and the plates were treated according to the manufacturer's recommendations. Absorbance of the wells was measured at 450 nm using a multi-well plate reader, Victor³ (Perkin Elmer).

As positive control, a periplasmic fraction also containing the PSMA binding molecule Z03938 expressed as AQHDEALE-[Z03938]-VDYV-[Z03639]-YVPG (SEQ ID NO: 769) was assayed against 5 μg/ml biotinylated PSMA protein. As negative control; the same periplasmic preparation was assayed against complement protein hC5. Sequencing was performed for the clones with positive absorbance values against hC5.

Sequencing: PCR fragments were amplified from single colonies using a standard PCR program and the primers AFFI-21 (5′-tgcttccggctcgtatgttgtgtg) (SEQ ID NO: 770) and AFFI-22 (5′-cggaaccagagccaccaccgg) (SEQ ID NO: 771). Sequencing of amplified fragments was performed using the biotinylated oligonucleotide AFFI-72 (5′-biotin-cggaaccagagccaccaccgg) (SEQ ID NO: 772) and a BigDye® Terminator v3.1 Cycle Sequencing Kit (Applied Biosystems), used in accordance with the manufacturer's protocol. The sequencing reactions were purified by binding to magnetic streptavidin coated beads (Detach Streptavidin Beads, Nordiag, cat. no. 2012-01) using a Magnatrix 8000 (Magnetic Biosolution), and analyzed on ABI PRISM® 3100 Genetic Analyzer (PE Applied Biosystems).
Blocking ELISA: Clones found positive for hC5 in the ELISA screening were subjected to an ELISA blocking assay in order to elucidate if their target binding was affected by the presence of the hC5 binding proteins OmCI and/or hMG7 binding protein. The blocking ELISA was run using Z variants expressed in periplasmic fractions as described in the section for ELISA screening above, but setting up 5 ml cultures in 12 ml round-bottom tubes and using 2 ml PBST 0.05% for pellet dissolution. The ELISA blocking assay was run as the ELISA screening assay, with a protocol modification introduced at the target step; OmCI or hMG7 binding protein were mixed with the target protein before addition to the assay plate. 5 μg/ml biotinylated hC5 was mixed with 5 times or 20 times molar excess of OmCI or hMG7 binding protein, respectively, then incubated 1 h at RT to allow complex formation before addition to the plate. For each clone, a reference (1), a negative control (2) and a background (3) response/signal, respectively, were obtained as follows: at the target step, solely hC5 was added to the Z variants (as in the screening ELISA) (1); the irrelevant protein PSMA (in house produced) was added to complement protein hC5, instead of OmCI or hMG7 binding protein (2); only buffer was added to the Z variants (3).

### Results

Phage display selection of complement protein C5 binding polypeptides: Individual clones were obtained after two-four cycles of phage display selections against biotinylated hC5.
ELISA screening of Z variants: The clones obtained after four cycles of selection were produced in 96-well plates and screened for complement protein C5 binding activity in ELISA. In total, nearly 400 clones were screened. The absorbance measurements showed many clearly hC5 positive clones. The result from a selection of clones is displayed in Table 1; the Z05363 (SEQ ID NO:510) variant is tagged with ABD, whereas the other listed Z variants are tagged with the Taq binding molecule Z03639 as described in the methods section. The PSMA specific molecule Z03938 used as a negative control gave a positive signal for PSMA, whereas no signal was obtained against hC5.
Blocking ELISA: Clones positive for hC5 were subjected to a blocking assay using the hC5 binding proteins OmCI and hMG7 binding protein. For five clones, the binding signal to complement protein C5 was completely extinguished by the presence of OmCI, reaching the same level as the background (Table 1). One of these clones, namely the Z05363 variant (SEQ ID NO:510), was also tested for its ability to bind hC5 in the presence of hMG7 binding protein. The hMG7 binding protein did not inhibit the binding of Z05363 to hC5.

TABLE 1
Response in ELISA to target, with or without
blocking molecule for a number of Z variants.

| Z variant | SEQ ID NO: # | hC5 (OD 450 nm) | OmCI-block |
|---|---|---|---|
| Z05363 | SEQ ID NO: 510 | 3.143 | complete |
| Z05477 | SEQ ID NO: 509 | 2.872 | complete |
| Z05483 | SEQ ID NO: 511 | 0.531 | complete |
| Z05538 | SEQ ID NO: 512 | 0.099 | complete |
| Z05692 | SEQ ID NO: 513 | 0.944 | complete |

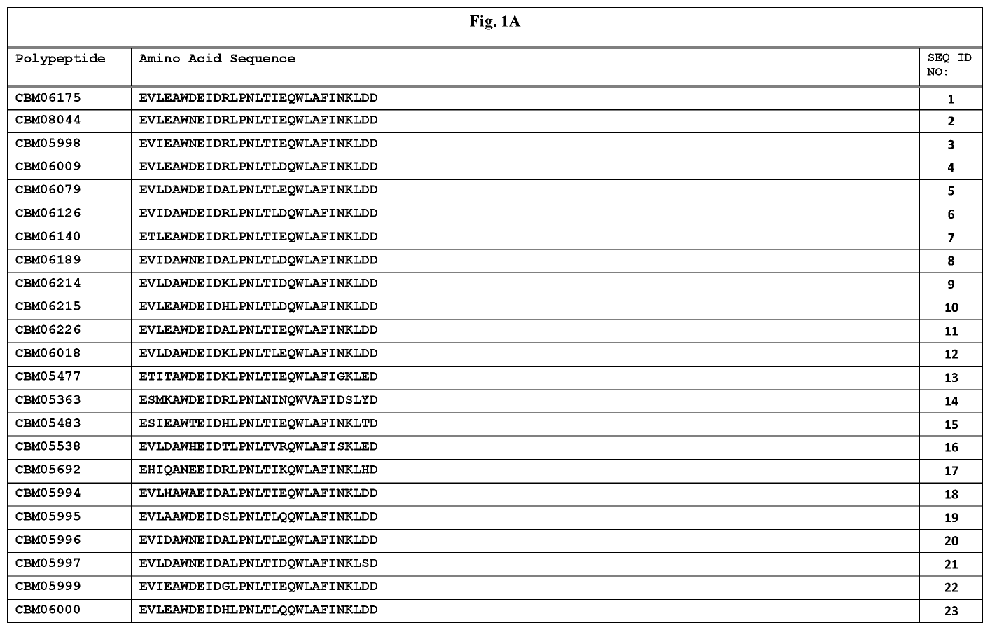

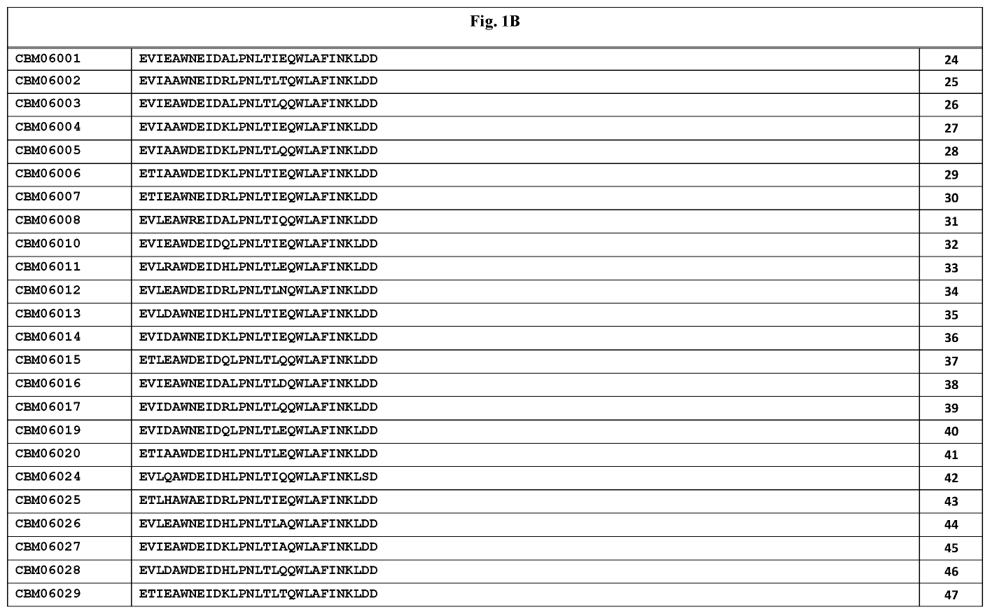

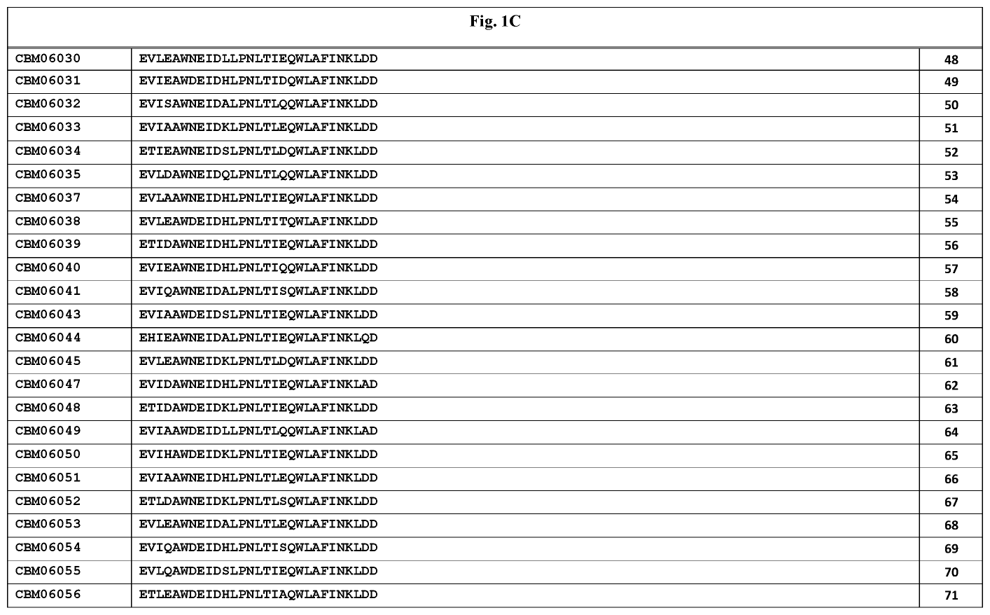

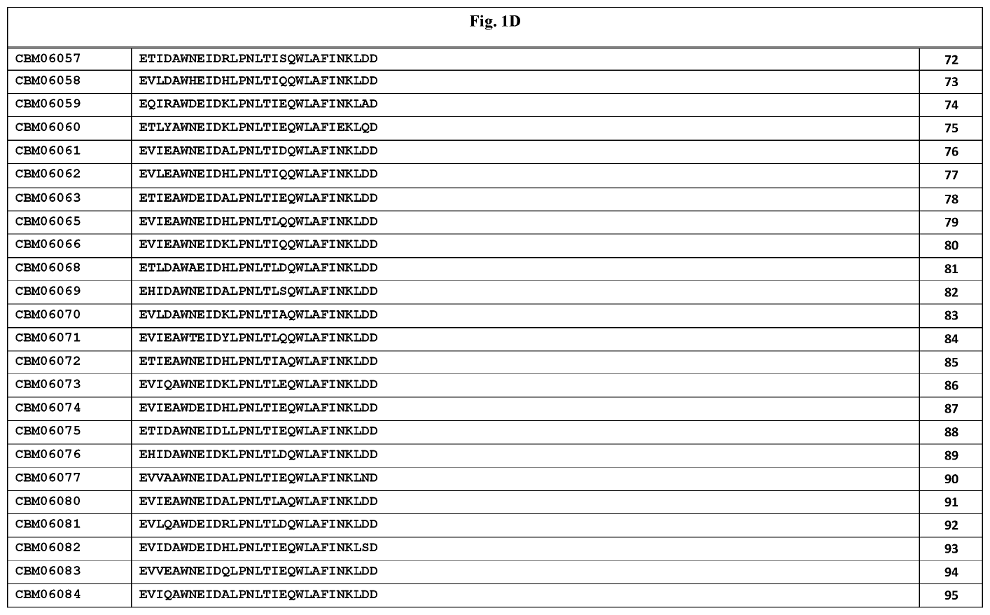

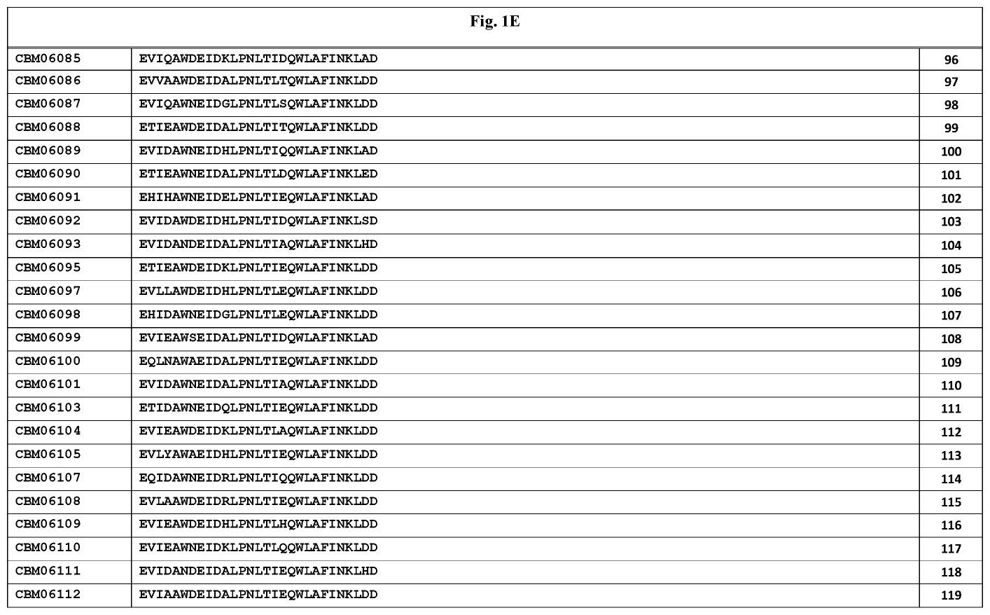

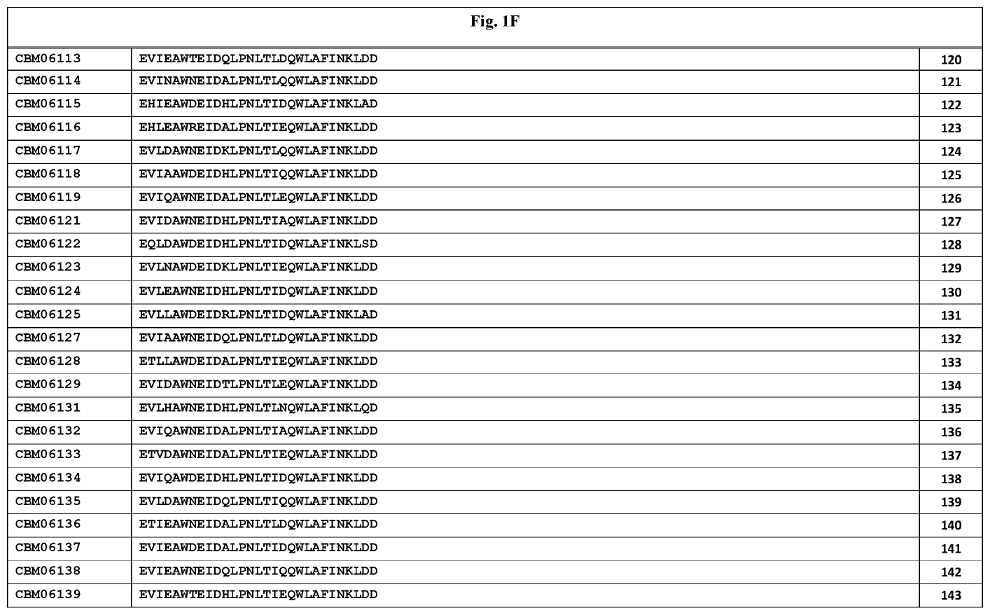

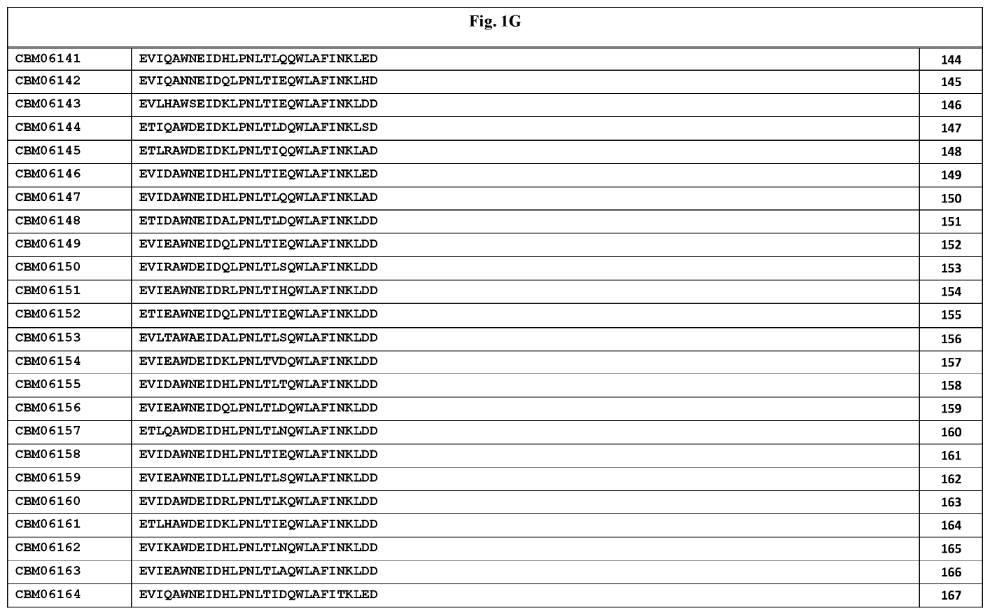

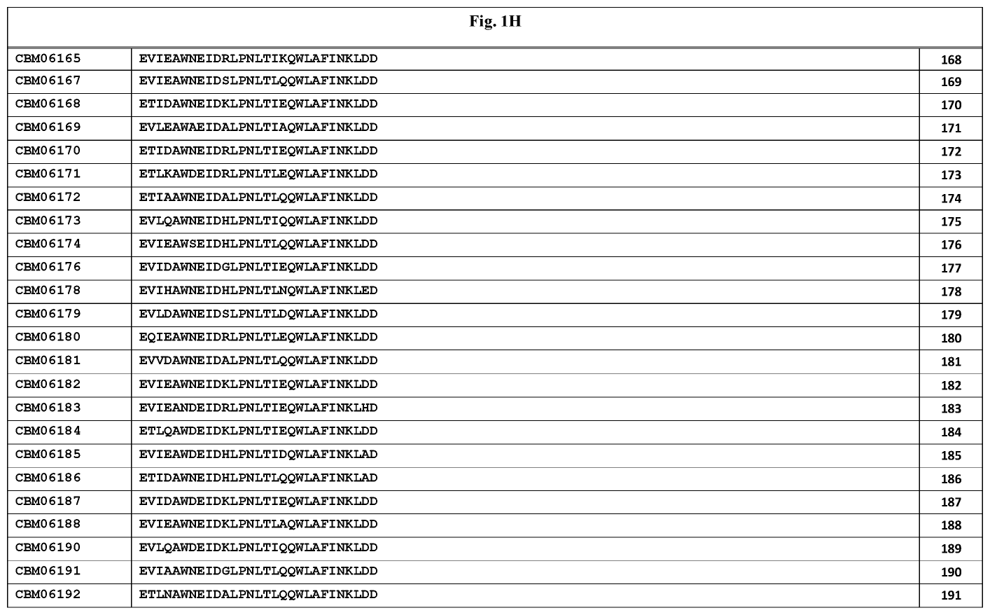

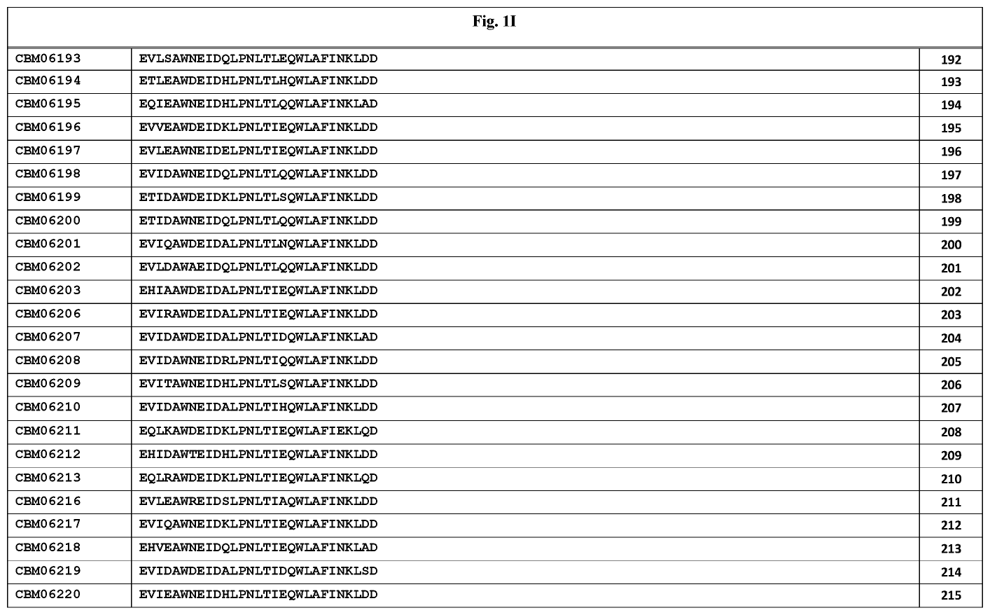

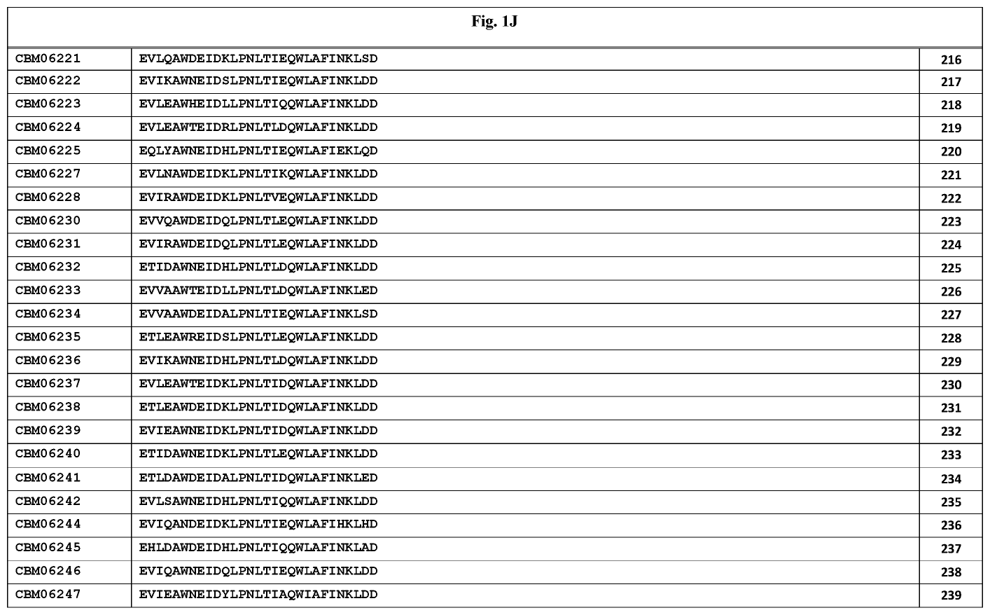

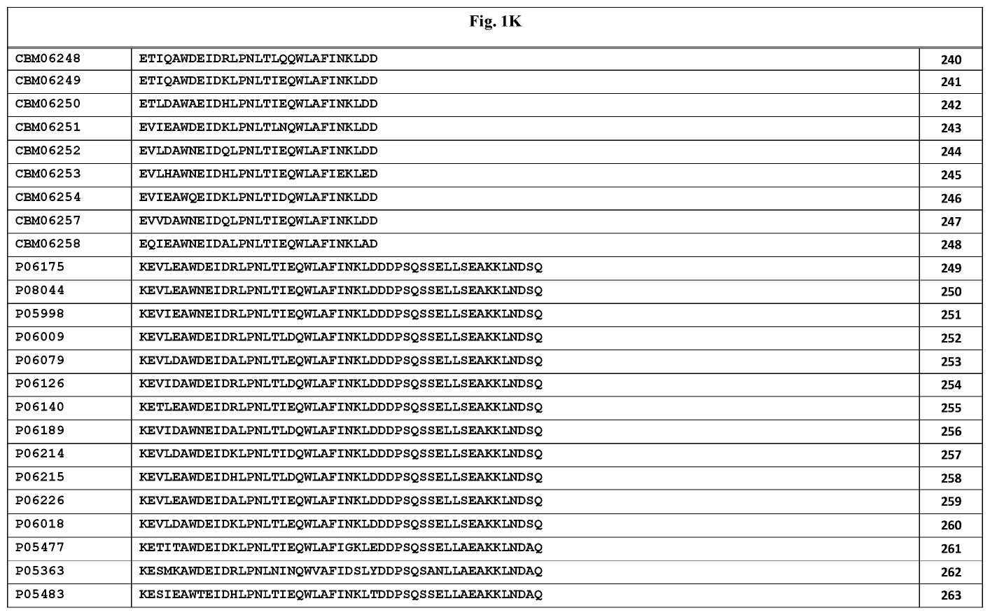

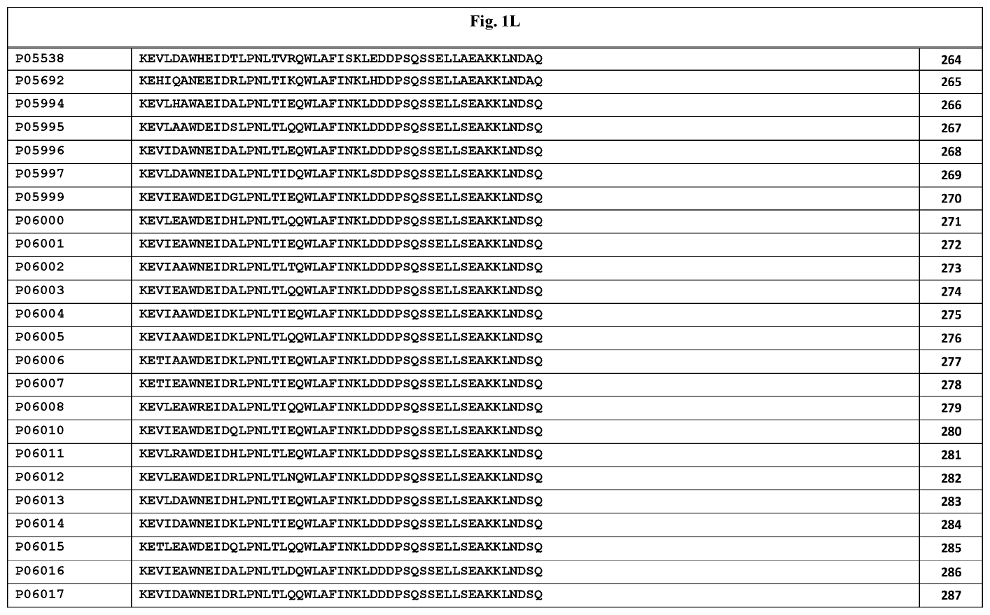

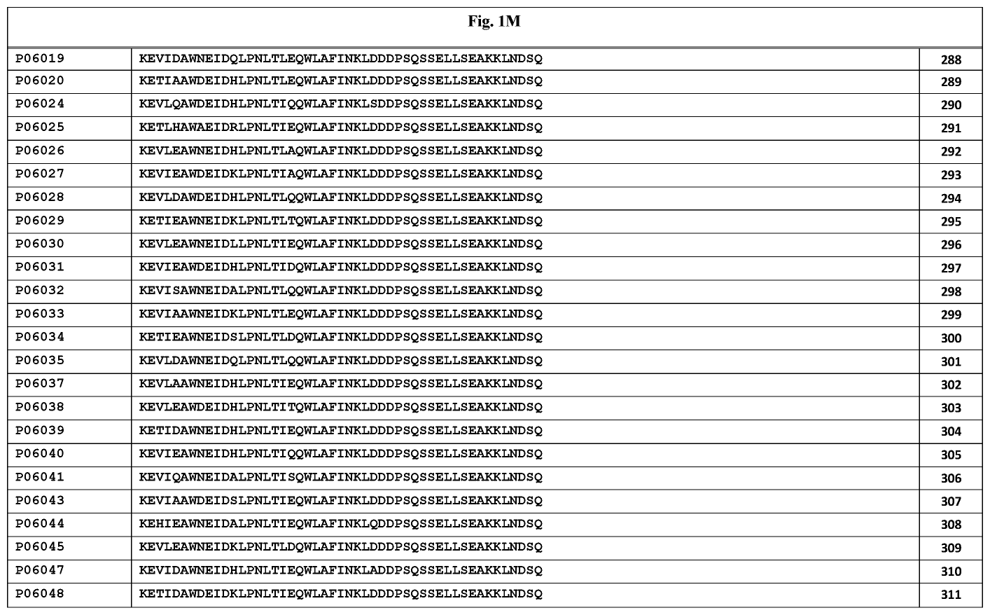

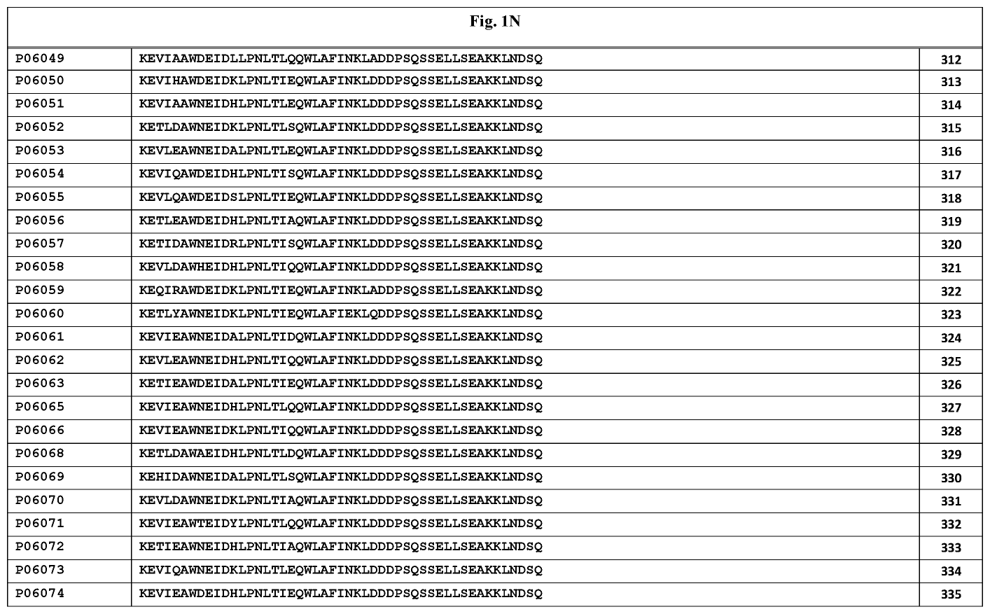

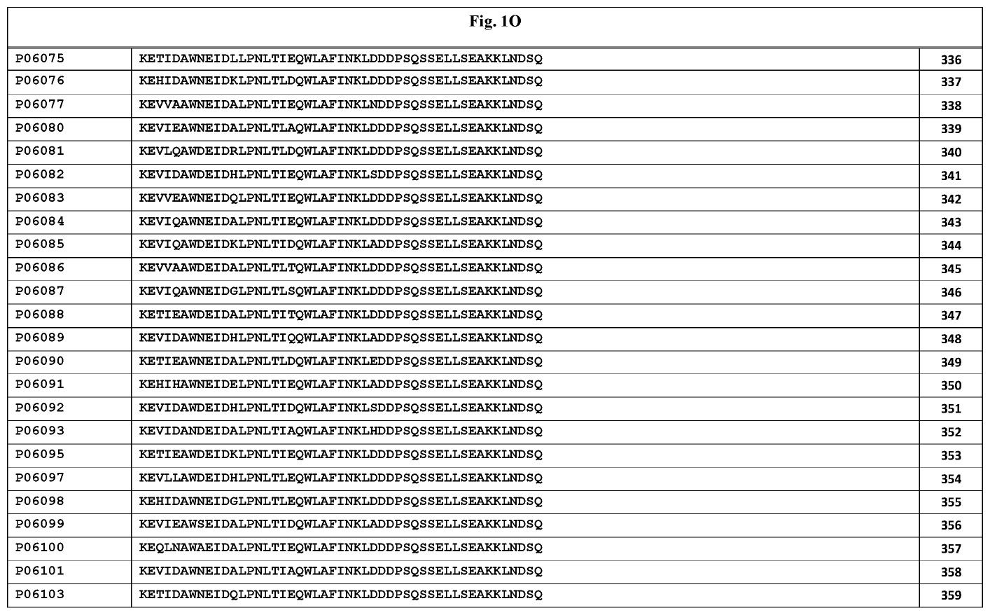

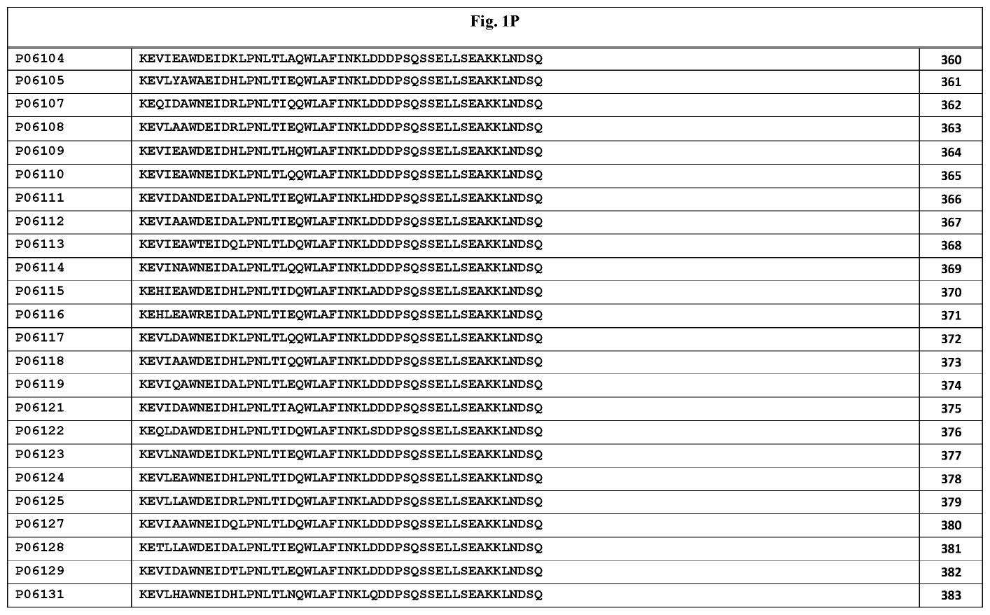

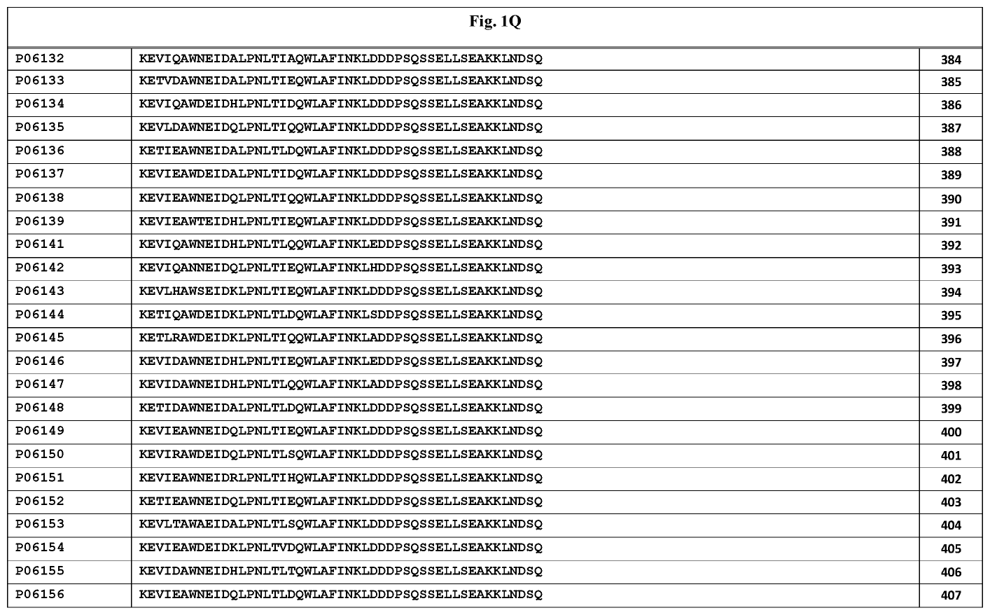

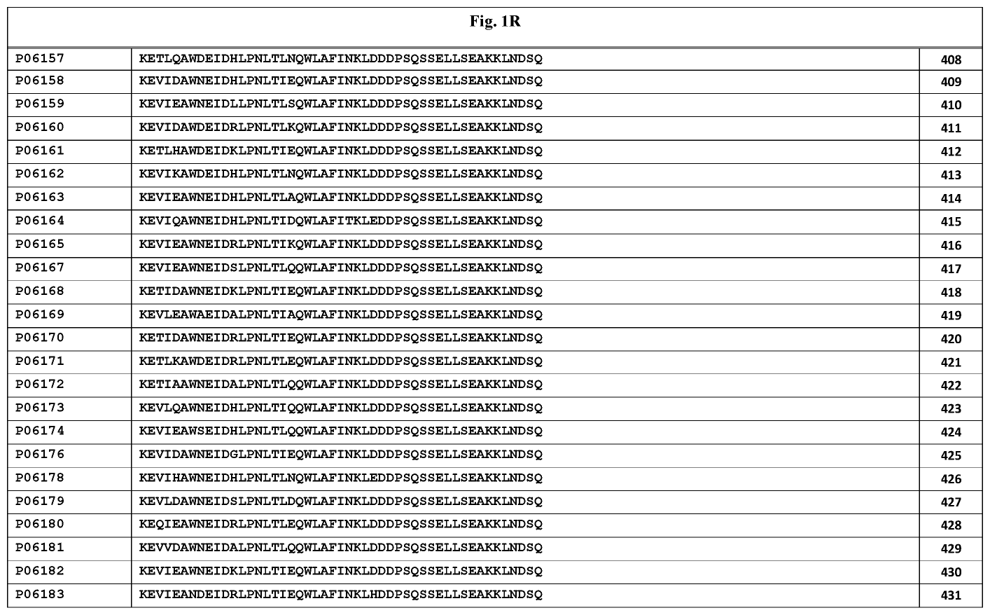

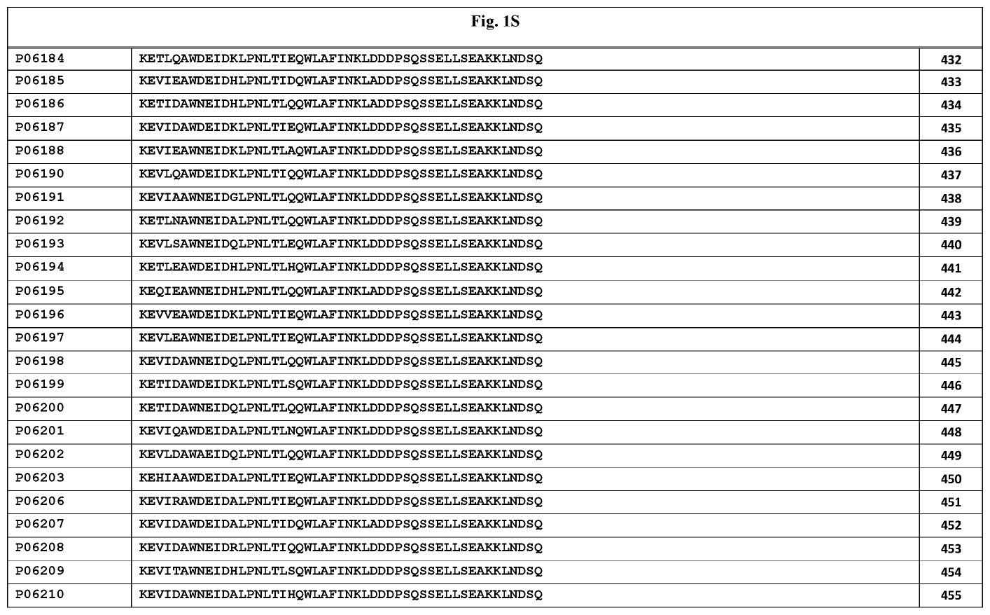

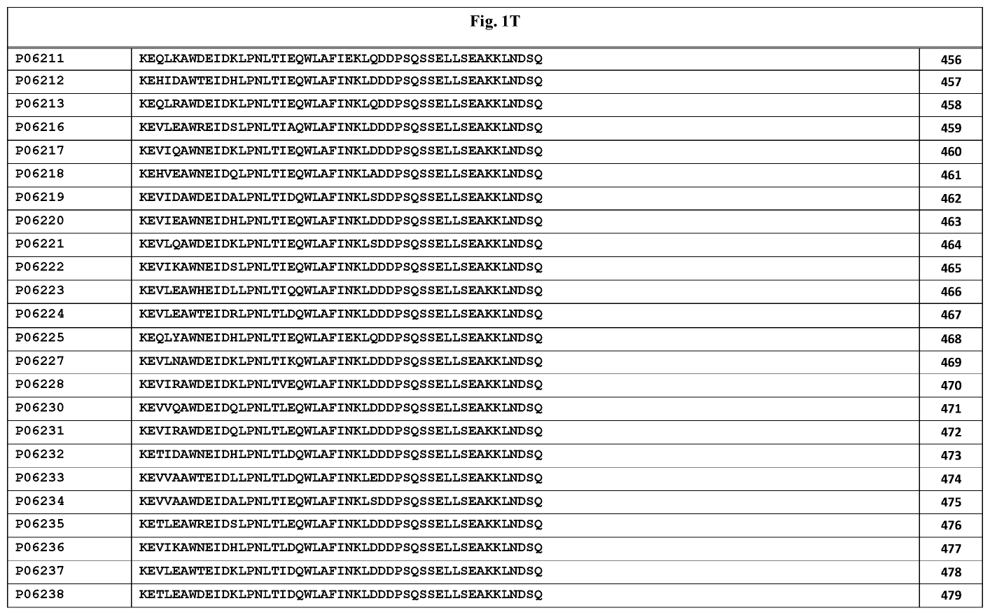

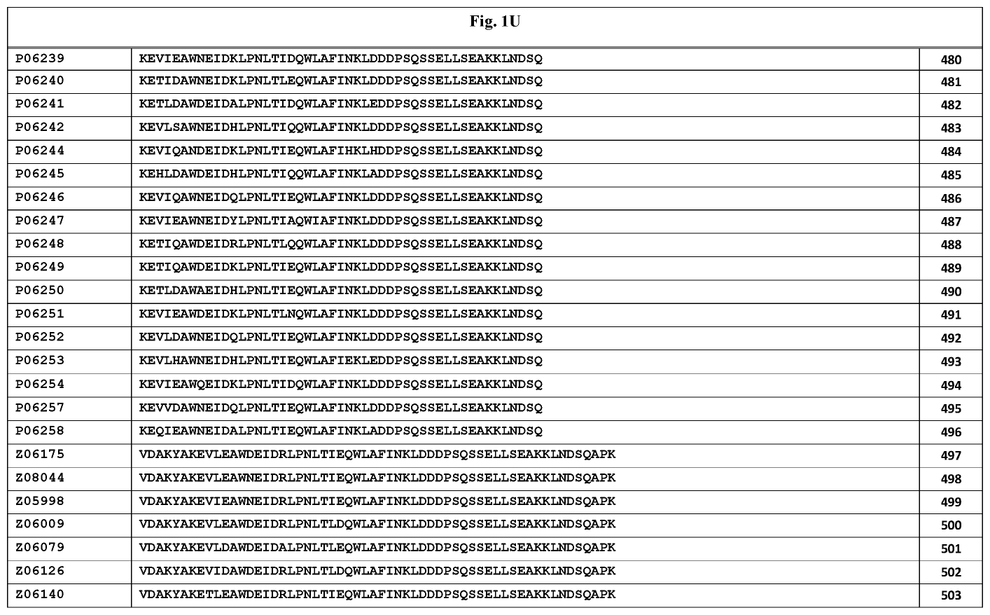

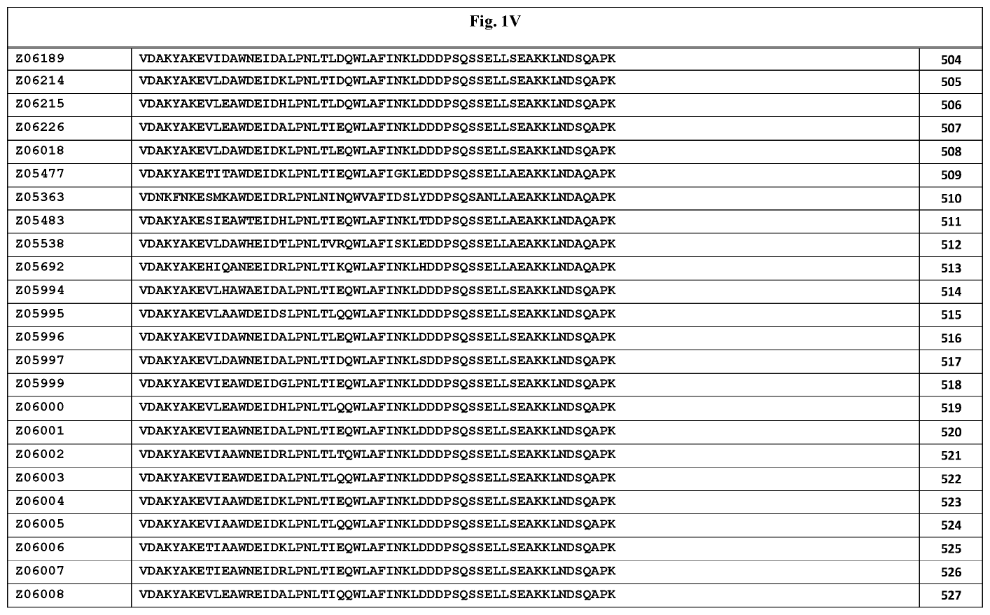

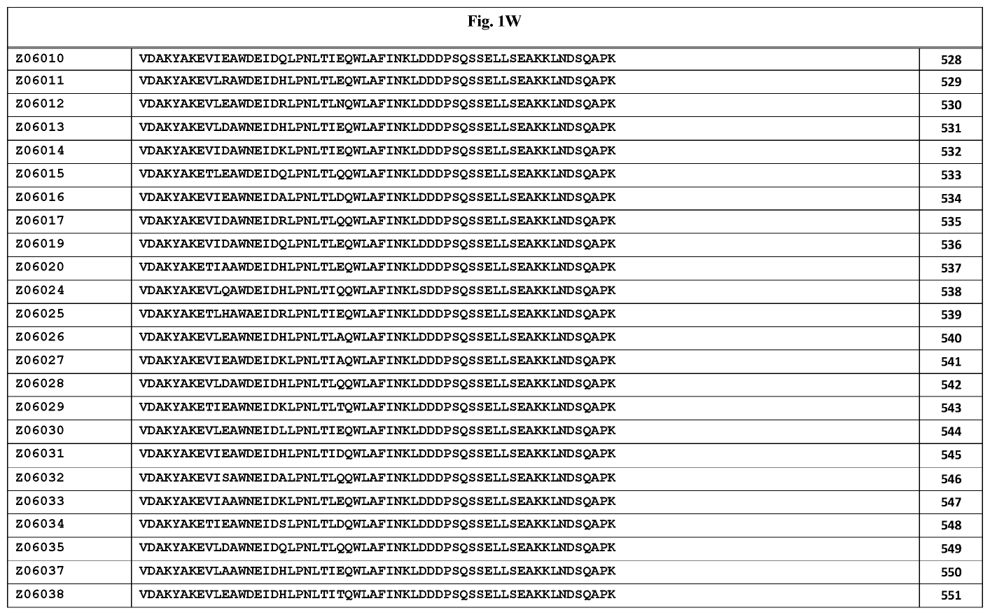

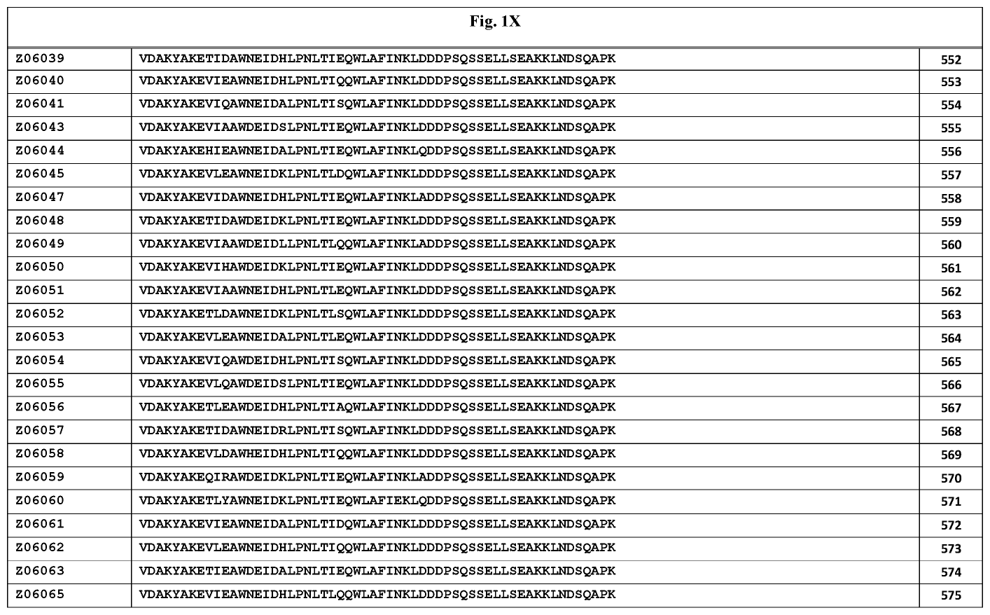

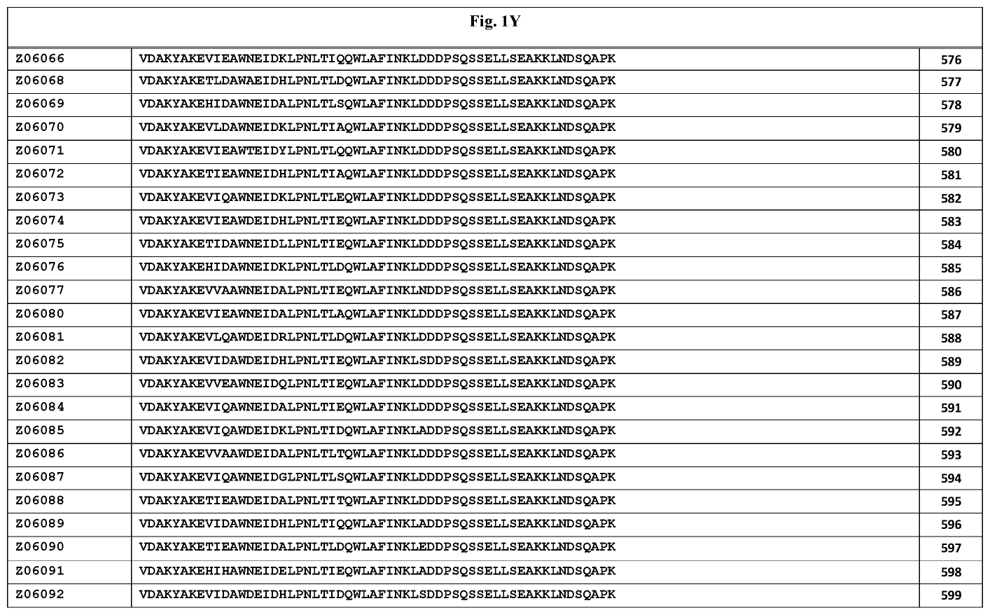

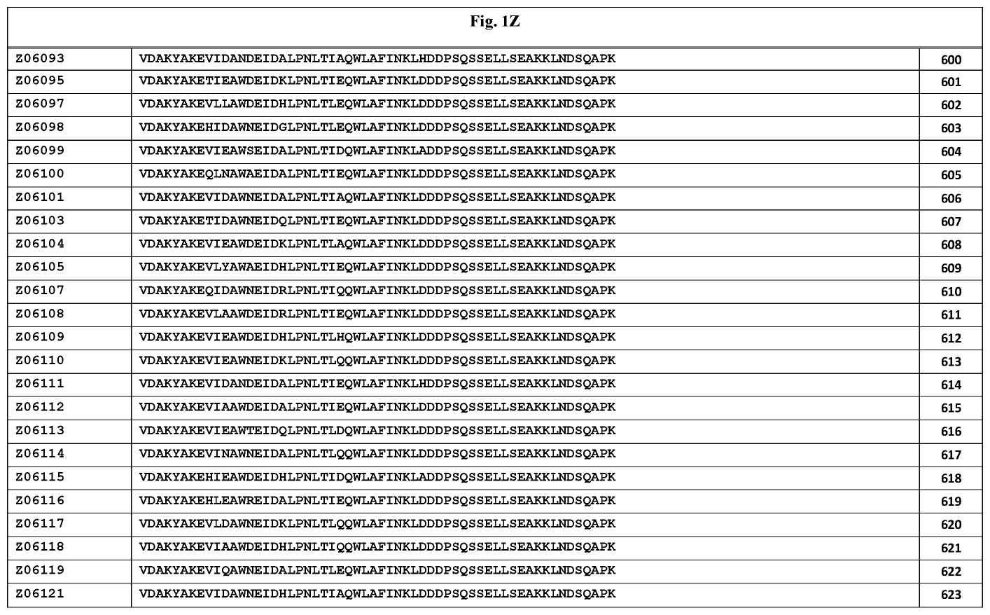

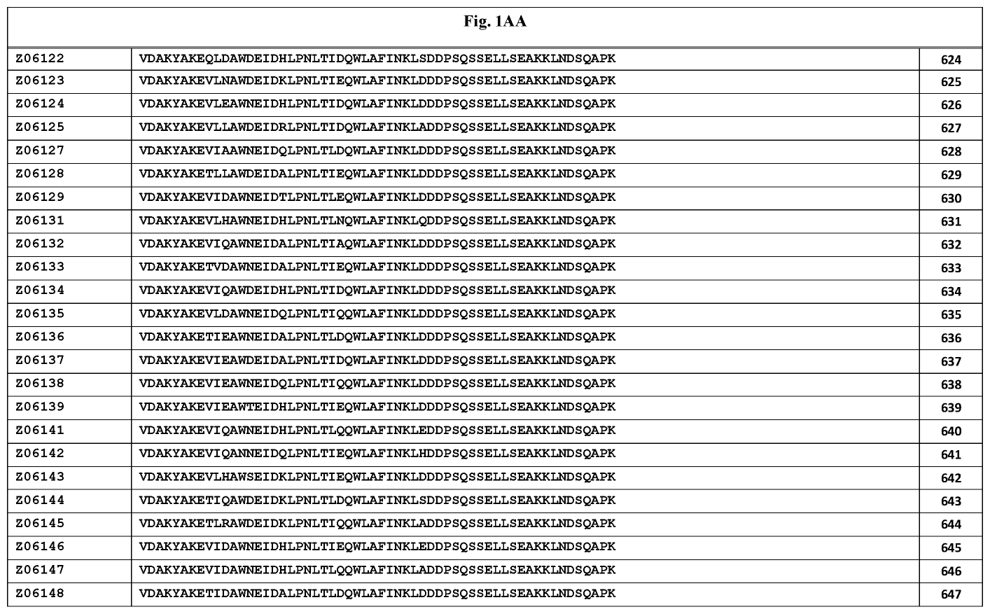

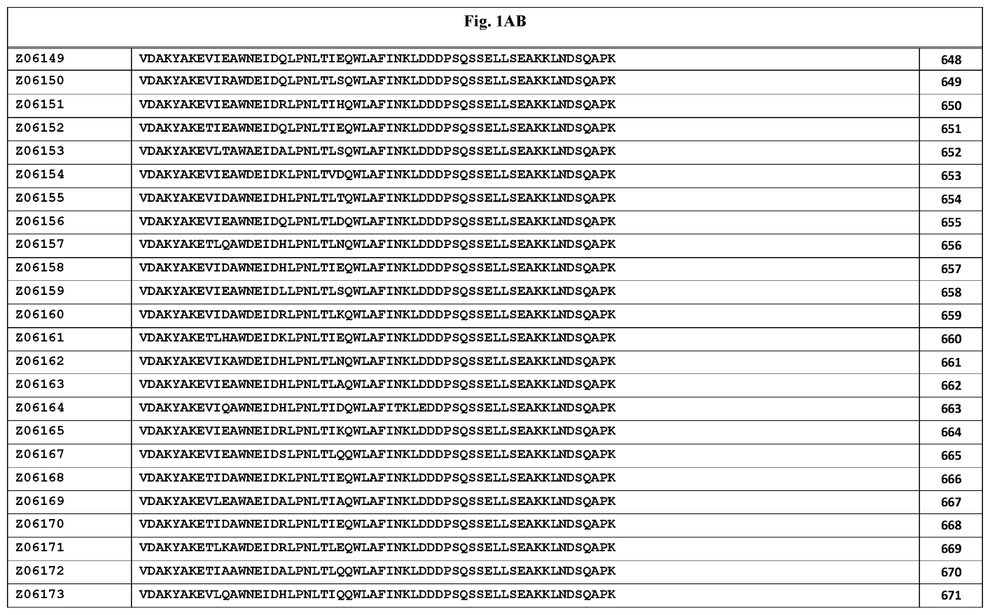

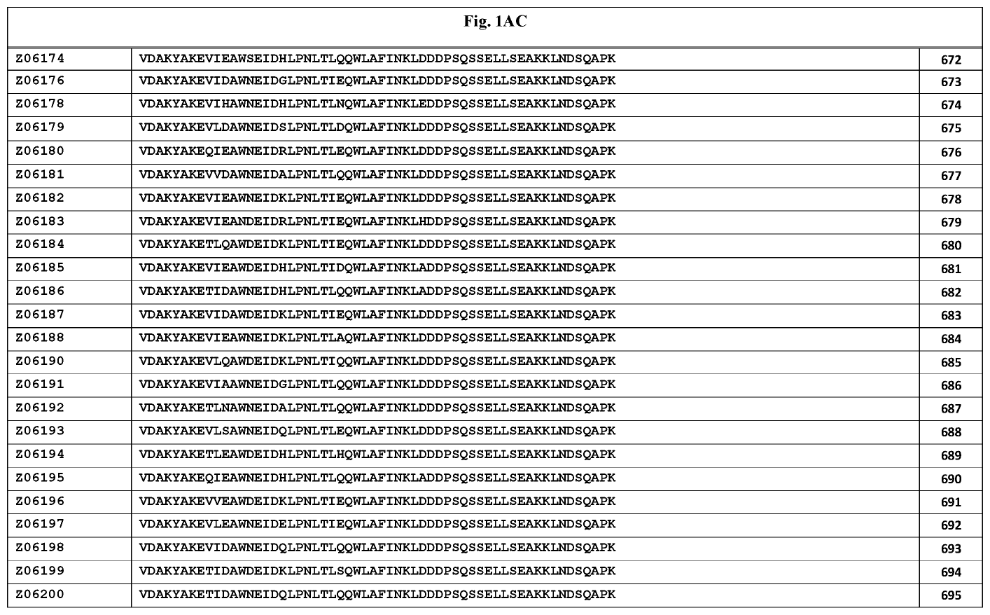

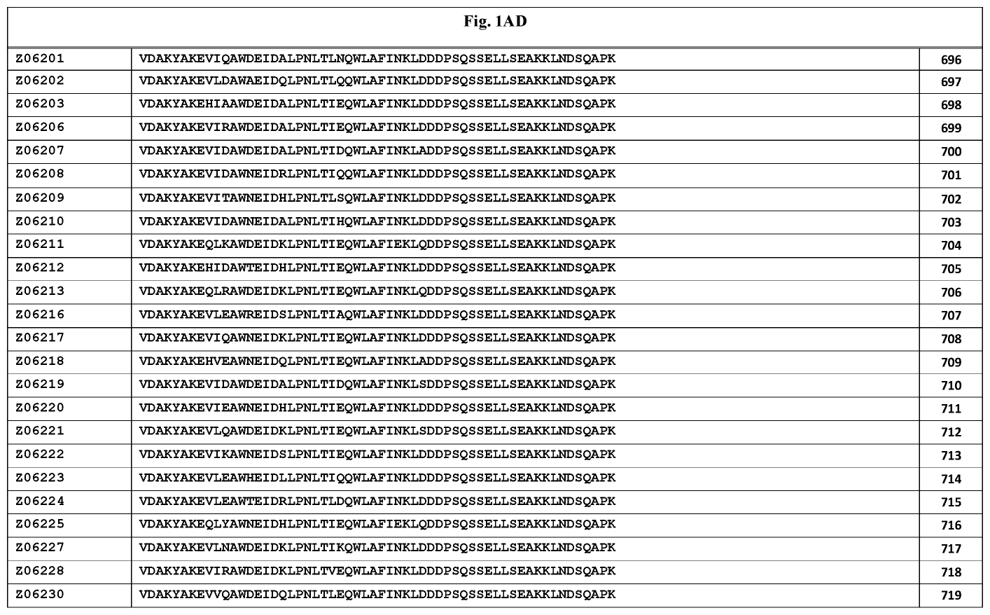

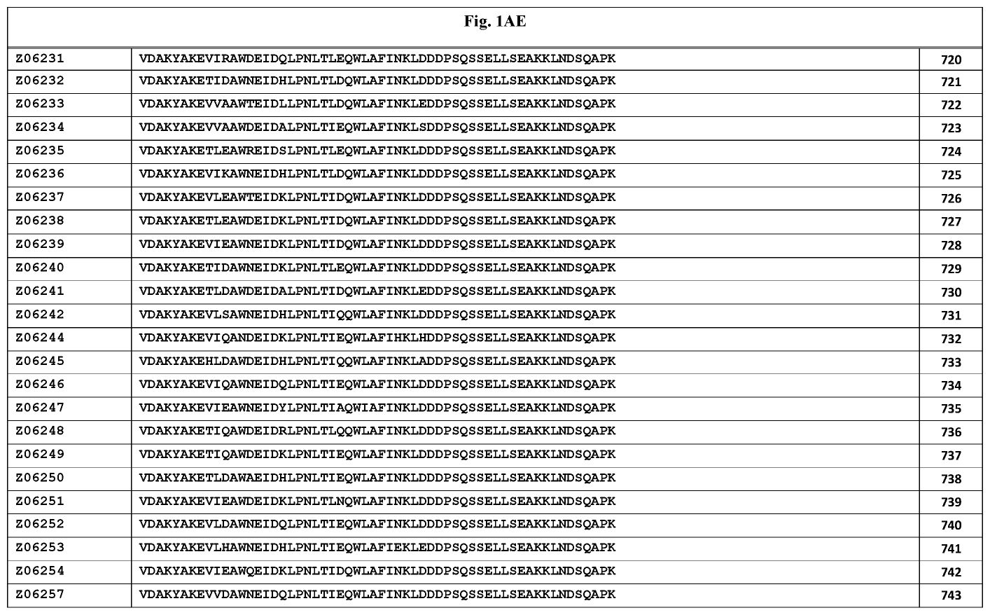

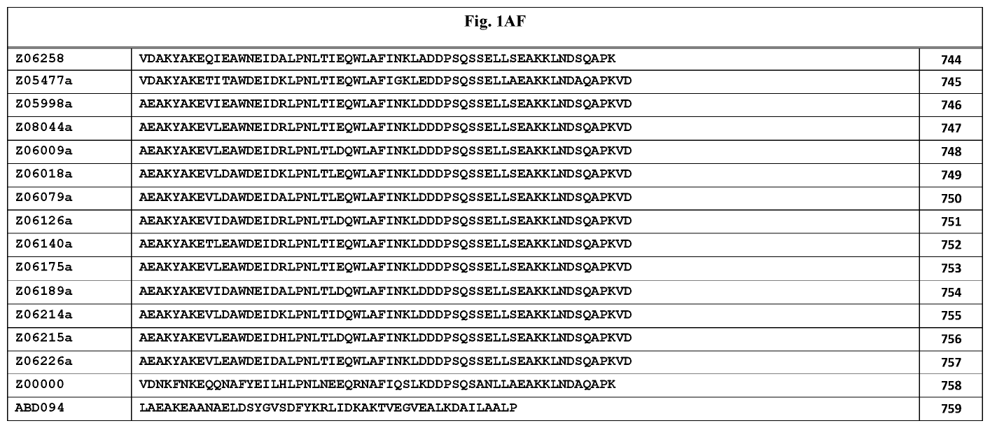

Sequencing: Sequencing was performed for the clones with positive absorbance values against complement protein C5 in the ELISA screening. Each variant was given a unique identification number #####, and individual variants are referred to as Z #####. The amino acid sequences of the 58 amino acid residues long Z variants are listed in FIG. 1 and in the sequence listing as SEQ ID NO:509-513. The deduced complement protein C5 binding motifs of these Z variants are listed in FIG. 1 and in the sequence listing as SEQ ID NO:13-17. The amino acid sequences of the 49 amino acid residues long polypeptides predicted to constitute the complete three-helix bundle within each of these Z variants are listed in FIG. 1 and in the sequence listing as SEQ ID NO:261-265.

### Example 2: Production and Characterization of Z Variants

### Materials and Methods

### Subcloning of Z Variants, Protein Expression and Purification:

Five complement protein C5 binding Z variants (Z05363 (SEQ ID NO:510); Z05477 (SEQ ID NO:509); Z05483 (SEQ ID NO:511); Z05538 (SEQ ID NO:512) and Z05692 (SEQ ID NO:513)) were amplified from pAffil/pAY00065/pAY02947 library vectors. A subcloning strategy for construction of dimeric Affibody molecules with N-terminal His₆ tags was applied using standard molecular biology techniques and as described in detail in WO 2009/077175. The Z gene fragments were subcloned into the expression vector pAY01448 resulting in the encoded sequence MGSSHHHHHHLQ-[Z #####][Z #####]-VD (SEQ ID NO. 773).

The subcloned Z variants were transformed into E. coli BL21(DE3) and expressed in the multifermenter system Greta (Belach Bioteknik). In brief, cultures were grown at 37° C. in 800 ml TSB-YE-medium containing 50 μg/ml kanamycin. At an OD₆₀₀ of ˜1, the cultures were induced through the automatic addition of IPTG to a final concentration of 0.05 mM. Cultures were cooled down to approximately 10° C. after 5 h of induction, and harvested by centrifugation (20 min, 15,900 g). Supernatants were discarded and the cell pellets were collected and stored at −20° C. until further use. Expression levels and the degree of solubility were estimated by SDS-PAGE analysis on 4-12% NuPAGE™ gels (Invitrogen) using Coomassie blue staining.

For Z variants expressed mainly as soluble protein, the cell pellets were resuspended in binding buffer (20 mM sodium phosphate, 0.5 M NaCl, 20 mM imidazole, pH 7.4) with an addition of 1000 U Benzonase® (Merck, cat. no. 1.01654.001) and disrupted by ultrasonication. For each of the Z variants, the sonicated suspension was clarified by centrifugation (40 min, 25,000 g, 4° C.) and the supernatant was loaded onto a 1 ml His GraviTrap™ column (GE Healthcare). The column was washed with wash buffer (20 mM sodium phosphate, 0.5 M NaCl, 60 mM imidazole, pH 7.4), before eluting the Z variants with 3 ml elution buffer (20 mM sodium phosphate, 0.5 M NaCl, 0.5 M imidazole, pH 7.4). Z variants which expressed mainly as insoluble protein were purified likewise, but 8 M urea was included in the binding and wash buffer. If required, the Z variants were further purified by reversed phase chromatography (RPC) on 1 ml Resource™ columns (GE Healthcare) using water including 0.1% TFA (trifluoroacetic acid) as mobile phase and elution with an appropriate gradient (typically 0-50% over 20 column volumes) of acetonitrile including 0.1% TFA.

The buffer was exchanged to PBS using PD-10 columns (GE Healthcare).

Protein characterization: The concentration of the purified Z variants was determined by absorbance measurements at 280 nm using theoretical extinction coefficients. The purity was estimated by SDS-PAGE analysis on 4-12% NuPAGE™ gels (Invitrogen) using Coomassie blue staining. To verify the identity and to determine the molecular weights of purified Z variants, LC/MS-analyses were performed on an Agilent 1100 LC/MSD system (Agilent Technologies).
CD analysis: The purified Z variants were diluted to 0.5 mg/ml in PBS. For each diluted Z variant, a CD spectrum was recorded between 250-195 nm at a temperature of 20° C. In addition, a variable temperature measurement (VTM) was performed to determine the melting temperature (Tm). In the VTM, the absorbance was measured at 221 nm while the temperature was raised from 20 to 90° C., with a temperature slope of 5° C./min. The ability of the Z variant to refold was assessed by collecting an additional CD spectrum at 250-195 nm after cooling to 20° C. The CD measurements were performed on a Jasco J-810 spectropolarimeter (Jasco Scandinavia AB) using a cell with an optical path length of 1 mm.
Biacore binding analysis: The interactions of the five subcloned His₆-tagged dimeric hC5-binding Z variants with hC5, cC5, rC5, hMG7 and hIgG (Sigma, cat. no. G4386) were analyzed in a Biacore instrument (GE Healthcare). The Z variants were immobilized in different flow cells on the carboxylated dextran layer of several CM5 chip surfaces (GE Healthcare). The immobilization was performed using amine coupling chemistry according to the manufacturer's protocol. One flow cell surface on each chip was activated and deactivated for use as blank during analyte injections. The analytes, diluted in HBS-EP running buffer (GE Healthcare) to a final concentration of 100 nM, were injected at a flow rate of 10 μl/min for 1 min. After 2 min of dissociation, the surfaces were regenerated with one injection of 10 mM HCl. The results were analyzed in BiaEvaluation software (GE Healthcare). Curves of the blank surface were subtracted from the curves of the ligand surfaces.

### Results

Subcloning of Z variants: Five selected unique clones (Z05477 (SEQ ID NO:509), Z05363 (SEQ ID NO:510), Z05483 (SEQ ID NO:511), Z05538 (SEQ ID NO:512) and Z05692 (SEQ ID NO:513)) were chosen for subcloning as dimers in the expression vector pAY01448 and were subsequently verified by sequencing.
Protein production: The histidine-tagged dimeric Z variants yielded acceptable expression levels of soluble gene product. The purity of produced batches was estimated to exceed 90% as assessed by SDS-PAGE analysis. LC/MS analysis verified the correct molecular weight for all Z variant molecules.
CD analysis: The melting temperatures (Tm) of the different Z variants were calculated by determining the midpoint of the transition in the CD signal vs. temperature plot. The results for a number of reversibly folding Z variants are summarized in Table 2 below.

TABLE 2
Melting temperatures for a number of Z variants.

| | SEQ ID NO: # | Tm |
|---|---|---|
| Z variant | of monomeric Z variant | (° C.) |
| His₆-(Z05477)₂ | SEQ ID NO: 509 | 45 |
| His₆-(Z05363)₂ | SEQ ID NO: 510 | 35 |
| His₆-(Z05483)₂ | SEQ ID NO: 511 | 44 |
| His₆-(Z05538)₂ | SEQ ID NO: 512 | 54 |
| His₆-(Z05692)₂ | SEQ ID NO: 513 | 52 |

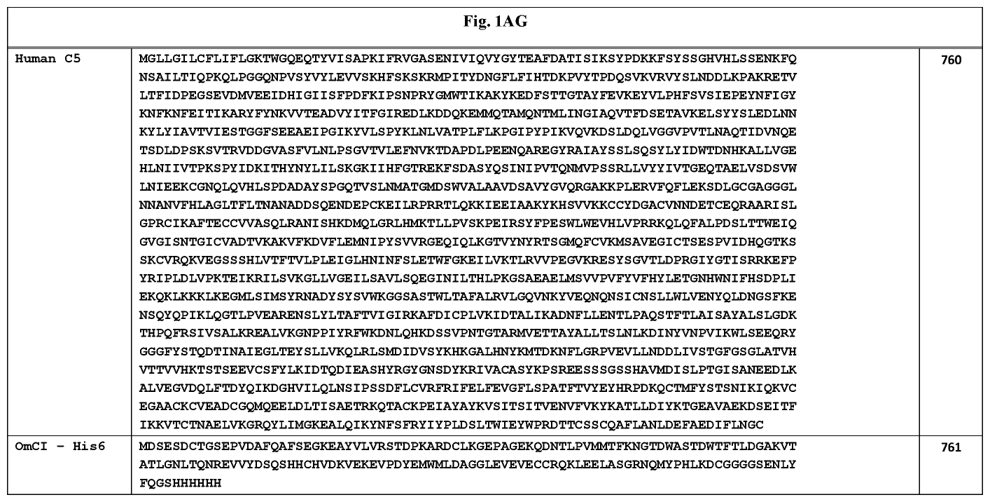

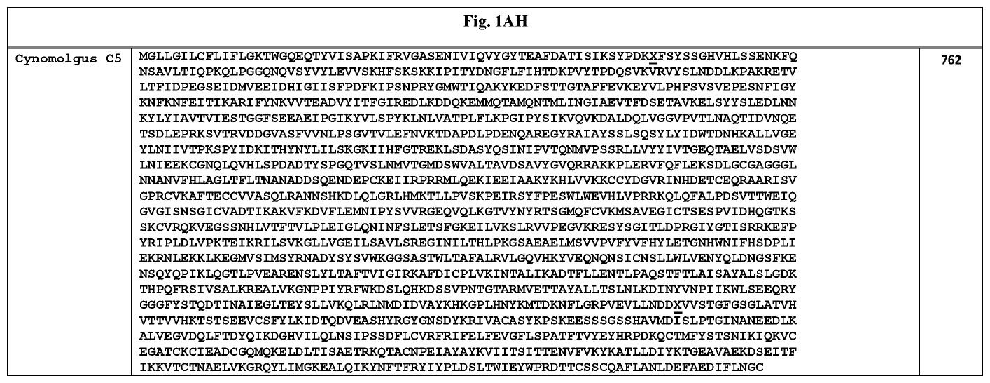

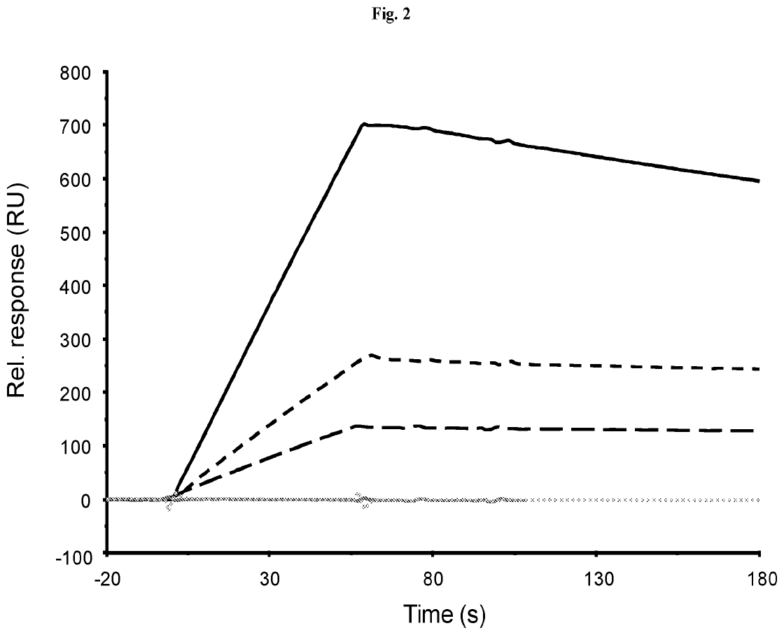

Biacore binding analysis: The binding of the five subcloned dimeric Z variants to different species of C5 and MG7, a subdomain of hC5, as well as the background binding to IgG was tested in a Biacore instrument by injecting the different proteins over surfaces containing the Z variants. The ligand immobilization levels for the different Z variants on the surfaces were: Z05363: 2080 RU, Z05477: 2180 RU, Z05483: 2010 RU, Z05538: 2570 RU and Z05692: 3270 RU. The different Z variants were tested for binding to different sets of proteins injected at concentrations of 100 nM, see Table 3. The result for the tested Z variants is displayed in the table as a +/− outcome for each protein. As an example of the Biacore binding analysis, FIG. 2 shows the sensorgrams obtained from immobilized dimeric Z05477 assayed against hC5, cC5, rC5, hMG7 and hIgG.

TABLE 3
Biacore response of different Z variants against C5 from various
species and relevant selected background proteins.

| | SEQ ID NO: # of | | | | | |
|---|---|---|---|---|---|---|
| Z variant | monomeric Z variant | hC5 | cC5 | rC5 | hMG7 | hIgG |
| His₆-(Z05477)₂ | SEQ ID NO: 509 | + | + | + | − | − |
| His₆-(Z05363)₂ | SEQ ID NO: 510 | + | + | + | − | − |
| His₆-(Z05483)₂ | SEQ ID NO: 511 | + | + | + | − | − |
| His₆-(Z05538)₂ | SEQ ID NO: 512 | + | + | + | − | − |
| His₆-(Z05692)₂ | SEQ ID NO: 513 | + | + | − | − | − |

### Example 3: Design and Construction of a Maturated Library of Complement Protein C5 Binding Z Variants

In this Example, a maturated library was constructed. The library was used for selections of hC5-binding polypeptides. Selections from maturated libraries are usually expected to result in binders with increased affinity (Orlova et al. Cancer Res 2006, 66(8):4339-48). In this study, randomized double stranded linkers were generated by the Slonomics® technology which enables incorporation of randomized sets of trinucleotide building blocks using ligations and restrictions of the subsequently built up double stranded DNA.

### Materials and Methods

Library design: The library was based on a selection of sequences of the hC5 binding Z variants described in Examples 1 and 2. In the new library, 13 variable positions in the Z molecule scaffold were biased towards certain amino acid residues, according to a strategy based on the Z variant sequences defined in SEQ ID NO:509-513 (Z05477, Z05363, Z05483, Z05538, Z05692). A SlonoMax® library of double-stranded DNA, containing the 147 bp partially randomized helix 1 and 2 of the amino acid sequence 5′-AA ATA AAT CTC GAG GTA GAT GCC AAA TAC GCC AAA GAA/GAG NNN NNN NNN GCA/GCC NNN NNN GAG/GAA ATC/ATT NNN NNN TTA/CTG CCT AAC TTA ACC/ACT NNN NNN CAA/CAG TGG NNN GCC/GCG TTC ATC/ATT NNN AAA/AAG TTA/CTG NNN GAT/GAC GAC CCA AGC CAG AGC TCA TTA TTT A-3′(SEQ ID NO: 774) (randomized codons are illustrated as NNN) flanked with restriction sites XhoI and SacI, was ordered from Sloning BioTechnology GmbH (Pucheim, Germany). The theoretical distributions of amino acid residues in the new library finally including 12 variable Z positions are given in Table 4.

TABLE 4
Library design.

| Amino acid position in | Randomization (amino acid | No of amino | |
|---|---|---|---|
| the Z variant molecule | abbreviations) | acids | Proportion |
| 9 | H, Q, S, T, V | 5 | 1/5 |
| 10 | I, L, V, W | 4 | 1/4 |
| 11 | A, D, E, H, K, L, N, R, S, T, Y | 12 | 1/12 |
| 13 | N, Q, W, Y | 4 | 1/4 |
| 14 | A, D, E, H, I, K, L, N, Q, R, S, T, V, W, Y | 15 | 1/14 |
| 17 | D, E | 2 | 1/2 |
| 18 | A, D, E, G, H, I, K, L, Q, R, S, T, V, Y | 14 | 1/14 |
| 24 | I, L, V | 3 | 1/3 |
| 25 | A, D, E, H, K, N, Q, R, S, T, Y | 11 | 1/11 |
| 28 | I, L, V | 3 | 1/3 |
| 32 | A, D, E, F, G, H, K, L, N, Q, R, S, T, V | 14 | 1/14 |
| 35 | A, D, E, H, K, N, Q, R, S, T, W, Y | 12 | 1/12 |

Library construction: The library was amplified using AmpliTaq Gold polymerase (Applied Biosystems, cat. no. 4311816) during 12 cycles of PCR and pooled products were purified with QIAquick PCR Purification Kit (QIAGEN, cat. no. 28106) according to the supplier's recommendations. The purified pool of randomized library fragments was digested with restriction enzymes XhoI and SacI (New England Biolabs, cat. no. R01460L, and cat. no. R0156L) and purified once more with PCR Purification Kit. Subsequently, the product was purified using preparative gel electrophoresis on a 1% agarose gel.

The phagemid vector pAY02592 (essentially as pAffil described in Grönwall et al. supra) was restricted with the same enzymes, purified using phenol/chloroform extraction and ethanol precipitation. The restricted fragments and the restricted vector were ligated in a molar ratio of 5:1 with T4 DNA ligase (New England Biolabs, cat. no. M0202S), for 2 hours at RT followed by overnight incubation at 4° C. The ligated DNA was recovered by phenol/chloroform extraction and ethanol precipitation, followed by dissolution in 10 mM Tris-HCl, pH 8.5.

The ligation reactions (approximately 250 ng DNA/transformation) were electroporated into electrocompetent E. coli RR1ΔM15 cells (100 μl). Immediately after electroporation, approximately 1 ml of SOC medium (TSB-YE media, 1% glucose, 50 μM MgCl₂, 50 μM MgSO₄, 50 μM NaCl and 12.5 μM KCl) was added. The transformed cells were incubated at 37° C. for 50 min. Samples were taken for titration and for determination of the number of transformants. The cells were thereafter pooled and cultivated overnight at 37° C. in 71 of TSB-YE medium, supplemented with 2% glucose and 100 μg/ml ampicillin. The cells were pelleted for 15 min at 4,000 g, resuspended in a PBS/glycerol solution (approximately 40% glycerol). The cells were aliquoted and stored at −80° C. Clones from the library of Z variants were sequenced in order to verify the content and to evaluate the outcome of the constructed library vis-à-vis the library design. Sequencing was performed as described in Example 1 and the amino acid distribution was verified.

Preparation of phage stock: Cells from the glycerol stock containing the C5 phagemid library were inoculated in 20 1 of a defined proline free medium (described in Example 1) supplemented with 100 μg/ml ampicillin, and grown at 37° C. in a fermenter (Belach Bioteknik, BR20). All steps were performed as described in Example 1 for the library Zlib006Naive.II. After cultivation, the cells were pelleted by centrifugation at 15,900 g and the phage particles remaining in the medium were thereafter precipitated twice in PEG/NaCl, filtered and dissolved in PBS and glycerol as described in Example 1. Phage stocks were stored at −80° C. until use in selection.

### Results

Library construction: The new library was designed based on a set of OmCI-blocked C5 binding Z variants with verified binding properties (Example 1 and 2). The theoretical size of the designed library was 6.7×10⁹ Z variants. The actual size of the library, determined by titration after transformation to E. coli. RR1ΔM15 cells, was 1.4×10⁹ transformants.

The library quality was tested by sequencing of 64 transformants and by comparing their actual sequences with the theoretical design. The contents of the actual library compared to the designed library were shown to be satisfying. The locked position in the designed amino acid sequence (W in position 27) was reflected in the actual sequence in that only the expected amino acid occurred in that position. A maturated library of hC5 binding polypeptides was thus successfully constructed.

### Example 4: Selection, Screening and Characterization of Z Variants from a Maturated Library

### Materials and Methods

Phage display selection of complement protein C5 binding polypeptides: The target protein hC5 was biotinylated as described in Example 1. Phage display selections were performed against hC5 essentially as described in Example 1 using the new library of Z variant molecules described in Example 3. E. coli XL1-Blue was used for phage amplification. Selection was initially performed in two parallel tracks. In one track, the time of selection was 2 h, while in the other track, shorter selection times were used: 20 min in the first cycle and 10 min for subsequent cycles 2-4. These two tracks (1 and 2) were further divided in the second cycle, resulting in totally six tracks (1a-c and 2a-c, differing in target concentration and wash conditions). Selection was performed in a total of four cycles. In cycle 1 of the selections, 25 nM complement protein C5 was used and five washes with PBST 0.1% were performed. An increased stringency, using a lowered target concentration and an increased number of washes, was applied in the subsequent three cycles. In cycle 2, 3 and 4; 10, 5 or 2.5 nM complement protein C5, 4, 1 or 0.25 nM complement protein C5 and 1.6, 0.2 or 0.05 nM complement protein C5 were used. In cycle 2, 3 and 4; 10, 15 and 20 washes were performed using PBST 0.1%. In addition, the second last wash was prolonged to 3 h with a 50×excess of non-biotinylated hC5 in the washing solution for two of the tracks (1c and 2c).
Sequencing of potential binders: Individual clones from the different selection tracks were picked for sequencing. All clones run in the ELISA screening were sequenced. Amplification of gene fragments and sequence analysis of gene fragments were performed as described in Example 1.
ELISA screening of Z variants: Single colonies containing Z variants were randomly picked from the selected clones of the complement protein C5 maturated library and grown in 1 ml cultivations as described in Example 1. Periplasmic proteins were released by 8 repeated freeze-thawing cycles. ELISA screenings were performed essentially as described in Example 1 with the following exceptions. Half-area 96-well ELISA plates were coated with 2 μg/ml of an ABD specific goat antibody (in house produced) diluted in coating buffer. Biotinylated hC5 was used at a concentration of 0.15 μg/ml and incubation performed for 1.5-2 h. Streptavidin conjugated HRP was obtained from Thermo Scientific (cat. no. N100). The Z variant Z05363 (SEQ ID NO:510) originating from the primary selections (Example 1) was used as a positive control as well as a negative control omitting hC5.

Selected maturated Z variants were subjected to a second screen against hC5 at a lower concentration and compared to rC5. The assay was essentially performed as described above. hC5 and rC5 was used at a concentration of 0.05 μg/ml and 4 μg/ml, respectively. The Z variant Z05363 (SEQ ID NO:510) was used as a positive control in this experiment as well. As a negative control, a Z variant binding to PDGF-Rβ (Z01977; described in WO 2009/077175) was assayed against biotinylated hC5 or rC5.

In deep sequence analysis of selected Z variants and correlation of amino acids in the 13 randomized positions with measured melting temperatures and IC₅₀ values for human C5 and mouse C5 in the hemolysis assay (described in Example 6) suggested a favorable Z variant not identified among the 558 sequenced clones. Based on the Z variant Z05998 (SEQ ID No:499), a single amino acid, Ile in position 10 was substituted with Leu using conventional technology for site directed mutagenesis. The new variant is referred to as Z08044 (SEQ ID NO:498). The deduced complement protein C5 binding motif of this Z variant is listed in FIG. 1 and in the sequence listing as SEQ ID NO:2. The amino acid sequences of the 49 amino acid residues long polypeptide predicted to constitute the complete three-helix bundle within these Z variant is listed in FIG. 1 and in the sequence listing as SEQ ID NO:250.

### Results

Phage display selection of complement protein C5 binding polypeptides: Selection was performed in totally six parallel tracks containing four cycles each. The different selection tracks differed in target concentration and wash conditions as follows: 1a) 2 h selection time, high concentration, standard wash, 1b) 2 h selection time, low concentration, standard wash, 1c) 2 h selection time, medium concentration, long wash, 2a) 10 min selection time, high concentration, standard wash, 2b) 10 min selection time, low concentration, standard wash, and 2c) 10 min selection time, medium concentration, long wash. For each selection cycle, the target concentration was decreased and the washing conditions were more stringent. All tracks gave in each round sufficient amounts of phage particles in the eluate. Most phage particles were found in tracks 1a and 2a, representing the highest target concentration and mildest wash conditions.
Sequencing: Randomly picked clones (558) were sequenced Each individual Z variant was given an identification number, Z #####, as described in Example 1. In total, 242 new unique Z variant molecules were identified. The amino acid sequences of the 58 amino acid residues long Z variants are listed in FIG. 1 and in the sequence listing as SEQ ID NO:497, SEQ ID NO:499-508 and SEQ ID NO:514-744. The deduced complement protein C5 binding motifs of these Z variants are listed in FIG. 1 and in the sequence listing as SEQ ID NO:1, SEQ ID NO:3-12 and SEQ ID NO:18-248. The amino acid sequences of the 49 amino acid residues long polypeptides predicted to constitute the complete three-helix bundle within each of these Z variants are listed in FIG. 1 and in the sequence listing as SEQ ID NO:249, SEQ ID NO:251-260 and SEQ ID NO:266-496. Among the sequenced clones, 63 sequences occurred two or more times.
ELISA screening of Z variants: Clones obtained after four selection cycles were produced in 96-well plates and screened for hC5-binding activity using ELISA. All randomly picked clones were analyzed. 229 of the 242 unique Z variants were found to give a higher response (0.3-3.1 AU) against hC5 at a concentration of 0.15 μg/ml compared to the positive control clone Z05363 (SEQ ID NO:510; an average absorbance signal of 0.3 AU), obtained from the primary selections (Example 1). Clones from all selection tracks showed positive signals. The negative controls had an absorbance of approximately 0.1 AU.

Z variants were selected based on their performance in the ELISA screen against hC5 and the occurrence frequency. 43 unique Z variants were assayed against a lower concentration of hC5 (0.05 μg/ml) as well as rC5 (4 μg/ml). A positive result against rC5 was obtained for 40 of the tested Z variants, defined as 2× the signal for the negative control (0.4 AU). The results for all the tested Z variants against the lower concentration of hC5 as well as against rC5 are shown in FIG. 3.

### Example 5: Subcloning, Production and Characterization of a Subset of Complement Protein C5 Binding Z Variants

### Materials and Methods

Subcloning of Z variant molecules into expression vectors: Based on sequence analysis and the performance in the ELISA against human and rat complement protein C5, 45 clones were selected for subcloning into the expression vector pAY01448. Monomer Z variant fragments were amplified from the phagemid vector pAY02592 and the subcloning into pAY01448 was performed as described in Example 2, resulting in a vector encoding the protein sequence MGSSHHHHHHLQ-[Z #####]-VD (SEQ ID NO: 775).
Protein expression and purification: The 45 Z variants in the His₆-(Z #####) format, were expressed in an automated multifermenter system as described in Example 2 or similarly in a small scale set-up of 100 ml cultures in shaker flasks induced manually with IPTG to a final concentration of 0.4 mM. Purification was performed using 1 ml HisGraviTrap™ Columns essentially as described in Example 2 or in a smaller scale using 0.1 ml His SpinTrap (GE Healthcare, cat. no. 284013-53). Buffer was exchanged to PBS using PD-10 columns or PD SpinTrap G-25 (GE Healthcare, cat. no. 28-9180-04) according to the manufacturer's instructions. The concentration of purified Z variants was determined by absorbance measurements at 280 nm and the purity and identity was assessed by SDS-PAGE and LC/MS as described in Example 2. Samples were aliquoted and stored at −80° C. until further use.
CD analysis: The CD analysis for determination of melting temperatures and folding reversibility was performed as described in Example 2.

### Results

Protein expression and purification: All 45 subcloned Z variants could be expressed and the in vitro solubility for all purified variants was good. The purity was estimated by LC/MS to exceed 90% for all variants. The correct molecular weights were verified by LC-MS.
CD analysis: CD spectrum measurements performed at 20° C. confirmed the α-helical structure of the Z variants at this temperature. An overlay of the spectrums obtained after the variable temperature measurements (heating to 90° C. followed by cooling to 20° C.) on the spectrums obtained before the variable temperature measurement showed that all Z variants fold back completely, or nearly completely, to their α-helical structures after heating to 90° C. (results not shown). The melting temperatures for a set of Z variants were determined from the variable temperature measurements and are shown in Table 5.

TABLE 5
Melting temperatures of maturated Z variants with
a histidine tag fused directly to the amino terminus
of SEQ ID NO: 497 and SEQ ID NO: 499-508.

| | SEQ ID NO: # of | Tm |
|---|---|---|
| Z variant | Z variant | (° C.) |
| His₆-Z06175 | SEQ ID NO: 497 | 44 |
| His₆-Z05998 | SEQ ID NO: 499 | 45 |
| His₆-Z06009 | SEQ ID NO: 500 | 45 |
| His₆-Z06079 | SEQ ID NO: 501 | 46 |
| His₆-Z06126 | SEQ ID NO: 502 | 44 |
| His₆-Z06140 | SEQ ID NO: 503 | 42 |
| His₆-Z06189 | SEQ ID NO: 504 | 47 |
| His₆-Z06214 | SEQ ID NO: 505 | 44 |
| His₆-Z06215 | SEQ ID NO: 506 | 41 |
| His₆-Z06226 | SEQ ID NO: 507 | 44 |
| His₆-Z06018 | SEQ ID NO: 508 | 46 |

### Example 6: In Vitro Characterization of C5 Binding Z Variants

### Materials and Methods

Cloning and protein production: DNA encoding a subset of C5 binding Z variants (SEQ ID NO:745-757) where E. coli codon optimized and synthesized by GeneArt, GmbH. The synthetic genes representing the C5 binding Z variants were subcloned and expressed in E. coli. The expression vectors encoding constructs of monomers or dimers of Z variants optionally linked to an albumin binding domain (ABD094, SEQ ID NO:759) are schematically illustrated in FIG. 4.

Intracellularly expressed Z variants were purified using conventional chromatography methods. Homogenization and clarification was performed by sonication followed by centrifugation and filtration. Anion exchange chromatography was used as capture step. Further purification was obtained by hydrophobic interaction chromatography. The purifications were executed at acidic conditions (pH 5.5). Polishing and buffer exchange was performed by size exclusion chromatography. Before concentration to final protein content, the endotoxin level was reduced by polymyxin B affinity chromatography. Produced proteins were analyzed by MALDI-TOF MS and on SDS-PAGE.

In addition, recombinantly expressed OmCI protein (SEQ ID NO:761) was used as a reference molecule in the in vitro studies.

Inhibition of hemolysis: For studies of classical complement pathway function and inhibition thereof by C5 binding polypeptides, sheep erythrocytes were prepared from fresh sheep whole blood in Alsever's solution (Swedish National Veterinary Institute) and thereafter treated with rabbit anti-sheep erythrocyte antiserum (Sigma) to become antibody sensitized sheep erythrocyte (EA). The whole process was conducted under aseptic conditions. All other reagents were from commercial sources.

The in vitro assay was run in 96-well U-form microtiter plate by consecutive additions of a test protein, a complement serum and EA suspension. The final concentrations of all reagents, in a total reaction volume of 50 μl per well and at pH 7.3-7.4, were: 0.15 mM CaCl₂); 0.5 mM MgCl₂; 3 mM NaN₃; 138 mM NaCl; 0.10% gelatin; 1.8 mM sodium barbital; 3.1 mM barbituric acid; 5 million EA; complement protein C5 serum at suitable dilution, and C5 binding Z variant at desired concentrations. Different species of complement sera were used in the assay to define cross-species potencies of the Z variants. For mouse serum, a C5 depleted human serum (C5D from Quidel cat. no. A501) had to be supplemented in an equal amount.

The Z variants were pre-incubated with the above described complement serum for 20 min on ice prior to starting the reaction by the addition of EA suspension. The hemolytic reaction was allowed to proceed at 37° C. during agitation for 45 min and was then optionally ended by addition of 100 μl ice-cold saline containing 0.02% Tween 20. The cells were centrifuged to the bottom and the upper portion, corresponding to 100 μl supernatant, was transferred to a transparent microplate having half-area and flat-bottom wells. The reaction results were analyzed as optical density using a microtiter plate reader at a wavelength of 415 nm.

On all test occasions, controls, vehicle and OmCI (SEQ ID NO:761), were included in each plate to define the values of uninhibited and fully inhibited reactions, respectively. These values were used to calculate the % inhibition of the complement hemolysis at any given sample concentration. The inhibitory potencies (IC₅₀ values) of tested Z variants were defined by applying the same assay in the presence of a controlled concentration of human C5 added to C5 depleted serum. For highly potent inhibitors (low nanomolar to sub-nanomolar), a final C5 concentration of the reaction mixture was controlled at 0.1 nM, which was optionally established by using C5 depleted or deficient sera.

In vitro kinetics and affinity of C5 binding Z variants to immobilized hC5: The binding affinity of a number of C5 binding Z variants (SEQ ID NO:748-757) to hC5 were analyzed using a Biacore T200 instrument (GE Healthcare). Human C5 (A403, Quidel Corporation) was coupled to a CM5 sensor chip (900 RU) using amine coupling chemistry according to the manufacturer's protocol. The coupling was performed by injecting hC5 at a concentration of 7.5 μg/ml in 10 mM Na-acetate buffer pH 5 (GE Healthcare). The reference cell was treated with the same reagents but without injecting human C5.

All experiments were performed in 10 mM HEPES pH 7.4, 150 mM NaCl, 3 mM EDTA, 0.005% Surfactant P20 (HBS-EP buffer, GE Healthcare). For kinetic analyses, the flow rate was 30 μl/min and data were collected at 25° C. Data from the reference cell were subtracted to compensate for bulk refractive index changes. In most cases, an injection of HBS-EP was also included as control so that the sensorgrams were double blanked. The surfaces were regenerated in HBS-EP buffer.

Binding of Z variants to immobilized hC5 was studied with the single cycle kinetics method, in which five concentrations of sample are injected one after the other in the same cycle without regeneration between injections. Kinetic constants were calculated from the sensorgrams using the Langmuir 1:1 or bivalent analyte model of Biacore T200 Evaluation Software version 1.0.

In vitro kinetics and affinity of C5 binding Z-ABD molecules to immobilized hC5: Binding of Z-ABD molecules (SEQ ID NO:748-757 fused to ABD094 (SEQ ID NO:759) by a GS linker), to immobilized hC5 was evaluated using a Biacore T200 instrument (GE Healthcare).

Z-ABD constructs where Z06175a (SEQ ID NO:753) as a monomer or dimer have been fused to ABD094 (SEQ ID NO:759) either in the N-terminus or the C-terminus via different linkers as specified in FIG. 4 (constructs 2, 7, 5 and 4) were also pre-incubated with recombinant human albumin (Cell Prime rAlbumin AF-G, 9301, Novozymes), diluted and then injected over immobilized human C5 according to the single-cycle kinetics method as described above. As a comparison, the same constructs were injected in the absence of HSA. Two constructs, Z06175a-GS (FIG. 4, construct 1) and Z06175a-GSGGGGSGGGGS-ABD094 (SEQ ID NO: 776 (FIG. 4, construct 3) were only tested in the absence of HSA.

Steady state binding of C5 binding Z variants to C5 coated ECL plates: The affinity of a number of C5 binding constructs comprising Z variants (SEQ ID NO:745, SEQ ID NO:748-757 optionally fused to ABD094 (SEQ ID NO:759) in constructs as specified in FIG. 4) to human C5 was measured by displacement of a ruthenium labeled C5 binding Z-ABD variant (SEQ ID NO:748 fused to SEQ ID NO:759 by a GS-linker).

The Z-ABD variant (SEQ ID NO:748 fused to SEQ ID NO:759 by a GS-linker) to be used as tracer was labeled at a molar ratio 1:12 to 1:20 (protein: SULFO-TAG NHS-Ester, Meso Scale Discovery, cat. no. R91AN-1). The labeling reaction was performed on ice for two hours. Unbound SULFO-TAG was removed using a Zeba™ spin desalting column (Thermo Scientific, cat no. 89889) and final protein concentration was measured by using Bradford reagent (Bradford, M. M., Anal. Biochem. 72: 248-254, 1976). The affinity (dissociation constant, K_{D}) of the SULFO-TAG labeled Z-ABD variant was determined by saturation binding analysis of increasing concentrations of the labeled Z-ABD variant to C5 coated electrochemoluminescence wells (ECL, Meso Scale Discovery). The labeled Z-ABD variant was further analyzed by LC/MS in order to determine the distribution of SULFO-TAG molecules on the Z-ABD variant.

Displacement was carried out by coating ECL, Multi-array 96-well high-bind, non-coated (Meso Scale Discovery, cat. no. L15XB) plates with 50 fmol/well hC5 overnight at 4° C. Subsequently, non-specific sites were blocked with PBS with 1% Casein for two hours at RT. Different Z variants optionally fused with ABD094 (SEQ ID NO:759) (see FIG. 4) were incubated at different concentrations along with approximately 100 μM of the SULFO-TAG labeled C5 binding Z-ABD variant in PBS with 1% Casein. Incubation lasted three hours at RT while agitating the plate at 300 rpm. Finally, incubation was terminated by washing 3 times with 150 μl ice-cold PBS-Tween20. Immediately after the final wash, 150 μl 2× reading buffer (4× reading buffer T, Meso Scale Discovery cat. no. R92TC-3 diluted 1:1 in ultrapure H₂O) was added to each well and the signal was detected using a plate reader (SECTOR Imager 2400, Meso Scale Discovery). The naturally occurring C5 binding protein OmCI (Nunn et al. supra, SEQ ID NO:761) was included in the displacement assay as a positive control. Binding affinity of competing C5 binding constructs and controls to C5 was determined by non-linear regression analysis using Excel plugin XLfit5 and GraphPad Prism 4.

Selectivity of Z-ABD binding to C5 over C3, C4 and IgG: Binding of one Z-ABD variant (SEQ ID NO:748 fused to SEQ ID NO:759 by a GS-linker) to the closely related complement proteins C3 and C4 from human as well as binding to human IgG (since the origin of the Z-domain, Staphylococcal protein A, is an IgG binding protein) was addressed by surface plasmon resonance (SPR) using a Biacore 2000 instrument (GE Healthcare). The Z-ABD construct was immobilized on a CM5 chip (GE-Healthcare) using amine coupling (70 RU). 40 nM and 400 nM of each of human C3 (A401, Quidel), C4 (A402, Quidel) and IgG (I2511, Sigma) diluted in HBS-P buffer (GE Healthcare) were injected over the surface. Each injection was followed by a regeneration cycle with 20 mM NaOH injected for 30 s. Human C5 at the same concentrations was run in parallel as a positive control.

### Results

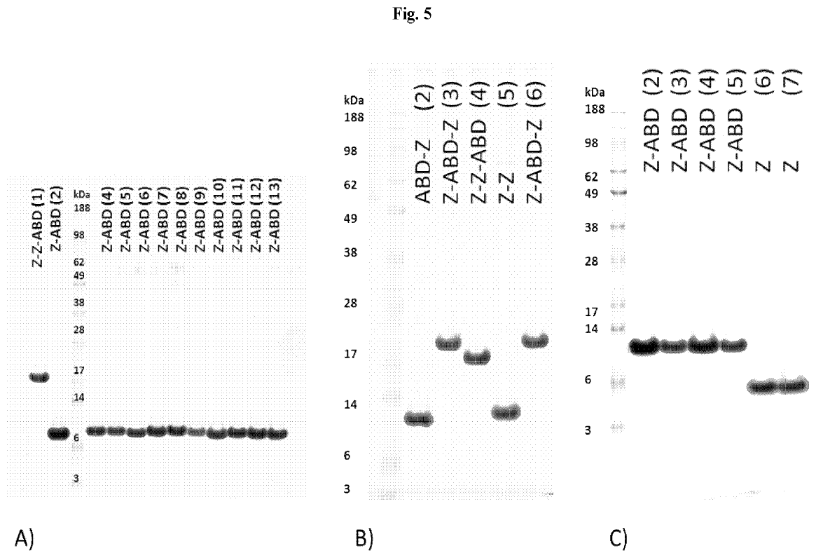

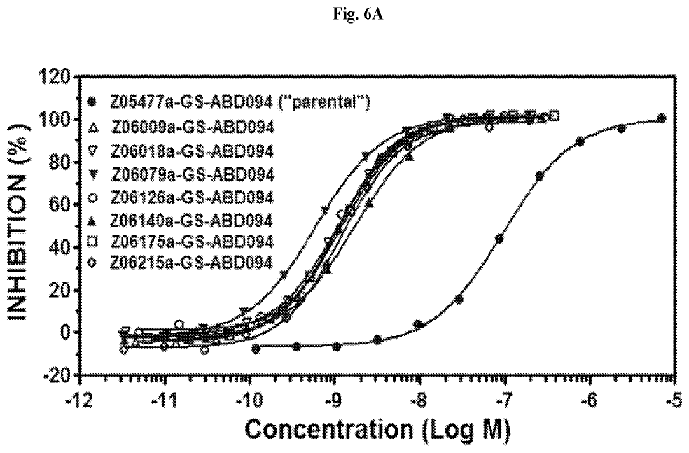

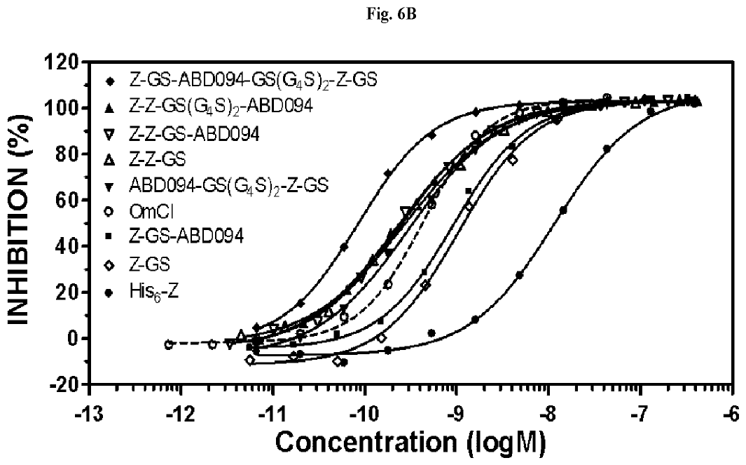

Cloning and protein production: Produced protein variants as schematically described in FIG. 4 where “Z” can be represented by SEQ ID NO:745 and SEQ ID NO:748-757 were analyzed by MALDI-TOF MS and on SDS-PAGE. (FIG. 5)
Inhibition of hemolysis: A subset of C5 binding Z variants were assayed for C5 binding activity in vitro and inhibition of hemolysis in sheep erythrocytes. The concentration of Z variant resulting in 50% inhibition of hemolysis (IC₅₀) or 50% inhibition of tracer binding to human C5 was calculated. Representative concentration-response curves for Z variants shown as SEQ ID NO:745 and SEQ ID NO:748-757 inhibiting hemolysis as described in the methods section are shown in FIG. 6A and FIG. 6B. The result for different Z variants fused to ABD094 (SEQ ID NO:759) via a short GS-linker are shown in FIG. 6A.

The parental Z variant Z05477a (SEQ ID NO:745) fused to ABD094 (SEQ ID NO:759) separated by a short GS linker exhibited an IC₅₀ value of about 100 nM, whereas the tested second-generation C5 binding Z-ABD variants typically inhibited hemolysis with IC₅₀ values around or below 1 nM. This suggests a more than 100-fold increase in potency for the C5 binding Z variants identified in the maturation selection and subsequent screening.

In FIG. 6B, C5 binding is shown for various combinations of one representative Z variant (Z06175a; SEQ ID NO:753) alone, as a dimer and in fusion with ABD094 (SEQ ID NO:759) either in the N-terminus or the C-terminus via different linkers as specified in the figure. The C5 binding combinations exhibited IC₅₀ values ranging from 86 μM to 12 nM with human serum as measured using the above described assay. The corresponding value for the tic protein OmCI was typically 300 to 500 pM.

In vitro kinetics: Kinetic studies of binding characteristics for a number of Z variants (SEQ ID NO:748-757) optionally fused to ABD094 (SEQ ID NO:759), to immobilized hC5, as well as to C5 in the presence of human albumin, were performed using the Biacore T200 instrument.

Data for ten different Z variants fused to ABD094 via a GS linker are presented in Table 6.

TABLE 6
Human C5-binding characteristics for different Z-ABD fusions

| | SEQ ID NO: # | | | |
|---|---|---|---|---|
| Construct | of Z variant | kₐ (1/Ms) | k_{d} (1/s) | K_{D} (M) |
| Z-GS-ABD094 | SEQ ID NO: 748 | 6.93 × 10⁵ | 9.04 × 10⁻⁴ | 1.31 × 10⁻⁹ |
| | SEQ ID NO: 749 | 6.75 × 10⁵ | 1.23 × 10⁻³ | 1.83 × 10⁻⁹ |
| | SEQ ID NO: 750 | 7.65 × 10⁵ | 1.34 × 10⁻³ | 1.75 × 10⁻⁹ |
| | SEQ ID NO: 751 | 6.90 × 10⁵ | 1.29 × 10⁻³ | 1.87 × 10⁻⁹ |
| | SEQ ID NO: 752 | 7.02 × 10⁵ | 1.81 × 10⁻³ | 2.58 × 10⁻⁹ |
| | SEQ ID NO: 753 | 7.90 × 10⁵ | 1.01 × 10⁻³ | 1.18 × 10⁻⁹ |
| | SEQ ID NO: 754 | 5.00 × 10⁵ | 1.14 × 10⁻³ | 2.28 × 10⁻⁹ |
| | SEQ ID NO: 755 | 6.84 × 10⁵ | 2.08 × 10⁻³ | 3.05 × 10⁻⁹ |
| | SEQ ID NO: 756 | 3.17 × 10⁵ | 6.37 × 10⁻³ | 2.01 × 10⁻⁹ |
| | SEQ ID NO: 757 | 4.63 × 10⁵ | 1.08 × 10⁻³ | 2.34 × 10⁻⁹ |

Binding of the same Z variant (SEQ ID NO:753) but in different constructs; i.e. with/without ABD and different linkers, were also analyzed using Biacore T200. In addition, the effect of albumin on some Z-ABD fusions was also assessed by running the same analysis in the absence and in the presence of human albumin. These data are presented below in Table 7.

TABLE 7
Human C5-binding characteristics for a Z-ABD fusion variant Z06175a
(SEQ ID NO: 753, abbreviated Z) comprised in different constructs.

| | Human | | | |
|---|---|---|---|---|
| Construct | albumin | kₐ (1/Ms) | k_{d} (1/s) | K_{D} (M) |
| Z-GS-ABD094 | − | 7.37 x 10⁵ | 1.06 × 10⁻³ | 1.43 × 10⁻⁹ |
| Z-GS-ABD094 | + | 6.74 × 10⁵ | 9.62 × 10⁻⁴ | 1.43 × 10⁻⁹ |
| Z-Z-GS-ABD094 | − | 5.93 × 10⁵ | 3.74 × 10⁻⁴ | 6.30 × 10⁻¹⁰ |
| Z-Z-GS-ABD094 | + | 6.02 × 10⁵ | 4.67 × 10⁻⁴ | 7.76 × 10⁻¹⁰ |
| Z-GS-ABD094- | - | 8.69 × 10⁵ | 5.75 × 10⁻⁴ | 6.62 × 10⁻¹⁰ |
| GSGGGGSGGGGS-Z | | | | |
| (SEQ ID NO: 777) | | | | |
| Z-GS-ABD094- | + | 6.55 × 10⁵ | 3.83 × 10⁻⁴ | 5.86 × 10⁻¹⁰ |
| GSGGGGSGGGGS-Z | | | | |
| (SEQ ID NO: 777) | | | | |
| Z-Z-GSGGGGSGGGGS- | - | 4.59 × 10⁵ | 6.32 × 10⁻⁴ | 1.38 × 10⁻⁹ |
| ABD094 (SEQ ID NO: 778) | | | | |
| Z-Z-GSGGGGSGGGGS- | + | 8.32 × 10⁵ | 9.39 × 10⁻⁴ | 1.13 × 10⁻⁹ |
| ABD094 (SEQ ID NO: 778) | | | | |
| Z-GS | - | 2.42 × 10⁶ | 1.40 × 10⁻³ | 5.79 × 10⁻¹⁰ |
| Z-GSGGGGSGGGGS- | - | 3.64 × 10⁵ | 1.37 × 10⁻³ | 3.75 × 10⁻⁹ |
| ABD094 (SEQ ID NO: 779) | | | | |

Surprisingly small effects could be seen when comparing the affinities of the constructs for hC5 (SEQ ID NO:760) in the presence and absence of albumin. This suggests that simultaneous binding of albumin to the ABD moiety of the constructs does not interfere with C5 interaction.

Steady state binding of C5 binding Z variants to C5 coated ECL plates: Steady state binding of C5 binding constructs composed of different Z variants (SEQ ID NO:745 and 748-757), optionally fused to ABD094 (SEQ ID NO:759) in constructs as specified in FIG. 4, to hC5 was assessed in a competition assay. By competing for binding to C5 coated on ECL plates with a SULFO-TAG labeled C5 binding Z variants (SEQ ID NO:748) fused to ABD (SEQ ID NO:759), steady state binding of the C5 constructs was evaluated. As a comparison the tic protein OmCI (SEQ ID NO:761) was also included. The labeled Z-ABD variant containing SEQ ID NO:748 had an affinity (K_{d}) of 0.9 nM for hC5. This labeled Z-ABD variant was further found to bind to an antibody specific for the constant region of Z variants in a concentration-dependent manner with a K_{d} of 0.34 nM.

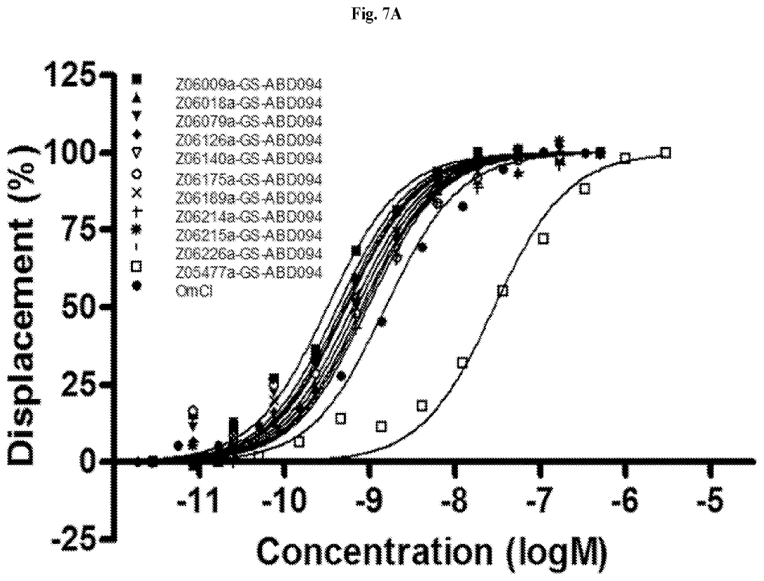

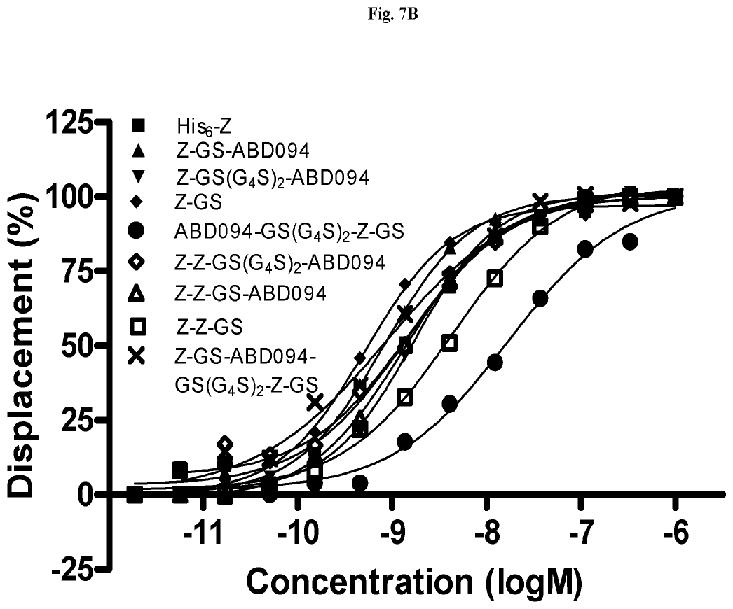

The C5 binding Z-variants (SEQ ID NO:748-757) fused in the carboxy terminus to ABD094 (SEQ ID NO:759) by a GS linker were found to displace 200 pM SULFO-TAG labeled Z-ABD variant with IC₅₀ values ranging from about 300 pM to 1 nM (FIG. 7A), whereas the corresponding construct containing the parental Z variant Z05477a (SEQ ID NO:745) exhibited an affinity IC₅₀ value of about 30 nM. In contrast, the naturally occurring C5 binding protein OmCI was found to bind hC5 with an IC₅₀ of 1.5 nM (FIG. 7A). Thus, all the tested second-generation Z variants (SEQ ID NO:748-757) exhibited a higher binding affinity for human C5 than the parental Z variant Z05477a (SEQ ID NO:745). In addition, the affinities were higher than that of OmCI binding to human C5 using the same method.

A number of different constructs containing the same C5 binding domain as a monomer, dimer, with or without ABD as well as a few different linkers between the different domains were also tested (FIG. 7B). Monomeric variants of Z06175a (SEQ ID NO:753, optionally fused to a His₆-tag or a C-terminal ABD) and the dimeric variants with a C-terminal ABD linker were found to displace 200 pM SULFO-TAG labeled Z-ABD variant with IC₅₀ values ranging from about 500 pM to 1.7 nM whereas the dimeric variant without an ABD and the monomeric variant with a N-terminal ABD displaced 200 pM SULFO-TAG labeled Z-ABD with IC₅₀ values of 4 nM and 17 nM, respectively.

Selectivity: Selectivity was addressed using SPR analysis and the surface with the immobilized Z-ABD variant (SEQ ID NO:748 fused to SEQ ID NO:759 by a GS-linker) displayed no significant SPR signal when subjected to 40 and 400 nM of the C5 paralogs human C3 and C4 as well as human IgG. As a comparison, 400 nM human C5 elicted an SPR response of about 450 RU showing that the tested Z-ABD variant indeed is selective for C5 over C3, C4 and IgG.

### Example 7: Interaction Studies of Z-ABD Variants with HSA, BSA and Serum Album from Rat and Mouse

### Materials and Methods

Two different methods, size exclusion chromatography and Biacore, were used to study the interaction between the albumin binding domain ABD094 fused to a C5 binding Z variants.

Size exclusion chromatography (SEC) was employed to study the interaction between Z06175a-GS-ABD094 (SEQ ID NO:753 fused to SEQ ID NO:759 by a GS linker) and HSA. Briefly, equimolar amounts of Z06175a-GS-ABD094 and recombinant HSA (Novozymes) were preincubated in PBS at room temperature for 60 minutes and subsequently run on a Superdex200 column (GE Healthcare) using the SMART system (GE Healthcare). Z06175a-GS-ABD094 and HSA were also run separately as controls.

Binding to immobilized albumin was studied using a Biacore 2000 instrument (GE Healthcare). Recombinant human albumin (Recombumin®, Novozymes) was coupled to a CM5 sensor chip (385 RU) using amine coupling chemistry as described by the manufacturer. The coupling was performed by injecting human albumin in 10 mM Na-acetate buffer pH 4.5 (GE Healthcare). The reference cell was treated with the same reagents but without injecting human albumin. Injection of HBS-EP was also included as control so that the sensorgrams were double blanked. Experiments were performed in HBS-EP buffer, 10 mM glycine-HCl pH 2 (GE Healthcare) was used for regeneration, the flow rate was 30 μl/min and data were collected at 25° C. Two different constructs were tested, Z-ABD (Z06175a-GS-ABDO94) and Z-ABD-Z (Z06175a-GS-ABDO94-GSGGGGSGGGGS-Z06175a) (SEQ ID NO: 780) at three different concentrations; 25 nM, 100 nM and 400 nM. BIAevaluation version 4.1.1 was used for evaluation of sensorgram data. In a similar fashion, binding of Z-ABD (Z06175a-GS-ABDO94) to surfaces immobilized with serum albumin from rat (A4538, Sigma), mouse (A3559, Sigma), and cow (BSA, Sigma) was also investigated.

### Results

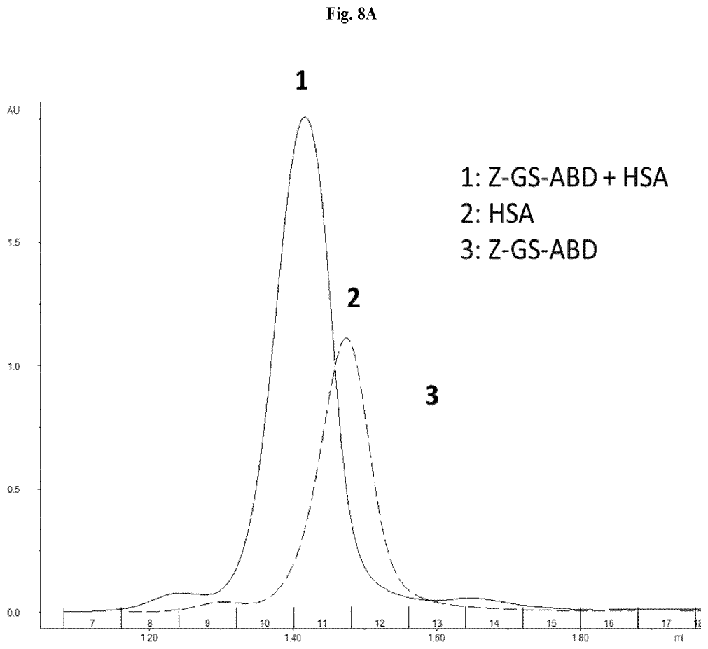

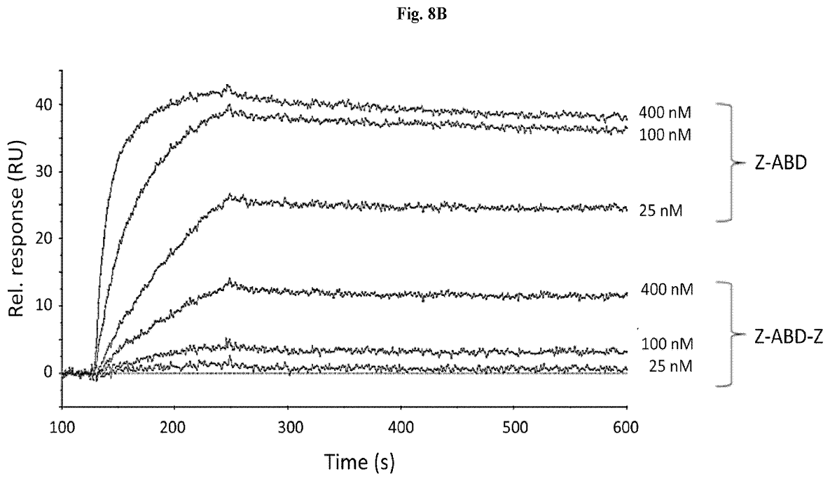

On a SEC column, larger molecules elute faster than small. As seen in FIG. 8A, the co-injected HSA+Z06175a-GS-ABD094 elute faster than when HSA is injected alone suggesting that the two molecules behave as a stable complex under these conditions. The smaller Z06175a-GS-ABD094 elute slower than either the complex or HSA alone showing that these proteins alone are smaller than the complex.

Biacore 2000 data for the analyzed Z-ABD and Z-ABD-Z variants show that the Z-ABD has a faster on-rate than when ABD is flanked by Z-domains on either side (FIG. 8B). Analysis of the binding affinity of ABD fused Z domains points at an affinity below 1 nM for Z-ABD whereas the Z-ABD-Z variant bind to immobilized HSA with a K_{D} above 1 nM.

Z06175a-GS-ABD094 bound to rat serum albumin with very high affinity (KD<100 pM) whereas the interaction with immobilized mouse serum albumin was weaker (KD of about 4 nM) than both with human and rat serum albumin. Interaction with bovine serum albumin was not measureable.

These data agree well with published data on an earlier variant of ABD (Jonsson et al. Protein Engineering, Design & Selection 2008, 21: 515-527) and show that the tested Z-ABD variant is strongly bound to serum albumin in human at clinically relevant concentrations as well as in mouse and rat allowing comparisons of pharmacokinetic data between animals and humans.

### Example 8: Pharmacokinetic Studies of C5 Binding Z Variant in Rats

### Materials and Methods

Rodent in-life phase: The pharmacokinetics of two C5 binding constructs Z-ABD (Z06175a-GS-ABD094; SEQ ID NO:753 fused to SEQ ID NO:759 by a GS linker, FIG. 4, construct 2) and Z-ABD-Z (Z06175a-GS-ABDO94-GSGGGGSGGGGS-Z06175a (SEQ ID NO: 781); (SEQ ID NO:753 fused to SEQ ID NO:759 by a GS linker, followed by a GS(G₄S)₂ linker and a second SEQ ID NO:753 motif, FIG. 4, construct 5) was studied in Male Sprague Dawley (SD) rats (250-300 g body weight). Each rat was given a single dose administration, i.v. (250 nmol/kg) or s.c. (500 nmol/kg), of Z-ABD or A-ABD-Z (n=3 per dose group). Blood samples (200 μL) were drawn at 5, 20, and 45 min, as well as 1.5, 4, 7, 24, 48, 72, 120, 168, 240, and 336 h following administration for the i.v. group and at 15 and 30 min, 1, 2, 4, 7, 24, 48, 72, 120, 168, 240, and 336 h following administration for the s.c. group. Blood was collected in tubes and placed in the fridge for 20 min to allow clotting. Serum was subsequently harvested following centrifugation at 4000 rpm for 10 minutes. Serum samples were kept at −70° C. pending analysis.
Determination of C5 binding Z variant concentrations in serum samples from animals using LC/LC/MS/MS: Serum concentrations of the administrated C5 binding constructs Z-ABD and Z-ABD-Z, as described above, were determined by mass spectrometry (LC/LC/MS/MS).

Serum or plasma samples (25 μl) were diluted with 150 μl of a pepsin agarose (7 mg/ml, Sigma, cat. no. P0609) suspended in 1 M ammonium formate buffer pH 3.0 in a 500 μl Eppendorftube. The tubes were capped and agitated in an Eppendorf thermomixer compact at 37° C. for 20 min. Following agitation, 25 μl of an internal standard solution I(¹³C₆;¹⁵N)NKLDDDPSQSSEL (SEQ ID NO: 782) (amino acids 31-44 of the SEQ ID NO:746-757) (Thermo Fisher Scientific GmbH), diluted to 0.5 μM in 0.1% trifluoroacetic acid (TFA), was added. Following addition of internal standard, the samples were mixed and filtered through 0.45 μm cellulose spin filters (Grace).

Standard samples for calibration were prepared by weighing 20 μl of protein stock solution with known protein concentration (5-10 mg/ml) followed by dilution with blank plasma from the species to be analyzed. The first stock plasma standard (3 μM) was diluted further down to 0.1 μM.

40 μl of the samples were injected into a coupled column system followed by tandem mass spectrometry with multiple reaction monitoring (MRM). The first column was an Ascentis RP-Amide column packed with 5 μm particles (2.1×150 mm, Supelco). An enrichment column; a Brownlee newgard column (3.2×15 mm) packed with 7 μm C18 particles, was used to trap the analyte peptide fraction from the first column. The effluent from the first column was diluted with 1 ml/min water pumped by Shimadzu pump into a whirl mixer (Lee Scientific). The last column was a mixed mode reversed phase and cation exchange column (2.1×100 mm) packed with 5 μm particles Primesep 100 (SIELC Inc).

The mobile phases for the first column (RP-Amide) provided on a first liquid chromatograph (Acquity UPLC) were A: 2% acetonitrile, 0.1% acetic acid, 0.1% TFA, and 97.8% water, and B: acetonitrile with 0.1% acetic acid and 0.02% TFA. The flow was 0.5 ml/min and a linear gradient was used for elution. The sample was eluted at isocratic conditions with 100% A for 1 min, followed by 80% A at 7.9 min. At 8.1 min, the column was washed with 100% B for one minute, followed by reconditioning with 100% A. The effluent from the column was connected to a Valco six port valve controlled from the mass spectrometer software.

The trap column (3.2×15 mm) was connected to the six port valve in back flush mode. The mobile phases for the second column, provided on a second liquid chromatograph (Agilent 1100), were A: 80% acetonitrile, 19.9% water, and 0.1% formic acid, and B: 80% acetonitrile, 19% water, 0.5% acetic acid and 0.5% TFA pumped by an Agilent 1100 liquid chromatogmph at 0.5 ml/min and eluted with the following gradient: 100% A during the first 5 minutes followed by B gradually being raised from 0 to 40% from 5 to 10 minutes followed by a raise to 100% B during the next 6 seconds (10 to 10.1 minutes). B was kept at 100% until 11.5 minutes followed by a drop to 0% (100% A) during the next 6 seconds (11.5 to 11.6 minute) and kept at 0% B throughout the cycle until stopped at 13 minutes.

The effluent from the last column was connected to a triple quadrupole mass spectrometer (Sciex API 4000) equipped with an electrospray ion source operated in positive ion mode. The MRM transitions were 780.9>814.4 for the analyte and 784.5>821.4 for the internal standard. The declustering potential was optimized at 55 V and the collision energy to 35 V. The effective collision energy was 70 eV since the precursor ion was doubly charged giving a singly charged fragment ion. The peak area ratios between the analyte and internal standard were used for quantification. Linear calibration curves were obtained with a recovery of 85% and a limit of quantification of about 40 nM.

Ex vivo hemolysis: An ex vivo hemolytic assay for complement activation was performed in order to optimally assemble in vivo conditions for the serum samples from the above described in vivo studies. The serum samples were 5× diluted in a total reaction volume of 25 μl/well comprising 5 million antibody sensitized sheep erythrocytes (EA). In general, a portion of 20 μl EA suspension containing all other components (see Example 6) was mixed (agitation 10 minutes) with 5 μl serum sample to initiate the hemolytic activation at 37° C. For mouse serum samples, such as in example 11, however, 1 μl C5D had to be included in the 20 μl EA suspension. The ex vivo assay was performed essentially as described for the in vitro assay of Example 6. Calculations: Evaluation of the pharmacokinetic parameters was based on individual serum concentration data, the mean (±stdev) is reported for each dose group. Levels below lower limit of quantitation (LLOQ) appearing at terminal sampling points were omitted from the pharmacokinetic analysis. Maximum serum concentration, Cₘₐₓ, and time to observed maximum serum concentration, tₘₐₓ, were obtained directly from the serum concentration data. The pharmacokinetic parameters; area under curve (AUC, AUC_{0-∞} and AUC₀₋ₗₐₛₜ calculated by the linear trapezoidal method), subcutaneous bioavailability (F, calculated as (AUC_{sc}/AUCᵢᵥ)*(Doseᵢᵥ/Dose_{sc})), terminal serum half-life (T_{1/2,z}, calculated as ln 2/λ_{z} where estimation of terminal slope, λ_{z}, was based on at least 4 C=f(t) observations), mean residence time (MRT, calculated as AUMC/AUC), serum clearance (CL, calculated as Dose/AUC_{0-∞}), volume of distribution at steady state (Vₛₛ, calculated as CL*MRT) and volume of distribution at the terminal phase (V_{z}, calculated as CL/λ_{z}) were calculated using WinNonlin software version 5.2.1 (Pharsight Corp., USA), Non-Compartmental-Analysis.

### Results

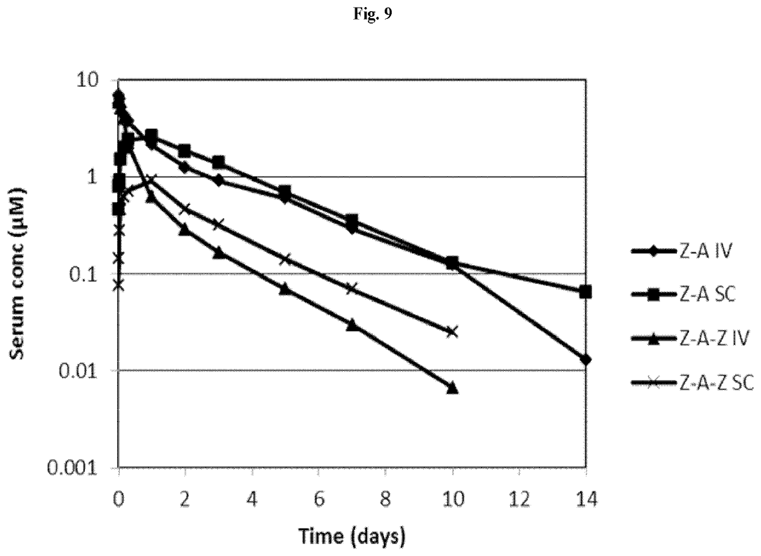

The pharmacokinetic data for Z-ABD and Z-ABD-Z following i.v. (250 nmol/kg) and s.c. (500 nmol/kg) administration are summarized in Table 8. Z-ABD was quantifiable in serum up to 10-14 days post dose in the i.v. group and 14 days in the s.c. group whereas Z-ABD-Z was quantifiable in serum up to 10 days post dose in both dose groups (FIG. 9). 14 days was the final sampling time point.

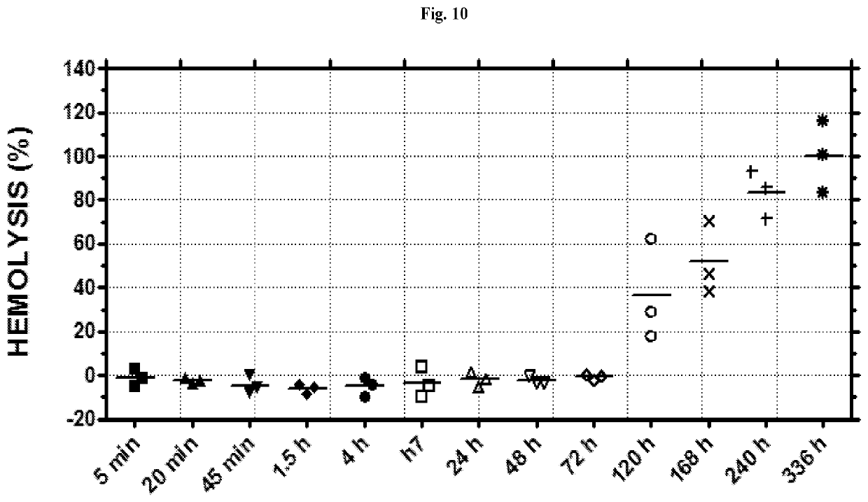

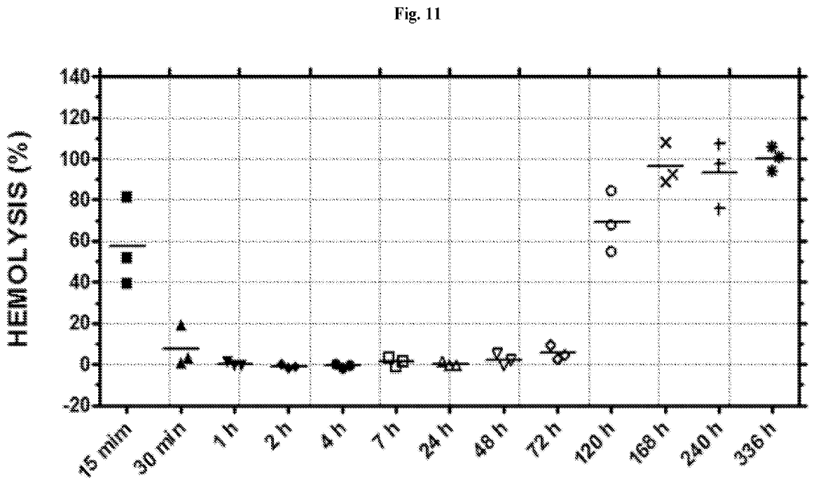

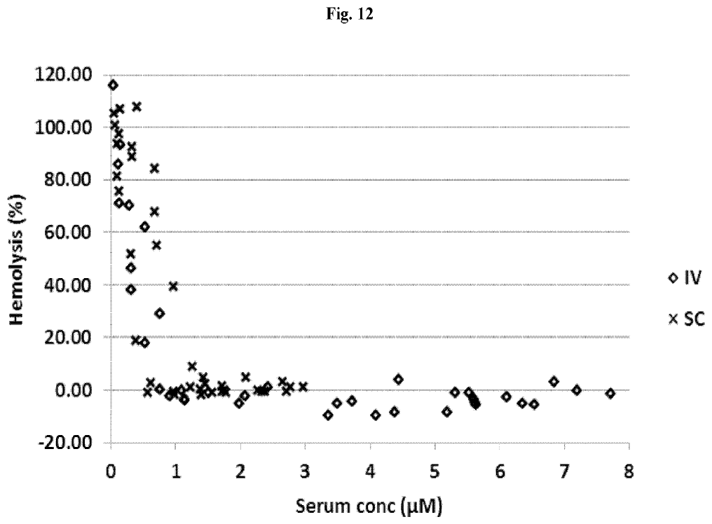

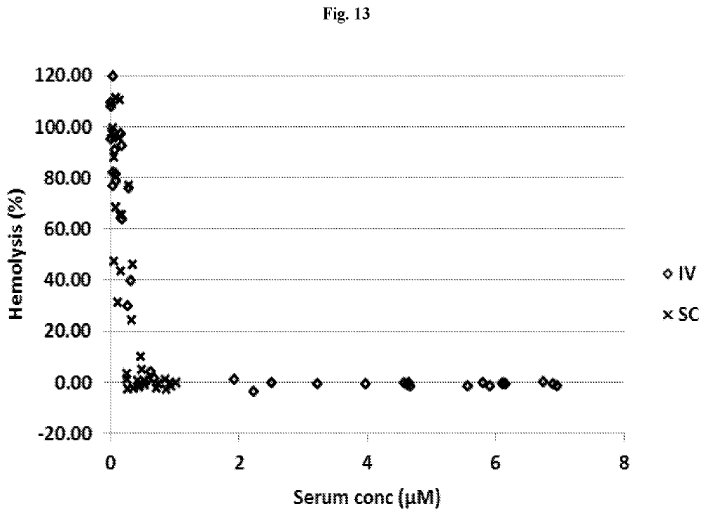

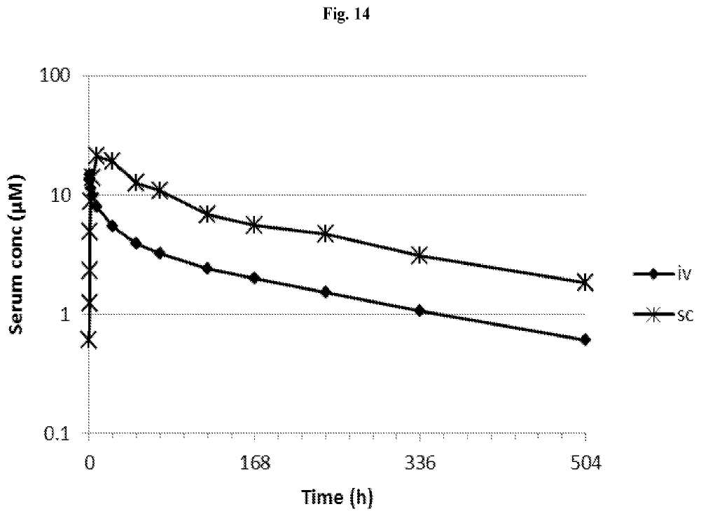

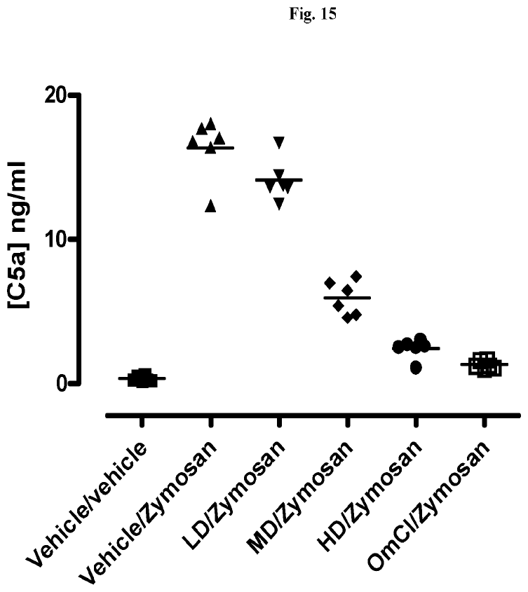

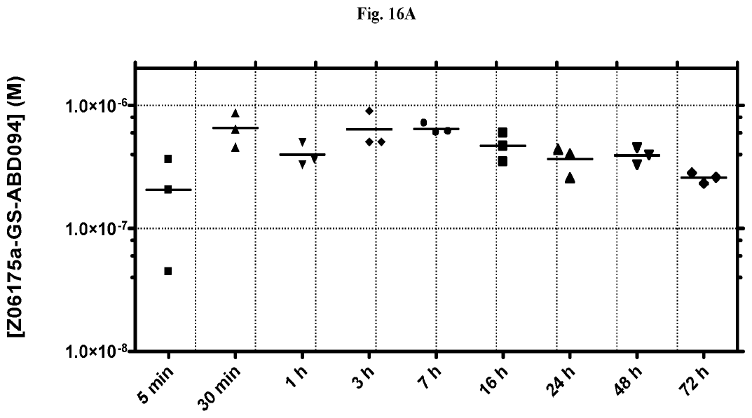

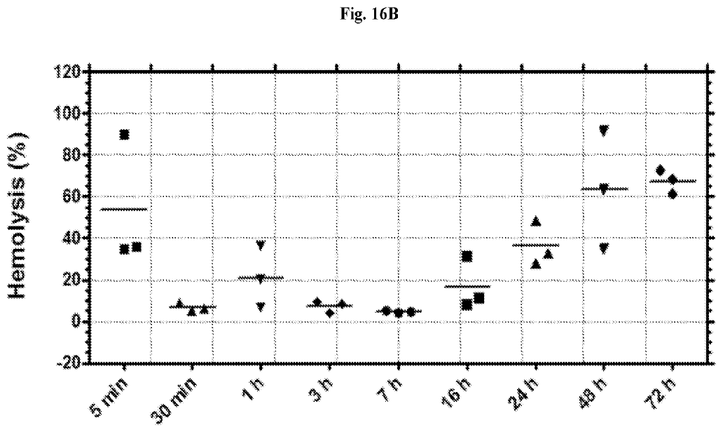

Correlating the serum concentration of C5 binding polypeptide with the amount of hemolysis in sheep erythrocytes, it was found that full inhibition of hemolysis under the conditions described (e.g. serum dilution 1:5) was obtained by Z-ABD at serum concentrations above 1 μM (FIG. 12) whereas Z-ABD-Z reached full inhibition at serum concentrations around 0.5 μM (FIG. 13). Surprisingly, as seen in FIG. 9 and Table 8, Z-ABD has a lower serum clearance, a longer terminal serum half-life and a higher bioavailability than Z-ABD-Z. In terms of time this lead to full inhibition of hemolysis for about three days after administration of 250 nmol/kg Z-ABD (FIG. 10) i.v. or 500 nmol/kg s.c (FIG. 11) to S.D.rats.

TABLE 8
Mean (±stdev) pharmacokinetics of Z-ABD and Z-ABD-Z following
i.v. and s.c. administration in male Sprague Dawley rats.

| | | Z-ABD | | | | Z-ABD-Z | | | |
|---|---|---|---|---|---|---|---|---|---|
| Administration route | | i.v. | | s.c. | | i.v. | | s.c. | |
| Dose | nmol/kg | 250 | | 500 | | 250 | | 500 | |
| Cₘₐₓ | μM | | | 2.8 | (0.2) | | | 0.90 | (0.10) |
| Tₘₐₓ | h | | | 18 | (9.8) | | | 17 | (12) |
| AUC_{0-∞} | μM*h | 233 | (34) | 252 | (11) | 79 | (7.5) | 64 | (1.2) |
| AUC₀₋ₗₐₛₜ | μM*h | 226 | (37) | 247 | (11) | 79 | (6.9) | 63 | (1.0) |
| F | % | | | 55 | (3.1) | | | 41 | (2.6) |
| T_{1/2, z} | h | 58 | (4.6) | 57 | (4.2) | 36 | (0.6) | 46 | (1.2) |
| MRT | h | 69 | (2.6) | 80 | (4.6) | 27 | (1.5) | 63 | (2.6) |
| CL | mL/h*kg | 1.1 | (0.2) | | | 3.2 | (0.2) | | |
| Vₛₛ | mL/kg | 73 | (12) | | | 83 | (10) | | |
| V_{z} | mL/kg | 90 | (18) | | | 159 | (12) | | |

### Example 9: Pharmacokinetic Studies of C5 Binding Z Variants in Monkey

### Materials and Methods

The study in life phase was performed at Charles River, Nevada (www.criver.com), formulation of administered drug and analysis of serums samples were performed in house. The pharmacokinetics of a Z-ABD variant (Z06175a (SEQ ID NO:753) fused to ABD094 (SEQ ID NO:759) by a GS linker) was investigated in the male Cynomolgus monkey (n=3) following i.v. (intravenous) and s.c. (subcutaneous) administration. Evaluation of the pharmacokinetic parameters was performed according to Example 8, however following i.v. administration the initial serum half-life (T_{1/2α}) corresponding to the initial slope of the log-linear serum concentration-time curve, intermediate serum half-life (T_{1/2β}) corresponding to the slope of the log-linear serum concentration-time curve associated with the secondary (intermediate) phase and terminal serum half-life (T_{1/2γ}) corresponding to the terminal slope of the log-linear serum concentration-time curve was determined. T_{1/2} was calculated as ln 2/λ where estimation of the slope, λ, was based on at least 4 C=f(t) observations. The pharmacokinetic data presented for sc administration are compensated for pre-dose levels of Z-ABD while the graph displaying serum concentration versus time after sc administration show the actual serum concentrations determined. The monkeys were 2-4 years old with a body weight of 2.3-3 kg. Each monkey received a single i.v. dose (540 nmol/kg) followed by a single s.c. dose (1635 nmol/kg) three weeks after the i.v. administration. Blood samples were taken at 10 and 30 minutes and 1, 2, 4, 8, 24, 48, 72, 120, 168, 240, 336 and 504 hours post dose following both administrations. The blood samples were allowed to clot for 20-40 minutes in room temperature and then centrifuged at 1500 to 2200 RCF at 2-8° C. for 10-15 minutes before the serum was harvested and frozen.

The serum samples were stored at a temperature below −20° C. until analysis. Serum concentrations of Z-ABD were analyzed by LC/LC/MS/MS as described in Example 8. Serum concentrations determined by LC/LC/MS/MS were also confirmed by a quantitative sandwich enzyme immunoassay technique. A polyclonal antibody specific for the Z compartment of Z-ABD was coated on to a microplate. Unbound polyclonal antibody was washed away and casein was added as blocking agent to reduce unspecific binding to the plastic surface. Samples and standards were diluted in PBS containing 0.5% casein and between 1-5% monkey normal serum. After washing away unbound casein, standards and samples were pipetted to the wells allowing any Z-ABD, presumed mainly to be associated with serum albumin, present in the sample to bind to the immobilized antibody. After washing away any unbound Z-ABD, an HRP labeled polyclonal antibody specific for albumin was added to detect the immobilized Z-ABD-albumin complex by colorometric methods. Unbound polyclonal antibody was washed away and a substrate solution was added to the wells and color develops in proportion to the amount of Z-ABD bound. Evaluation and calculation of pharmacokinetic parameters were performed as described in Example 8.

Ex vivo hemolysis in serum from cynomolgus monkeys dosed with above described Z-ABD variant was monitored using the method described in Examples 6 and 8 with the modification that the monkey serum was diluted only two-fold compared to five-fold for rodent serum.

### Results

Data on the mean (±stdev) pharmacokinetics of each dose group are presented. Serum concentrations of Z-ABD were quantifiable at all time points following both i.v. and s.c. administration by LC/LC/MS/MS (FIG. 14). ELISA data and LC/LC/MS/MS data correlated linearly by a coefficient of 0.986 but LC/LC/MS/MS data were used for the calculations. Following i.v. administration of Z-ABD the initial serum half-life was 9.1 (0.8) hours, intermediate serum half-life was 84 (4) hours and the terminal serum half-life was 198 (51) hours. The mean residence time was 246 (62) hours. The volume of distribution, Vₛₛ and V_{z} was calculated to 110 (23) ml/kg and 127 (27) ml/kg respectively and clearance was estimated to 0.45 (0.02)mL/h*kg.

Following s.c. administration, and corrected for pre-dose serum levels remaining from the i.v. administration, maximum serum concentrations (mean Cₘₐₓ 21(3) μM) were reached at 8-24 h after dose. The terminal serum half-life was 206 (40) hours and the mean residence time was 250 (68) hours. The subcutaneous bioavailability was estimated to be above 70%.

The pharmacodynamic effect of the injected Z-ABD variant (Z06175a (SEQ ID NO:753) fused to ABD094 (SEQ ID NO:759) by a GS linker) was monitored by hemolysis. The hemolytic effect in cynomolgus monkey was completely suppressed (≤20% of pre-dose) for at least seven days after administration of 5 mg/kg Z-ABD i.v. and 15 mg/kg Z-ABD s.c.

### Example 10: In Vivo Studies Using Zymosan Induced Peritonitis

### Materials and Methods

Administration to mice: C57BL/6 female mice received different concentrations of a Z-ABD fusion molecule (Z06175a-GS-ABDO94, SEQ ID NO:753 fused to SEQ ID NO:759 by a GS linker) or the positive control OmCI intraperitoneally (i.p.) 1 hour before induction with zymosan, or subcutaneously (s.c.) 18 hours before induction with zymosan. 0.8 mg/mouse zymosan was administered i.p. 1 hour later orbital blood samples (in serum vials with coagulation activator) were taken under isoflurane anaesthesia. The animals were killed by cervical dislocation. A skin incision was made, and the abdominal muscular wall was visualized. PBS solution (including 2 mM EDTA) was gently injected into the abdominal cavity. The abdomen was massaged and a sample of fluid (1-2 ml) was withdrawn. The samples were transferred to test tubes and stored on wet ice before centrifugation at 600 g for 10 min. Total protein and C5a concentrations in the supernatant were analyzed.

Blood samples were kept in a refrigerator for at least 30 min and centrifugation was thereafter performed at 2000 g. Serum samples were stored in freezer (−70° C.) for later analysis of hemolytic activity and levels of Z06175a-GS-ABD094.

Analysis of hemolysis activity in serum samples from animals: Analysis of hemolysis activity was performed according to the hemolysis assay described in Examples 6 and 7.
Analysis of C5a concentration in lavage from mice dosed with zymosan and C5 binding Z-ABD fusion molecules: For detection of C5a in mouse peritoneal lavage samples, microtiter plates (MaxiSorp, Nunc) were coated overnight at 4° C. with 100 μl/well of anti-C5a antibody (cat. no. MAB21501, R&D Systems) at a concentration of 1 μg/ml in 0.05 M sodium carbonate-bicarbonate buffer, pH 9.6 (cat. no. C-3041, Sigma). The plates were washed three times with PBS containing 0.05% Tween 20 (PBST, cat. no. 09-9410-100, Medicago) and blocked with 200 μl/well of 1% BSA (cat. no. A7030, Sigma) in PBST for 1-1.5 h at RT during agitation at 450 rpm. The plate was again washed three times with PBST and then incubated with 100 μl/well of recombinant mouse C5a standard (cat. no. 2150-C5, R&D Systems) at various concentrations in PBST with 0.1% BSA or samples for 2 h at RT during agitation at 450 rpm. High concentration samples were also diluted in PBST with 0.1% BSA. The plate was once again washed three times with PBST and then incubated with 100 μl/well of biotinylated anti-C5a antibody (cat. no. BAF2150, R&D Systems) at a concentration of 0.1 μg/ml for 1.5 h at RT while shaking the plate at 450 rpm. Following 3× washing with PBST, the plate was incubated with 100 μl/well of streptavidin-HRP (cat. no. DY998, R&D Systems) at a 200 fold dilution in blocking buffer for 20 min at RT during agitation at 450 rpm. After three final washes, the plate was developed with 100 μl/well TMB substrate (cat. no. T0440, Sigma) and read after 20-30 min at 650 nm using a Spectramax Plus plate reader (Molecular Devices).

A standard curve was constructed by plotting the absorbance at 650 nm for each standard against its concentration (range 04000 μg/ml).

Determination of Z variant concentration in serum samples from animals using ECL: Serum concentrations of administrated C5 binding Z06175a-GS-ABD094 (SEQ ID NO:753 fused to SEQ ID NO:759 by a GS linker) and Z06175a-GS-ABD094-GSGGGGSGGGS-Z06175a (SE ID NO; 7890 9SEQ ID NO:753 fused to SEQ ID NO:759 by a GS linker, followed by a GS(G₄S)₂ (SEQ ID NO:785) linker and a second SEQ ID NO:753 motif, see FIG. 4 for construct description) were determined by ECL. Multi-array 96-well high-bind, non-coated (Meso Scale Discovery cat. no. L15XB) plates were coated with a goat anti-Affibody molecule Ig (Affibody AB, cat. no. 20.1000.01.0005).

In similarity with Example 6, a Z-ABD variant (Z06009a, SEQ ID NO:748 fused to ABD094, SEQ ID NO:759 Multi-array plates were coated with the goat anti-Affibody molecule IgG (Affibody AB) overnight at 4° C., and subsequently non-specific sites were blocked with PBS with 1% Casein for two hours at RT.

Meanwhile, serum samples were thawed from −70° C. and diluted in PBS with casein in serum from the same animal strain. Standards and controls were diluted in the corresponding buffer. Samples and standards were incubated for three hours at RT while shaking the plate at 300 rpm. Incubation was terminated by washing 3×150 μL ice-cold PBS-Tween20. Immediately after the final wash, 150 μl 2× reading buffer (4× reading buffer T, Meso Scale Discovery cat. no. R92TC-3 diluted 1:1 in ultrapure H₂O) was added to each well and the signal was detected using a plate reader (SECTOR Imager 2400, Meso Scale Discovery).

In an alternative experiment, plates were coated with human C5 (SEQ ID NO:760, 1 μmol/well). Prior to addition to the coated plate, serum samples and standards, diluted in serum or in serum and PBS with casein (all samples and standards were matched to the same serum concentration), were heated to 60° C. for 30 min in order to denature endogenous C5. This alternative experiment provided a method for exclusive detection of C5 binding proteins, whereas the antibody dependent strategy described above can be applied to all proteins binding to that particular antibody.

### Results

Analysis of serum concentrations of Z-ABD and hemolysis activity in serum samples from animals: The serum concentrations as well as the ability to affect hemolysis in sheep erythrocytes of the Z-ABD fusion molecule (Z06175a-GS-ABD094, SEQ ID NO:753 fused to SEQ ID NO:759 by a GS linker)) was assessed after administration of a low (20 nmol/kg), medium (100 nmol/kg) and high dose (500 nmol/kg). The serum concentrations were relatively linear with dose, and inhibition of hemolysis confirmed that the molecules in serum were active and that the inhibition of hemolysis indeed also was concentration dependent.
Analysis of C5a concentration in lavage from mice dosed with zymosan and C5 binding Z-ABD fusion molecules: The pro-inflammatory molecule zymosan was administered i.p. and in FIG. 15 the effect on the highly inflammatory C5 cleavage product C5a in lavage as a function of zymosan dosing alone and zymosan dosed after a dosing of a C5 binding Z variant at 20, 100 and 500 nmol/kg administered s.c. 18 h before zymosan treatment or OmCI administered i.p. 1 h before zymosan treatment, is shown. Zymosan administration alone leads to a potent elevation of C5a in the lavage. This effect is blocked in a dose dependent manner by the presented C5 binding Z-ABD fusion molecule.

### Example 11: Pharmacokinetic Studies of C5 Binding Protein in Mice Following Intratracheal Administration

### Materials and Methods

The pharmacokinetic profile of the C5 binding construct Z06175a-GS-ABD094 (SEQID NO: 753 fused to SEQ ID NO:759 by a GS linker) following intratracheal administration to female C57bl mice was studied. Temperature, relative humidity and lighting was set to maintain 22±1° C., 55±5% and a 12 h light-12 h dark cycle and diet and water was provided ad libitum. Animals were anesthetized with isoflurane and dosed directly into the lungs using a microspray with 500 nmol/kg Z06175a-GS-ABD094. As much blood as possible was drawn, under anesthesia by isoflurane, from vena cava at 5 min, 30 min, 1 h, 3 h, 7 h, 16 h, 24 h, 48 h and 72 h (three animals/time point) for preparation of serum samples. Serum samples were prepared by collecting blood in tubes and placing the tubes in the fridge for 20 min. Subsequently, the tubes were centrifuged at 4000 rpm for 10 minutes. A minimum of 100 μl serum was prepared from each blood sample. Serum samples were kept at −70° C. prior to analysis. Serum concentrations of Z06175a-GS-ABD094 in each sample was determined by ECL as described in Example 10 and the ability of serum samples to affect hemolysis in sheep erythrocytes was determined as described in Examples 6 and 8.

### Results

The serum concentration in each sample and the corresponding ability to affect hemolysis in sheep erythrocytes are described in FIG. 16A and FIG. 16B, respectively. Within 30 minutes, a plateau is reached with serum concentrations ranging from 300 to 1000 nM where hemolysis is nearly completely blocked. In serum sampled at time points later than 7 h post-administration, hemolysis is gradually reoccurring. At the final time point three days after dosing, hemolysis was about 70% of control (FIG. 16B). These data clearly demonstrate absorption of Z06175a-GS-ABD094 into the systemic circulation following intratracheal administration and that the molecule functionally inhibits hemolysis.

### Example 12: Pharmacokinetic Studies of C5 Binding Z Variant in Rabbit Eve Following Topical and Intravitreal Administration

### Materials and Methods

Rabbit in-life phase: The pharmacokinetics of a Z variant (Z06175a, SEQ ID NO:753 followed by GS (FIG. 4, construct 1)) was studied in rabbit eye following intra-vitreal administration.

The study in-life phase and dissection of eyes from dosed animals (pigmented rabbits, 2-2.5 kg) was performed at Iris Pharma, La Gaude, France (www.iris-pharma.com). Animals were housed individually at 20±2° C. at 55±10% relative humidity with access to food and water adlib.

Animals were divided in three groups: 1) intravitreal administration (50 μl in each eye, n=3, six eyes totally) followed by dissection and serum sampling after one day, 2) intravitreal administration (50 μl in each eye, n=3) followed by dissection and serum sampling after four days and 3) untreated animals (n=5).

Four distinct eye compartments were dissected (aqueous humor, vitreous, neuro-retina and RPE-choroid) and immediately frozen at −80° C. Formulation of administered drug (20.2 mg/ml in 10 mM phosphate buffer, 145 mM NaCl, pH 7.4) and analysis of drug in various eye compartments were performed in house.

Analysis of Z-variant in dissected eve compartments: Dissected eye compartments were shipped on dry ice and stored at −80° C. until analysis. The retina and choroid samples were thawed in 10 times (volume/weight) PBS containing 1% human serum albumin in Lysing Matrix D tubes (MP Biomedical) containing ceramic beads and agitated at speed 4 for 2×20 s in a Savant Bio 101 homogenizer. The homogenate was removed from the beads using a pipette and transferred to a 1.5 ml Eppendorf tube and centrifuged at 900 rpm for ten minutes. The aqueous humor and vitreous samples were treated the same way as retina and choroid with the exception that no homogenization was needed. The vitreous samples from groups one and two were diluted 10 times further in the same buffer as above. Five standards were prepared in PBS with HSA (35.8 μM, 3.58 μM, 358 nM, 35.8 nM and 17.9 nM). Subsequently, standards and samples were subjected to pepsin digestion and analysis of the concentration of Z variant in tissue extracts was determined using the LC/LC/MS/MS method described in Example 8.

### Results

The concentrations of Z variant after intravitreal administration were high in all compartments after one day (6-200 μM) and, surprisingly, remained high 4 days post-administration (1.5-78 μM). In particular, the concentration of the Z molecule in the vitreous ranged from 118 to 201 μM (average 161 μM, n=6 eyes) one day after injection and remained at 26 to 78 μM (average 46 μM, n=6) four days post-injection, pointing at a T_{1/2} of several days. There appears to be an inverse relationship between size and elimination of drugs after intravitreal injection in rabbit eye described by the following examples; Moxifloxacin (MW<0.35 kDa, T1/2=1.72 h, Mohan et al. Trans Am Ophthalmol Soc 2005, 103:76-83), ESBA105 (MW=26 kDa, T1/2=25 h, Ottiger et al. Investigative Ophthalmology & Visual Science 2009, 50: 779-786) and Ranibizumab (MW=48 kDa, T1/2=2.88 days, Bakri et al. American Academy of Ophthalmology 2007, 114:2179-2182). The Z variant tested here had a molecular weight of 7.0 kDa, suggesting that the elimination of the Z molecule was slower than what would be expected for such a small molecule in vitreous.

The invention claimed is:

1-110. (canceled)

111. A method of detecting C5 in a sample, the method comprising:
(a) contacting the sample with a C5 binding polypeptide comprising a C5 binding motif, wherein the C5 binding motif comprises SEQ ID NO. 763, and
(b) detecting binding of the C5 binding polypeptide to the sample.

112. A multimeric C5 binding polypeptide comprising at least two C5 binding polypeptide monomers, wherein each monomer comprises a C5 binding motif comprising any one of SEQ ID NOs. 1-248.

113. A composition comprising:
(a) a C5 binding polypeptide or C5 binding compound, and
(b) a therapeutic agent, wherein the therapeutic agent is an immunostimulatory agent or a radionuclide.
